(19) United States
(12) Patent Application Publication
(10) Pub. No.: US 2014/0228384 A1
(43) Pub. Date: Aug. 14, 2014

(54) ASSAYS AND BIOMARKERS FOR LRRK2

(71) Applicant: Genentech, Inc., South San Francisco, CA (US)

(72) Inventors: Daisy Bustos, San Mateo, CA (US); Donald Kirkpatrick, San Mateo, CA (US); Tracy Kleinheinz, Daly City, CA (US); John Moffat, San Francisco, CA (US); Zejuan Sheng, San Mateo, CA (US); Shuo Zhang, Belmont, CA (US); Haitao Zhu, Palo Alto, CA (US)

(21) Appl. No.: 13/904,922

(22) Filed: May 29, 2013

Related U.S. Application Data
(63) Continuation of application No. PCT/US2011/062468, filed on Nov. 29, 2011.

(51) Int. Cl. G01N 33/573

(52) U.S. Cl. CPC G01N 33/573; G01N 2430/00; G01N 2500/04
USPC 514/275; 435/15; 435/7.4

(57) ABSTRACT

Biomarkers for screening subjects for Parkinson's disease and providing companion diagnostic tools for therapies using LRRK2 modulators, and assays for screening compounds and compositions for modulation of LRRK2 activity.

### RELATED APPLICATION

This application claims the benefit of International Patent Application No. PCT/US2011/062468 filed 29 Nov. 2013 which claims priority to U.S. Provisional Application Ser. No. 61/418,208 filed on 30 Nov. 2010, which is incorporated by reference in its entirety.

### FIELD OF THE INVENTION

This invention pertains to assays for discovering compounds that modulate the function of LRRK2 and biomarkers for in vivo evaluation of LRRK2 activity.

### BACKGROUND OF THE INVENTION

Neurodegenerative diseases such as Parkinson's disease, Lewy body dementia and Huntington's disease affect millions of individuals. Parkinson's disease is a chronic, progressive motor system disorder that afflicts approximately one out of every 1000 people, with hereditary Parkinson's disease accounting for 5-10% of all of patients. Parkinson's disease is caused by progressive loss of mid-brain dopamine neurons, leaving patients with impaired ability to direct and control their movements. The primary Parkinson's disease symptoms are trembling, rigidity, slowness of movement, and impaired balance. Many Parkinson's disease patients also experience other symptoms such as emotional changes, memory loss, speech problems, and sleeping disorders.

The gene encoding the leucine-rich repeat kinase 2 protein (LRRK2) has been identified in association with hereditary Parkinson's disease (Paisan-Ruiz et al., Neuron, Vol. 44(4), 2004, pp 595-600; Zimprich et al., Neuron, Vol. 44(4), 2004, 601-607). In-vitro studies show that LRRK2 proteins containing Parkinson's disease-associated mutations generally have an increased LRRK2 kinase activity and a decreased rate of GTP hydrolysis compared to the wild-type protein (Guo et al., Experimental Cell Research, Vol. 313(16), 2007, pp. 3658-3670. Anti-LRRK2 antibodies have been used to label brainstem Lewy bodies associated with Parkinson's disease and cortical antibodies associated with Lewis body dementia, suggesting that LRRK2 may play an important role in Lewy body formation and pathogenesis associated with these diseases (Zhou et al., Molecular Degeneration, 2006, 1:17 doi:10.1186/1750-1326-1-17). LRRK2 has also been identified as a gene potentially associated with increased susceptibility to Crohn's disease and susceptibility to leprosy (Zhang et al., New England J. Med. Vol. 361 (2009) pp. 2609-2618.

Accordingly, compounds and compositions effective at modulating LRRK2 activity may provide a treatment for neurodegenerative diseases such as Parkinson's disease and Lewy body dementia and for diseases such as leprosy and Crohn's disease. Particularly, there is a need for assays that enable the identification and evaluation of compounds capable of inhibiting LRRK2 kinase activity in cells. There is also a critical requirement for biomarkers that will enable the efficacy of inhibitors of LRRK2 kinase activity to be evaluated in human patients and in animal models of Parkinson's disease. Finally, there is a continuing demand for techniques and biomarkers which might permit the identification of subjects at risk for Parkinson's Disease (PD) or with Parkinson's disease that may be responsive to treatment with LRRK2 inhibitors. The effectors of LRRK2 activity are unknown, which has impaired the ability of researchers to assess the ability of compounds to inhibit LRRK2 kinase activity in cells or in vivo. (N. Dzamko, 2010, Biochemical J.) Autophosphorylation is known to play an important role in the biological activity of many kinases, and it has been previously established that LRRK2 is capable of autophosphorylation at many sites in vitro. (Gloekner, 2010, J. Proteosome Res.; Kamakikawaji, 2009, Biochemistry). These studies have found that LRRK2 functions primarily as a serine/threonine kinase, and it has been suggested that phosphorylation at threonine sites is preferred. Importantly however, while several dozen autophosphorylation sites have been reported following in vitro studies, the vast majority of these sites are appear not to be phosphorylated in cells (Reference). Furthermore, there has been no report describing a site of autophosphorylation that can be identified in animals. An authentic autophosphorylation site that can be easily monitored in cells and in animals, as well as assays that allow the degree of autophosphorylation to be monitored under various conditions, will be a critical tool in the development of therapeutics targeting LRRK2.

### SUMMARY OF THE INVENTION

Mutations in Leucine Rich Repeat Kinase 2 (LRRK2) gene are the most common cause of familial Parkinson's disease (PD). Mutations in the LRRK2 gene associated with Parkinson's disease modulate autophosphorylation of LRRK2. Monitoring changes in autophosphorylation of LRRK2 thus provides a tool for identifying or evaluating inhibitors of LRRK2 in vitro, and provides a potential biomarker for monitoring LRRK2 activity in vivo.

The invention provides an assay/biomarkercapable of assessing LRRK2 activity in the brain. Quantitative studies by the inventors demonstrated that LRRK2 autophosphorylation on Ser1292 is among the most abundant autophosphorylation events in vitro and is detectable in transfected cells. Phosphospecific antibodies directed against pSer1292 were generated and used to show that LRRK2 familial mutations, including R1441G, Y1699C, G2019S and I2020T, all show increased pSer1292. Autophosphorylation of Ser1292 depends on LRRK2 kinase activity and GTP binding, and most likely occurs in cis. Mutation of Ser1292 to alanine (S1292A) abolished the neuronal toxicity caused by LRRK2 PD mutation in cell-based models. In addition, expression of LRRK2 PD mutant proteins in neurons altered basal autophagy activity. S1292A mutation also partially ameliorates this phenotype. Use of LRRK2 kinase inhibitors show that LRRK2 kinase inhibition effectively blocks S1292 autophosphorylation in cultured cells and in LRRK2 BAC transgenic animals. Accordingly, S1292 autophosphorylation represents a biomarker of LRRK2 kinase activity in vivo and may contribute to the mechanism through which LRRK2 causes Parkinson's disease. The terms “phosphorylation” and “autophosphorylation” are used interchangeably herein.

In one aspect of the invention, there is provided a method for identifying patients or subjects having Parkinson's disease or that are at risk of developing Parkinson's disease, the method comprising determining the level of S1292 phosphorylation or autophosphorylation in said patient, wherein an elevated level correlates with having Parkinson's disease or that are at risk of developing Parkinson's disease.

The method may further comprise obtaining or acquiring a LRRK2-containing sample from the subject.

The method may further comprise measuring, monitoring or otherwise detecting the amount or level of S1292 autophosphorylation in the subject sample. The method may further comprise comparing the amount or level of S1292 autophosphorylation in the subject sample with the amount or level of S1292 autophosphorylation in one or more control samples.

In one aspect of the invention, there is provided a method for predicting the sensitivity of a subject to a LRRK2 modulator, or efficacy of a LRRK2 modulator for treating Parkinson's disease, the method comprising determining the level of S1292 phosphorylation or autophosphorylation in said patient, whereby elevated levels of S1292 phosphorylation or autophosphorylation correlate with efficacy of said inhibitor for treating said disease.

The method may further comprise obtaining or acquiring a first LRRK2-containing sample from a subject.

The method may further comprise administering a LRRK2 modulator to the subject.

The method may further comprise obtaining or acquiring a second LRRK2-containing sample from the subject after administering the LRRK2 modulator to the subject.

The method may further comprise measuring, monitoring or otherwise detecting the amount or level of S1292 autophosphorylation in the first subject sample.

The method may further comprise measuring, monitoring or otherwise detecting the amount or level of S1292 autophosphorylation in the second subject sample.

The method may further comprise comparing the amount or level of S1292 autophosphorylation in the first subject sample with the amount or level of S1292 autophosphorylation in the second subject sample.

The invention also provides a method of selecting a patient to receive a LRRK2 inhibitor comprising measuring a patient's S1292 phosphorylation or autophosphorylation profile, whereby a patient having an elevated S1292 autophosphorylation profile is selected. The term “S1292 profile” refers to the amount of S1292 autophosphorylation in a patient's sample compared to the amount in a control sample(s). The S1292 profile can be expressed in terms of a ratio (e.g. amount of S1292 autophosphorylation in a patient sample or cell divided by amount of S1292 autophosphorylation in a control sample or cell) or a subtraction (e.g. amount of S1292 in a patient sample or cell minus the amount of S1292 autophosphorylation in a control sample or cell). An “increased S1292 profile” refers to a S1292 autophosphorylation profile that is determined to have S1292 autophosphorylation levels that are substantially higher in the than control samples. In one example, a S1292 autophosphorylation profile is about 5% to 100% greater than than in the control sample.

In the subject methods, the LRRK2 modulator may be a compound of formula I or formula II, or one of the LRRK2 modulators disclosed herein.

In one aspect of the invention there is provided a method for identifying subjects having Parkinson's disease or that are at risk of developing Parkinson's disease, the method comprising:

obtaining or acquiring a LRRK2-containing sample from a subject;

measuring, monitoring or otherwise detecting the amount or level of S1292 autophosphorylation in the subject sample; and

comparing the amount or level of S1292 autophosphorylation in the subject sample with the amount or level of S1292 autophosphorylation in one or more control samples.

The method may further comprise determining if the amount or level of S1292 autophosphorylation in the subject sample is elevated with respect to or otherwise greater than that of the control sample(s). If the amount or level of S1292 autophosphorylation in the subject sample is elevated with respect to or greater than that of the control sample(s), the subject may be identified as having Parkinson's disease or as being at risk of developing Parkinson's disease.

By way of example and not of limitation, the LRRK2-containing subject sample may comprise cells, cell lysate, or solutions or suspensions thereof, obtained from tissue or serum from the subject. In one embodiment the subject sample may comprise monocytes (white blood cells) or lysate thereof. The sample may be obtained from the subject via syringe or tissue extraction device. The sample thus obtained may be subject to cell separation, buffering, solvent extraction and/or exposure to lysing enzyme(s) to expose or otherwise make accessible the LRRK2 protein contained therein.

In certain embodiments the measuring of the level of S1292 autophosphorylation in the subject sample may be carried out using an antibody that associates with S1292 (i.e., an anti-phospho-S1292-LRRK2 antibody), and detecting the antibody. The measuring may thus comprise:

transferring, affixing or immobilizing cells or cell lysate from the subject sample onto an assay plate;

applying an anti-phospho-S1292-LRRK2 antibody to the assay plate;

removing or washing away excess anti-phospho-S1292-LRRK2 antibody; and

detecting anti-phospho-S1292-LRRK2 antibody bound to the assay plate.

In certain embodiments the anti-phospho-S1292-LRRK2 antibody may be a monoclonal or polyclonal antibody that binds to an antigen peptide or protein comprising the sequence PNEMGKLSKIWDLPL, or CPNEMGKLSKIWDLPL, or a fragment thereof. In the above sequence the serine residue “S” is phosphorylated or phosphorolatable. The antigen peptide or protein thus can be represented as PNEMGKL-pS-KIWDLPL or CPNEMGKL-pS-KIWDLPL wherein “pS” is phosphorylated serine.

In another embodiment the antigen peptide or protein is between 12 and 20 residues in length, or between 14 and 18 residues in length, includes a phosphorylated serine, and has 90% homology with PNEMGKL-pS-KIWDLPL. In still another embodiment the antigen peptide or protein is between 12 and 20 residues in length, or between 14 and 18 residues in length, includes a phosphorylated serine, and contains at least 10 consecutive residues from within PNEMGKL-pS-K1WDLPL. The anti-phospho-S1292-LRRK2 antibody may be applied to the assay plate as a solution.

In certain embodiments, detection of the anti-phospho-S1292-LRRK2 antibody may utilize ELISA detection, DELFIA detection, fluorescence detection, electrochemiluminescense detection, or other electrical, optical or radioisotope techniques capable of quantitative measurement or detection of antibodies on an assay plate.

In certain embodiments, the comparing of the amount or level of S1292 autophosphorylation in the subject sample with the amount or level of S1292 autophosphorylation in one or more control samples may be embodied in logical elements of computer software stored in a computer-readable medium. The comparing may thus comprise, for example: inputting, via computer user interface, data representing the amount or level of S1292 autophosphorylation in the subject sample; inputting, via computer user interface, data representing the amount or level of S1292 autophosphorylation in the control sample(s); executing programming stored in the computer memory to generate comparison data; and displaying, by computer visual interface, the comparison data, in alphanumeric and/or graphical format, with the comparison data displayed in a manner that shows or determines whether or not the level of S1292 autophosphorylation in the subject sample is elevated with respect to or greater than that of the control sample(s).

Autophosphorylation level of LRRK2 S1292 may also be used as a clinical pharmacodynamic marker or companion diagnostic tool for determining if patient subjects will react favorably to administration of a particular LRRK2 modulator therapy, and/or what dosing level of a LRRK2 modulator or inhibitor may be optimal or desirable for a subject. In this regard the invention provides a method for determining subject response to therapy involving a LRRK2 modulator, the method comprising:

obtaining or acquiring a first LRRK2-containing sample from a subject;

administering a LRRK2 modulator to the subject;

obtaining or acquiring a second LRRK2-containing sample from the subject after administering the LRRK2 modulator to the subject;

measuring, monitoring or otherwise detecting the amount or level of S1292 autophosphorylation in the first subject sample;

measuring, monitoring or otherwise detecting the amount or level of S1292 autophosphorylation in the second subject sample; and

comparing the amount or level of S1292 autophosphorylation in the first subject sample with the amount or level of S1292 autophosphorylation in the second subject sample.

The method may further comprise determining if the amount or level of S1292 autophosphorylation in the second subject sample is less than the amount or level of S1292 autophosphorylation in the first subject sample. A reduction in S1292 autophosphorylation in the second subject sample with respect to the first subject sample indicates that the LRRK2 modulator is effective. The amount or level of reduction of S1292 autophosphorylation further is indicative of how effective the LRRK2 modulator is for the subject, and thus what dosing levels may be appropriate for the subject.

By way of example and not of limitation, the first and second LRRK2-containing subject samples may comprise cells, cell lysate, or solutions or suspensions thereof, obtained from tissue or serum from the subject. In one embodiment the subject samples may comprise monocytes (white blood cells) or lysate thereof. The samples may be obtained from the subject via syringe or tissue extraction device. The samples thus obtained may be subject to cell separation, buffering, solvent extraction and/or exposure to lysing enzyme(s) to expose or otherwise make accessible the LRRK2 protein contained therein.

In certain embodiments the measuring of the level of S1292 autophosphorylation in the first and second subject samples may be carried out using an antibody that associates with S1292 (i.e., an anti-phospho-S1292-LRRK2 antibody), and detecting the antibody. The measuring may thus comprise

transferring, affixing or immobilizing cells or cell lysate from the first and second samples onto an assay plate or plates;

applying an anti-phospho-S1292-LRRK2 antibody to the assay plate or plates;

removing or washing away excess anti-phospho-S1292-LRRK2 antibody; and

detecting anti-phospho-S1292-LRRK2 antibody bound to the assay plate or plates.

In certain embodiments the anti-phospho-S1292-LRRK2 antibody may be a monoclonal or polyclonal antibody that binds to an antigen peptide or protein comprising the sequence PNEMGKL-pS-KIWDLPL (PNEMGKLSKIWDLPL), CPNEMGKL-pS-KIWDLPL (CPNEMGKLSKIWDLPL), or a fragment thereof, wherein “pS” is phosphorylated serine as noted above. In another embodiment the antigen peptide or protein is between 12 and 20 residues in length, or between 14 and 18 residues in length, includes a phosphorylated serine, and has 90% homology with PNEMGKL-pS-KIWDLPL. In still another embodiment the antigen peptide or protein is between 12 and 20 residues in length, or between 14 and 18 residues in length, includes a phosphorylated serine, and contains at least 10 consecutive residues from within PNEMGKL-pS-KIWDLPL. The anti-phospho-S1292-LRRK2 antibody may be applied to the assay plate(s) as a solution.

In certain embodiments, detection of the anti-phospho-S1292-LRRK2 antibody from the first and second subject samples may utilize ELISA detection, DELFIA detection, fluorescence detection, electrochemiluminescense detection, or other electrical, optical or radioisotope techniques capable of quantitative measurement or detection of antibodies on an assay plate or plates.

In certain embodiments, the comparing of the amount or level of S1292 autophosphorylation in the first subject sample with the amount or level of S1292 autophosphorylation in the second subject sample may be embodied in logical elements of computer software stored in a computer-readable medium. The comparing may thus comprise, for example: inputting, via computer user interface, data representing the amount or level of S1292 autophosphorylation in the first subject sample; inputting, via computer user interface, data representing the amount or level of S1292 autophosphorylation in the second subject sample; executing programming stored in the computer memory to generate comparison data; and displaying, by computer visual interface, the comparison data, in alphanumeric and/or graphical format, with the comparison data displayed in a manner that shows or determines whether or not the level of S1292 autophosphorylation in the second subject sample is less than or otherwise reduced with respect to that in the first subject sample.

The subject invention may be embodied in a kit that comprises:

one or more syringes or tools for acquiring a tissue sample or samples from a subject;

an assay plate(s); and

a solution of anti-phospho-S1292-LRRK2 antibody.

The kit may further comprise buffer solution(s) for stabilizing sample(s) from the subject.

The kit may further comprise a solution(s) of cell lysing reagent.

The kit may further comprise solution(s) or reagent(s) capable of transferring, affixing or immobilizing cells or cell lysate from the subject sample onto the assay plate(s).

The kit may further comprise a solution for rinsing or removing excess anti-phospho-S1292-LRRK2 antibody from the assay plate(s).

The kit may further comprise a LRRK2 modulator solution and a syringe or other device for administration of the LRRK2 modulator solution.

The invention also provides an anti-phospho-S1292-LRRK2 antibody characterized in that it is capable of binding to an antigen peptide or protein comprising the sequence CPNEMGKL-pS-KIWDLPL (CPNEMGKLSKIWDLPL), or a fragment thereof, wherein “pS” is phosphorylated serine. In another embodiment, the anti-phospho-S1292-LRRK2 antibody is characterized in that it is capable of binding to an antigen peptide or protein comprising the sequence PNEMGKL-pS-KIWDLPL (PNEMGKLSKIWDLPL), or a fragment thereof, wherein “pS” is phosphorylated serine. In another embodiment the antigen peptide or protein is between 12 and 20 residues in length, or between 14 and 18 residues in length, includes a phosphorylated serine, and has 90% homology with PNEMGKL-pS-KIWDLPL. In still another embodiment the antigen peptide or protein is between 12 and 20 residues in length, or between 14 and 18 residues in length, includes a phosphorylated serine, and contains at least 10 consecutive residues from within PNEMGKL-pS-KIWDLPL.

One aspect of the invention is an assay for screening compounds and compositions for inhibition of LRRK2 activity, the method comprising: exposing LRRK2 or a fragment thereof to a potential inhibitor compound; and detecting the level or amount of phosphorylation associated with a phosphorylation site on the LRRK2 protein.

In certain embodiments, the LRRK2 phoshorylation site may be Serine 1292 (Ser1292 or S1292).

In certain embodiments, monitoring phosphorylation at the LRRK2 phosphorylation site may be carried out using an antibody associated with the LRRK2 autophosphorylation site (anti-phospho-LRRK2 antibody). Detection of the anti-phospho antibody may utilize ELISA, DELFIA, fluorescence, electrochemiluminescense, or other electrical, optical or radioisotope techniques. In one embodiment the antibody used is anti-phospho-Serine 1292-LRRK2 antibody.

In certain embodiments, the LRRK2 used in the assay may be wild type or a mutated form associated with Parkinson's disease, such as the R1441G, R1441C, Y1699C, G2019S, 12020T mutations. In other embodiments, the LRRK2 protein used in the assay may feature combinations of Parkinson's Disease associated mutations (i.e. R1441G/G2019S or R1441G/Y1699C/G2019S). In further embodiments, the assay may feature mutations known to impact the kinase or GTPase activity of LRRK2 (i.e. D1994A). The LRRK2 may be of human, rabbit, mouse or other mammalian origin. The LRRK2 protein used in the assay may be prepared by recombinant techniques using HEK293, CHO or other suitable cell line transfected or virally transduced with cDNA encoding all or part of LRRK2. The LRRK2 may include an epitope tag to facilitate affinity purification, antibody detection, or immunoprecipitation.

In one embodiment, the subject method comprises: recombinantly producing LRRK2 in cells; exposing the cells to one or more potential LRRK2 inhibitor compounds; lysing the cells; exposing the cell lysate(s) to an anti-phospho-LRRK2 antibody; and detecting anti-phospho-LRRK2 antibody bound to LRRK2 in the cell lysate.

In another embodiment, the method comprises: recombinantly producing LRRK2 in cells; exposing the cells to one or more potential LRRK2 inhibitor compounds; lysing the cells; immobilizing LRRK2 from the lysed cells; exposing the cell lysate(s) to an anti-phospho-LRRK2 antibody; and detecting anti-phospho-LRRK2 antibody bound to LRRK2 in the cell lysate.

In another embodiment, the method comprises: recombinantly producing LRRK2 in cells; immobilizing the cells in a plurality of wells of a first plate; exposing the cells in each well to a potential LRRK2 inhibitor compound; lysing the cells in each well; immobilizing LRRK2 from the lysed cells from each well of the first plate in a corresponding well of a second plate; exposing the immobilized LRRK2 to an anti-phospho-LRRK2 antibody; and detecting anti-phospho-LRRK2 antibody bound to LRRK2 Immobilizing the LRRK2 from the lysed cells may comprise, for example, seeding or immobilizing an antibody for LRRK2 or an epitope tag thereon in the wells of the second plate, and transferring the cell lysate from the wells of the first plate to corresponding wells of the second plate.

Another aspect of the invention comprises the utilization of a phosphorylation site of LRRK2 as a biomarker method for the in vivo detection of Parkinson's disease mutations in LRRK2 and for evaluation of the in vivo efficacy of LRRK2 inhibitors. The method may comprise: administering a LRRK2 inhibitor to a subject; acquiring a tissue or serum sample from the subject; and detecting the level or amount of phosphorylation associated with a phosphorylation site on LRRK2 from the sample.

In one embodiment, the biomarker phosphorylation site may be Serine 1292 (Ser1292 or S1292) of the LRRK2 protein.

Monitoring phosphorylation at the LRRK2 phosphorylation site may be carried out by detection of antibody associated with the LRRK2 autophosphorylation site (anti-phospho-LRRK2 antibody) such as anti-phospho-Serine 1292-LRRK2 antibody. anti-phospho-Serine 1292-LRRK2 may be detected using standard procedures such as ELISA, DELFIA, fluorescence, electrochemiluminescense, or other electrical, optical or radioisotope techniques in combination with an anti-phospho-Serine 1292-LRRK2 antibody.

Another aspect of the invention is an antibody for an antigen comprising the sequence CPNEMGKL-pS-KIWDLPL, or a fragment thereof, wherein “pS” is phosphorylated serine. In another embodiment the antigen is between 14 and 18 residues in length, includes a phosphorylated serine, and has 90% homology with CPNEMGKL-pS-KIWDLPL.

Another aspect of the invention is a kit or kits for in vitro evaluation of LRRK2 inhibitors. Such kits may comprise, for example: a solution containing LRRK2 protein; and a solution containing an anti-phospho-LRRK2 antibody such as anti-phospho-Ser1292-LRRK2 antibody. The solutions may be stored in suitable tubes or containers. and the kit may further comprise containers, multiwell plates, buffer solutions and tools configured for carrying out the assay and biomarker methods of the invention described herein.

### BRIEF DESCRIPTION OF THE DRAWINGS

The invention will be more fully understood by reference to the following drawings, which are for illustrative purposes.

FIG. 1. Identification of an in vivo LRRK2 autophosphorylation site. (A) Schematic diagram depicting the domain architecture of LRRK2. (B, C) Annotated MS/MS spectra collected which unambiguously identify pSer1292 and pThr1343. Comparison of these spectra to MS/MS derived from synthetic internal standards provides additional confirmation. (D, E) Extracted ion chromatograms for pSer1292 SFPNEMGKLS#K and pThr1343 LMIVGNT#GSGK peptides from reactions of G2019S-LRRK2 at times ranging from 0 to 60 min. The isotopically labeled internal standards of pSer1292 (¹³C₆¹⁵N₁ Leu; +7.017 Da) and pThr1343 (¹³C₅¹⁵N₁ Val; +6.014 Da) are shown at the back of each 3D plot, with the signal in the front representing the light analyte peptide derived from the digested sample. (F, G) Extracted ion chromatograms for light and heavy pSer1292 peptides from FLAG-LRRK2 immunoprecipiated from transfected 293 cells. Light pSer1292 can be detected from G2019S-LRRK2, but not kinase dead D1994A LRRK2.

FIG. 2. Kinase activity of LRRK2 is required for S1292 phosphorylation (A) Western blots detecting LRRK2 autophosphorylation in lysates of HEK 293 cells transiently expressing FLAG tagged LRRK2 WT, S1292A and D1994A. (B) Western blots detecting LRRK2 autophosphorylation in the brain lysates of BAC transgenic mice over expressing LRRK2 WT or kinase-dead mutant.

FIG. 3. Familial Parkinson's disease mutations increase S1292 autophosphorylation (A) Representative Western blot image detecting pS1292 and total LRRK2 signal from lysate of HEK293 cells transiently transfected with WT, R1441G, Y1699C, G20195, 12020T, G2385R and R1441G/G2019S (RG/GS) LRRK2. (B) Quantitation of average fold of LRRK2 pS1292 signal in mutants compared to wild type, signals were average of 4 independent experiments. (C) Representative Western blot image detecting pS1292 and total LRRK2 from tissue lysate from different brain regions of BAC transgenic mice expressing Flag-tagged wild type (WT) and G2019S LRRK2. (D) Quantitation of fold of pS1292 change in total brain lysate from BAC transgenic mice expressing Flag-tagged wild type (WT) and G2019S LRRK2. N=5 animals in each genotype.

FIG. 4. Biochemical mechanisms of LRRK2 autophosphorylation on S1292 (A) GTP binding is required for S1292 autophosphorylation. Representative western blot image detecting pS1292 and total LRRK2 from lysates transiently expressing Flag-tagged wild type, T1348N, G2019S and T1348N/G2019S LRRK2. (B) LRRK2 S1292A protein cannot phosphorylate S1292 residue on LRRK2 kinase-dead mutant (D1994A). Schematic of two constructs co-transfected in HEK293 cells (left). Representative image of Western blot detecting pS1292 from lysates of HEK293 cells transiently expressing Flag-tagged LRRK2 R1441G/Y1699C/G2019S (3M), 3M/S1292A, D1994A, and D1994A with 3M/S1292A (right). (C) Full-length LRRK2 cannot phosphorylate S1292 residue on a truncated kinase-dead LRRK2 (D1994A). Schematic of two co-transfected constructs (left). Representative image of Western blot detecting pS1292 and total LRRK2 from HEK293 cells expressing full-length and truncated LRRK2 carrying D1994A mutation.

FIG. 5. S1292 is required for LRRK2 PD mutation induced neurite outgrowth defects. (A) Neurolucida tracing of a representative GFP transfected hippocampal neuron. GFP signal was used for neurite tracing with Neurolucida software. An overlay of concentric circles, spaced at 20 μm intervals, centered at the cell body was used for Sholl analysis. (B) Representative Neurolucida tracing of GFP and LRRK2 co-transfected hippocampal neurons. GFP were co-transfected with empty vector, LRRK2 Wild-type, R1441G G2019S, S1292A R1441G G2019S and R1441G G2019S D1994A alleles into hippocampal cultures at day 6 in vitro (DIV 6). At DIV 17, neurons were fixed and stained with GFP and MAP2. Images of transfected neurons were taken with confocal microscopy and dendrites were traced with Neurolucida software by an observer blind to the identity of the allele. Scale bar: 100 μm. (C) Sholl analysis. Mean (±SEM) number of intersections with each circle in LRRK2 transfected cells were counted. Student t-test p values between R1441G G2019S and wild type, R1441G G2019S D1994A, S1292A R1441G G2019S were less than 0.05 (indicated by *). N=22-30 cells for each constructs. (D). Quantification of total dendritic length as in (B). n=40-85 cells in three experiments. Mean diamonds were shown for each group. Student t-test p values between R1441G G2019S (red circle) and wild type, R1441G G2019S D1994A, S1292A R1441G G2019S were less than 0.001.

FIG. 6A-D. S1292A mutation partially ameliorates autophagy impairment induced by LRRK2 PD mutations.

FIG. 7. LRRK2 kinase inhibitors block pS1292 autophorsphorylation in a cell-based assay. (A) Utilizing the pLRRK2 electrochemiluminescent 96 well plate based assay, an increased pLRRK(Ser1292) signal was detected in LRRK2 cell lysates induced with 1 ug/mL doxycline for 16 hours compared to non-induced LRRK cell lysates. (B) Dose dependent reduction of pS1292 signal in doxycline induced LRRK2 cells incubated with Sutent (IC₅₀ 0.10 uM) and G1023 (IC₅₀ 0.0016 uM) for 16 hours Inhibition was normalized to non-induced cell lysates as 100% inhibition. (C) 600 compound data set showing robust correlation of cellular pLRRK(Ser1292) inhibitor IC50 values and biochemical LRRK2 Ki values. Slope=0.95, R²=0.77. Open symbols are out of range IC50 values and were excluded from the regression fit.

FIG. 8. LRRK2 kinase inhibitor G1023 reduces pS1292 autophorsphorylation in vivo. Image of Western blot detecting pS1292 and total LRRK2 from tissue lysates of spleen (A) and brain (B) from Flag-tagged G2019S LRRK2BAC transgenic animals. Animals were dosed with vehicle alone (2 animals), 10, 30 or 100 mg/kg of G01581023 (3 animals each for the compound dosed groups). (C) Quantitation of pS1292/total LRRK2 signal in (A) and (B). (D), (E).

FIG. 9A, 9B. LRRK2 kinase inhibitor G1023 ameliorates neurite outgrowth defect of G2019S neurons cultured from LRRK2 BAC transgenic mice.

### DETAILED DESCRIPTION OF THE INVENTION

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

The definitions of certain terms as used in this specification are provided below. Definitions of other terms may be found in the glossary provided by the U.S. Department of Energy, Office of Science, Human Genome Project (http: www.ornl.gov/sci/techresources/Human_Genome/glossary/). In practicing the present invention, many conventional techniques in molecular biology, microbiology and recombinant DNA are used. These techniques are well-known and are explained in, e.g., Current Protocols in Molecular Biology, Vols. I-III, Ausubel, ed. (1997); Sambrook et al., Molecular Cloning: A Laboratory Manual, Second Edition (Cold Spring Harbor Laboratory Press, Cold Spring Harbor, N.Y., 1989); DNA Cloning: A Practical Approach, Vols. I and II, Glover D, ed. (1985); Oligonucleotide Synthesis, Gait, ed. (1984); Nucleic Acid Hybridization, Hames & Higgins, eds. (1985); Transcription and Translation, Hames & Higgins, eds. (1984); Animal Cell Culture, Freshney, ed. (1986); Immobilized Cells and Enzymes (IRL Press, 1986); Perbal, A Practical Guide to Molecular Cloning; the series, Methods in Enzymol. (Academic Press, Inc., 1984); Gene Transfer Vectors for Mammalian Cells, Miller & Calos, eds. (Cold Spring Harbor Laboratory, N.Y., 1987); and Methods in Enzymology, Vols. 154 and 155, Wu & Grossman, and Wu, eds., respectively.

As used herein, the term “allele” means a particular form of a gene or DNA sequence at a specific chromosomal location (locus).

As used herein, the term “antibody” includes, but is not limited to, polyclonal antibodies, monoclonal antibodies, humanized or chimeric antibodies and biologically functional antibody fragments sufficient for binding of the antibody fragment to the protein. Antibodies can be used in assays to determine the presence of variant proteins and peptides where the genetic polymorphisms of the invention are in the coding region of the gene.

As used herein, the term “clinical response” means any or all of the following: a quantitative measure of the response, no response, and adverse response (i.e., side effects).

As used herein, the term “clinical trial” means any research study designed to collect clinical data on responses to a particular treatment, and includes but is not limited to phase I, phase II and phase III clinical trials. Standard methods are used to define the patient population and to enrol subjects.

As used herein, the term “effective amount” of a compound is a quantity sufficient to achieve a desired therapeutic and/or prophylactic effect, for example, an amount which results in the prevention of or a decrease in the symptoms associated with a disease that is being treated, e.g., the diseases associated with LRRK2 mutant polynucleotides and mutant polypeptides identified herein (particularly Alzheimer's disease and Parkinson's disease). The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compounds of the present invention, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. The compounds of the present invention can also be administered in combination with each other, or with one or more additional therapeutic compounds.

As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the term “genotype” means an unphased 5′ to 3′ sequence of nucleotide pairs found at one or more polymorphic sites in a locus on a pair of homologous chromosomes in an individual. As used herein, genotype includes a full-genotype and/or a sub-genotype.

As used herein, the term “locus” means a location on a chromosome or DNA molecule corresponding to a gene or a physical or phenotypic feature, in particular the LRRK2 gene.

As used herein, the term “LRRK2 modulating agent” is any compound that alters (e.g., increases or decreases) the expression level or biological activity level of LRRK2 polypeptide compared to the expression level or biological activity level of LRRK2 polypeptide in the absence of the LRRK2 modulating agent. LRRK2 modulating agent can be a small molecule, polypeptide, carbohydrate, lipid, nucleotide, or combination thereof. The LRRK2 modulating agent may be an organic compound or an inorganic compound.

As used herein, the term “mutant” means any heritable variation from the wild-type that is the result of a mutation, e.g., single nucleotide polymorphism. The term “mutant” is used interchangeably with the terms “marker”, “biomarker”, and “target” throughout the specification.

As used herein, the term “medical condition” includes, but is not limited to, any condition or disease manifested as one or more physical and/or psychological symptoms for which treatment is desirable, and includes previously and newly identified diseases and other disorders.

As used herein, the term “nucleotide pair” means the nucleotides found at a polymorphic site on the two copies of a chromosome from an individual.

As used herein, the term “polymorphic site” means a position within a locus at which at least two alternative sequences are found in a population, the most frequent of which has a frequency of no more than 99%.

As used herein, the term “phased” means, when applied to a sequence of nucleotide pairs for two or more polymorphic sites in a locus, the combination of nucleotides present at those polymorphic sites on a single copy of the locus is known.

As used herein, the term “polymorphism” means any sequence variant present at a frequency of >1% in a population. The sequence variant may be present at a frequency significantly greater than 1% such as 5% or 10% or more. Also, the term may be used to refer to the sequence variation observed in an individual at a polymorphic site. Polymorphisms include nucleotide substitutions, insertions, deletions and microsatellites and may, but need not, result in detectable differences in gene expression or protein function.

As used herein, the term “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “polypeptide” means any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well-known in the art. Such modifications are well described in basic texts and in more detailed monographs, as well as in a voluminous research literature.

As used herein, the terms “phosphorylation” and “autophosphorylation”, which are used interchangeably herein refer generally to the adding of a phosphate group to LRRK2 at a phosphorylation site thereon, such as Serine 1292 (Ser 1292 or S1292).

As used herein, the term “SNP nucleic acid” means a nucleic acid sequence, which comprises a nucleotide that is variable within an otherwise identical nucleotide sequence between individuals or groups of individuals, thus existing as alleles. Such SNP nucleic acids are preferably from about 15 to about 500 nucleotides in length. The SNP nucleic acids may be part of a chromosome, or they may be an exact copy of a part of a chromosome, e.g., by amplification of such a part of a chromosome through PCR or through cloning. The SNP nucleic acids are referred to hereafter simply as “SNPs”. A SNP is the occurrence of nucleotide variability at a single position in the genome, in which two alternative bases occur at appreciable frequency (i.e., >1%) in the human population. A SNP may occur within a gene or within intergenic regions of the genome. SNP probes according to the invention are oligonucleotides that are complementary to a SNP nucleic acid.

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

As used herein, “Parkinson's disease” (“PD”) means a degenerative disorder of the central nervous system that impairs motor skills, speech, and/or cognitive function. Symptoms of Parkinson's disease may include, for example, muscle rigidity, tremor, slowing of physical movement (bradykinesia) and loss of physical movement (akinesia).

As used herein, “Lewy body disease” also called “Lewy body demntia”, diffuse Lewy body disease”, cortical Lewy body disease”, means a neurogenerative disorder characterized anatomically by the presence of Lewy bodies in the brain.

As used herein, “Subject” means mammals and non-mammals. Mammals means any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

As used herein, “Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The terms “those defined above” and “those defined herein” when referring to a variable incorporates by reference the broad definition of the variable as well as particular definitions, if any.

“Treating” or “treatment” of a disease state includes, inter alia, inhibiting the disease state, i.e., arresting the development of the disease state or its clinical symptoms, and/or relieving the disease state, i.e., causing temporary or permanent regression of the disease state or its clinical symptoms.

All patents and publications identified herein are incorporated herein by reference in their entirety.

### LRRK2

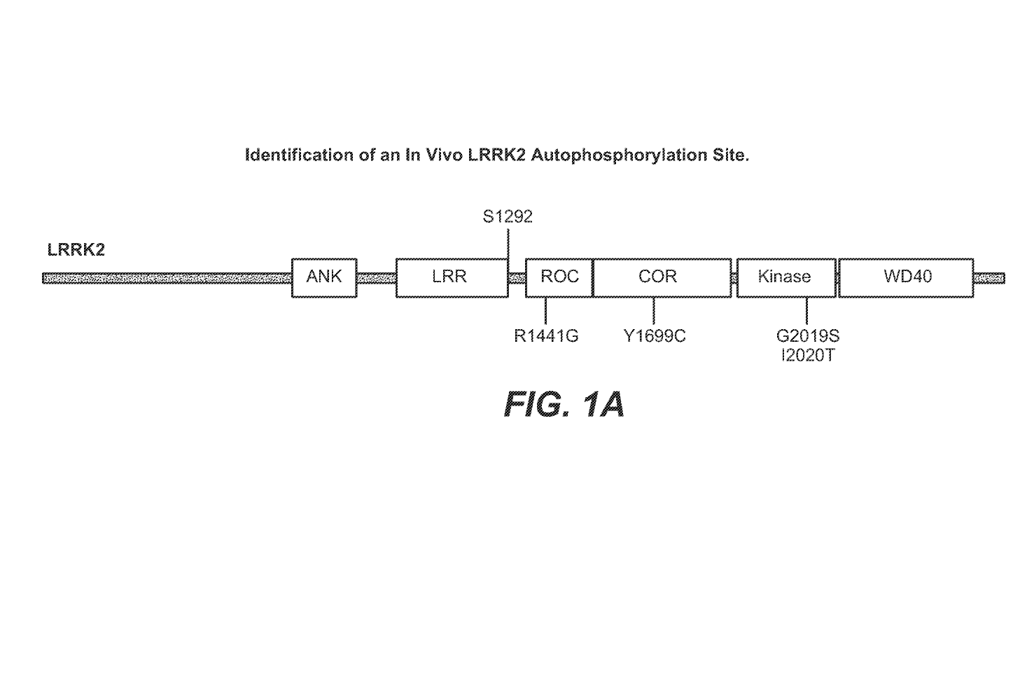

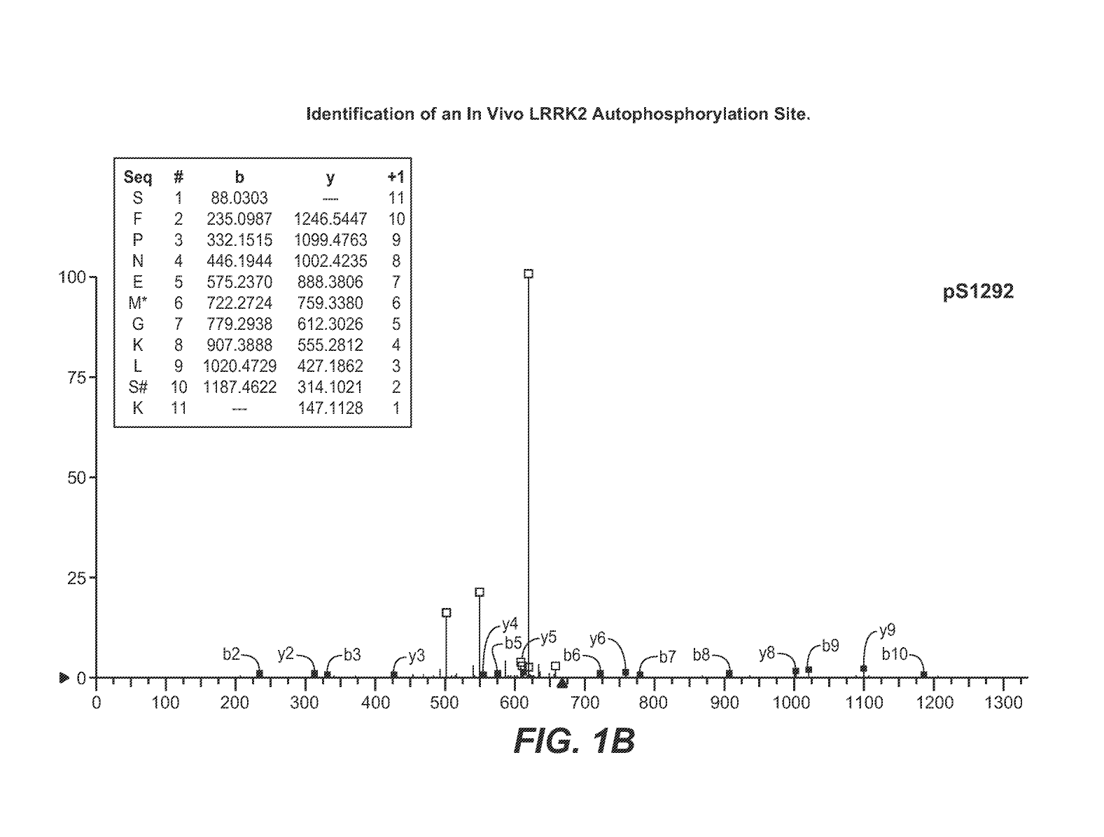

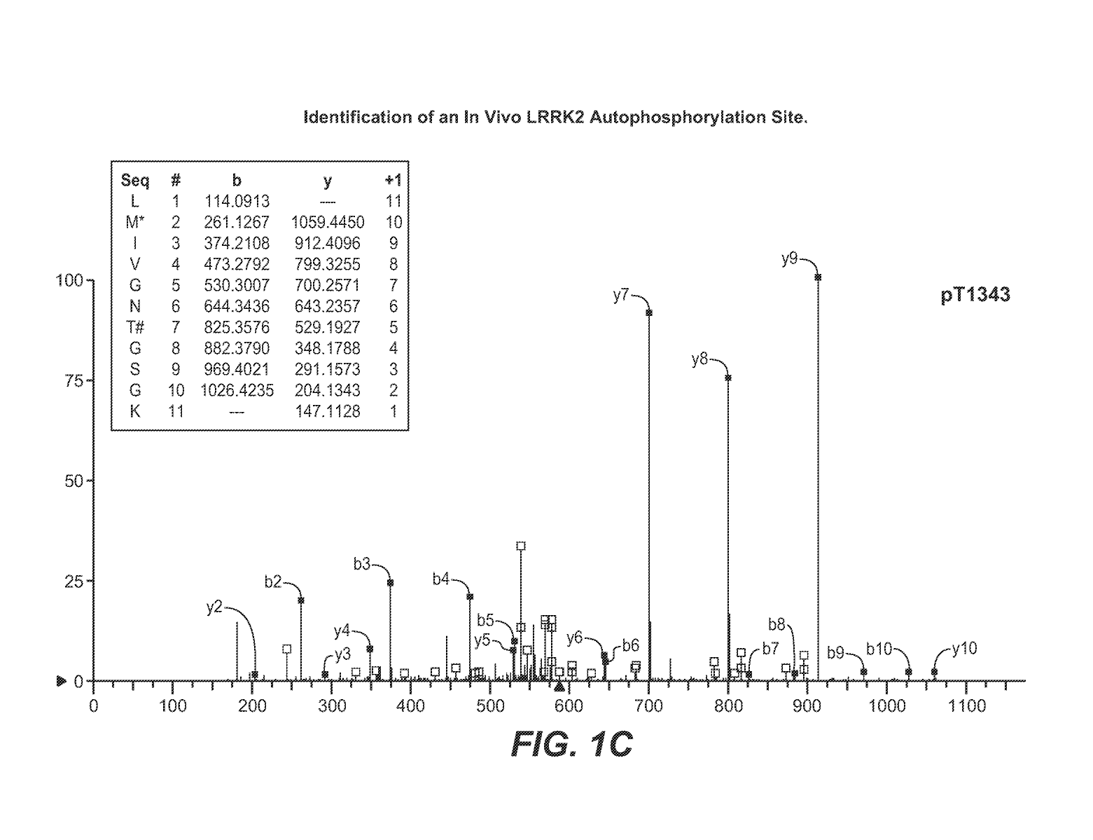

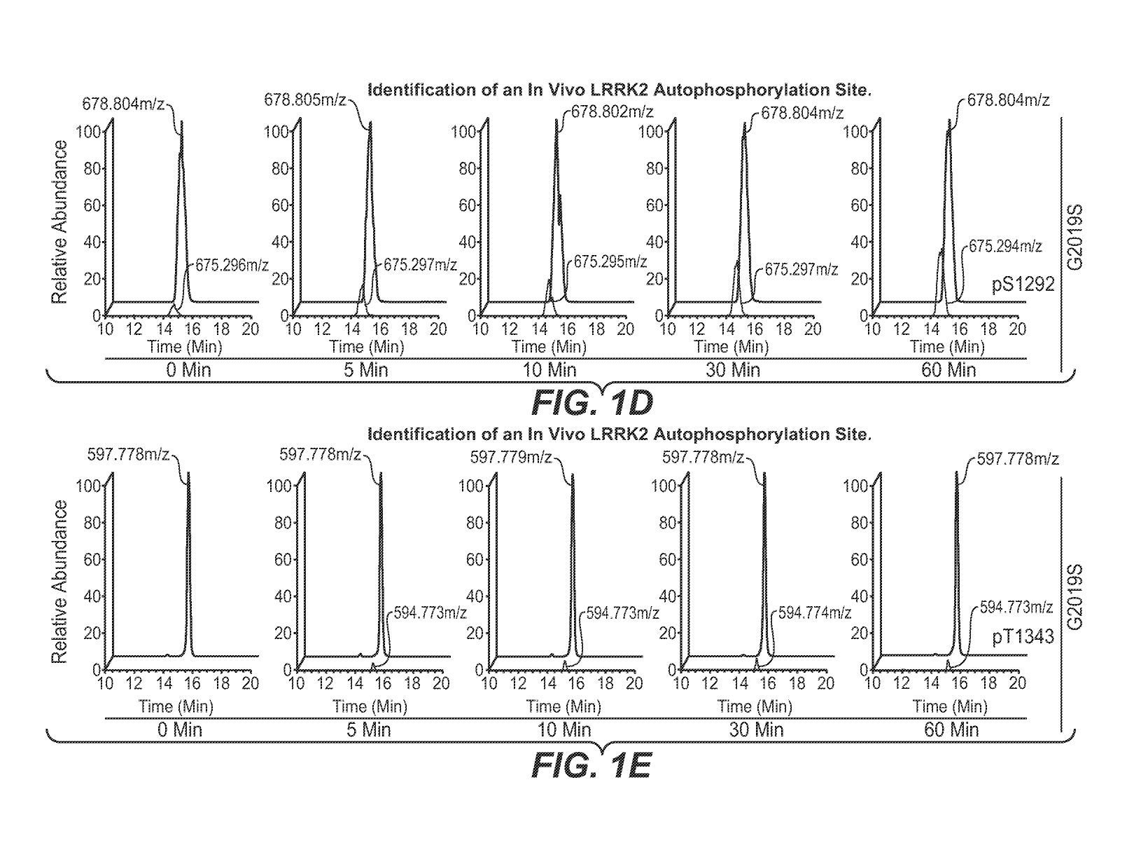

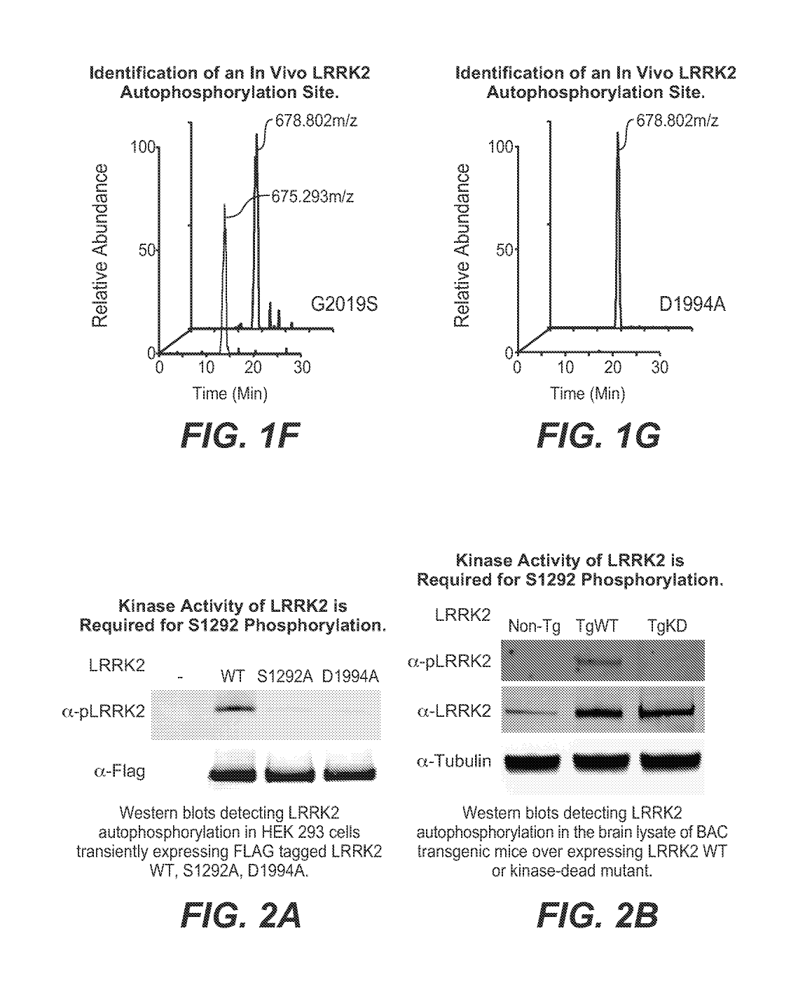

Referring to FIG. 1, there is shown a schematic representation of the LRRK2 protein with N-terminus at the left and C-Terminus at the right. The LRRK2 protein is 2527 amino acids in length and belongs to the ROCO-protein superfamily. LRRK2 includes an ANK (Ankyrin repeat) domain, a LRR (Leucine rich repeat) domain, a ROC (Ras in complex proteins) domain, a COR(C-terminal of Roc) linker domain, a kinase domain, and a WD40 domain. The S1292 autophosphorylation site is located generally between the LRR and ROC domains as shown in FIG. 1. The R1441G mutation occurs in the ROC domain, while the Y1699C mutation is in the COR domain, and the G2019S and 12020T are found in the Kinase domain. The human LRRK2 protein and corresponding nucleic acid sequences are well known and are disclosed, for example, as SEQ. ID NO: 2 and SEQ ID NO: 1 respectively in published application US 2010/0035251.

### Identification of S1292 as an In Vivo Autophosphorylation Site

In vitro kinase assays were carried out using purified G2019S and kinase dead (D1994A) LRRK2. Reactions were performed in the presence or absence of ATP (1 mM) and GTPgS (10 mM), then separated by SDS-PAGE. Coomassie stained LRRK2 bands were subjected to in gel trypsin digestion followed by liquid-chromatography tandem mass spectrometry (LC-MS/MS, Aqua peptides (Sigma-Aldrich)). Database searching revealed a series of spectra which confidently matching to phosphopeptides of LRRKs. Of the identified sites, none were observed from the reactions with kinase dead D1994A LRRK2. Noteworthy were a cluster of phosphopeptides beginning with pSer1292 at the C-terminal portion of the LRR domain and extending through the ROC domain (FIGS. 1B, 1C). Phosphorylation was also observed on at least one of the residues within the kinase activation loop (pSer2031; data not shown). These results support a previous indicating that LRRK2 preferentially modifies Thr residues over Ser, at least within the context of autophosphorylation activity.

Among the identified LRRK2 autophosphorylation sites, an unusual frequency of Met residues was noted in close proximity to LRRK2 phosphorylation sites, with 9/14 peptides displaying a Met between one and five positions upstream of the modified residue. While none of the phosphopeptides were observed in the reactions lacking ATP and GTPgS, the single exception was a spectra matching to the sequence SFPNEMGKLS#K, corresponding to pSer1292 (FIG. 1B).

To assess the relative abundance of phosphorylation in vitro and assist with validating these modifications in vivo, a series of internal standard peptides were synthesized. These peptides comprised the same sequences as identified LRRK2 phosphopeptides, but included a single amino acid which was enriched in stable isotopes Included among these were isotopically labeled peptides directed towards pSer1292 were SFPNEMGKLS#K (¹³C₆¹⁵N₁ Leu; +7.017 Da), pThr1343 LMIVGNT#GSGK (¹³C₅¹⁵N₁ Val; +6.014 Da), pThr1357 TTLLQQLMKT#K (¹³C₆¹⁵N₁ Leu+7.017 Da), and pThr1368 SDLGMQSAT#VGIDVK (¹³C₅¹⁵N₁ Val+6.014 Da). To comprehensively assess phosphorylation status of pSer1357 and pSer1368, additional peptides were required due address ragged-end termini flanking these sequences.

In vitro kinase reactions were then performed using purified WT, G2019S, and R1441C LRRK2 protein. Timepoints were collected at 0, 5, 10, 30 and 60 min, separated by SDS-PAGE and Coomassie stained. Extracted ion chromatograms for pSer1292 and pThr1343 peptides from the G2019S reactions are shown relative to their corresponding internal standards (FIG. 1D, 1E). As noted in the phosphomapping studies, a small fraction of Ser1292 was phosphorylated in the purified G2019S LRRK2 at the onset of the kinase reaction. The raw MS data indicate that each phosphopeptide increases in abundance, with pSer1292 displaying a steady increase from 5 to 60 min compared to pThr1343 which increases from 0 to 10 min before rapidly reaching a plateau. At each position monitored, G2019S appears to undergo more rapid autophosphorylation than either WT or R1441C LRRK2 (FIG. 1E, 1F, 1G, 1H). Interestingly, individual sites display unique kinetics, with autophosphorylation of pThr1343 and pThr1357 rapidly reaching plateaus, while phosphorylation at Ser1292 and Thr1368 continue robustly even out to the 60 min timepoint (FIG. 1E, 1F, 1G, 1H). A modest but consistent difference is also observed between the activity of WT and R1441C LRRK2 (FIG. 1E, 1F, 1G, 1H).

In order to validate LRRK2 autophosphorylation activity in living cells, an experiment on FLAG-LRRK2 overexpressed in HEK293 cells using our quantitative LC-MS/MS assay was performed. The majority of autophosphorylation events identified from in vitro kinase assays were undetectable in immunoprecipitated LRRK2, even with the use of LRRK2 isotopic standards as sentries. The one exception was pSer1292. Extracted ion chromatograms of the heavy pSer1292 signal revealed a low level of pSer1292 on FLAG-^{G2019S}LRRK2 (FIG. 1F), but not FLAG-^{D1994A}LRRK2. An MS/MS acquired on the internal standard confirmed the identity of the heavy peptide, with perfect coresolution of the native peptide essential to confirming the presence of pSer1292 from 293 cells. Neatly, an analogous MS/MS spectrum was collected from the light form of this peptide, but had been coisolated with another ion, confounding automated identification efforts. Manual inspection and comparison of these two spectra permitted deconvolution and additional validation of pSer1292 within this sample.

Since pSer1292 was the most robust autophosphorylation site in vitro and in cultured cells, a rabbit polyclonal antibody was generated that specifically recognizes this phospho-epitope (anti-pLRRK2 antibody). 293 Cells were transfected with wild type, S1292A (phospho-site dead) and D1994A (kinase dead) LRRK2 constructs. It was found that anti-pLRRK2 antibody detect a specific signal from lysates of cells transfected with wild type LRRK2, but not those with either S1292A or D1994A mutations (FIG. 2A), demonstrating that the anti-pLRRK2 antibody is specific to the phosphorylated S1292 epitope. Furthermore, the anti-pLRRK2 antibody detected a specific signal from brain lysates of BAC transgenic mice expressing wild type LRRK2, but not those expressing kinase-dead LRRK2 (FIG. 2B). These results demonstrate that LRRK2 kinase activity is required for phosphorylation of S 1292 residue in vivo.

### Parkinson's Mutations Increase LRRK2 Autophosphorylation on Ser1292

The most common familial mutation of LRRK2 G2019S has been shown to increase its kinase activity in a variety of in vitro kinase assays, including autophosphorylation, phosphorylation of MBP and LRRKtide. Beside G2019S, most published results show that other PD mutations, including R1441G/C, Y1699C and I2020T, do not increase LRRK2 kinase activity in vitro kinase. How PD mutations affect LRRK2 kinase activity in vivo remains unknown because the lack of in vivo substrates.

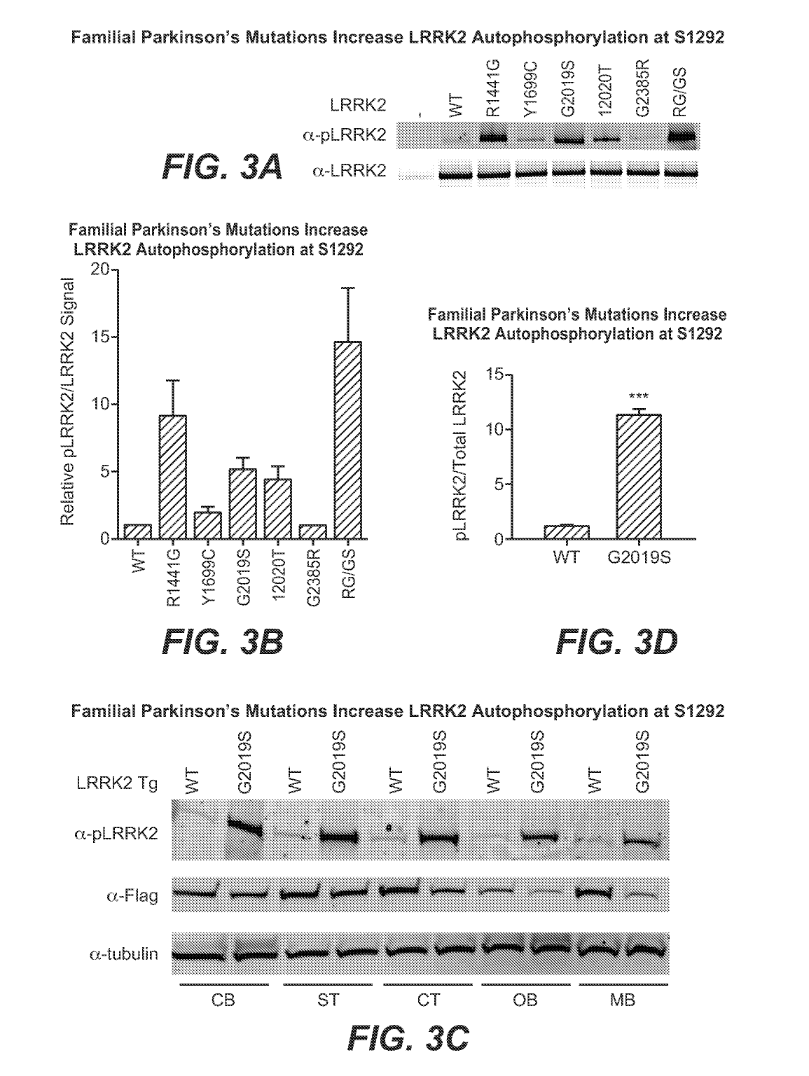

Since LRRK2 autophosphorylation on Ser 1292 is dependent on its kinase activity, the affect of different LRRK2 PD mutations on LRRK2 autophosphorylation levels was tested by transfecting 293 cells with Flag-tagged LRRK2 constructs containing different Parkinson's mutations. The levels of total LRRK2 and pS1292 in whole cell lysates were assessed by Western blotting. We found that all of the four confirmed LRRK2 familial Parkinson's mutations, including R1441G, Y1699C, G2019S and I2020T, showed increased LRRK2 autophosphorylation at S1292 site comparing to wild type LRRK2. Furthermore, combining two PD mutations R1441G and G2019S in the same construct increased LRRK2 autophosphorylation level even higher (FIG. 3A, B). Notably, G2385R, a PD risk factor mutation found in Asian population, did not increase autophosphorylation on Ser1292.

We also tested whether PD mutations increase LRRK2 autophosphorylation in transgenic animals. We compared LRRK2 autophosphorylation levels in the brain tissues from transgenic animals expressing wild type or G2019S LRRK2 proteins. We found that total LRRK2 protein levels were comparable in the two lines of transgenic mice, while the phospho-LRRK2 level is much higher in tissues from LRRK2 G2019S BAC transgenic mice in all brain regions examined (FIG. 3C). Quantitatively, LRRK2 autophosphorylation on S1292 is more than 10 times higher in total brain lysates of transgenic animals expressing G2019S mutant LRRK2 than the ones expressing wild type LRRK2 (FIG. 3D). Together, these data demonstrate that Parkinson's mutations of LRRK2 increase LRRK2 autophosphorylation on S1292 both in cultured cells and in animals.

### Biochemical Mechanisms of LRRK2 Ser1292 Autophosphorylation

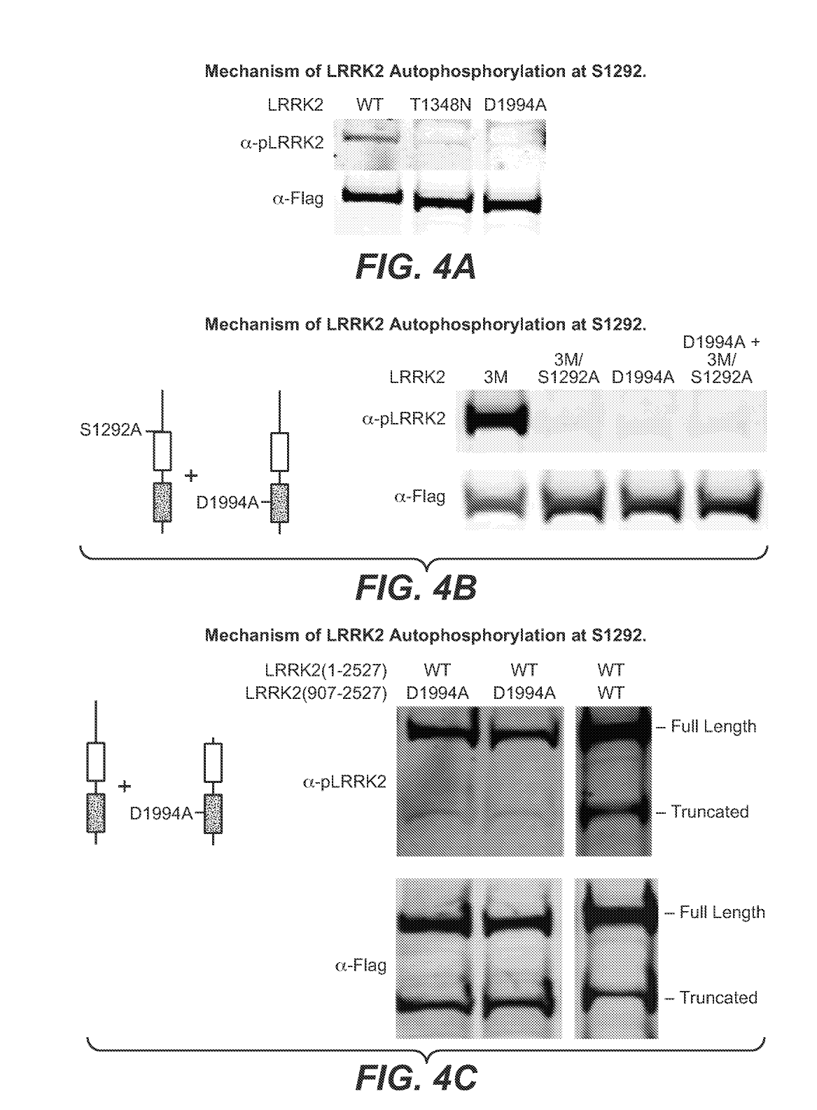

GTP binding is recognized as being required for LRRK2 kinase activation in vitro. LRRK2 T1348N mutation has been shown to abolish GTP binding in vitro. However, it is unclear how GTP binding affects LRRK2 kinase activity in vivo. To test whether GTP binding is required for LRRK2 autophosphorylation in vivo, LRRK2 autophosphorylation levels in 293 cells transfected with LRRK2 T1348N and LRRK2 G2019S/T1348N mutant constructs were examined LRRK2 S1292 autophosphorylation was not detectable in lysates form cells expressing T1348N mutation. S1292 phosphorylation is also greatly reduced in LRRK2 G2019S/T1348N transfected cells compared to those expressing LRRK2 G2019S (FIG. 4A). These results demonstrate that in vivo LRRK2 autophosphorylation on S1292 requires GTP binding.

The mechanism of LRRK2 autophosphorylation may occur by two possible routes. One possibility is that one LRRK2 molecule may phosphorylate S1292 on another LRRK2 molecule, i.e. intermolecular phosphorylation. The other possibility is that the kinase domain of a LRRK2 molecule may phosphorylate its own S1292, i.e. intramolecular phosphorylation. To distinguish these two possibilities, LRRK2 S1292A mutant was transfected with LRRK2 D1994A mutant. S1292A mutation abolishes the autophosphorylation site, while D1994A mutation abolishes LRRK2 kinase activity. If LRRK2 autophosphorylation is inter-molecular, LRRK2 S1292A can phosphorylate LRRK2 D1994A when these two constructs are co-transfected, and phospho-LRRK2 signal would be observed. However, LRRK2 autophosphorylation was not observed in lysates of 293 cells co-transfected with S1292A and D1994A mutation (FIG. 4B). This data suggest that LRRK2 autophosphorylation is likely to be intra-molecular.

To further validate intra-molecular autophosphorylation, phosphorylation of a N-terminal-truncated LRRK2 by full-length wild type LRRK2 was tested. Wild type LRRK2 and N-terminal truncated LRRK2 (907-2527) were co-transfected with D1994A mutation. Little S1292 phosphorylation was detected in truncated LRRK2 D1994A by Western blot (FIG. 4C). Therefore, the full-length wild type LRRK2 could not phosphorylate truncated LRRK2. Taken together, these data demonstrated that LRRK2 autophosphorylation is most likely intra-molecular.

### Ser1292 is Required for Neuronal Toxicity Induced by LRRK2 Parkinson's Mutations.

Ser1292 residue is located at the junction of LRR domain and GTPase domain of LRRK2. A protein homology analysis showed that S1292 is conserved from worm to human, while the overall amino acid identities are limited. The importance of Ser1292 autophosphorylation for the biological function of LRRK2 was thus evaluated.

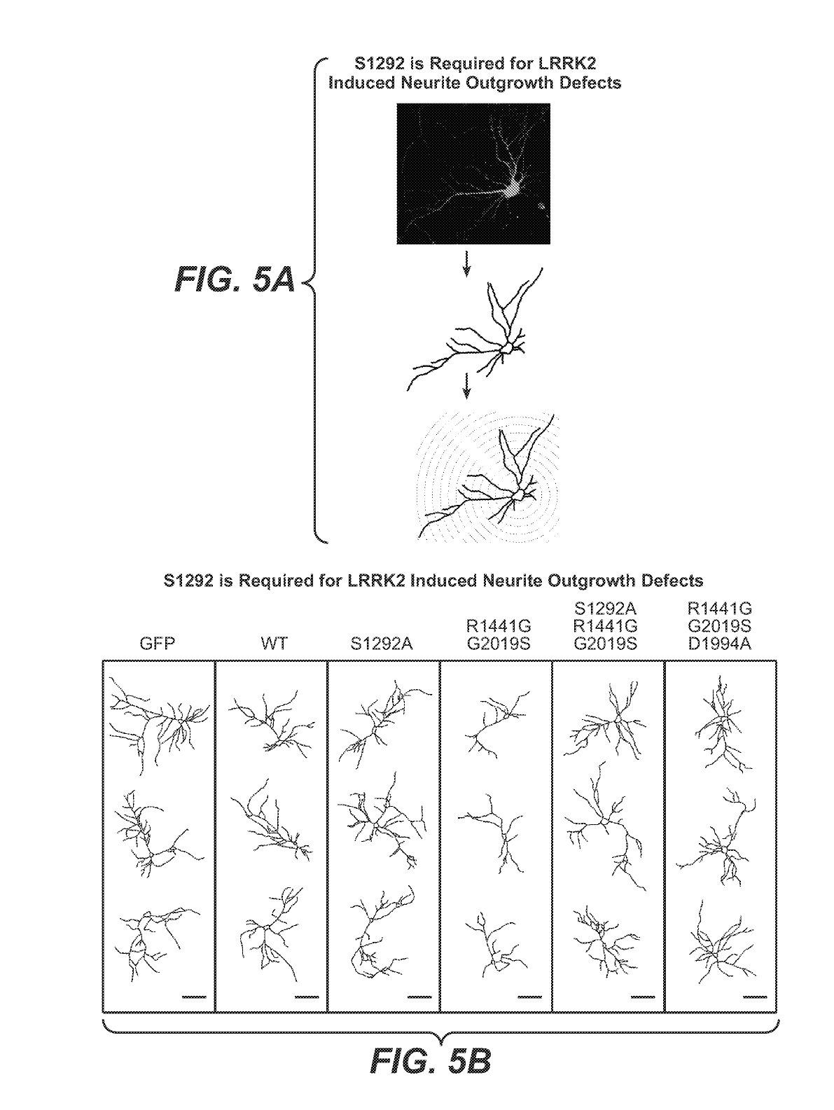

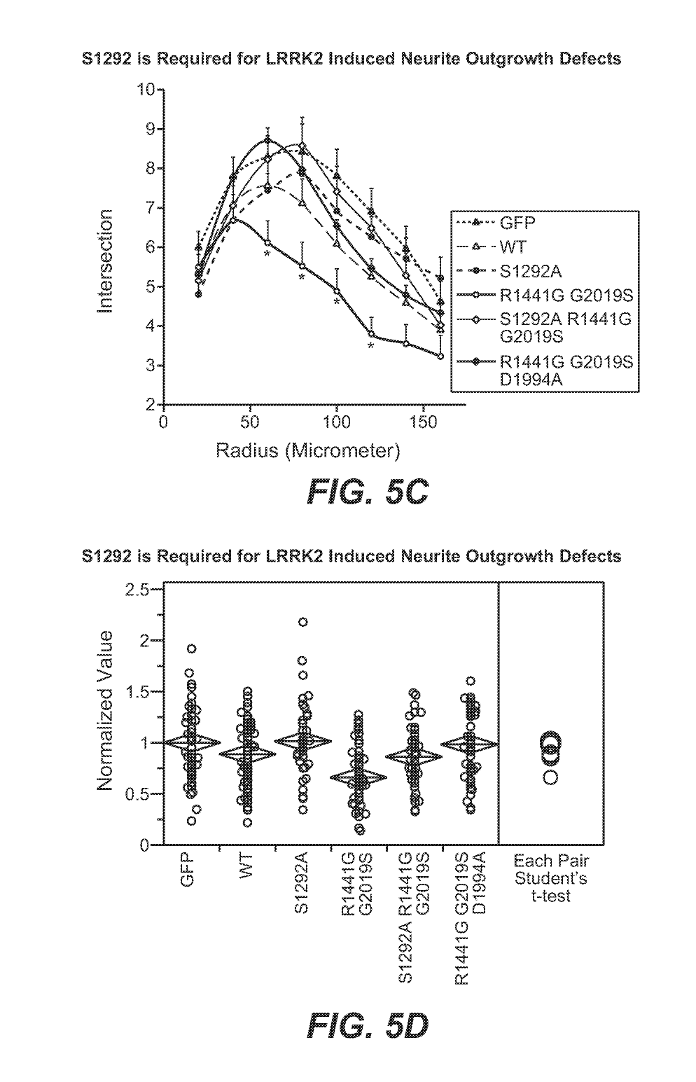

Expression of LRRK2 protein carrying PD mutations causes neuronal toxicity in cultured primary neurons. Furthermore, the observed neuronal toxicity requires the kinase activity of LRRK2. Transfection of cultured primary hippocampal neurons from E18 rat embryos with LRRK2 cDNA carrying different PD mutations and examined neurite morphology was carried out (FIG. 5A) to show that hippocampal neurons expressing LRRK2 R1441G/G2019S double mutation exhibit robust neurite outgrowth defect compared with those expressing wild type LRRK2 or GFP only. Consistent with published reports, introducing a kinase-dead mutation D1994A to LRRK2 R1441G/G2019S double mutation abolished this phenotype. Furthermore, mutating Ser 1292 to Alanine in LRRK2 R1441G/G2019S double mutation also abolished the neurite outgrowth defects, suggesting Ser1292 phosphorylation is important for LRRK2 R1441G/G2019S induced neurite outgrowth phenotypes (FIG. 5 B, D).

In addition to causing shortening of total neurite length, expressing LRRK2 R1441G/G2019S double mutant protein also reduced the complexities of the dendritic trees in cultured primary hippocampal neurons, as shown by Sholl analysis. Furthermore, LRRK2 G2019S/R1441G/D1994A mutant protein restored dendritic complexity of the neurons, suggesting that LRRK2 kinase activity is required. Importantly, mutating LRRK2 autophosphorylation site serine 1292 to alanine also ameliorated this phenotype (FIG. 5C). Taken together, these data strongly suggest that autophosphorylation of Ser1292 on LRRK2 plays important role in mediating LRRK2 PD mutation induced neuronal toxicity.

### Ser1292 Autophosphorylation is Important for LRRK2 PD Mutation Induced Autophagy Impairment

The effect of Ser1292 autophosphorylation on neuronal toxicity induced by LRRK2 PD mutations was examined We first considered whether Ser1292 autophosphorylation affects LRRK2 kinase activity. Flag-tagged wt and S1292A LRRK2 protein was purified from transiently transfected 293 cells. Kinase activities were compared in an in vitro kinase assay with LRRKtide as the substrate. No significant difference between LRRK2 WT and S1292A protein were observed.

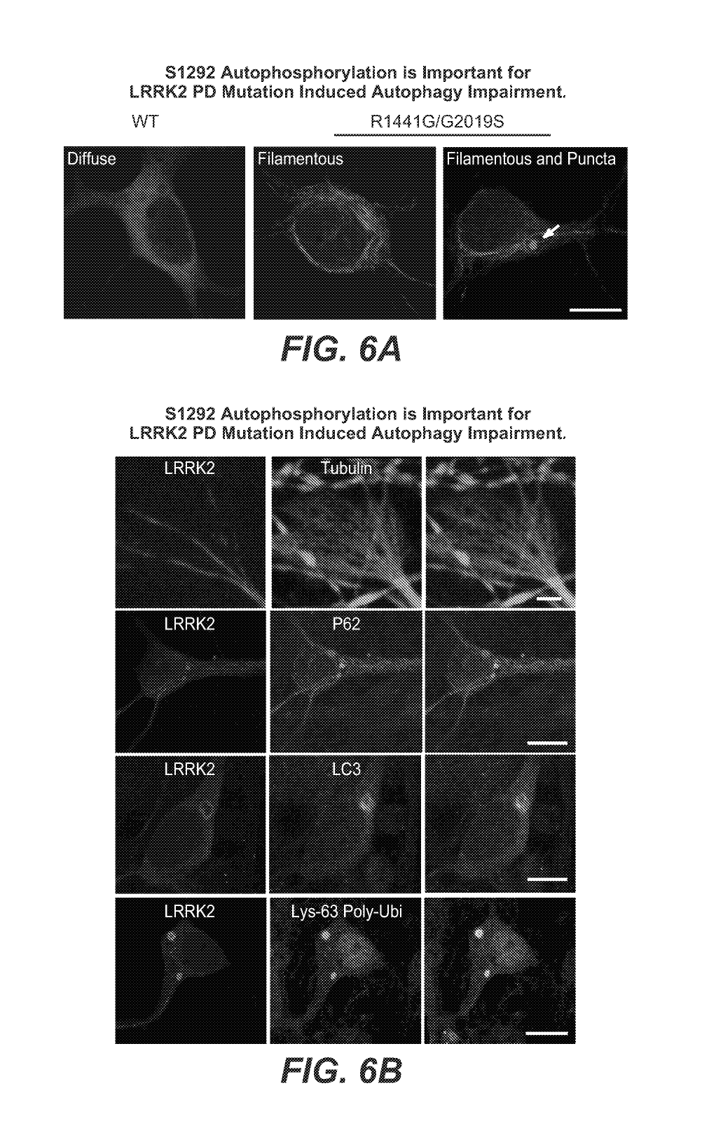

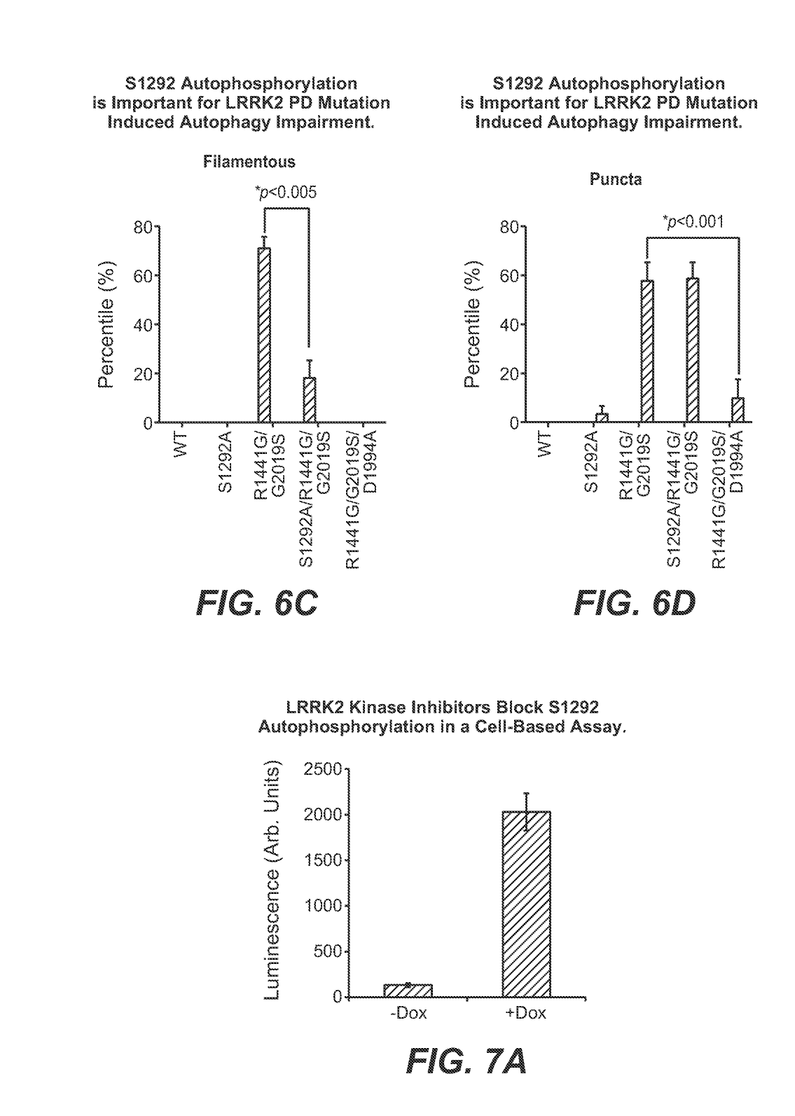

Subcellular localization of LRRK2 PD mutant proteins was next examined. Flag-tagged LRRK2 R1441G/G2019S double mutant construct was transfected into cultured primary cortical neurons Immunofluorescent cytochemistry was used to detect Flag-tagged LRRK2 proteins in transfected neurons. WT LRRK2 protein was found to show diffused distribution in neurons. However, in more than 80% of neurons transfected with LRRK2 R1441G/G2019S double mutant, LRRK2 protein showed filamentous distribution of LRRK that co-localized with microtubule markers (FIG. 6A). Furthermore, LRRK2 mutant protein also formed large puncta in some of the neurons (FIG. 6B). To identify the nature of the filaments and the puncta, these neurons were co-stained with different celluar markers. LRRK2 filaments was found to be co-localized with microtubule, as shown by co-staining with tubulin. Furthermore, both LRRK2 filaments and puncta co-localized with autophagy adaptor protein p62 (FIG. 6A, 6B). Additional cellular marker for autophagy were examined. LRRK2 puncta were also positive for LC3 and K63 poly-ubiquitin, which are markers for autophagosomes. These data suggest that LRRK2 PD mutation impaired basal autophagy activity in neurons.

To test whether LRRK2 kinase activity and/or LRRK2 S1292 autophosphorylation modulate the effect of LRRK2 PD mutations on autophagy, LRRK2 R1441G/G2019S/D1994A and LRRK2 R1441G/G2019S/S1292A mutant proteins were expressed in cultured cortical neurons. Loss of kinase activity (by D1994A) abolished LRRK2 localization to microtubules and to autophagosomes (FIG. 6C, 6D), indicating that kinase activity is important for impaired autophagy activity caused by LRRK2 PD mutations. Interestingly, S1292A mutation substantially reduced LRRK2 localization to microtubules, but had little effect on the formation of LRRK2 positive autophagosomes (FIG. 6D). These data suggest that LRRK2 autophosphorylation on S1292 contributes to impaired autophagy activity induced by LRRK2 PD mutations.

### LRRK2 Kinase Inhibitors Block S1292 Autophosphorylation in Cells

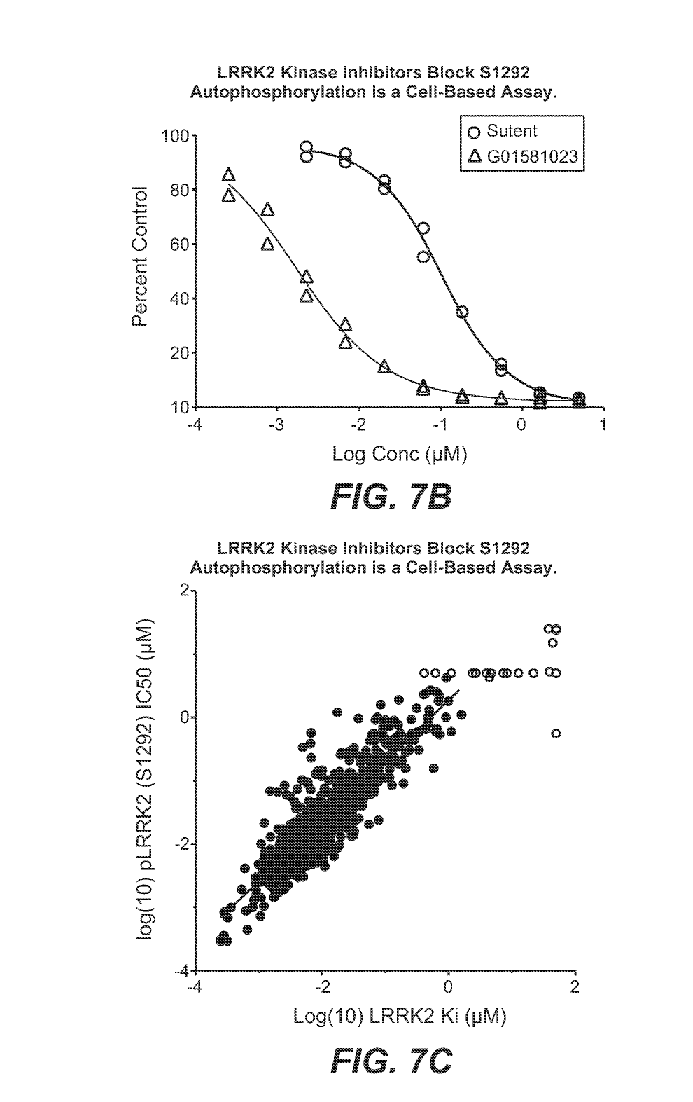

To test whether LRRK2 kinase inhibitors can block LRRK2 autophosphorylation in cells, an inducible HEK293 cell line expressing FLAG epitope-tagged LRRK2 R1441G/Y1699C/G2019S triple mutant protein (referred here as LRRK2 cells) was generated. This cell lines was then used to develop a 96-well plate-based assay to quantify phospho-S1292 in cell lysates. The assay used electrochemiluminescent detection technology (Meso-Scale Discovery, MSD) in which LRRK2 was captured with solid phase-immobilized anti-FLAG monoclonal antibody, followed by detection with anti-pSer1292 antibody and a Ruthenium-conjugated anti-rabbit secondary antibody. We were thus able detect a significant increase in pS1292 signal in lysates of cells induced with doxycycline (FIG. 7A). Further, when doxycycline-induced LRRK2 cells were incubated with Sutent, a known LRRK2 kinase inhibitor (Ki ref), we observed a dose-dependent reduction of pS1292 signal, with maximal inhibition equivalent to the signal detected in lysate from non-induced cells (FIG. 7B). Subsequently, we tested over 600 compounds with varying potencies in the LRRK2 kinase assay on these cells. The IC50s for reduction of pS1292 signal correlated robustly with the biochemical Ki of LRRK2 for the compounds (FIG. 7C). These data demonstrate that LRRK2 kinase inhibitors block S1292 autophosphorylation in cells, indicating that pS1292 can therefore be used as a marker for LRRK2 kinase inhibition in vivo.

### A Potent, Selective and Brain-Penetrable LRRK2 Kinase Inhibitor Blocks Phospho-S 1292 in LRRK2 G2019S BAC Transgenic Mouse Brain

Through a high-throughput chemical screen and lead optimization, we identified a highly potent and selective LRRK2 small molecule inhibitor, [3-methoxy-4-(4-methylamino-5-trifluoromethyl-pyrimidin-2-ylamino)-phenyl]-morpholin-4-yl-methanone (G1023). The biochemical Ki of G1023 is about 4 nm. In LRRK2 cell-based assay, G1023 efficiently reduced pLRRK2 levels in a dose-dependent manner, with observed cellular IC50 at 10 nM (FIG. 7B).

To test whether G1023 can inhibit LRRK2 kinase activity in vivo, BAC transgenic mice expressing LRRK2 G2019S protein were injected with a single dose of this compound at 10, 30 and 100 mg/kg. Periphery and brain tissues were harvested 6 hrs after injection to measure compound concentration as well as phospho-LRRK2 levels.

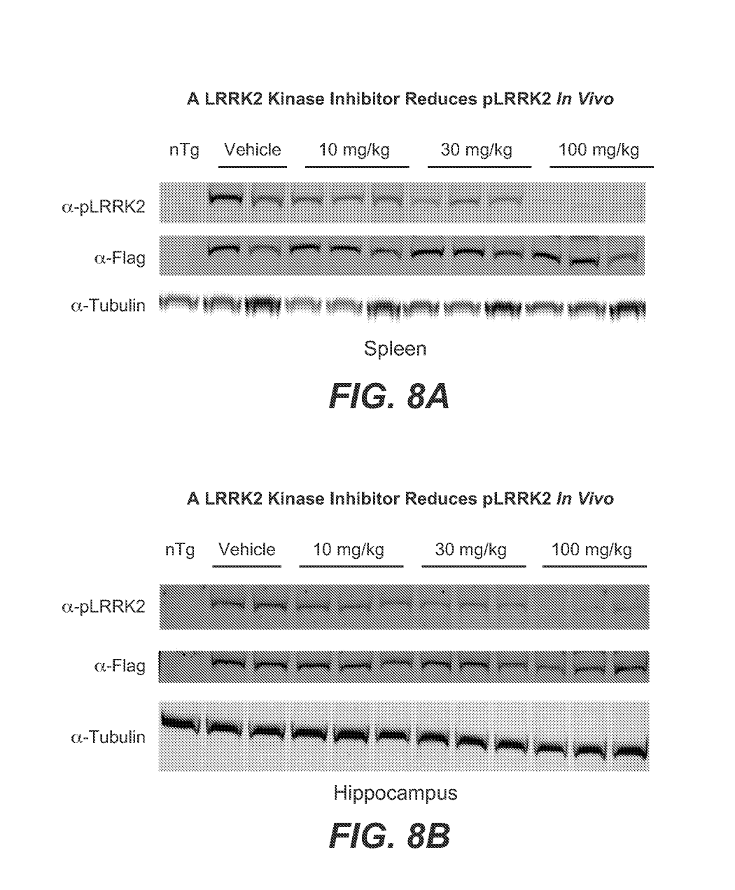

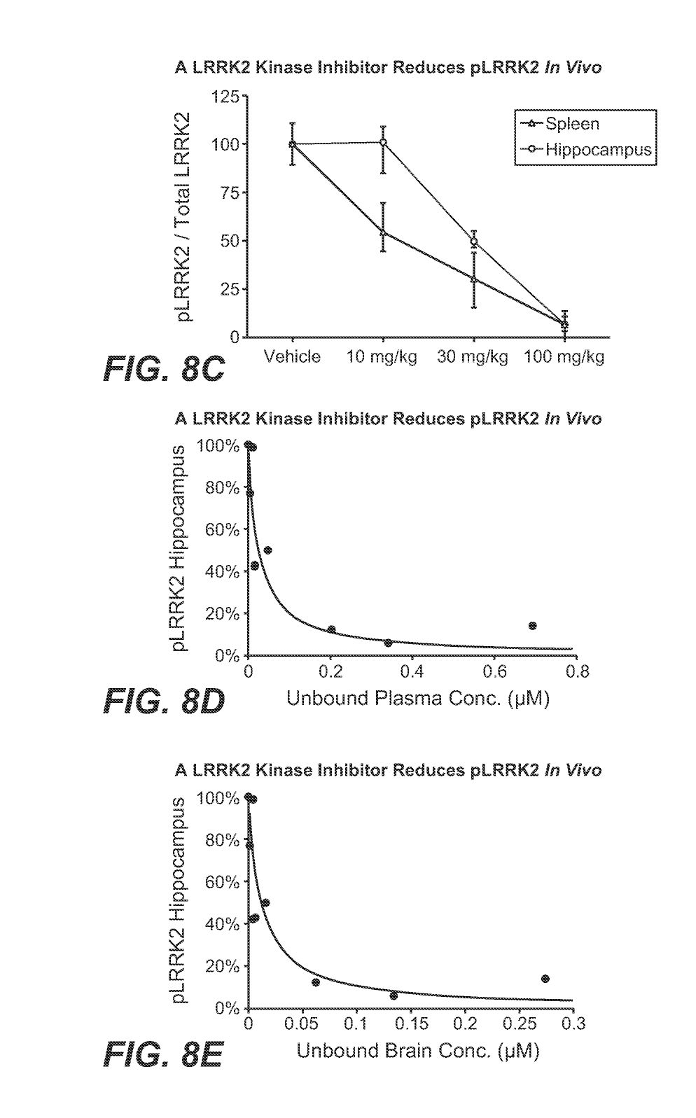

To examine LRRK2 kinase inhibition in G1023 injected animals, spleen, lymph node and brain tissue were harvested 6 hrs after compound injection to determine phospho-LRRK2 levels. In periphery tissues, including spleen and lymph node, G1023 reduced phospho-S1292 in a dose-dependent manner (FIG. 8A, 8C). In brain tissue, G1023 also reduced phospho-S 1292 level effectively, although the dosage required for reduction is higher (FIG. 8B, 8C). The calculated unbound concentration of G1023 in the brain required for phospho-S1292 reduction is consistent with the cellular IC50 for this compound. These data demonstrate that G1023 has high brain exposure and blocks LRRK2 kinase activity in vivo. Furthermore, these data also demonstrate that LRRK2 autophosphorylation on Ser1292 can be used as a pharmacodynamic marker to measure the efficacy of LRRK2 kinase inhibitors in vivo.

### LRRK2 Kinase Inhibitor Attenuates Parkinson's Mutation Induced Neurite Outgrowth Defect.

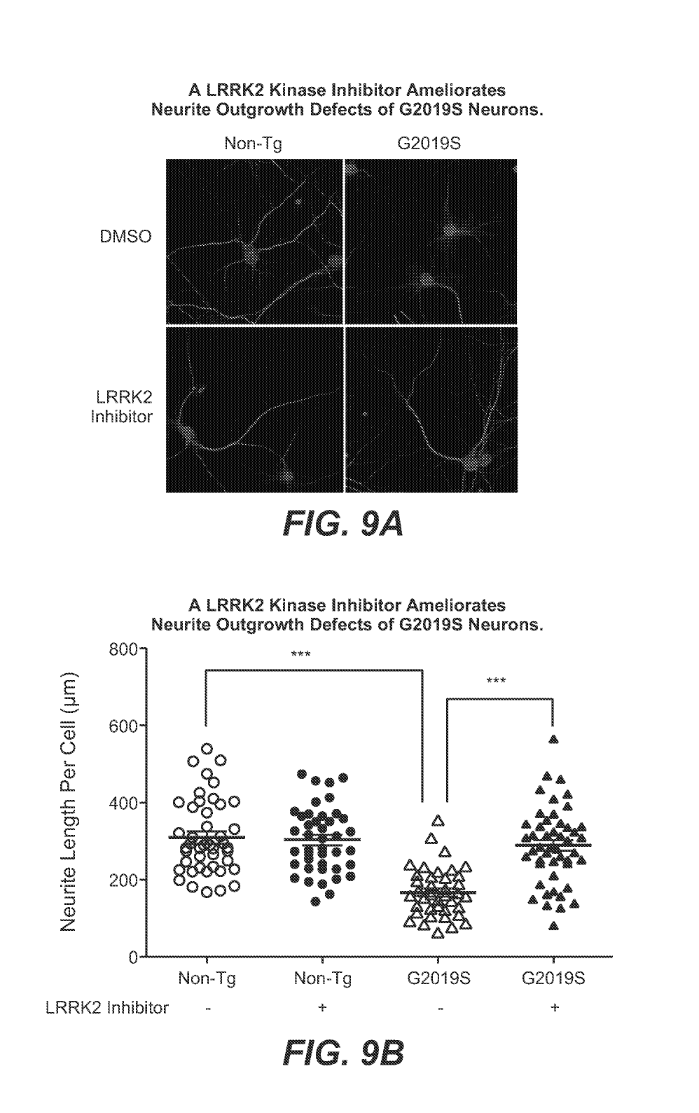

To test whether inhibition of LRRK2 kinase activity ameliorates PD mutations induced neuronal toxicity, we used cultured embryonic hippocampal neurons derived from LRRK2 BAC transgenic mice. These neurons showed neurite outgrowth defects as demonstrated by shorter total neurite length when compared with hippocampal neurons derived from non-transgenic littermates (FIG. 9). We treated the neuronal cultures with LRRK2 kinase inhibitor G1023. We found that G1023 reversed neurite outgrowth defect of neurons derived from LRRK2 BAC transgenic mice in a dose-dependent manner. These data show that chemical inhibition of LRRK2 kinase activity can functionally block LRRK2 PD mutation induced neuronal toxicity.

### S1292 is an In Vivo LRRK2 Autophosphorylation Site

From the results disclosed herein, it can be seen that S1292 is an in vivo LRRK2 autophosphorylation site. Several lines of evidence support that LRRK2 kinase activity is required for phosphorylation of S 1292. First, the kinase-dead mutations abolished S1292 phosphorylation in in vitro autophosphrylation assay (FIG. 1), transfected heterologous cells (FIG. 2A), and tissues from BAC transgenic mice overexpressing LRRK2 (FIG. 2B) Second, a highly specific LRRK2 inhibitor blocks S1292 phosphorylation in both a LRRK2 cell line (FIG. 7B) and a LRRK2 transgenic mice (FIG. 8). Finally, many structurally diversified small molecule LRRK2 kinase inhibitors blocks S1292 phosphorylation in cell-based assay, providing chemical validation that LRRK2 kinase activity is required for S1292 phosphorylation in cells. Importantly, the concentrations required for pLRRK2 reduction are closely related to LRRK2 biochemical Ki of the compounds. Taken together, all the evidence suggests that S1292 is an in vivo LRRK2 autophosphoryaltion site.

While many other LRRK2 autophosphorylation sites have been reported previously, none were detectable in cells or animal models, even in overexpression situations. S1292 autophosphorylation is readily detectable in lysates from transfected cells and transgenic animals. S1292 is the only LRRK2 autophosphorylation site yet identified that exists in significant amount in vivo. Identification of the S1292 autophosphorylation site provides a powerful biomarker tool for monitoring LRRK2 kinase activity in mammalian subjects.

### Familial Parkinson's Mutations Increase LRRK2 Autophosphorylation on S1292

All the confirmed familial Parkinson's disease mutations of LRRK2, including R1441G, Y1699C, G2019S and 12020T, increase S1292 autophosphorylation when expressed in 293T cells. Of these familial mutations, only G2019S has been concretely shown to increase LRRK2 kinase activity in vitro. G2019S is located in kinase activation loop. It has been suggested that G2019S mutation may change the conformation of the kinase activation loop hence activate LRRK2 kinase. However, 12020T, which is also near kinase activation loop, was not consistently found to increase LRRK2 kinase activity in vitro. One possible explanation is that the substrates used in the biochemical assays were not endogenous substrates. Therefore phosphorylation of these proteins may not accurately represent in vivo LRRK2 kinase activity. Herein it is shown that both mutations can increase S1292 phosphorylation in cell-based systems, suggesting that both PD mutations in LRRK2 kinase domain increase its kinase activity. R1441G and Y1699C mutations are located in the ROC-COR domain. Both mutations have been shown to reduce LRRK2 GTPase activity. GTP binding has been shown to be required for LRRK2 kinase activity in vitro. This disclosure demonstrates that GTP binding is required for S1292 autophosphorylation in cell-based system (FIG. 5A). Therefore, reduction of GTPase activity caused by R1441G and Y1699C mutation would result in more LRRK2 protein in GTP-bound state, hence increase S1292 autophosphorylation.

Notably, the confirmed PD risk factor mutation G2385R located in WD40 domain, was not found to increase S1292 autophosphorylation. One simple explanation could be that G2385R mutation may not affect LRRK2 kinase activity or autophosphorylation. Alternatively, heterologous expression of LRRK2 in 293 cells may not provide the suitable cellular environment for LRRK2 autophosphorylation. When comparing S1292 autophosphorylation level of G2019S mutation in vitro, in 293 cells and in animal models, the levels of increase were 3, 5 and 10, respectively. This suggests that in vivo S1292 autophosphorylation is higher than in vitro.

### The Role of S1292 Autophosphorylation in LRRK2 PD Mutation Induced Neuronal Toxicity

Given that increased S1292 autophosphorylation is a unifying theme for all the familial Parkinson's mutations, it is reasonable to hypothesize that S1292 autophosphorylation may be important for LRRK2 function and its role in PD pathogenesis. In cell-based LRRK2 models, Parkinson's mutations have been shown to cause cellular toxicity in both cell lines and primary neurons. Importantly, kinase activity has been shown to be required for the toxicity. The fact that expression of LRRK2 R1441G/G2019S mutations in embryonic hippocampal neurons reduces neurite length and complexity confirms the published results. Also consistent with the published data, kinase-dead mutation was found to ameliorate the neurite outgrowth phenotypes. Importantly, S1292A mutation also reduces neurite outgrowth phenotypes. S1292 autophosphorylation may be part of the mechanisms downstream of LRRK2 kinase activity to induce cellular toxicity. Notably, S1292 is located at the junction of LRR domain and ROC domain. Phosphorylation of this site may induce protein conformation change and result in change of protein-protein interaction.

The mechanisms through which LRRK2 PD mutations induce neuronal toxicity remain unknown. LRRK2 proteins carrying PD mutations were found to accumulate on microtubules when expressed in neurons. This is consistent with results in cell lines and primary neurons. These aggregates are positive for selective autophagy substrate p62 (SQSTM1). Accumulation of p62 positive aggregates is a characteristic of impaired autophagy function. Since autophagy cargos in neurons are thought to traffick along the microtubule network en route to lysosomes, accumulation of p62 on microtubules may suggest impaired autophagy trafficking in neurons expressing LRRK2 PD mutations. Since LRRK2 was observed accumulating in large puncta that stain positive for p62, LC3 and Lys-63 poly-ubiquitin, autolysosomal degradation pathway may be impaired. Autophagy is an important “house keeping” function in differentiated neurons, while loss of autophagy leads to neurodegeneration and results in neurologic defects. This suggests that LRRK2 PD mutations may cause cellular toxicity through autophagy impairment. Interestingly, since the S1292A mutation does not ameliorate appearance of LRRK2-positive autolysomes, Ser1292 phosphorylation may function upstream of autolysomal degradation. Importantly, kinase-dead mutation (D1994A) abolished both microtubule and autolysosome accumulation of LRRK2, suggesting that kinase activity is required for both. It remains likely that substrates of the LRRK2 remain to be identified.

### LRRK2 Autophosphorylation as a Marker for In Vivo LRRK2 Kinase Activity

S1292 autophosphorylation serves as a robust marker for in vivo LRRK2 kinase activity. Furthermore, S 1292 autophosphorylation can be easily detected in tissue lysates from LRRK2 transgenic animals. This provides a pharmacodynamic assay to monitor the knockdown of LRRK2 kinase activity in vivo. Taken together, S1292 autophosphorylation can be used to develop compound screening tools for LRRK2 kinase inhibitors.

### LRRK2 Modulators

LRRK2 modulators or inhibitors usable with the invention may comprise compounds of formula I or compounds of formula II.

In one embodiment, the LRRK2 modulators are of formula I:

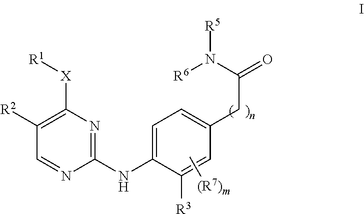

or pharmaceutically acceptable salts thereof,
wherein:

m is from 0 to 3;

X is: —NR^{a}—; —O—; or —S(O)ᵣ— wherein r is from 0 to 2 and R^{a} is hydrogen or C₁₋₆alkyl;

R¹ is: C₁₋₆alkyl; halo-C₁₋₆alkyl; C₂₋₆alkenyl; C₂₋₆alkynyl; halo-C₁₋₆alkyl; C₁₋₆alkoxy-C₁₋₆alkyl; hydroxy-C₁₋₆alkyl; amino-C₁₋₆alkyl; C₁₋₆alkylsulfonyl-C₁₋₆alkyl; C₃₋₆cycloalkyl optionally substituted with C₁₋₆alkyl or halo; C₃₋₆cycloalkyl-C₁₋₆alkyl wherein the C₃₋₆cycloalkyl portion is optionally substituted with C₁₋₆alkyl; tetrahydropyranyl; tetrahydrofuranyl; tetrahydrofuranyl-C₁₋₆alkyl; oxetanyl; or oxetan-C₁₋₆alkyl;

or R¹ and R^{a} together with the atoms to which they are attached may form a three to six membered ring that may optionally include an additional heteroatom selected from O, N and S, and which is substituted with oxo, halo or C₁₋₆alkyl;

R² is: halo; C₁₋₆alkoxy; cyano; C₂₋₆alkynyl; C₂₋₆alkenyl; halo-C₁₋₆alkyl; halo-C₁₋₆alkoxy; C₃₋₆cycloalkyl wherein the C₃₋₆cycloalkyl portion is optionally substituted with C₁₋₆alkyl; C₃₋₆cycloalkyl-C₁₋₆alkyl wherein the C₃₋₆cycloalkyl portion is optionally substituted with C₁₋₆alkyl; tetrahydrofuranyl; tetrahydrofuranyl-C₁₋₆alkyl; acetyl; oxetanyl; or oxetan-C₁₋₆alkyl;

R³ is: —OR⁴; halo; cyano; C₁₋₆alkyl; halo-C₁₋₆alkyl; C₃₋₆cycloalkyl optionally substituted with C₁₋₆alkyl; C₃₋₆cycloalkyl-C₁₋₆alkyl wherein the C₃₋₆cycloalkyl portion is optionally substituted with C₁₋₆alkyl; tetrahydrofuranyl; tetrahydrofuranyl-C₁₋₆alkyl; oxetanyl; or oxetan-C₁₋₆alkyl;

R⁴ is: hydrogen; C₁₋₆alkyl; halo-C₁₋₆alkyl; C₁₋₆alkoxy-C₁₋₆alkyl; C₃₋₆cycloalkyl optionally substituted with C₁₋₆alkyl or halo; C₃₋₆cycloalkyl-C₁₋₆alkyl wherein the C₃₋₆cycloalkyl portion is optionally substituted with C₁₋₆alkyl or halo; tetrahydrofuranyl; tetrahydrofuranyl-C₁₋₆alkyl; oxetanyl; or oxetan-C₁₋₆alkyl;

R⁵ is: hydrogen; or C₁₋₆alkyl;

n is 0 or 1;

R⁶ is: hydrogen; C₁₋₆alkyl; C₁₋₆alkoxy-C₁₋₆alkyl; hydroxy-C₁₋₆alkyl; amino-C₁₋₆alkyl; C₃₋₆cycloalkyl; C₃₋₆cycloalkyl-C₁₋₆alkyl; heterocyclyl; or heterocyclyl-C₁₋₆alkyl; wherein the C₃₋₆cycloalkyl, C₃₋₆cycloalkyl-C₁₋₆alkyl, heterocyclyl and heterocyclyl-C₁₋₆alkyl each may be optionally substituted with one, two, three or four groups groups independently selected from: C₁₋₆alkyl; halo-C₁₋₆alkyl; C₁₋₆alkoxy; halo-C₁₋₆alkoxy; hydroxy; hydroxy-C₁₋₆alkyl; halo; nitrile; C₁₋₆alkyl-carbonyl; C₁₋₆alkyl-sulfonyl; C₃₋₆cycloalkyl; C₃₋₆cycloalkyl-C₁₋₆alkyl; C₃₋₆cycloalkyl-carbonyl; amino; or heterocyclyl; or two of the groups together with the atoms to which they are attached may form a five or six-membered ring;

or R⁵ and R⁶ together with the nitrogen atom to which they are attached form a three- to seven-membered ring that optionally includes an additional heteroatom selected from O, N and S(O)ₙ and which is optionally substituted with one, two, three or four groups independently selected from: C₁₋₆alkyl; halo-C₁₋₆alkyl; C₁₋₆alkoxy; halo-C₁₋₆alkoxy; hydroxy; C₁₋₆alkoxy-C₁₋₆alkyl; hydroxy-C₁₋₆alkyl; halo, nitrile; C₁₋₆alkyl-carbonyl; C₁₋₆alkyl-sulfonyl; C₃₋₆cycloalkyl; C₃₋₆cycloalkyl-C₁₋₆alkyl; C₃₋₆cycloalkyl-carbonyl; amino; or heterocyclyl; or two of the groups together with the atoms to which they are attached may form a five or six-membered ring; and

R⁷ is: halo; C₁₋₆alkyl; C₁₋₆alkoxy; halo-C₁₋₆alkyl; or halo-C₁₋₆alkoxy.

In certain embodiments of formula I, n is 0.

In certain embodiments of formula I, n is 1.

In certain embodiments of formula I, m is from 0 to 2.

In certain embodiments of formula I, m is 0 or 1.

In certain embodiments of formula I, m is 0.

In certain embodiments of formula I, m is 1.

In certain embodiments of formula I, r is 0.

In certain embodiments of formula I, r is 2.

In certain embodiments of formula I, X is —NR^{a}— or —O—.

In certain embodiments of formula I, X is —NR^{a}.

In certain embodiments of formula I, X is —O—.

In certain embodiments of formula I, X is —S(O)—.

In certain embodiments of formula I, X is —NH— or —O—.

In certain embodiments of formula I, R^{a} is hydrogen.

In certain embodiments of formula I, R^{a} is C₁₋₆alkyl.

In certain embodiments of formula I, R¹ is: C₁₋₆alkyl; halo-C₁₋₆alkyl; C₁₋₆alkoxy-C₁₋₆alkyl; amino-C₁₋₆alkyl; C₁₋₆alkylsulfonyl-C₁₋₆alkyl; C₃₋₆cycloalkyl; or C₃₋₆cycloalkyl-C₁₋₆alkyl.

In certain embodiments of formula I, R¹ is: C₁₋₆alkyl; C₃₋₆cycloalkyl optionally substituted with C₁₋₆alkyl; or C₃₋₆cycloalkyl-C₁₋₆alkyl wherein the C₃₋₆cycloalkyl portion is optionally substituted with C₁₋₆alkyl.

In certain embodiments of formula I, R¹ is: C₁₋₆alkyl; halo-C₁₋₆alkyl; C₁₋₆alkoxy-C₁₋₆alkyl; amino-C₁₋₆alkyl; C₁₋₆alkylsulfonyl-C₁₋₆alkyl; tetrahydrofuranyl; tetrahydrofuranyl-C₁₋₆alkyl; oxetanyl; or oxetan-C₁₋₆alkyl.

In certain embodiments of formula I, R¹ is: C₁₋₆alkyl; halo-C₁₋₆alkyl; C₁₋₆alkoxy-C₁₋₆alkyl; amino-C₁₋₆alkyl; or C₁₋₆alkylsulfonyl-C₁₋₆alkyl.

In certain embodiments of formula I, R¹ is C₁₋₆alkyl.

In certain embodiments of formula I, R¹ is halo-C₁₋₆alkyl.

In certain embodiments of formula I, R¹ is C₁₋₆alkoxy-C₁₋₆alkyl.

In certain embodiments of formula I, R¹ is amino-C₁₋₆alkyl.

In certain embodiments of formula I, R¹ is C₁₋₆alkylsulfonyl-C₁₋₆alkyl optionally substituted with C₁₋₆alkyl.

In certain embodiments of formula I, R¹ is C₃₋₆cycloalkyl optionally substituted with C₁₋₆alkyl.

In certain embodiments of formula I, R¹ is C₃₋₆cycloalkyl-C₁₋₆alkyl wherein the C₃₋₆cycloalkyl portion is optionally substituted with C₁₋₆alkyl.

In certain embodiments of formula I, R¹ is tetrahydropyranyl.

In certain embodiments of formula I, R¹ is tetrahydrofuranyl.

In certain embodiments of formula I, R¹ is tetrahydrofuranyl-C₁₋₆alkyl; oxetanyl.

In certain embodiments of formula I, R¹ is or oxetan-C₁₋₆alkyl.

In certain embodiments of formula I, R¹ is: methyl; ethyl; n-propyl; isopropyl; isobutyl; 3,3-dimethylpropyl; cyclopropyl; cyclobutyl; cyclopentyl; cyclohexyl; cyclopropylmethyl; cyclobutylmethyl; cyclopentylmethyl; cyclopropylethyl; methoxyethyl; oxetanyl; or tetrahydrofuranylmethyl.

In certain embodiments of formula I, R¹ is: methyl; ethyl; n-propyl; isopropyl; isobutyl; 3,3-dimethylpropyl; cyclopentyl; cyclohexyl; cyclopropylmethyl; cyclobutylmethyl; cyclopentylmethyl; cyclopropylethyl; methoxyethyl; oxetanyl; or tetrahydrofuranylmethyl.

In certain embodiments of formula I, R¹ is: methyl; ethyl; n-propyl; isopropyl; isobutyl; 3,3-dimethylpropyl; cyclopentyl; cyclohexyl; cyclopentylmethyl; methoxyethyl; oxetanyl; or tetrahydrofuranylmethyl.

In certain embodiments of formula I, R¹ is: methyl; ethyl; n-propyl; isopropyl; or isobutyl.

In certain embodiments of formula I, R¹ is methyl or ethyl.

In certain embodiments of formula I, R¹ is methyl.

In certain embodiments of formula I, R¹ is ethyl.

In certain embodiments of formula I, R¹ is: cyclopropyl; cyclobutyl; cyclopentyl; cyclohexyl; cyclopropylmethyl; cyclobutylmethyl; cyclopentylmethyl; or cyclopropylethyl.

In certain embodiments of formula I, R¹ is: cyclopentyl; cyclohexyl; or cyclopentylmethyl.

In certain embodiments of formula I, R² is: halo; C₁₋₆alkoxy; halo-C₁₋₆alkyl; halo-C₁₋₆alkoxy; C₃₋₆cycloalkyl wherein the C₃₋₆cycloalkyl portion is optionally substituted with C₁₋₆alkyl; C₃₋₆cycloalkyl-C₁₋₆alkyl wherein the C₃₋₆cycloalkyl portion is optionally substituted with C₁₋₆alkyl; tetrahydrofuranyl; tetrahydrofuranyl-C₁₋₆alkyl; oxetanyl; or oxetan-C₁₋₆alkyl.

In certain embodiments of formula I, R² is: halo; C₁₋₆alkoxy; halo-C₁₋₆alkyl; cyano; C₂₋₆alkynyl; C₂₋₆alkenyl; C₃₋₆cycloalkyl; or C₃₋₆cycloalkyl-C₁₋₆alkyl.

In certain embodiments of formula I, R² is: halo; C₁₋₆alkoxy; halo-C₁₋₆alkyl; cyano; C₃₋₆cycloalkyl; or C₃₋₆cycloalkyl-C₁₋₆alkyl.

In certain embodiments of formula I, R² is: halo; C₁₋₆alkoxy; halo-C₁₋₆alkyl; C₃₋₆cycloalkyl; or C₃₋₆cycloalkyl-C₁₋₆alkyl.

In certain embodiments of formula I, R² is: halo; halo-C₁₋₆alkyl; or cyano.

In certain embodiments of formula I, R² is: halo; or halo-C₁₋₆alkyl.

In certain embodiments of formula I, R² is halo.

In certain embodiments of formula I, R² is C₁₋₆alkoxy.

In certain embodiments of formula I, R² is halo-C₁₋₆alkoxy.

In certain embodiments of formula I, R² is halo-C₁₋₆alkyl.

In certain embodiments of formula I, R² is C₃₋₆cycloalkyl.

In certain embodiments of formula I, R² is C₃₋₆cycloalkyl-C₁₋₆alkyl.

In certain embodiments of formula I, R² is tetrahydrofuranyl.

In certain embodiments of formula I, R² is tetrahydrofuranyl-C₁₋₆alkyl.

In certain embodiments of formula I, R² is oxetanyl.

In certain embodiments of formula I, R² is oxetan-C₁₋₆alkyl.

In certain embodiments of formula I, R² is halo, trifluoromethyl or cyano.

In certain embodiments of formula I, R² is chloro, trifluoromethyl or cyano.

In certain embodiments of formula I, R² is fluoro, chloro or bromo.

In certain embodiments of formula I, R² is chloro.

In certain embodiments of formula I, R² is fluoro.

In certain embodiments of formula I, R² is bromo.

In certain embodiments of formula I, R² is trifluoromethyl.

In certain embodiments of formula I, R² is methoxy.

In certain embodiments of formula I, R² is cyano.

In certain embodiments of formula I, R² is C₂₋₆alkynyl.

In certain embodiments of formula I, R² is C₂₋₆alkenyl.

In certain embodiments of formula I, R³ is —OR⁴.

In certain embodiments of formula I, R³ is: C₁₋₆alkyl; or halo-C₁₋₆alkyl.

In certain embodiments of formula I, R³ is: halo; or —OR⁴.

In certain embodiments of formula I, R³ is: halo; C₁₋₆alkoxy; halo-C₁₋₆alkoxy; C₃₋₆cycloalkyloxy; or C₃₋₆cycloalkyl-C₁₋₆alkyloxy.

In certain embodiments of formula I, R³ is: C₁₋₆alkoxy; halo-C₁₋₆alkoxy; C₃₋₆cycloalkyloxy; or C₃₋₆cycloalkyl-C₁₋₆alkyloxy.

In certain embodiments of formula I, R³ is: halo; C₁₋₆alkoxy; cyano; or halo-C₁₋₆alkoxy.

In certain embodiments of formula I, R³ is: halo; C₁₋₆alkoxy; or halo-C₁₋₆alkoxy.

In certain embodiments of formula I, R³ is: methoxy; halo; trifluoromethoxy; difluoromethoxy; 2-halo-ethoxy or 2,2,2-trihaloethoxy.

In certain embodiments of formula I, R³ is: methoxy; or halo.

In certain embodiments of formula I, R³ is: methoxy; chloro; or fluoro.

In certain embodiments of formula I, R³ is methoxy.

In certain embodiments of formula I, R³ is chloro.

In certain embodiments of formula I, R³ is fluoro.

In certain embodiments of formula I, R³ is: C₁₋₆alkoxy; cyano; or halo-C₁₋₆alkoxy.

In certain embodiments of formula I, R³ is: C₁₋₆alkoxy; or halo-C₁₋₆alkoxy.

In certain embodiments of formula I, R³ is C₁₋₆alkoxy.

In certain embodiments of formula I, R³ is methoxy.

In certain embodiments of formula I, R³ is cyano.

In certain embodiments of formula I, R³ is C₃₋₆cycloalkyl.

In certain embodiments of formula I, R³ is C₃₋₆cycloalkyl-C₁₋₆alkyl.

In certain embodiments of formula I, R³ is tetrahydrofuranyl. In certain embodiments of formula I, R³ is tetrahydrofuranyl-C₁₋₆alkyl.

In certain embodiments of formula I, R³ is oxetanyl.

In certain embodiments of formula I, R³ is oxetan-C₁₋₆alkyl.

In certain embodiments of formula I, R⁴ is: C₁₋₆alkyl; halo-C₁₋₆alkyl; C₁₋₆alkoxy-C₁₋₆alkyl; C₃₋₆cycloalkyl optionally substituted with C₁₋₆alkyl; C₃₋₆cycloalkyl-C₁₋₆alkyl wherein the C₃₋₆cycloalkyl portion is optionally substituted with C₁₋₆alkyl; tetrahydrofuranyl; tetrahydrofuranyl-C₁₋₆alkyl; oxetanyl; or oxetan-C₁₋₆alkyl;

In certain embodiments of formula I, R⁴ is: C₁₋₆alkyl; halo-C₁₋₆alkyl; C₁₋₆alkoxy-C₁₋₆alkyl; C₃₋₆cycloalkyl; or C₃₋₆cycloalkyl-C₁₋₆alkyl.

In certain embodiments of formula I, R⁴ is: C₁₋₆alkyl; halo-C₁₋₆alkyl; or C₃₋₆cycloalkyl.

In certain embodiments of formula I, R⁴ is C₁₋₆alkyl.

In certain embodiments of formula I, R⁴ is halo-C₁₋₆alkyl.

In certain embodiments of formula I, R⁴ is C₁₋₆alkoxy-C₁₋₆alkyl.

In certain embodiments of formula I, R⁴ is C₃₋₆cycloalkyl.

In certain embodiments of formula I, R⁴ is C₃₋₆cycloalkyl-C₁₋₆alkyl.

In certain embodiments of formula I, R⁴ is tetrahydrofuranyl.

In certain embodiments of formula I, R⁴ is tetrahydrofuranyl-C₁₋₆alkyl.

In certain embodiments of formula I, R⁴ is oxetanyl.

In certain embodiments of formula I, R⁴ is or oxetan-C₁₋₆alkyl.

In certain embodiments of formula I, R⁴ is: methyl; ethyl; isopropyl; cyclopropyl; cyclobutyl; cyclopropylmethyl; cyclobutylmethyl; 2-haloethyl; or 2,2,2-trihaloethyl.

In certain embodiments of formula I, R⁴ is methyl.

In certain embodiments of formula I, R⁵ is hydrogen.

In certain embodiments of formula I, R⁵ is C₁₋₆alkyl.

In certain embodiments of formula I, R⁵ is methyl.

In certain embodiments of formula I, R⁵ is ethyl.

In certain embodiments of formula I, R⁶ is hydrogen.

In certain embodiments of formula I, R⁶ is C₁₋₆alkyl.

In certain embodiments of formula I, R⁶ is C₁₋₆alkoxy-C₁₋₆alkyl.

In certain embodiments of formula I, R⁶ is hydroxy-C₁₋₆alkyl.

In certain embodiments of formula I, R⁶ is amino-C₁₋₆alkyl.

In certain embodiments of formula I, R⁶ is C₃₋₆cycloalkyl optionally substituted with one, two or three groups independently selected from: C₁₋₆alkyl; halo-C₁₋₆alkyl; C₁₋₆alkoxy; halo-C₁₋₆alkoxy; hydroxy; hydroxy-C₁₋₆alkyl; halo; nitrile; C₁₋₆alkyl-carbonyl; C₁₋₆alkyl-sulfonyl; C₃₋₆cycloalkyl; C₃₋₆cycloalkyl-C₁₋₆alkyl; C₃₋₆cycloalkyl-carbonyl; amino; or heterocyclyl; or two of the groups together with the atoms to which they are attached may form a five or six-membered ring.

In certain embodiments of formula I, R⁶ is C₃₋₆cycloalkyl-C₁₋₆alkyl wherein the C₃₋₆cycloalkyl portion thereof is optionally substituted with one, two or three groups independently selected from: C₁₋₆alkyl; halo-C₁₋₆alkyl; C₁₋₆alkoxy; halo-C₁₋₆alkoxy; hydroxy; hydroxy-C₁₋₆alkyl; halo; nitrile; C₁₋₆alkyl-carbonyl; C₁₋₆alkyl-sulfonyl; C₃₋₆cycloalkyl; C₃₋₆cycloalkyl-C₁₋₆alkyl; C₃₋₆cycloalkyl-carbonyl; amino; or heterocyclyl; or two of the groups together with the atoms to which they are attached may form a five or six-membered ring.

In embodiments of formula I wherein R⁶ is heterocyclyl, such heterocycle may be: azetidinyl; pyrrolidinyl; piperidinyl; piperazinyl; morpholinyl; thiomorpholinyl; 3-oxa-8-aza-bicyclo[3.2.1]oct-8-yl; 2-oxa-5-aza-bicyclo[2.2.1]hept-5-yl; or 8-oxa-3-aza-bicyclo[3.2.1]oct-3-yl; each optionally substituted as defined herein.

In embodiments of formula I wherein R⁶ is heterocyclyl, such heterocycle may be: azetidinyl; pyrrolidinyl; piperidinyl; piperazinyl; or morpholinyl; each optionally substituted as defined herein, i.e., such heterocycyl is optionally substituted with one, two or three groups independently selected from: C₁₋₆alkyl; halo-C₁₋₆alkyl; C₁₋₆alkoxy; halo-C₁₋₆alkoxy; hydroxy; hydroxy-C₁₋₆alkyl; halo; nitrile; C₁₋₆alkyl-carbonyl; C₁₋₆alkyl-sulfonyl; C₃₋₆cycloalkyl; C₃₋₆cycloalkyl-C₁₋₆alkyl; C₃₋₆cycloalkyl-carbonyl; amino; or heterocyclyl; or two of the groups together with the atoms to which they are attached may form a five or six-membered ring.

In certain embodiments of formula I, R⁶ is heterocyclyl optionally substituted with one, two or three groups independently selected from: C₁₋₆alkyl; halo-C₁₋₆alkyl; C₁₋₆alkoxy; halo-C₁₋₆alkoxy; hydroxy; hydroxy-C₁₋₆alkyl; halo; nitrile; C₁₋₆alkyl-carbonyl; C₁₋₆alkyl-sulfonyl; C₃₋₆cycloalkyl; C₃₋₆cycloalkyl-C₁₋₆alkyl; C₃₋₆cycloalkyl-carbonyl; amino; or heterocyclyl; or two of the groups together with the atoms to which they are attached may form a five or six-membered ring.

In embodiments of formula I wherein R⁶ is heterocyclyl-C₁₋₆alkyl, the heterocyclyl portion thereof may be: azetidinyl; pyrrolidinyl; piperidinyl; piperazinyl; morpholinyl; thiomorpholinyl; 3-oxa-8-aza-bicyclo[3.2.1]oct-8-yl; 2-oxa-5-aza-bicyclo[2.2.1]hept-5-yl; or 8-oxa-3-aza-bicyclo[3.2.1]oct-3-yl; each optionally substituted as defined herein, i.e., such heterocycyl portion is optionally substituted with one, two or three groups independently selected from: C₁₋₆alkyl; halo-C₁₋₆alkyl; C₁₋₆alkoxy; halo-C₁₋₆alkoxy; hydroxy; hydroxy-C₁₋₆alkyl; halo; nitrile; C₁₋₆alkyl-carbonyl; C₁₋₆alkyl-sulfonyl; C₃₋₆cycloalkyl; C₃₋₆cycloalkyl-C₁₋₆alkyl; C₃₋₆cycloalkyl-carbonyl; amino; or heterocyclyl; or two of the groups together with the atoms to which they are attached may form a five or six-membered ring.

In embodiments of formula I wherein R⁶ is heterocyclyl-C₁₋₆alkyl, the heterocyclyl portion thereof may be: azetidinyl; pyrrolidinyl; piperidinyl; piperazinyl; or morpholinyl; each optionally substituted as defined herein.

In certain embodiments of formula I, R⁶ is heterocyclyl-C₁₋₆alkyl wherein the heterocyclyl portion thereof is optionally substituted with one, two or three groups independently selected from: C₁₋₆alkyl; halo-C₁₋₆alkyl; C₁₋₆alkoxy; halo-C₁₋₆alkoxy; hydroxy; hydroxy-C₁₋₆alkyl; halo; nitrile; C₁₋₆alkyl-carbonyl; C₁₋₆alkyl-sulfonyl; C₃₋₆cycloalkyl; C₃₋₆cycloalkyl-C₁₋₆alkyl; C₃₋₆cycloalkyl-carbonyl; amino; or heterocyclyl; or two of the groups together with the atoms to which they are attached may form a five or six-membered ring.

In certain embodiments of formula I, R⁶ is: hydrogen; methyl; ethyl; isopropyl; or cyclopropyl.

In certain embodiments of formula I, R⁶ is: hydrogen; methyl; ethyl; isopropyl; 2-amino-propyl; oxetan-3-yl; 2-methoxy-ethyl; 2-hydroxy-ethyl; cyclopropyl; piperidin-4-yl; 1-methyl-piperidin-4-yl; tert-butyl; 2-hydroxy-2-methyl-propyl; cyclobutyl; 1-methyl-cyclobutyl; 2-hydroxy-propyl; 1-cyano-cyclopropyl; 3,3-difluoro-cyclobutyl; cyclopropylmethyl; 3-fluoro-cyclobutyl; or 2,2-difluoroethyl;

In certain embodiments of formula I, R⁶ is hydrogen.

In certain embodiments of formula I, R⁶ is methyl.

In certain embodiments of formula I, R⁶ is ethyl.

In certain embodiments of formula I, R⁶ is isopropyl.

In certain embodiments of formula I, R⁶ is 2-amino-propyl.

In certain embodiments of formula I, R⁶ is oxetan-3-yl.

In certain embodiments of formula I, R⁶ is 2-methoxy-ethyl.

In certain embodiments of formula I, R⁶ is 2-hydroxy-ethyl.

In certain embodiments of formula I, R⁶ is cyclopropyl.

In certain embodiments of formula I, R⁶ is piperidin-4-yl.

In certain embodiments of formula I, R⁶ is 1-methyl-piperidin-4-yl.

In certain embodiments of formula I, R⁶ is tert-butyl.

In certain embodiments of formula I, R⁶ is 2-hydroxy-2-methyl-propyl.

In certain embodiments of formula I, R⁶ is cyclobutyl.

In certain embodiments of formula I, R⁶ is 1-methyl-cyclobutyl.

In certain embodiments of formula I, R⁶ is 2-hydroxy-propyl.

In certain embodiments of formula I, R⁶ is 1-cyano-cyclopropyl.

In certain embodiments of formula I, R⁶ is 3,3-difluoro-cyclobutyl.

In certain embodiments of formula I, R⁶ is cyclopropylmethyl.

In certain embodiments of formula I, R⁶ is 3-fluoro-cyclobutyl.

In certain embodiments of formula I, R⁶ is 2,2-difluoroethyl.

In certain embodiments of formula I, R⁵ and R⁶ together with the nitrogen atom to which they are attached form a three- to seven-membered ring that is optionally includes an additional heteroatom selected from O, N and S(O), and which is optionally substituted with one, two or three groups independently selected from C₁₋₆alkyl, halo-C₁₋₆alkyl, C₁₋₆alkoxy, halo-C₁₋₆alkoxy, hydroxy, hydroxy-C₁₋₆alkyl, halo, nitrile, C₁₋₆alkyl-carbonyl, C₁₋₆alkyl-sulfonyl, C₃₋₆cycloalkyl, C₃₋₆cycloalkyl-C₁₋₆alkyl, C₃₋₆cycloalkyl-carbonyl, or heterocyclyl, or two of the groups together with the atoms to which they are attached may form a five or six-membered ring.

In embodiments of formula I wherein R⁵ and R⁶ together with the nitrogen atom to which they are attached form a three- to seven-membered ring that is optionally includes an additional heteroatom selected from O, N and S(O), such ring may be: azetidinyl; pyrrolidinyl; piperidinyl; piperazinyl; morpholinyl; thiomorpholinyl; azepinyl; 3-oxa-8-aza-bicyclo[3.2.1]oct-8-yl; 2-oxa-5-aza-bicyclo[2.2.1]hept-5-yl; or 8-oxa-3-aza-bicyclo[3.2.1]oct-3-yl; each optionally substituted as defined herein.

In embodiments of formula I wherein R⁵ and R⁶ together with the nitrogen atom to which they are attached form a three- to seven-membered ring that is optionally includes an additional heteroatom selected from O, N and S(O), such ring may be: azetidinyl; pyrrolidinyl; piperidinyl; piperazinyl; or morpholinyl; each optionally substituted as defined herein.

In certain embodiments of formula I, R⁵ and R⁶ together with the nitrogen atom to which they are attached form a morpholinyl group that is optionally substituted once or twice with groups independently selected from C₁₋₆alkyl, halo-C₁₋₆alkyl, C₁₋₆alkoxy, halo-C₁₋₆alkoxy, hydroxy, hydroxy-C₁₋₆alkyl, halo, nitrile, C₁₋₆alkyl-carbonyl, C₁₋₆alkyl-sulfonyl, C₃₋₆cycloalkyl, C₃₋₆cycloalkyl-C₁₋₆alkyl, C₃₋₆cycloalkyl-carbonyl, amino, or heterocyclyl, or the two groups together with the atoms to which they are attached may form a five or six-membered ring.

In certain embodiments of formula I, R⁵ and R⁶ together with the nitrogen atom to which they are attached form a piperidinyl group that is optionally substituted once or twice with groups independently selected from C₁₋₆alkyl, halo-C₁₋₆alkyl, C₁₋₆alkoxy, halo-C₁₋₆alkoxy, hydroxy, hydroxy-C₁₋₆alkyl, halo, nitrile, C₁₋₆alkyl-carbonyl, C₁₋₆alkyl-sulfonyl, C₃₋₆cycloalkyl, C₃₋₆cycloalkyl-C₁₋₆alkyl, C₃₋₆cycloalkyl-carbonyl, amino, or heterocyclyl, or the two groups together with the atoms to which they are attached may form a five or six-membered ring.

In certain embodiments of formula I, R⁵ and R⁶ together with the nitrogen atom to which they are attached form a piperazinyl group that is optionally substituted once or twice with groups independently selected from C₁₋₆alkyl, halo-C₁₋₆alkyl, C₁₋₆alkoxy, halo-C₁₋₆alkoxy, hydroxy, hydroxy-C₁₋₆alkyl, halo, nitrile, C₁₋₆alkyl-carbonyl, C₁₋₆alkyl-sulfonyl, C₃₋₆cycloalkyl, C₃₋₆cycloalkyl-C₁₋₆alkyl, C₃₋₆cycloalkyl-carbonyl, amino, or heterocyclyl, or the two groups together with the atoms to which they are attached may form a five or six-membered ring.

In certain embodiments of formula I, R⁵ and R⁶ together with the nitrogen atom to which they are attached form a pyrrolidinyl group that is optionally substituted once or twice with groups independently selected from C₁₋₆alkyl, halo-C₁₋₆alkyl, C₁₋₆alkoxy, halo-C₁₋₆alkoxy, hydroxy, hydroxy-C₁₋₆alkyl, halo, nitrile, C₁₋₆alkyl-carbonyl, C₁₋₆alkyl-sulfonyl, C₃₋₆cycloalkyl, C₃₋₆cycloalkyl-C₁₋₆alkyl, C₃₋₆cycloalkyl-carbonyl, amino, or heterocyclyl, or the two groups together with the atoms to which they are attached may form a five or six-membered ring.

In certain embodiments of formula I, R⁵ and R⁶ together with the nitrogen atom to which they are attached form a group selected from: morpholin-4-yl; 4-hydroxy-piperidin-1-yl; octahydro-pyrido[1,2-a]pyrazin-2-yl; 2-hydroxy-piperidin-1-yl; 4,4-dimethyl-piperidin-1-yl; 3,5-dimethyl-piperidin-1-yl; 1-hydroxy-1-methyl-ethyl)-piperidin-1-yl; 3-hydroxy-pyrrolidin-1-yl; 4-methyl-piperidin-1-yl; piperidin-1-yl; azetidin-1-yl; 4,4-difluoro-piperidin-1-yl; 3-methyl-piperidin-1-yl; 4-methoxy-piperidin-1-yl; 3,3-difluoro-piperidin-1-yl; 4-cyano-piperidin-1-yl; 4-fluoro-piperidin-1-yl; 3-methoxy-piperidin-1-yl; 4-ethyl-piperazin-1-yl; 4-acetyl-piperazin-1-yl; 3-trifluoromethyl-piperidin-1-yl; 4-tert-butyl-piperidin-1-yl; 2-hydroxy-ethyl)-piperazin-1-yl; 2-methyl-pyrrolidin-1-yl; 4-hydroxymethyl-piperidin-1-yl; 2-methyl-piperidin-1-yl; pyrrolidin-1-yl; 4-methanesulfonyl-piperazin-1-yl; 3-trifluoromethyl-pyrrolidin-1-yl; 4-(2,2,2-trifluoro-ethyl)-piperazin-1-yl; 2-methyl-morpholin-4-yl; (2,6-dimethyl-morpholin-4-yl; 2,2-diethyl-morpholin-4-yl; 3-hydroxymethyl-morpholin-4-yl; 2-isobutyl-morpholin-4-yl; 2-hydroxymethyl-morpholin-4-yl; 3,3-dimethyl-morpholin-4-yl; 4-methyl-piperazin-1-yl; 4-isopropyl-piperazin-1-yl; piperazin-1-yl; 3-oxa-8-aza-bicyclo[3.2.1]oct-8-yl; (S)-3-methyl-morpholin-4-yl; 2-oxa-5-aza-bicyclo[2.2.1]hept-5-yl; 8-oxa-3-aza-bicyclo[3.2.1]oct-3-yl; (R)-3-methyl-morpholin-4-yl; 4-cyclopropanecarbonyl-piperazin-1-yl; 4-(1-hydroxy-1-methyl-ethyl)-piperidin-1-yl; 4-cyclobutyl-piperazin-1-yl; (R)-3-hydroxy-pyrrolidin-1-yl; 4-oxetan-3-yl-piperazin-1-yl; 3-morpholin-4-yl-azetidin-1-yl; 4-(1-methyl-piperidin-4-yl)-piperazin-1-yl; 3,3-difluoro-azetidin-1-yl; 4-dimethylamino-piperidin-1-yl; 4-piperidin-4-yl-piperazin-1-yl; (4,4-difluoro-piperidin-1-yl; (3-morpholin-4-yl-azetidin-1-yl; 2-oxa-6-aza-spiro[3.3]hept-6-yl; 2-oxa-5-aza-bicyclo[2.2.1]hept-5-yl); 4-methoxy-piperidin-1-yl); [1,4]oxazepan-4-yl; 2R,6S)-2,6-dimethyl-morpholin-4-yl; 3-hydroxy-azetidin-1-yl; 3-cyano-pyrrolidin-1-yl; 3,5-dimethyl-piperazin-1-yl; (3R,5S)-dimethyl-piperazin-1-yl; 3-Fluoro-pyrrolidin-1-yl; (S)-3-Fluoro-pyrrolidin-1-yl; piperazin-1-yl; 3,3-Difluoro-pyrrolidin-1-yl; 3,3-Difluoro-azetidin-1-yl; 2,2,6,6-tetrafluoro-morpholin-4-yl; 2-methoxymethyl-pyrrolidin-1-yl; (S)-2-methoxymethyl-pyrrolidin-1-yl; (1S,4S)-2-oxa-5-azabicyclo[2.2.1]heptan-5-yl; (3S,4S)-3,4-difluoropyrrolidin-1-yl; 3,4-difluoropyrrolidin-1-yl; and 3-methoxypyrrolidin-1-yl.

In certain embodiments of formula I, R⁵ and R⁶ together with the nitrogen atom to which they are attached form a group selected from: morpholin-4-yl; 4-hydroxy-piperidin-1-yl; octahydro-pyrido[1,2-a]pyrazin-2-yl; 2-hydroxy-piperidin-1-yl; 4,4-dimethyl-piperidin-1-yl; 3,5-dimethyl-piperidin-1-yl; 1-hydroxy-1-methyl-ethyl)-piperidin-1-yl; 3-hydroxy-pyrrolidin-1-yl; 4-methyl-piperidin-1-yl; piperidin-1-yl; azetidin-1-yl; 4,4-difluoro-piperidin-1-yl; 3-methyl-piperidin-1-yl; 4-methoxy-piperidin-1-yl; 3,3-difluoro-piperidin-1-yl; 4-cyano-piperidin-1-yl; 4-fluoro-piperidin-1-yl; 3-methoxy-piperidin-1-yl; 4-ethyl-piperazin-1-yl; 4-acetyl-piperazin-1-yl; 3-trifluoromethyl-piperidin-1-yl; 4-tert-butyl-piperidin-1-yl; 2-hydroxy-ethyl)-piperazin-1-yl; 2-methyl-pyrrolidin-1-yl; 4-hydroxymethyl-piperidin-1-yl; 2-methyl-piperidin-1-yl; pyrrolidin-1-yl; 4-methanesulfonyl-piperazin-1-yl; 3-trifluoromethyl-pyrrolidin-1-yl; 4-(2,2,2-trifluoro-ethyl)-piperazin-1-yl; 2-methyl-morpholin-4-yl; (2,6-dimethyl-morpholin-4-yl; 2,2-diethyl-morpholin-4-yl; 3-hydroxymethyl-morpholin-4-yl; 2-isobutyl-morpholin-4-yl; 2-hydroxymethyl-morpholin-4-yl; 3,3-dimethyl-morpholin-4-yl; 4-methyl-piperazin-1-yl; 4-isopropyl-piperazin-1-yl; piperazin-1-yl; 3-oxa-8-aza-bicyclo[3.2.1]oct-8-yl; (S)-3-methyl-morpholin-4-yl; 2-oxa-5-aza-bicyclo[2.2.1]hept-5-yl; 8-oxa-3-aza-bicyclo[3.2.1]oct-3-yl; (R)-3-methyl-morpholin-4-yl; 4-cyclopropanecarbonyl-piperazin-1-yl; 4-(1-hydroxy-1-methyl-ethyl)-piperidin-1-yl; 4-cyclobutyl-piperazin-1-yl; (R)-3-hydroxy-pyrrolidin-1-yl; 4-oxetan-3-yl-piperazin-1-yl; 3-morpholin-4-yl-azetidin-1-yl; 4-(1-methyl-piperidin-4-yl)-piperazin-1-yl; 3,3-difluoro-azetidin-1-yl; 4-dimethylamino-piperidin-1-yl; and 4-piperidin-4-yl-piperazin-1-yl.

In certain embodiments of formula I, R⁵ and R⁶ together with the nitrogen atom to which they are attached form morpholin-4-yl.

In certain embodiments of formula I, R⁵ and R⁶ together with the nitrogen atom to which they are attached form 4-hydroxy-piperidin-1-yl.

In certain embodiments of formula I, R⁵ and R⁶ together with the nitrogen atom to which they are attached form octahydro-pyrido[1,2-a]pyrazin-2-yl.

In certain embodiments of formula I, R⁵ and R⁶ together with the nitrogen atom to which they are attached form 2-hydroxy-piperidin-1-yl.

In certain embodiments of formula I, R⁵ and R⁶ together with the nitrogen atom to which they are attached form 4,4-dimethyl-piperidin-1-yl.

In certain embodiments of formula I, R⁵ and R⁶ together with the nitrogen atom to which they are attached form 3,5-dimethyl-piperidin-1-yl.

In certain embodiments of formula I, R⁵ and R⁶ together with the nitrogen atom to which they are attached form 1-hydroxy-1-methyl-ethyl)-piperidin-1-yl.

In certain embodiments of formula I, R⁵ and R⁶ together with the nitrogen atom to which they are attached form 3-hydroxy-pyrrolidin-1-yl.

In certain embodiments of formula I, R⁵ and R⁶ together with the nitrogen atom to which they are attached form 4-methyl-piperidin-1-yl.

In certain embodiments of formula I, R⁵ and R⁶ together with the nitrogen atom to which they are attached form piperidin-1-yl.

In certain embodiments of formula I, R⁵ and R⁶ together with the nitrogen atom to which they are attached form azetidin-1-yl.

In certain embodiments of formula I, R⁵ and R⁶ together with the nitrogen atom to which they are attached form 4,4-difluoro-piperidin-1-yl.

In certain embodiments of formula I, R⁵ and R⁶ together with the nitrogen atom to which they are attached form 3-methyl-piperidin-1-yl.

In certain embodiments of formula I, R⁵ and R⁶ together with the nitrogen atom to which they are attached form 4-methoxy-piperidin-1-yl.

In certain embodiments of formula I, R⁵ and R⁶ together with the nitrogen atom to which they are attached form 3,3-difluoro-piperidin-1-yl.

In certain embodiments of formula I, R⁵ and R⁶ together with the nitrogen atom to which they are attached form 4-cyano-piperidin-1-yl.

In certain embodiments of formula I, R⁵ and R⁶ together with the nitrogen atom to which they are attached form 4-fluoro-piperidin-1-yl.

In certain embodiments of formula I, R⁵ and R⁶ together with the nitrogen atom to which they are attached form 3-methoxy-piperidin-1-yl.

In certain embodiments of formula I, R⁵ and R⁶ together with the nitrogen atom to which they are attached form 4-ethyl-piperazin-1-yl.

In certain embodiments of formula I, R⁵ and R⁶ together with the nitrogen atom to which they are attached form 4-acetyl-piperazin-1-yl.

In certain embodiments of formula I, R⁵ and R⁶ together with the nitrogen atom to which they are attached form 3-trifluoromethyl-piperidin-1-yl.

In certain embodiments of formula I, R⁵ and R⁶ together with the nitrogen atom to which they are attached form 4-tert-butyl-piperidin-1-yl.

In certain embodiments of formula I, R⁵ and R⁶ together with the nitrogen atom to which they are attached form.

In certain embodiments of formula I, R⁵ and R⁶ together with the nitrogen atom to which they are attached form 2-hydroxy-ethyl)-piperazin-1-yl.

In certain embodiments of formula I, R⁵ and R⁶ together with the nitrogen atom to which they are attached form 2-methyl-pyrrolidin-1-yl.

In certain embodiments of formula I, R⁵ and R⁶ together with the nitrogen atom to which they are attached form 4-hydroxymethyl-piperidin-1-yl.

In certain embodiments of formula I, R⁵ and R⁶ together with the nitrogen atom to which they are attached form 2-methyl-piperidin-1-yl.

In certain embodiments of formula I, R⁵ and R⁶ together with the nitrogen atom to which they are attached form pyrrolidin-1-yl.

In certain embodiments of formula I, R⁵ and R⁶ together with the nitrogen atom to which they are attached form 4-methanesulfonyl-piperazin-1-yl.

In certain embodiments of formula I, R⁵ and R⁶ together with the nitrogen atom to which they are attached form 3-trifluoromethyl-pyrrolidin-1-yl.

In certain embodiments of formula I, R⁵ and R⁶ together with the nitrogen atom to which they are attached form 4-(2,2,2-trifluoro-ethyl)-piperazin-1-yl.

In certain embodiments of formula I, R⁵ and R⁶ together with the nitrogen atom to which they are attached form 2-methyl-morpholin-4-yl.

In certain embodiments of formula I, R⁵ and R⁶ together with the nitrogen atom to which they are attached form (2,6-dimethyl-morpholin-4-yl.

In certain embodiments of formula I, R⁵ and R⁶ together with the nitrogen atom to which they are attached form 2,2-diethyl-morpholin-4-yl.

In certain embodiments of formula I, R⁵ and R⁶ together with the nitrogen atom to which they are attached form 3-hydroxymethyl-morpholin-4-yl.

In certain embodiments of formula I, R⁵ and R⁶ together with the nitrogen atom to which they are attached form 2-isobutyl-morpholin-4-yl.

In certain embodiments of formula I, R⁵ and R⁶ together with the nitrogen atom to which they are attached form 2-hydroxymethyl-morpholin-4-yl.

In certain embodiments of formula I, R⁵ and R⁶ together with the nitrogen atom to which they are attached form 3,3-dimethyl-morpholin-4-yl.

In certain embodiments of formula I, R⁵ and R⁶ together with the nitrogen atom to which they are attached form 4-methyl-piperazin-1-yl.

In certain embodiments of formula I, R⁵ and R⁶ together with the nitrogen atom to which they are attached form 4-isopropyl-piperazin-1-yl.

In certain embodiments of formula I, R⁵ and R⁶ together with the nitrogen atom to which they are attached form piperazin-1-yl.

In certain embodiments of formula I, R⁵ and R⁶ together with the nitrogen atom to which they are attached form 3-oxa-8-aza-bicyclo[3.2.1]oct-8-yl.

In certain embodiments of formula I, R⁵ and R⁶ together with the nitrogen atom to which they are attached form (S)-3-methyl-morpholin-4-yl.

In certain embodiments of formula I, R⁵ and R⁶ together with the nitrogen atom to which they are attached form 2-oxa-5-aza-bicyclo[2.2.1]hept-5-yl.

In certain embodiments of formula I, R⁵ and R⁶ together with the nitrogen atom to which they are attached form 8-oxa-3-aza-bicyclo[3.2.1]oct-3-yl.

In certain embodiments of formula I, R⁵ and R⁶ together with the nitrogen atom to which they are attached form (R)-3-methyl-morpholin-4-yl.

In certain embodiments of formula I, R⁵ and R⁶ together with the nitrogen atom to which they are attached form 4-cyclopropanecarbonyl-piperazin-1-yl.

In certain embodiments of formula I, R⁵ and R⁶ together with the nitrogen atom to which they are attached form 4-(1-hydroxy-1-methyl-ethyl)-piperidin-1-yl.

In certain embodiments of formula I, R⁵ and R⁶ together with the nitrogen atom to which they are attached form 4-cyclobutyl-piperazin-1-yl.

In certain embodiments of formula I, R⁵ and R⁶ together with the nitrogen atom to which they are attached form (R)-3-hydroxy-pyrrolidin-1-yl.

In certain embodiments of formula I, R⁵ and R⁶ together with the nitrogen atom to which they are attached form 4-oxetan-3-yl-piperazin-1-yl.

In certain embodiments of formula I, R⁵ and R⁶ together with the nitrogen atom to which they are attached form 3-morpholin-4-yl-azetidin-1-yl.

In certain embodiments of formula I, R⁵ and R⁶ together with the nitrogen atom to which they are attached form 4-(1-methyl-piperidin-4-yl)-piperazin-1-yl.

In certain embodiments of formula I, R⁵ and R⁶ together with the nitrogen atom to which they are attached form 3,3-difluoro-azetidin-1-yl.

In certain embodiments of formula I, R⁵ and R⁶ together with the nitrogen atom to which they are attached form 4-dimethylamino-piperidin-1-yl.

In certain embodiments of formula I, R⁵ and R⁶ together with the nitrogen atom to which they are attached form 4-piperidin-4-yl-piperazin-1-yl.

In certain embodiments of formula I, R⁵ and R⁶ together with the nitrogen atom to which they are attached form (4,4-difluoro-piperidin-1-yl.

In certain embodiments of formula I, R⁵ and R⁶ together with the nitrogen atom to which they are attached form (3-morpholin-4-yl-azetidin-1-yl.

In certain embodiments of formula I, R⁵ and R⁶ together with the nitrogen atom to which they are attached form 2-oxa-6-aza-spiro[3.3]hept-6-yl.

In certain embodiments of formula I, R⁵ and R⁶ together with the nitrogen atom to which they are attached form 2-oxa-5-aza-bicyclo[2.2.1]hept-5-yl). In certain embodiments of formula I, R⁵ and R⁶ together with the nitrogen atom to which they are attached form 4-methoxy-piperidin-1-yl).

In certain embodiments of formula I, R⁵ and R⁶ together with the nitrogen atom to which they are attached form [1,4]oxazepan-4-yl.

In certain embodiments of formula I, R⁵ and R⁶ together with the nitrogen atom to which they are attached form 2R,6S)-2,6-dimethyl-morpholin-4-yl.

In certain embodiments of formula I, R⁵ and R⁶ together with the nitrogen atom to which they are attached form 3-hydroxy-azetidin-1-yl.

In certain embodiments of formula I, R⁵ and R⁶ together with the nitrogen atom to which they are attached form 3-cyano-pyrrolidin-1-yl.

In certain embodiments of formula I, R⁵ and R⁶ together with the nitrogen atom to which they are attached form 3,5-dimethyl-piperazin-1-yl.

In certain embodiments of formula I, R⁵ and R⁶ together with the nitrogen atom to which they are attached form (3R,5S)-dimethyl-piperazin-1-yl.

In certain embodiments of formula I, R⁵ and R⁶ together with the nitrogen atom to which they are attached form 3-Fluoro-pyrrolidin-1-yl.

In certain embodiments of formula I, R⁵ and R⁶ together with the nitrogen atom to which they are attached form (S)-3-Fluoro-pyrrolidin-1-yl.

In certain embodiments of formula I, R⁵ and R⁶ together with the nitrogen atom to which they are attached form piperazin-1-yl.

In certain embodiments of formula I, R⁵ and R⁶ together with the nitrogen atom to which they are attached form 3,3-difluoro-pyrrolidin-1-yl.

In certain embodiments of formula I, R⁵ and R⁶ together with the nitrogen atom to which they are attached form 3,3-difluoro-azetidin-1-yl.

In certain embodiments of formula I, R⁵ and R⁶ together with the nitrogen atom to which they are attached form 2,2,6,6-tetrafluoro-morpholin-4-yl.

In certain embodiments of formula I, R⁵ and R⁶ together with the nitrogen atom to which they are attached form 2-methoxymethyl-pyrrolidin-1-yl. In certain embodiments of formula I, R⁵ and R⁶ together with the nitrogen atom to which they are attached form (S)-2-methoxymethyl-pyrrolidin-1-yl.

In certain embodiments of formula I, R⁵ and R⁶ together with the nitrogen atom to which they are attached form (1S,4S)-2-oxa-5-azabicyclo[2.2.1]heptan-5-yl.

In certain embodiments of formula I, R⁵ and R⁶ together with the nitrogen atom to which they are attached form (3S,4S)-3,4-difluoropyrrolidin-1-yl; 3,4-difluoropyrrolidin-1-yl; and 3-methoxypyrrolidin-1-yl.

In certain embodiments of formula I, R⁷ is halo.

In certain embodiments of formula I, R⁷ is C₁₋₆alkyl.

In certain embodiments of formula I, R⁷ is C₁₋₆alkoxy.

In certain embodiments of formula I, R⁷ is halo-C₁₋₆alkyl.

In certain embodiments of formula I, R⁷ is halo-C₁₋₆alkoxy.

In certain embodiments of formula I, R⁷ is halo or methoxy.

In certain embodiments of formula I, R⁷ is fluoro, chloro or methoxy.

In certain embodiments of formula I, R⁷ is fluoro or chloro.

In certain embodiments of formula I, R⁷ is methoxy.

In certain embodiments of formula I, R⁷ is chloro.

In certain embodiments of formula I, R⁷ is fluoro.

In certain embodiments of formula I, the LRRK2 modulator compound is:
[4-(5-Fluoro-4-methylamino-pyrimidin-2-ylamino)-3-methoxy-phenyl]-morpholin-4-yl-methanone
[4-(5-Fluoro-4-methylamino-pyrimidin-2-ylamino)-3-methoxy-phenyl]-(4-hydroxy-piperidin-1-yl)-methanone
[4-(5-Chloro-4-methylamino-pyrimidin-2-ylamino)-3-methoxy-phenyl]-morpholin-4-yl-methanone
4-(5-Chloro-4-methylamino-pyrimidin-2-ylamino)-3-methoxy-N,N-dimethyl-benzamide
4-(5-Chloro-4-methylamino-pyrimidin-2-ylamino)-N-cyclopropyl-3-methoxy-benzamide
[4-(5-Chloro-4-methylamino-pyrimidin-2-ylamino)-3-methoxy-phenyl]-(4-hydroxy-piperidin-1-yl)-methanone
[4-(5-Chloro-4-methylamino-pyrimidin-2-ylamino)-3-difluoromethoxy-phenyl]-morpholin-4-yl-methanone
[4-(5-Chloro-4-methylamino-pyrimidin-2-ylamino)-3-methoxy-phenyl]-(octahydro-pyrido[1,2-a]pyrazin-2-yl)-methanone
[4-(5-Chloro-4-methylamino-pyrimidin-2-ylamino)-3-methoxy-phenyl]-(2-hydroxy-piperidin-1-yl)-methanone
[4-(5-Chloro-4-methylamino-pyrimidin-2-ylamino)-3-methoxy-phenyl]-(4,4-dimethyl-piperidin-1-yl)-methanone
[4-(5-Chloro-4-methylamino-pyrimidin-2-ylamino)-3-methoxy-phenyl]-(3,5-dimethyl-piperidin-1-yl)-methanone
[4-(5-Chloro-4-methylamino-pyrimidin-2-ylamino)-3-methoxy-phenyl]-[4-(1-hydroxy-1-methyl-ethyl)-piperidin-1-yl]-methanone
[4-(5-Chloro-4-methylamino-pyrimidin-2-ylamino)-3-methoxy-phenyl]-(3-hydroxy-pyrrolidin-1-yl)-methanone
[4-(5-Chloro-4-methylamino-pyrimidin-2-ylamino)-3-methoxy-phenyl]-(4-methyl-piperidin-1-yl)-methanone
[4-(5-Chloro-4-methylamino-pyrimidin-2-ylamino)-3-methoxy-phenyl]-piperidin-1-yl-methanone
Azetidin-1-yl-[4-(5-chloro-4-methylamino-pyrimidin-2-ylamino)-3-methoxy-phenyl]-methanone
[4-(5-Chloro-4-methylamino-pyrimidin-2-ylamino)-3-methoxy-phenyl]-(4,4-difluoro-piperidin-1-yl)-methanone
[4-(5-Chloro-4-methylamino-pyrimidin-2-ylamino)-3-methoxy-phenyl]-(3-methyl-piperidin-1-yl)-methanone
[4-(5-Chloro-4-methylamino-pyrimidin-2-ylamino)-3-methoxy-phenyl]-(4-methoxy-piperidin-1-yl)-methanone
[4-(5-Chloro-4-methylamino-pyrimidin-2-ylamino)-3-methoxy-phenyl]-(3,3-difluoro-piperidin-1-yl)-methanone
1-[4-(5-Chloro-4-methylamino-pyrimidin-2-ylamino)-3-methoxy-benzoyl]-piperidine-4-carbonitrile
[4-(5-Chloro-4-methylamino-pyrimidin-2-ylamino)-3-methoxy-phenyl]-(4-fluoro-piperidin-1-yl)-methanone
[4-(5-Chloro-4-methylamino-pyrimidin-2-ylamino)-3-methoxy-phenyl]-(3-methoxy-piperidin-1-yl)-methanone
[4-(5-Chloro-4-methylamino-pyrimidin-2-ylamino)-3-methoxy-phenyl]-(4-ethyl-piperazin-1-yl)-methanone
1-{4-[4-(5-Chloro-4-methylamino-pyrimidin-2-ylamino)-3-methoxy-benzoyl]-piperazin-1-yl}-ethanone
[4-(5-Chloro-4-methylamino-pyrimidin-2-ylamino)-3-methoxy-phenyl]-(3-trifluoromethyl-piperidin-1-yl)-methanone
(4-tert-Butyl-piperidin-1-yl)-[4-(5-chloro-4-methylamino-pyrimidin-2-ylamino)-3-methoxy-phenyl]-methanone
[4-(5-Chloro-4-methylamino-pyrimidin-2-ylamino)-3-methoxy-phenyl]-[4-(2-hydroxy-ethyl)-piperazin-1-yl]-methanone
[4-(5-Chloro-4-methylamino-pyrimidin-2-ylamino)-3-methoxy-phenyl]-(2-methyl-pyrrolidin-1-yl)-methanone
[4-(5-Chloro-4-methylamino-pyrimidin-2-ylamino)-3-methoxy-phenyl]-(4-hydroxymethyl-piperidin-1-yl)-methanone
[4-(5-Chloro-4-methylamino-pyrimidin-2-ylamino)-3-methoxy-phenyl]-(2-methyl-piperidin-1-yl)-methanone
[4-(5-Chloro-4-methylamino-pyrimidin-2-ylamino)-3-methoxy-phenyl]-pyrrolidin-1-yl-methanone
[4-(5-Chloro-4-methylamino-pyrimidin-2-ylamino)-3-methoxy-phenyl]-(4-methane sulfonyl-piperazin-1-yl)-methanone
[4-(5-Chloro-4-methylamino-pyrimidin-2-ylamino)-3-methoxy-phenyl]-(3-trifluoromethyl-pyrrolidin-1-yl)-methanone
[4-(5-Chloro-4-methylamino-pyrimidin-2-ylamino)-3-methoxy-phenyl]-[4-(2,2,2-trifluoro-ethyl)-piperazin-1-yl]-methanone
[4-(5-Bromo-4-methylamino-pyrimidin-2-ylamino)-3-methoxy-phenyl]-morpholin-4-yl-methanone
[4-(5-Chloro-4-methylamino-pyrimidin-2-ylamino)-3-methoxy-phenyl]-(2-methyl-morpholin-4-yl)-methanone
[4-(5-Chloro-4-methylamino-pyrimidin-2-ylamino)-3-methoxy-phenyl]-(2,6-dimethyl-morpholin-4-yl)-methanone
[4-(5-Chloro-4-methylamino-pyrimidin-2-ylamino)-3-methoxy-phenyl]-(2,2-diethyl-morpholin-4-yl)-methanone
[4-(5-Chloro-4-methylamino-pyrimidin-2-ylamino)-3-methoxy-phenyl]-(3-hydroxymethyl-morpholin-4-yl)-methanone
[4-(5-Chloro-4-methylamino-pyrimidin-2-ylamino)-3-methoxy-phenyl]-(2-isobutyl-morpholin-4-yl)-methanone
[4-(5-Chloro-4-methylamino-pyrimidin-2-ylamino)-3-methoxy-phenyl]-(2-hydroxymethyl-morpholin-4-yl)-methanone
[4-(5-Chloro-4-methylamino-pyrimidin-2-ylamino)-3-methoxy-phenyl]-(3,3-dimethyl-morpholin-4-yl)-methanone
[4-(5-Chloro-4-methylamino-pyrimidin-2-ylamino)-3-methoxy-phenyl]-(4-methyl-piperazin-1-yl)-methanone
[4-(5-Chloro-4-methylamino-pyrimidin-2-ylamino)-3-methoxy-phenyl]-(4-isopropyl-piperazin-1-yl)-methanone
[4-(5-Bromo-4-methoxy-pyrimidin-2-ylamino)-3-methoxy-phenyl]-morpholin-4-yl-methanone
[4-(5-Chloro-4-methylamino-pyrimidin-2-ylamino)-3-methoxy-phenyl]-piperazin-1-yl-methanone
[4-(5-Chloro-4-methylamino-pyrimidin-2-ylamino)-3-difluoromethoxy-phenyl]-(4-hydroxy-piperidin-1-yl)-methanone
[4-(5-Chloro-4-methylamino-pyrimidin-2-ylamino)-3-ethoxy-phenyl]-morpholin-4-yl-methanone
[3-Methoxy-4-(4-methylamino-5-trifluoromethyl-pyrimidin-2-ylamino)-phenyl]-morpholin-4-yl-methanone
4-(5-Chloro-4-methylamino-pyrimidin-2-ylamino)-3-methoxy-benzamide
4-(5-Chloro-4-methylamino-pyrimidin-2-ylamino)-N-ethyl-3-methoxy-benzamide
4-(5-Chloro-4-methylamino-pyrimidin-2-ylamino)-N-isopropyl-3-methoxy-benzamide
4-(5-Chloro-4-methylamino-pyrimidin-2-ylamino)-3-methoxy-N-methyl-benzamide
[4-(5-Chloro-4-prop oxy-pyrimidin-2-ylamino)-3-methoxy-phenyl]-morpholin-4-yl-methanone
[4-(5-Chloro-4-ethylamino-pyrimidin-2-ylamino)-3-methoxy-phenyl]-morpholin-4-yl-methanone
[4-(5-Chloro-4-methylamino-pyrimidin-2-ylamino)-3-methoxy-phenyl]-(3-oxa-8-aza-bicyclo[3.2.1]oct-8-yl)-methanone
[4-(5-Chloro-4-ethoxy-pyrimidin-2-ylamino)-3-methoxy-phenyl]-morpholin-4-yl-methanone
[4-(5-Chloro-4-isopropoxy-pyrimidin-2-ylamino)-3-methoxy-phenyl]-morpholin-4-yl-methanone
[4-(5-Chloro-4-methylamino-pyrimidin-2-ylamino)-3-methoxy-phenyl]-((S)-3-methyl-morpholin-4-yl)-methanone
[4-(5-Chloro-4-methoxy-pyrimidin-2-ylamino)-3-methoxy-phenyl]-morpholin-4-yl-methanone
[4-(5-Chloro-4-methylamino-pyrimidin-2-ylamino)-3-methoxy-phenyl]-(2-oxa-5-aza-bicyclo[2.2.1]hept-5-yl)-methanone
[4-(5-Chloro-4-methylamino-pyrimidin-2-ylamino)-3-methoxy-phenyl]-(8-oxa-3-aza-bicyclo[3.2.1]oct-3-yl)-methanone
[4-(5-Chloro-4-methylamino-pyrimidin-2-ylamino)-3-methoxy-phenyl]-((R)-3-methyl-morpholin-4-yl)-methanone
N-(3-Amino-propyl)-4-(5-chloro-4-methylamino-pyrimidin-2-ylamino)-3-methoxy-benzamide
[4-(5-Cyclopropyl-4-methylamino-pyrimidin-2-ylamino)-3-methoxy-phenyl]-morpholin-4-yl-methanone
[4-(5-Chloro-4-isobutylamino-pyrimidin-2-ylamino)-3-methoxy-phenyl]-morpholin-4-yl-methanone
[4-(5-Chloro-4-propylamino-pyrimidin-2-ylamino)-3-methoxy-phenyl]-morpholin-4-yl-methanone
[4-(5-Chloro-4-isopropylamino-pyrimidin-2-ylamino)-3-methoxy-phenyl]-morpholin-4-yl-methanone
[4-(5-Chloro-4-methylamino-pyrimidin-2-ylamino)-3-methoxy-phenyl]-(4-cyclopropanecarbonyl-piperazin-1-yl)-methanone
[4-(5-Bromo-4-methylamino-pyrimidin-2-ylamino)-3-methoxy-phenyl]-[4-(1-hydroxy-1-methyl-ethyl)-piperidin-1-yl]-methanone
[4-(5-Bromo-4-methylamino-pyrimidin-2-ylamino)-3-methoxy-phenyl]-(3-trifluoromethyl-pyrrolidin-1-yl)-methanone
1-[4-(5-Bromo-4-methylamino-pyrimidin-2-ylamino)-3-methoxy-benzoyl]-piperidine-4-carbonitrile
[4-(5-Bromo-4-methylamino-pyrimidin-2-ylamino)-3-methoxy-phenyl]-(4-cyclobutyl-piperazin-1-yl)-methanone
[4-(5-Bromo-4-methylamino-pyrimidin-2-ylamino)-3-methoxy-phenyl]-[4-(2,2,2-trifluoro-ethyl)-piperazin-1-yl]-methanone
[4-(5-Bromo-4-methylamino-pyrimidin-2-ylamino)-3-methoxy-phenyl]-(4-methoxy-piperidin-1-yl)-methanone
[4-(5-Bromo-4-methylamino-pyrimidin-2-ylamino)-3-methoxy-phenyl]-((R)-3-hydroxy-pyrrolidin-1-yl)-methanone
[4-(5-Bromo-4-methylamino-pyrimidin-2-ylamino)-3-methoxy-phenyl]-(4-oxetan-3-yl-piperazin-1-yl)-methanone
[4-(5-Chloro-4-methylamino-pyrimidin-2-ylamino)-3-isopropoxy-phenyl]-morpholin-4-yl-methanone
{4-[5-Chloro-4-(cyclopropylmethyl-amino)-pyrimidin-2-ylamino]-3-methoxy-phenyl}-morpholin-4-yl-methanone
[4-(5-Chloro-4-cyclobutylamino-pyrimidin-2-ylamino)-3-methoxy-phenyl]-morpholin-4-yl-methanone
[4-(5-Chloro-4-methylamino-pyrimidin-2-ylamino)-3-hydroxy-phenyl]-morpholin-4-yl-methanone
[4-(5-Bromo-4-methoxy-pyrimidin-2-ylamino)-3-methoxy-phenyl]-(4-ethyl-piperazin-1-yl)-methanone
[4-(5-Chloro-4-methylamino-pyrimidin-2-ylamino)-2-fluoro-5-methoxy-phenyl]-morpholin-4-yl-methanone
[4-(5-Bromo-4-methoxy-pyrimidin-2-ylamino)-3-cyclobutoxy-phenyl]-morpholin-4-yl-methanone
[4-(5-Chloro-4-methylamino-pyrimidin-2-ylamino)-3-cyclobutoxy-phenyl]-morpholin-4-yl-methanone
[4-(5-Chloro-4-ethylamino-pyrimidin-2-ylamino)-2-fluoro-5-methoxy-phenyl]-morpholin-4-yl-methanone
[4-(5-Bromo-4-methoxy-pyrimidin-2-ylamino)-3-methoxy-phenyl]-(3-morpholin-4-yl-azetidin-1-yl)-methanone
[4-(5-Chloro-4-methylamino-pyrimidin-2-ylamino)-3-methoxy-phenyl]-(3-morpholin-4-yl-azetidin-1-yl)-methanone
[4-(5-Chloro-4-cyclopentylamino-pyrimidin-2-ylamino)-3-methoxy-phenyl]-morpholin-4-yl-methanone
{4-[5-Chloro-4-(1-methyl-cyclobutylamino)-pyrimidin-2-ylamino]-3-methoxy-phenyl}-morpholin-4-yl-methanone
[4-(5-Chloro-4-cyclohexylamino-pyrimidin-2-ylamino)-3-methoxy-phenyl]-morpholin-4-yl-methanone
(4-{5-Chloro-4-[(tetrahydro-furan-3-ylmethyl)-amino]-pyrimidin-2-ylamino}-3-methoxy-phenyl)-morpholin-4-yl-methanone
[4-(5-Iodo-4-methylamino-pyrimidin-2-ylamino)-3-methoxy-phenyl]-morpholin-4-yl-methanone
[4-(5-Bromo-4-isopropoxy-pyrimidin-2-ylamino)-3-methoxy-phenyl]-morpholin-4-yl-methanone
[4-(5-Bromo-4-ethoxy-pyrimidin-2-ylamino)-3-methoxy-phenyl]-morpholin-4-yl-methanone
[4-(5-Bromo-4-methoxy-pyrimidin-2-ylamino)-3-methoxy-phenyl]-(4,4-difluoro-piperidin-1-yl)-methanone
(4-{5-Chloro-4-[(tetrahydro-furan-2-ylmethyl)-amino]-pyrimidin-2-ylamino}-3-methoxy-phenyl)-morpholin-4-yl-methanone
{4-[5-Chloro-4-(cyclobutylmethyl-amino)-pyrimidin-2-ylamino]-3-methoxy-phenyl}-morpholin-4-yl-methanone
[4-(5-Iodo-4-methoxy-pyrimidin-2-ylamino)-3-methoxy-phenyl]-morpholin-4-yl-methanone
[4-(5-Cyclobutyl-4-methylamino-pyrimidin-2-ylamino)-3-methoxy-phenyl]-morpholin-4-yl-methanone
[4-(5-Cyclopropyl-4-methylamino-pyrimidin-2-ylamino)-3-methoxy-phenyl]-morpholin-4-yl-methanone
{4-[5-Chloro-4-(2-cyclopropyl-ethylamino)-pyrimidin-2-ylamino]-3-methoxy-phenyl}-morpholin-4-yl-methanone
{4-[5-Chloro-4-(2-methoxy-ethylamino)-pyrimidin-2-ylamino]-3-methoxy-phenyl}-morpholin-4-yl-methanone
{4-[5-Chloro-4-(cyclopentylmethyl-amino)-pyrimidin-2-ylamino]-3-methoxy-phenyl}-morpholin-4-yl-methanone
[4-(5-Bromo-4-methoxy-pyrimidin-2-ylamino)-3-methoxy-phenyl]-((R)-2,2-di-deutero-3-methyl-morpholin-4-yl)-methanone
[4-(5-Chloro-4-methylamino-pyrimidin-2-ylamino)-3-(2,2,2-trifluoro-ethoxy)-phenyl]-morpholin-4-yl-methanone
[4-(5-Chloro-4-(2-methoxy-ethylamino)-pyrimidin-2-ylamino]-3-(2,2,2-trifluoro-ethoxy)-phenyl]-morpholin-4-yl-methanone
[4-(5-Bromo-4-methoxy-pyrimidin-2-ylamino)-2-fluoro-5-methoxy-phenyl]-morpholin-4-yl-methanone
[4-(5-Bromo-4-methoxy-pyrimidin-2-ylamino)-3-methoxy-phenyl]-((S)-2,2-di-deutero-3-methyl-morpholin-4-yl)-methanone
[4-(5-Chloro-4-methylamino-pyrimidin-2-ylamino)-3-methoxy-phenyl]-[4-(1-methyl-piperidin-4-yl)-piperazin-1-yl]-methanone
[4-(5-Chloro-4-methylamino-pyrimidin-2-ylamino)-3-trifluoromethoxy-phenyl]-morpholin-4-yl-methanone
[4-(5-Cyclobutyl-4-methoxy-pyrimidin-2-ylamino)-3-methoxy-phenyl]-morpholin-4-yl-methanone
[3-Chloro-4-(5-chloro-4-methylamino-pyrimidin-2-ylamino)-phenyl]-morpholin-4-yl-methanone
[4-(5-Bromo-4-methoxy-pyrimidin-2-ylamino)-3-ethoxy-phenyl]-morpholin-4-yl-methanone
[4-(5-Chloro-4-methylamino-pyrimidin-2-ylamino)-3-methoxy-phenyl]-(3,3-difluoro-azetidin-1-yl)-methanone
{4-[5-Bromo-4-(2-methoxy-ethylamino)-pyrimidin-2-ylamino]-3-ethoxy-phenyl}-morpholin-4-yl-methanone
[4-(5-Chloro-4-methylamino-pyrimidin-2-ylamino)-3-methoxy-phenyl]-(4-dimethylamino-piperidin-1-yl)-methanone
4-(5-Chloro-4-methylamino-pyrimidin-2-ylamino)-3-methoxy-N-oxetan-3-yl-benzamide
4-(5-Chloro-4-methylamino-pyrimidin-2-ylamino)-3-methoxy-N-(tetrahydro-pyran-3-yl)-benzamide
{4-[5-Chloro-4-(tetrahydro-pyran-3-ylamino)-pyrimidin-2-ylamino]-3-methoxy-phenyl}-morpholin-4-yl-methanone
{4-[5-Chloro-4-(2-methoxy-ethylamino)-pyrimidin-2-ylamino]-3-cyclobutoxy-phenyl}-morpholin-4-yl-methanone
{4-[5-Bromo-4-(2-methoxy-ethylamino)-pyrimidin-2-ylamino]-3-cyclobutoxy-phenyl}-morpholin-4-yl-methanone
[4-(5-Chloro-4-methylamino-pyrimidin-2-ylamino)-3-methoxy-phenyl]-(4-piperidin-4-yl-piperazin-1-yl)-methanone
{4-[5-Chloro-4-(2,2,2-trifluoro-ethylamino)-pyrimidin-2-ylamino]-3-methoxy-phenyl}-morpholin-4-yl-methanone
{4-[5-Chloro-4-(2,2-difluoro-ethylamino)-pyrimidin-2-ylamino]-3-methoxy-phenyl}-morpholin-4-yl-methanone
[4-(5-Bromo-4-methoxy-pyrimidin-2-ylamino)-3-isopropoxy-phenyl]-morpholin-4-yl-methanone
[3-Bromo-4-(5-chloro-4-methylamino-pyrimidin-2-ylamino)-phenyl]-morpholin-4-yl-methanone
[4-(5-Bromo-4-methoxy-pyrimidin-2-ylamino)-3-methoxy-phenyl]-[4-(1-hydroxy-1-methyl-ethyl)-piperidin-1-yl]-methanone
(4-(5-bromo-4-methoxypyrimidin-2-ylamino)-3-methoxyphenyl)(3-(trifluoromethyl)pyrrolidin-1-yl)methanone
1-(4-(5-bromo-4-methoxypyrimidin-2-ylamino)-3-methoxybenzoyl)piperidine-4-carbonitrile
(3-methoxy-4-(5-methoxy-4-(methylamino)pyrimidin-2-ylamino)phenyl)(morpholino)methanone
N-(3-aminopropyl)-4-(5-chloro-4-(methylamino)pyrimidin-2-ylamino)-3-methoxybenzamide
[3-Methoxy-4-(4-methylamino-5-prop-1-ynyl-pyrimidin-2-ylamino)-phenyl]-morpholin-4-yl-methanone
[3-Methoxy-4-(4-methoxy-5-prop-1-ynyl-pyrimidin-2-ylamino)-phenyl]-morpholin-4-yl-methanone
[5-Chloro-4-(5-chloro-4-methylamino-pyrimidin-2-ylamino)-2-methoxy-phenyl]-morpholin-4-yl-methanone
{4-[5-Bromo-4-(2-methoxy-ethoxy)-pyrimidin-2-ylamino]-3-methoxy-phenyl}-morpholin-4-yl-methanone
5-Chloro-4-(5-chloro-4-methylamino-pyrimidin-2-ylamino)-2-methoxy-N,N-dimethyl-benzamide
5-Chloro-4-(5-chloro-4-methylamino-pyrimidin-2-ylamino)-2-methoxy-benzamide
5-Chloro-4-(5-chloro-4-methylamino-pyrimidin-2-ylamino)-N-(2-hydroxy-ethyl)-2-methoxy-benzamide
5-Chloro-4-(5-chloro-4-methylamino-pyrimidin-2-ylamino)-2-methoxy-N-(2-methoxy-ethyl)-benzamide
5-Chloro-4-(5-chloro-4-methylamino-pyrimidin-2-ylamino)-2-methoxy-N-methyl-benzamide
5-Chloro-4-(5-chloro-4-methylamino-pyrimidin-2-ylamino)-N-cyclopropyl-2-methoxy-benzamide
[5-Chloro-4-(5-chloro-4-methylamino-pyrimidin-2-ylamino)-2-methoxy-phenyl]-(4,4-difluoro-piperidin-1-yl)-methanone
[5-Chloro-4-(4-ethylamino-5-trifluoromethyl-pyrimidin-2-ylamino)-2-methoxy-phenyl]-morpholin-4-yl-methanone
5-Chloro-N-cyclopropyl-4-(4-ethylamino-5-trifluoromethyl-pyrimidin-2-ylamino)-2-methoxy-benzamide
5-Chloro-4-(4-ethylamino-5-trifluoromethyl-pyrimidin-2-ylamino)-2-methoxy-N-(2-methoxy-ethyl)-benzamide
[5-Chloro-4-(5-chloro-4-methylamino-pyrimidin-2-ylamino)-2-methoxy-phenyl]-piperazin-1-yl-methanone
[5-Chloro-4-(5-chloro-4-methylamino-pyrimidin-2-ylamino)-2-methoxy-phenyl]-(4-dimethylamino-piperidin-1-yl)-methanone
[5-Chloro-4-(5-chloro-4-methylamino-pyrimidin-2-ylamino)-2-methoxy-phenyl]-(3-hydroxy-pyrrolidin-1-yl)-methanone
5-Chloro-4-(5-chloro-4-methylamino-pyrimidin-2-ylamino)-2-methoxy-N-oxetan-3-yl-benzamide
5-Chloro-N-cyclopropyl-2-methoxy-4-(4-methylamino-5-trifluoromethyl-pyrimidin-2-ylamino)-benzamide
5-Chloro-4-(5-chloro-4-methylamino-pyrimidin-2-ylamino)-2-methoxy-N-(1-methyl-piperidin-4-yl)-benzamide
[5-Chloro-4-(5-chloro-4-methylamino-pyrimidin-2-ylamino)-2-methoxy-phenyl]-pyrrolidin-1-yl-methanone
N-tert-Butyl-5-chloro-4-(5-chloro-4-methylamino-pyrimidin-2-ylamino)-2-methoxy-benzamide
5-Chloro-2-methoxy-N,N-dimethyl-4-(4-methylamino-5-trifluoromethyl-pyrimidin-2-ylamino)-benzamide
5-Chloro-4-(5-chloro-4-methylamino-pyrimidin-2-ylamino)-2-methoxy-N-(2-methoxy-ethyl)-N-methyl-benzamide
5-Chloro-4-(5-chloro-4-methylamino-pyrimidin-2-ylamino)-N-(2-hydroxy-2-methyl-propyl)-2-methoxy-benzamide
[5-Chloro-4-(5-chloro-4-methylamino-pyrimidin-2-ylamino)-2-methoxy-phenyl]-(4-hydroxy-piperidin-1-yl)-methanone
[5-Chloro-4-(5-chloro-4-methylamino-pyrimidin-2-ylamino)-2-methoxy-phenyl]-(2-hydroxymethyl-morpholin-4-yl)-methanone
5-Chloro-4-(5-chloro-4-methylamino-pyrimidin-2-ylamino)-N-(2-hydroxy-ethyl)-2-methoxy-N-methyl-benzamide
5-Chloro-4-(5-chloro-4-methylamino-pyrimidin-2-ylamino)-2-methoxy-N-(1-methyl-cyclobutyl)-benzamide
[5-Chloro-4-(5-chloro-4-methylamino-pyrimidin-2-ylamino)-2-methoxy-phenyl]-[1,4]oxazepan-4-yl-methanone
[5-Chloro-4-(5-chloro-4-methylamino-pyrimidin-2-ylamino)-2-methoxy-phenyl]-((2R,6S)-2,6-dimethyl-morpholin-4-yl)-methanone
1-[5-Chloro-4-(5-chloro-4-methylamino-pyrimidin-2-ylamino)-2-methoxy-benzoyl]-piperidine-4-carbonitrile
5-Chloro-4-(5-chloro-4-methylamino-pyrimidin-2-ylamino)-N-(2-hydroxy-propyl)-2-methoxy-benzamide
[5-Chloro-4-(5-chloro-4-methylamino-pyrimidin-2-ylamino)-2-methoxy-phenyl]-(3-hydroxy-azetidin-1-yl)-methanone
5-Chloro-4-(5-chloro-4-methylamino-pyrimidin-2-ylamino)-N-(1-cyano-cyclopropyl)-2-methoxy-benzamide
1-[5-Chloro-4-(5-chloro-4-methylamino-pyrimidin-2-ylamino)-2-methoxy-benzoyl]-pyrrolidine-3-carbonitrile
[5-Chloro-4-(5-chloro-4-methylamino-pyrimidin-2-ylamino)-2-methoxy-phenyl]-((3R,5S)-dimethyl-piperazin-1-yl)-methanone
5-Chloro-2-methoxy-4-(4-methylamino-5-trifluoromethyl-pyrimidin-2-ylamino)-N-(1-methyl-piperidin-4-yl)-benzamide
5-Chloro-2-methoxy-4-(4-methylamino-5-trifluoromethyl-pyrimidin-2-ylamino)-N-oxetan-3-yl-benzamide
5-Chloro-N-(2-hydroxy-2-methyl-propyl)-2-methoxy-4-(4-methylamino-5-trifluoromethyl-pyrimidin-2-ylamino)-benzamide
[5-Chloro-2-methoxy-4-(4-methylamino-5-trifluoromethyl-pyrimidin-2-ylamino)-phenyl]-piperazin-1-yl-methanone
1-[5-Chloro-2-methoxy-4-(4-methylamino-5-trifluoromethyl-pyrimidin-2-ylamino)-benzoyl]-pyrrolidine-3-carbonitrile
1-[2-Fluoro-5-methoxy-4-(4-methylamino-5-trifluoromethyl-pyrimidin-2-ylamino)-benzoyl]-pyrrolidine-3-carbonitrile
[5-Chloro-2-ethoxy-4-(4-methylamino-5-trifluoromethyl-pyrimidin-2-ylamino)-phenyl]-morpholin-4-yl-methanone
2-Fluoro-5-methoxy-N-methyl-4-(4-methylamino-5-trifluoromethyl-pyrimidin-2-ylamino)-benzamide
5-Chloro-N-(2-hydroxy-2-methyl-propyl)-2-methoxy-N-methyl-4-(4-methylamino-5-trifluoromethyl-pyrimidin-2-ylamino)-benzamide
(4-(5-cyclopropyl-4-methoxypyrimidin-2-ylamino)-3-methoxyphenyl)(morpholino)methanone
1-{2-[2-Methoxy-4-(morpholine-4-carbonyl)-phenylamino]-4-methylamino-pyrimidin-5-yl}-ethanone
{4-[5-Chloro-4-(2-methoxy-ethoxy)-pyrimidin-2-ylamino]-3-methoxy-phenyl}-morpholin-4-yl-methanone
{4-[5-Bromo-4-(2-methoxy-ethylamino)-pyrimidin-2-ylamino]-2-fluoro-5-methoxy-phenyl}-morpholin-4-yl-methanone
{4-[5-Bromo-4-(2-methoxy-ethylamino)-pyrimidin-2-ylamino]-3-isopropoxy-phenyl}-morpholin-4-yl-methanone
{4-[5-Chloro-4-(2-methoxy-ethoxy)-pyrimidin-2-ylamino]-3-cyclobutoxy-phenyl}-morpholin-4-yl-methanone
[4-(5-Chloro-4-methylamino-pyrimidin-2-ylamino)-3-cyclopentyloxy-phenyl]-(2-oxa-6-aza-spiro[3.3]hept-6-yl)-methanone
[4-(5-Chloro-4-methylamino-pyrimidin-2-ylamino)-3-cyclopentyloxy-phenyl]-morpholin-4-yl-methanone
[4-(5-Chloro-4-methylamino-pyrimidin-2-ylamino)-3-methoxy-phenyl]-(2-oxa-6-aza-spiro[3.3]hept-6-yl)-methanone
2-(2-methoxy-4-(2,2,6,6-tetrafluoromorpholine-4-carbonyl)phenylamino)-4-(methylamino)pyrimidine-5-carbonitrile
2-(4-(4,4-difluoropiperidine-1-carbonyl)-2-methoxyphenylamino)-4-(methylamino)pyrimidine-5-carbonitrile
[4-(5-Chloro-4-methylamino-pyrimidin-2-ylamino)-3-cyclopropyl-phenyl]-morpholin-4-yl-methanone
4-(5-Cyano-4-methylamino-pyrimidin-2-ylamino)-N-(3,3-difluoro-cyclobutyl)-3-methoxy-benzamide
4-(5-Cyano-4-methylamino-pyrimidin-2-ylamino)-N-cyclopropylmethyl-3-methoxy-benzamide
2-[4-((S)-3-Fluoro-pyrrolidine-1-carbonyl)-2-methoxy-phenylamino]-4-methylamino-pyrimidine-5-carbonitrile
4-(5-Cyano-4-methylamino-pyrimidin-2-ylamino)-3-methoxy-N-methyl-benzamide
4-(5-Cyano-4-methylamino-pyrimidin-2-ylamino)-3-methoxy-N,N-dimethyl-benzamide
4-(5-Cyano-4-methylamino-pyrimidin-2-ylamino)-N,N-di ethyl-3-methoxy-benzamide
4-(5-Cyano-4-methylamino-pyrimidin-2-ylamino)-N-isopropyl-3-methoxy-benzamide
2-[2-Methoxy-4-(pyrrolidine-1-carbonyl)-phenylamino]-4-methylamino-pyrimidine-5-carbonitrile
2-[4-(3-Fluoro-azetidine-1-carbonyl)-2-methoxy-phenylamino]-4-methylamino-pyrimidine-5-carbonitrile
4-(5-Cyano-4-methylamino-pyrimidin-2-ylamino)-N-ethyl-3-methoxy-benzamide
2-[2-Methoxy-4-(piperidine-1-carbonyl)-phenylamino]-4-methylamino-pyrimidine-5-carbonitrile
4-(5-Cyano-4-methylamino-pyrimidin-2-ylamino)-N-(2,2-difluoro-ethyl)-3-methoxy-benzamide
N-tert-Butyl-4-(5-cyano-4-methylamino-pyrimidin-2-ylamino)-3-methoxy-benzamide
4-(5-Cyano-4-methylamino-pyrimidin-2-ylamino)-N-ethyl-N-isopropyl-3-methoxy-benzamide
2-[4-(3,3-Difluoro-pyrrolidine-1-carbonyl)-2-methoxy-phenylamino]-4-methylamino-pyrimidine-5-carbonitrile
2-[4-(3,3-Difluoro-azetidine-1-carbonyl)-2-methoxy-phenylamino]-4-methylamino-pyrimidine-5-carbonitrile
2-[4-((R)-3-Fluoro-pyrrolidine-1-carbonyl)-2-methoxy-phenylamino]-4-methylamino-pyrimidine-5-carbonitrile
4-(5-Cyano-4-methylamino-pyrimidin-2-ylamino)-N-ethyl-3-methoxy-N-methyl-benzamide
2-[4-(Azetidine-1-carbonyl)-2-methoxy-phenylamino]-4-methylamino-pyrimidine-5-carbonitrile
4-(5-Cyano-4-methylamino-pyrimidin-2-ylamino)-N-isopropyl-3-methoxy-N-methyl-benzamide
{4-[5-Chloro-4-(tetrahydro-furan-3-ylamino)-pyrimidin-2-ylamino]-3-methoxy-phenyl}-morpholin-4-yl-methanone
[3-Isopropoxy-4-(4-methylamino-5-trifluoromethyl-pyrimidin-2-ylamino)-phenyl]-morpholin-4-yl-methanone
[3-Ethoxy-4-(4-methylamino-5-trifluoromethyl-pyrimidin-2-ylamino)-phenyl]-morpholin-4-yl-methanone
[4-(5-Chloro-4-methylamino-pyrimidin-2-ylamino)-3-cyclopropoxy-phenyl]-morpholin-4-yl-methanone
[4-(5-Bromo-4-methoxy-pyrimidin-2-ylamino)-3-cyclopropoxy-phenyl]-morpholin-4-yl-methanone
{4-[5-Chloro-4-(2-methanesulfonyl-ethylamino)-pyrimidin-2-ylamino]-3-methoxy-phenyl}-morpholin-4-yl-methanone
2-[4-(5-Bromo-4-methylamino-pyrimidin-2-ylamino)-2,5-dimethoxy-phenyl]-1-morpholin-4-yl-ethanone
2-[4-(5-Chloro-4-methylamino-pyrimidin-2-ylamino)-2,5-dimethoxy-phenyl]-1-morpholin-4-yl-ethanone
2-[4-(5-Chloro-4-methylamino-pyrimidin-2-ylamino)-3-methoxy-phenyl]-1-morpholin-4-yl-ethanone
2-[3-Methoxy-4-(4-methylamino-5-trifluoromethyl-pyrimidin-2-ylamino)-phenyl]-1-morpholin-4-yl-ethanone
2-[4-(5-Chloro-4-methoxy-pyrimidin-2-ylamino)-2,5-dimethoxy-phenyl]-1-morpholin-4-yl-ethanone
2-[4-(5-Chloro-4-methoxy-pyrimidin-2-ylamino)-3-methoxy-phenyl]-1-morpholin-4-yl-ethanone
2-[4-(5-Bromo-4-methoxy-pyrimidin-2-ylamino)-3-methoxy-phenyl]-1-morpholin-4-yl-ethanone
2-[2,5-Dimethoxy-4-(4-methylamino-5-trifluoromethyl-pyrimidin-2-ylamino)-phenyl]-1-morpholin-4-yl-ethanone
N-(3-Amino-propyl)-5-chloro-4-(5-chloro-4-methylamino-pyrimidin-2-ylamino)-2-methoxy-benzamide
{4-[5-Bromo-4-(2-methoxy-ethylamino)-pyrimidin-2-ylamino]-3-methoxy-phenyl}-morpholin-4-yl-methanone
{4-[5-Chloro-4-(2-methoxy-propylamino)-pyrimidin-2-ylamino]-3-methoxy-phenyl}-morpholin-4-yl-methanone
4-(5-chloro-4-(methylamino)pyrimidin-2-ylamino)-N,N,3-trimethylbenzamide
(4-(5-chloro-4-(methylamino)pyrimidin-2-ylamino)-3-methylphenyl)(morpholino)methanone
(4-(5-chloro-4-(methylamino)pyrimidin-2-ylamino)-3-methylphenyl)(4-hydroxypiperidin-1-yl)methanone
(4-(4-(ethylamino)-5-(trifluoromethyl)pyrimidin-2-ylamino)-3-(trifluoromethoxy)phenyl)(morpholino)methanone
(4-(4-(methylamino)-5-(trifluoromethyl)pyrimidin-2-ylamino)-3-(trifluoromethoxy)phenyl)(morpholino)methanone
(5-chloro-2-methoxy-4-(4-(methylamino)-5-(trifluoromethyl)pyrimidin-2-ylamino)phenyl)(perdeuteromorpholino)methanone
(5-fluoro-2-methoxy-4-(4-(methylamino)-5-(trifluoromethyl)pyrimidin-2-ylamino)phenyl)(morpholino)methanone
(4-(4-(ethylamino)-5-(trifluoromethyl)pyrimidin-2-ylamino)-5-fluoro-2-methoxyphenyl)(morpholino)methanone
[2-Fluoro-5-methoxy-4-(4-methylamino-5-trifluoromethyl-pyrimidin-2-ylamino)-phenyl]-morpholin-4-yl-methanone
4-(5-Cyano-4-ethylamino-pyrimidin-2-ylamino)-2-fluoro-5-methoxy-N,N-dimethylbenzamide
(4-(4-(ethylamino)-5-(trifluoromethyl)pyrimidin-2-ylamino)-2-fluoro-3-methoxyphenyl)(morpholino)methanone
(1S,4S)-2-oxa-5-azabicyclo[2.2.1]heptan-5-yl(4-(4-(ethylamino)-5-(trifluoromethyl)pyrimidin-2-ylamino)-2-fluoro-3-methoxyphenyl)methanone
(1S,4S)-2-oxa-5-azabicyclo[2.2.1]heptan-5-yl(2-fluoro-3-methoxy-4-(4-(methylamino)-5-(trifluoromethyl)pyrimidin-2-ylamino)phenyl)methanone
5-chloro-4-(5-chloro-4-methoxypyrimidin-2-ylamino)-2-methoxy-N-methylbenzamide
2-(4-((3S,4S)-3,4-difluoropyrrolidine-1-carbonyl)-2-methoxyphenylamino)-4-(methylamino)pyrimidine-5-carbonitrile
(5-chloro-2-methoxy-4-(4-(methylamino)-5-(trifluoromethyl)pyrimidin-2-ylamino)phenyl)(3-methoxypyrrolidin-1-yl)methanone
(2-fluoro-5-methoxy-4-(4-(methylamino)-5-(trifluoromethyl)pyrimidin-2-ylamino)phenyl)(3-methoxypyrrolidin-1-yl)methanone
2-fluoro-N-(2-hydroxy-2-methylpropyl)-5-methoxy-N-methyl-4-(4-(methylamino)-5-(trifluoromethyl)pyrimidin-2-ylamino)benzamide
((2S,6R)-2,6-dimethylmorpholino)(2-fluoro-5-methoxy-4-(4-(methylamino)-5-(trifluoromethyl)pyrimidin-2-ylamino)phenyl)methanone
(2-fluoro-5-methoxy-4-(4-(2-methoxyethoxy)-5-(trifluoromethyl)pyrimidin-2-ylamino)phenyl)(morpholino)methanone
(2-ethoxy-5-fluoro-4-(4-(methylamino)-5-(trifluoromethyl)pyrimidin-2-ylamino)phenyl)(morpholino)methanone
(4-(4-(ethylamino)-5-(trifluoromethyl)pyrimidin-2-ylamino)-3-isopropoxyphenyl)(morpholino)methanone
(4-(4-(ethylamino)-5-(trifluoromethyl)pyrimidin-2-ylamino)-2-fluoro-3-isopropoxyphenyl)(morpholino)methanone
(4-(5-chloro-4-(piperidin-1-yl)pyrimidin-2-ylamino)-3-methoxyphenyl)(morpholino)methanone
(4-(5-chloro-4-(pyrrolidin-1-yl)pyrimidin-2-ylamino)-3-methoxyphenyl)(morpholino)methanone
[4-(4-Ethylamino-5-trifluoromethyl-pyrimidin-2-ylamino)-2-fluoro-5-methoxy-phenyl]-morpholin-4-yl-methanone
4-(4-Ethylamino-5-trifluoromethyl-pyrimidin-2-ylamino)-3-methoxy-phenyl]-morpholin-4-yl-methanone
[5-Chloro-4-(5-chloro-4-ethylamino-pyrimidin-2-ylamino)-2-methoxy-phenyl]-morpholin-4-yl-methanone
[4-(5-Chloro-4-methoxy-pyrimidin-2-ylamino)-2-fluoro-5-methoxy-phenyl]-morpholin-4-yl-methanone
[5-Chloro-4-(5-chloro-4-methoxy-pyrimidin-2-ylamino)-2-methoxy-phenyl]-morpholin-4-yl-methanone
[3-Chloro-4-(5-chloro-4-methoxy-pyrimidin-2-ylamino)-phenyl]-morpholin-4-yl-methanone
[2-Chloro-5-methoxy-4-(4-methoxy-5-trifluoromethyl-pyrimidin-2-ylamino)-phenyl]-morpholin-4-yl-methanone
[3-Methoxy-4-(4-methoxy-5-trifluoromethyl-pyrimidin-2-ylamino)-phenyl]-morpholin-4-yl-methanone
{4-[5-Chloro-4-(tetrahydro-pyran-4-yloxy)-pyrimidin-2-ylamino]-3-methoxy-phenyl}-morpholin-4-yl-methanone
5-Chloro-N-(1-cyano-cyclopropyl)-2-methoxy-4-(4-methylamino-5-trifluoromethyl-pyrimidin-2-ylamino)-benzamide
5-Chloro-N-(1-cyano-cyclopropyl)-2-methoxy-4-(4-methylamino-5-trifluoromethyl-pyrimidin-2-ylamino)-benzamide
[5-Chloro-2-methoxy-4-(4-methylamino-5-trifluoromethyl-pyrimidin-2-ylamino)-phenyl]-(2,6-dimethyl-morpholin-4-yl)-methanone
[3-Methoxy-4-(4-pyrrolidin-1-yl-5-trifluoromethyl-pyrimidin-2-ylamino)-phenyl]-morpholin-4-yl-methanone
[3-Cyclobutoxy-4-(4-methylamino-5-trifluoromethyl-pyrimidin-2-ylamino)-phenyl]-morpholin-4-yl-methanone
N-(3,3-Difluoro-cyclobutyl)-3-methoxy-4-(4-methylamino-5-trifluoromethyl-pyrimidin-2-ylamino)-benzamide
3-Methoxy-4-(4-methylamino-5-trifluoromethyl-pyrimidin-2-ylamino)-N-oxetan-3-yl-benzamide
[3-Methoxy-4-(4-methylamino-5-trifluoromethyl-pyrimidin-2-ylamino)-phenyl]-(8-oxa-3-aza-bicyclo[3.2.1]oct-3-yl)-methanone
[3-Methoxy-4-(4-methylamino-5-trifluoromethyl-pyrimidin-2-ylamino)-phenyl]-(2-oxa-5-aza-bicyclo[2.2.1]hept-5-yl)-methanone
[3-Methoxy-4-(4-methylamino-5-trifluoromethyl-pyrimidin-2-ylamino)-phenyl]-pyrrolidin-1-yl-methanone
N,N-Diethyl-3-methoxy-4-(4-methylamino-5-trifluoromethyl-pyrimidin-2-ylamino)-benzamide
(4-Dimethylamino-piperidin-1-yl)-[3-methoxy-4-(4-methylamino-5-trifluoromethyl-pyrimidin-2-ylamino)-phenyl]-methanone
(2,6-Dimethyl-morpholin-4-yl)-[3-methoxy-4-(4-methylamino-5-trifluoromethyl-pyrimidin-2-ylamino)-phenyl]-methanone
1-[3-Methoxy-4-(4-methylamino-5-trifluoromethyl-pyrimidin-2-ylamino)-benzoyl]-piperidine-4-carbonitrile
[3-Methoxy-4-(4-methylamino-5-trifluoromethyl-pyrimidin-2-ylamino)-phenyl]-[4-(2,2,2-trifluoro-ethyl)-piperazin-1-yl]-methanone
[3-Methoxy-4-(4-methylamino-5-trifluoromethyl-pyrimidin-2-ylamino)-phenyl]-(4-methoxy-piperidin-1-yl)-methanone
3-Methoxy-N-(2-methoxy-ethyl)-4-(4-methylamino-5-trifluoromethyl-pyrimidin-2-ylamino)-benzamide
3-Methoxy-4-(4-methylamino-5-trifluoromethyl-pyrimidin-2-ylamino)-N-(1-methyl-piperidin-4-yl)-benzamide
N-(4,4-Difluoro-cyclohexyl)-3-methoxy-4-(4-methylamino-5-trifluoromethyl-pyrimidin-2-ylamino)-benzamide
[4-(5-Chloro-4-methylamino-pyrimidin-2-ylamino)-3-(oxetan-3-yloxy)-phenyl]-morpholin-4-yl-methanone
[3-Cyclopropylmethoxy-4-(4-methylamino-5-trifluoromethyl-pyrimidin-2-ylamino)-phenyl]-morpholin-4-yl-methanone
[3-Cyclobutylmethoxy-4-(4-methylamino-5-trifluoromethyl-pyrimidin-2-ylamino)-phenyl]-morpholin-4-yl-methanone
N-Ethyl-3-methoxy-N-methyl-4-(4-methylamino-5-trifluoromethyl-pyrimidin-2-ylamino)-benzamide
[4-(5-Bromo-4-methylamino-pyrimidin-2-ylamino)-5-chloro-2-methoxy-phenyl]-morpholin-4-yl-methanone
[3-Cyclopropyl-4-(4-methylamino-5-trifluoromethyl-pyrimidin-2-ylamino)-phenyl]-morpholin-4-yl-methanone
[3-Bromo-4-(4-methylamino-5-trifluoromethyl-pyrimidin-2-ylamino)-phenyl]-morpholin-4-yl-methanone
[3-Chloro-4-(4-methylamino-5-trifluoromethyl-pyrimidin-2-ylamino)-phenyl]-morpholin-4-yl-methanone
[2-Chloro-5-methoxy-4-(4-methylamino-5-trifluoromethyl-pyrimidin-2-ylamino)-phenyl]-morpholin-4-yl-methanone
[2-Chloro-4-(5-chloro-4-methylamino-pyrimidin-2-ylamino)-5-methoxy-phenyl]-morpholin-4-yl-methanone
[4-(5-Bromo-4-methoxy-pyrimidin-2-ylamino)-2-chloro-5-methoxy-phenyl]-morpholin-4-yl-methanone
(5-chloro-4-(5-chloro-4-(methylamino)pyrimidin-2-ylamino)-2-methoxyphenyl)(morpholino)methanone
[5-Chloro-2-methoxy-4-(4-methylamino-5-trifluoromethyl-pyrimidin-2-ylamino)-phenyl]-morpholin-4-yl-methanone
[2-Fluoro-3-methoxy-4-(4-methylamino-5-trifluoromethyl-pyrimidin-2-ylamino)-phenyl]-morpholin-4-yl-methanone
[3-(2-Fluoro-ethoxy)-4-(4-methylamino-5-trifluoromethyl-pyrimidin-2-ylamino)-phenyl]-morpholin-4-yl-methanone
(4-(5-chloro-4-methoxypyrimidin-2-ylamino)-3-methoxyphenyl)(pyrrolidin-1-yl)methanone
[5-Ethoxy-2-fluoro-4-(4-methylamino-5-trifluoromethyl-pyrimidin-2-ylamino)-phenyl]-morpholin-4-yl-methanone
3-Methoxy-N-(2-methoxy-ethyl)-N-methyl-4-(4-methylamino-5-trifluoromethyl-pyrimidin-2-ylamino)-benzamide
4-(4-Ethylamino-5-trifluoromethyl-pyrimidin-2-ylamino)-3-methoxy-N-(2-methoxy-ethyl)-N-methyl-benzamide
2-Fluoro-5-methoxy-N-(2-methoxy-ethyl)-N-methyl-4-(4-methylamino-5-trifluoromethyl-pyrimidin-2-ylamino)-benzamide
4-(4-Ethylamino-5-trifluoromethyl-pyrimidin-2-ylamino)-2-fluoro-5-methoxy-N-(2-methoxy-ethyl)-N-methyl-benzamide
[2-Fluoro-5-methoxy-4-(4-methylamino-5-trifluoromethyl-pyrimidin-2-ylamino)-phenyl]-((S)-2-methoxymethyl-pyrrolidin-1-yl)-methanone
[4-(4-Ethylamino-5-trifluoromethyl-pyrimidin-2-ylamino)-2-fluoro-5-methoxy-phenyl]-((S)-2-methoxymethyl-pyrrolidin-1-yl)-methanone
N-Ethyl-4-(4-ethylamino-5-trifluoromethyl-pyrimidin-2-ylamino)-2-fluoro-5-methoxy-N-(2-methoxy-ethyl)-benzamide
N-Ethyl-2-fluoro-5-methoxy-N-(2-methoxy-ethyl)-4-(4-methylamino-5-trifluoromethyl-pyrimidin-2-ylamino)-benzamide
[5-Ethoxy-4-(4-ethylamino-5-trifluoromethyl-pyrimidin-2-ylamino)-2-fluoro-phenyl]-morpholin-4-yl-methanone
(4-(4-(ethylamino)-5-(trifluoromethyl)pyrimidin-2-yl-amino)-3-isopropoxyphenyl)(morpholino)methanone
(2-fluoro-3-isopropoxy-4-(4-(methylamino)-5-(trifluoromethyl)pyrimidin-2-ylamino)phenyl)(morpholino)methanone
(4-(4-(ethylamino)-5-(trifluoromethyl)pyrimidin-2-ylamino)-3-(trifluoromethoxy)phenyl)(morpholino)methanone
(2-fluoro-3-isopropoxy-4-(4-(methylamino)-5-(trifluoromethyl)pyrimidin-2-ylamino)phenyl)(morpholino)methanone
2-(2-methoxy-4-(morpholine-4-carbonyl)phenylamino)-4-(methylamino)pyrimidine-5-carbonitrile
4-Methoxy-2-[2-methoxy-4-(morpholine-4-carbonyl)-phenylamino]-pyrimidine-5-carbonitrile
4-Ethylamino-2-[5-fluoro-2-methoxy-4-(morpholine-4-carbonyl)-phenylamino]-pyrimidine-5-carbonitrile
2-[5-Fluoro-2-methoxy-4-(morpholine-4-carbonyl)-phenylamino]-4-methylamino-pyrimidine-5-carbonitrile
2-[4-((2R,6S)-2,6-Dimethyl-morpholine-4-carbonyl)-5-fluoro-2-methoxy-phenylamino]-4-methylamino-pyrimidine-5-carbonitrile
2-[4-((2R,6S)-2,6-Dimethyl-morpholine-4-carbonyl)-5-fluoro-2-methoxy-phenylamino]-4-ethylamino-pyrimidine-5-carbonitrile
4-(5-Cyano-4-methylamino-pyrimidin-2-ylamino)-2-fluoro-5-methoxy-N,N-dimethyl-benzamide
[5-Chloro-2-methoxy-4-(4-methylamino-5-trifluoromethyl-pyrimidin-2-ylamino)-phenyl]-[4-(2-methoxy-ethyl)-piperazin-1-yl]-methanone
[5-Chloro-2-methyl-4-(4-methylamino-5-trifluoromethyl-pyrimidin-2-ylamino)-phenyl]-morpholin-4-yl-methanone
[5-Methoxy-2-methyl-4-(4-methylamino-5-trifluoromethyl-pyrimidin-2-ylamino)-phenyl]-morpholin-4-yl-methanone
[2-Fluoro-5-methoxy-4-(4-methylamino-5-trifluoromethyl-pyrimidin-2-ylamino)-phenyl]-(hexahydro-pyrrolo[1,2-a]pyrazin-2-yl)-methanone
[2-Fluoro-5-methoxy-4-(4-methylamino-5-trifluoromethyl-pyrimidin-2-ylamino)-phenyl]-(1S,5R)-8-oxa-3-aza-bicyclo[3.2.1]oct-3-yl-methanone
[4-(4-Ethylamino-5-trifluoromethyl-pyrimidin-2-ylamino)-2-fluoro-5-methoxy-phenyl]-(1S,5R)-8-oxa-3-aza-bicyclo[3.2.1]oct-3-yl-methanone
2-Fluoro-5-methoxy-N-methyl-4-(4-methylamino-5-trifluoromethyl-pyrimidin-2-ylamino)-N-(1-methyl-piperidin-4-yl)-benzamide
[2-Fluoro-5-methoxy-4-(4-methylamino-5-trifluoromethyl-pyrimidin-2-ylamino)-phenyl]-(4-morpholin-4-yl-piperidin-1-yl)-methanone
[4-(5-Chloro-4-methoxy-pyrimidin-2-ylamino)-3-hydroxy-phenyl]-morpholin-4-yl-methanone
[5-Chloro-2-hydroxy-4-(4-methylamino-5-trifluoromethyl-pyrimidin-2-ylamino)-phenyl]-morpholin-4-yl-methanone
[2-Fluoro-5-methoxy-4-(4-methylamino-5-trifluoromethyl-pyrimidin-2-ylamino)-phenyl]-(1S,4S)-2-oxa-5-aza-bicyclo[2.2.1]hept-5-yl-methanone
[2,3-Difluoro-4-(4-methylamino-5-trifluoromethyl-pyrimidin-2-ylamino)-phenyl]-morpholin-4-yl-methanone
[4-(5-Chloro-4-methylamino-pyrimidin-2-ylamino)-2,3-difluoro-phenyl]-morpholin-4-yl-methanone
[4-(4-Ethylamino-5-trifluoromethyl-pyrimidin-2-ylamino)-2-fluoro-5-methoxy-phenyl]-(1S,4S)-2-oxa-5-aza-bicyclo[2.2.1]hept-5-yl-methanone
[5-Chloro-2-methoxy-4-(4-methylamino-5-trifluoromethyl-pyrimidin-2-ylamino)-phenyl]-(1S,4S)-2-oxa-5-aza-bicyclo[2.2.1]hept-5-yl-methanone
[2,5-Difluoro-4-(4-methylamino-5-trifluoromethyl-pyrimidin-2-ylamino)-phenyl]-morpholin-4-yl-methanone
[4-(5-Chloro-4-methylamino-pyrimidin-2-ylamino)-2,5-difluoro-phenyl]-morpholin-4-yl-methanone
4-(4-Ethylamino-5-trifluoromethyl-pyrimidin-2-ylamino)-2-fluoro-N-(2-hydroxy-2-methyl-propyl)-5-methoxy-benzamide
4-(4-Ethylamino-5-trifluoromethyl-pyrimidin-2-ylamino)-2-fluoro-N-(2-hydroxy-2-methyl-propyl)-5-methoxy-N-methyl-benzamide
[5-Chloro-2-methoxy-4-(4-methylamino-5-trifluoromethyl-pyrimidin-2-ylamino)-phenyl]-(3-morpholin-4-yl-azetidin-1-yl)-methanone
N-tert-Butyl-4-(4-ethylamino-5-trifluoromethyl-pyrimidin-2-ylamino)-3-methoxy-benzamide
[3-Fluoro-4-(4-methylamino-5-trifluoromethyl-pyrimidin-2-ylamino)-phenyl]-morpholin-4-yl-methanone
[5-Chloro-2-methoxy-4-(4-methylamino-5-trifluoromethyl-pyrimidin-2-ylamino)-phenyl]-(4-oxetan-3-yl-piperazin-1-yl)-methanone
[5-Chloro-2-methoxy-4-(4-methylamino-5-trifluoromethyl-pyrimidin-2-ylamino)-phenyl]-(3,3-difluoro-pyrrolidin-1-yl)-methanone
[4-(4-Cyclopropylamino-5-trifluoromethyl-pyrimidin-2-ylamino)-2-fluoro-5-methoxy-phenyl]-morpholin-4-yl-methanone
[4-(4-Azetidin-1-yl-5-trifluoromethyl-pyrimidin-2-ylamino)-2-fluoro-5-methoxy-phenyl]-morpholin-4-yl-methanone
5-Fluoro-N-(2-hydroxy-2-methyl-propyl)-2-methoxy-4-(4-methylamino-5-trifluoromethyl-pyrimidin-2-ylamino)-benzamide
4-(4-Ethylamino-5-trifluoromethyl-pyrimidin-2-ylamino)-5-fluoro-N-(2-hydroxy-2-methyl-propyl)-2-methoxy-benzamide
5-Fluoro-N-(2-hydroxy-2-methyl-propyl)-2-methoxy-N-methyl-4-(4-methylamino-5-trifluoromethyl-pyrimidin-2-ylamino)-benzamide
4-(4-Ethylamino-5-trifluoromethyl-pyrimidin-2-ylamino)-5-fluoro-N-(2-hydroxy-2-methyl-propyl)-2-methoxy-N-methyl-benzamide
[4-(4-Ethylamino-5-trifluoromethyl-pyrimidin-2-ylamino)-2,5-difluoro-phenyl]-morpholin-4-yl-methanone
[2-Fluoro-4-(4-isopropylamino-5-trifluoromethyl-pyrimidin-2-ylamino)-5-methoxy-phenyl]-morpholin-4-yl-methanone
[3-Methoxy-2-methyl-4-(4-methylamino-5-trifluoromethyl-pyrimidin-2-ylamino)-phenyl]-morpholin-4-yl-methanone
[4-(4-Ethylamino-5-trifluoromethyl-pyrimidin-2-ylamino)-2-fluoro-5-methoxy-phenyl]-(1-oxa-6-aza-spiro[3.3]hept-6-yl)-methanone
2-Fluoro-N-(2-hydroxy-2-methyl-propyl)-5-methoxy-4-(4-methylamino-5-trifluoromethyl-pyrimidin-2-ylamino)-benzamide
((3S,4S)-3,4-Difluoro-pyrrolidin-1-yl)-[2-fluoro-5-methoxy-4-(4-methylamino-5-trifluoromethyl-pyrimidin-2-ylamino)-phenyl]-methanone
4-(4-Ethylamino-5-trifluoromethyl-pyrimidin-2-ylamino)-2-fluoro-5-methoxy-N-methyl-benzamide
2-Fluoro-N-(2-hydroxy-2-methyl-propyl)-3-methoxy-4-(4-methylamino-5-trifluoromethyl-pyrimidin-2-ylamino)-benzamide
(3,3-Difluoro-pyrrolidin-1-yl)-[5-(2-fluoro-ethoxy)-2-methoxy-4-(4-methylamino-5-trifluoromethyl-pyrimidin-2-ylamino)-phenyl]-methanone
[5-Chloro-2-methoxy-4-(4-methylamino-5-trifluoromethyl-pyrimidin-2-ylamino)-phenyl]-(1-oxa-6-aza-spiro[3.3]hept-6-yl)-methanone
[4-(4-Ethylamino-5-trifluoromethyl-pyrimidin-2-ylamino)-2,3-difluoro-phenyl]-morpholin-4-yl-methanone
[4-(4-Cyclopropylamino-5-trifluoromethyl-pyrimidin-2-ylamino)-5-methoxy-2-methyl-phenyl]-morpholin-4-yl-methanone
[5-Chloro-4-(4-ethylamino-5-trifluoromethyl-pyrimidin-2-ylamino)-2-methoxy-phenyl]-(2-oxa-6-aza-spiro[3.3]hept-6-yl)-methanone
[4-(4-Ethylamino-5-trifluoromethyl-pyrimidin-2-ylamino)-5-methoxy-2-methyl-phenyl]-morpholin-4-yl-methanone
4-(4-Cyclopropylamino-5-trifluoromethyl-pyrimidin-2-ylamino)-2-fluoro-N-(2-hydroxy-2-methyl-propyl)-5-methoxy-benzamide
N-tert-Butyl-4-(5-cyano-4-ethylamino-pyrimidin-2-ylamino)-3-methoxy-benzamide
[3-Difluoromethoxy-4-(4-methylamino-5-trifluoromethyl-pyrimidin-2-ylamino)-phenyl]-morpholin-4-yl-methanone
[2-Fluoro-5-(2-fluoro-ethoxy)-4-(4-methylamino-5-trifluoromethyl-pyrimidin-2-ylamino)-phenyl]-morpholin-4-yl-methanone
[4-(4-Ethylamino-5-trifluoromethyl-pyrimidin-2-ylamino)-2-fluoro-5-(2-fluoro-ethoxy)-phenyl]-morpholin-4-yl-methanone
[2-Fluoro-5-methoxy-4-(4-methoxy-5-trifluoromethyl-pyrimidin-2-ylamino)-phenyl]-morpholin-4-yl-methanone
[4-(4-Cyclopropylamino-5-trifluoromethyl-pyrimidin-2-ylamino)-2,3-difluoro-phenyl]-morpholin-4-yl-methanone
[4-(4-Cyclopropylamino-5-trifluoromethyl-pyrimidin-2-ylamino)-2,5-difluoro-phenyl]-morpholin-4-yl-methanone
[2-Chloro-5-fluoro-4-(4-methylamino-5-trifluoromethyl-pyrimidin-2-ylamino)-phenyl]-morpholin-4-yl-methanone
4-Ethylamino-2-[4-((S)-3-fluoro-pyrrolidine-1-carbonyl)-2-methoxy-phenylamino]-pyrimidine-5-carbonitrile
5-Chloro-2-methoxy-N-methyl-4-(4-methylamino-5-trifluoromethyl-pyrimidin-2-ylamino)-N-(1-methyl-1H-pyrazol-4-yl)-benzamide
(3,3-Difluoro-pyrrolidin-1-yl)-[4-(4-ethylamino-5-trifluoromethyl-pyrimidin-2-ylamino)-2,5-dimethoxy-phenyl]-methanone
[4-(5-Chloro-4-methoxy-pyrimidin-2-ylamino)-3-(2-fluoro-ethoxy)-phenyl]-morpholin-4-yl-methanone
[2-Methoxy-5-methyl-4-(4-methylamino-5-trifluoromethyl-pyrimidin-2-ylamino)-phenyl]-morpholin-4-yl-methanone
[2-Chloro-4-(4-ethylamino-5-trifluoromethyl-pyrimidin-2-ylamino)-5-fluoro-phenyl]-morpholin-4-yl-methanone
[3-Chloro-2-fluoro-4-(4-methylamino-5-trifluoromethyl-pyrimidin-2-ylamino)-phenyl]-morpholin-4-yl-methanone
(3,3-Difluoro-pyrrolidin-1-yl)-[4-(4-ethylamino-5-trifluoromethyl-pyrimidin-2-ylamino)-5-(2-fluoro-ethoxy)-2-methoxy-phenyl]-methanone
[4-(4-Ethylamino-5-trifluoromethyl-pyrimidin-2-ylamino)-2,6-difluoro-3-methoxy-phenyl]-morpholin-4-yl-methanone
[2,6-Difluoro-3-methoxy-4-(4-methylamino-5-trifluoromethyl-pyrimidin-2-ylamino)-phenyl]-morpholin-4-yl-methanone
[4-(4-Ethoxy-5-trifluoromethyl-pyrimidin-2-ylamino)-2-fluoro-5-methoxy-phenyl]-morpholin-4-yl-methanone
[2,5-Dimethoxy-4-(4-methylamino-5-trifluoromethyl-pyrimidin-2-ylamino)-phenyl]-morpholin-4-yl-methanone
(3,3-Difluoro-pyrrolidin-1-yl)-[4-(4-ethylamino-5-trifluoromethyl-pyrimidin-2-ylamino)-5-fluoromethoxy-2-methoxy-phenyl]-methanone
[5-Methoxy-4-(4-methylamino-5-trifluoromethyl-pyrimidin-2-ylamino)-2-trifluoromethyl-phenyl]-morpholin-4-yl-methanone
[4-(5-Chloro-4-methylamino-pyrimidin-2-ylamino)-5-methoxy-2-trifluoromethyl-phenyl]-morpholin-4-yl-methanone
[5-Chloro-2-fluoro-4-(4-methylamino-5-trifluoromethyl-pyrimidin-2-ylamino)-phenyl]-morpholin-4-yl-methanone
[4-(4-Cyclopropylamino-5-trifluoromethyl-pyrimidin-2-ylamino)-2-fluoro-5-(2-fluoro-ethoxy)-phenyl]-morpholin-4-yl-methanone
[5-Chloro-4-(4-ethylamino-5-trifluoromethyl-pyrimidin-2-ylamino)-2-methyl-phenyl]-morpholin-4-yl-methanone
[5-Chloro-4-(4-ethylamino-5-trifluoromethyl-pyrimidin-2-ylamino)-2-fluoromethoxy-phenyl]-morpholin-4-yl-methanone
[5-Fluoro-2-methyl-4-(4-methylamino-5-trifluoromethyl-pyrimidin-2-ylamino)-phenyl]-morpholin-4-yl-methanone
[5-Chloro-4-(4-ethylamino-5-trifluoromethyl-pyrimidin-2-ylamino)-2-fluoro-phenyl]-morpholin-4-yl-methanone
{2-Fluoro-4-[4-((1R,2S)-2-fluoro-cyclopropylamino)-5-trifluoromethyl-pyrimidin-2-ylamino]-5-methoxy-phenyl}-morpholin-4-yl-methanone
(2,2-Dimethyl-morpholin-4-yl)-[2-fluoro-5-methoxy-4-(4-methylamino-5-trifluoromethyl-pyrimidin-2-ylamino)-phenyl]-methanone
[3-Cyclopropoxy-4-(4-methylamino-5-trifluoromethyl-pyrimidin-2-ylamino)-phenyl]-morpholin-4-yl-methanone
[5-Chloro-2-difluoromethoxy-4-(4-ethylamino-5-trifluoromethyl-pyrimidin-2-ylamino)-phenyl]-morpholin-4-yl-methanone
[5-Chloro-2-difluoromethoxy-4-(4-methylamino-5-trifluoromethyl-pyrimidin-2-ylamino)-phenyl]-morpholin-4-yl-methanone
4-Ethylamino-2-[5-fluoro-4-((S)-3-fluoro-pyrrolidine-1-carbonyl)-2-methoxy-phenylamino]-pyrimidine-5-carbonitrile
4-Ethylamino-2-[2-fluoro-4-((S)-3-fluoro-pyrrolidine-1-carbonyl)-5-methoxy-phenylamino]-pyrimidine-5-carbonitrile
[3-Chloro-2,6-dimethoxy-4-(4-methylamino-5-trifluoromethyl-pyrimidin-2-ylamino)-phenyl]-morpholin-4-yl-methanone
(2,2-Dimethyl-morpholin-4-yl)-[3-methoxy-4-(4-methylamino-5-trifluoromethyl-pyrimidin-2-ylamino)-phenyl]-methanone
[5-(2-Fluoro-ethoxy)-2-methyl-4-(4-methylamino-5-trifluoromethyl-pyrimidin-2-ylamino)-phenyl]-morpholin-4-yl-methanone
[5-(2-Fluoro-ethoxy)-2-methoxy-4-(4-methylamino-5-trifluoromethyl-pyrimidin-2-ylamino)-phenyl]-pyrrolidin-1-yl-methanone
(5-chloro-4-(4-(ethylamino)-5-(trifluoromethyl)pyrimidin-2-ylamino)-2-methoxyphenyl)(perdeuteromorpholino)methanone
[4-(5-Chloro-4-methoxy-pyrimidin-2-ylamino)-2,5-dimethoxy-phenyl]-morpholin-4-yl-methanone
[5-Fluoromethoxy-2-methyl-4-(4-methylamino-5-trifluoromethyl-pyrimidin-2-ylamino)-phenyl]-morpholin-4-yl-methanone
{4-[Ethyl-(4-methylamino-5-trifluoromethyl-pyrimidin-2-yl)-amino]-5-fluoromethoxy-2-methyl-phenyl}-morpholin-4-yl-methanone
[5-Fluoromethoxy-2-methoxy-4-(4-methylamino-5-trifluoromethyl-pyrimidin-2-ylamino)-phenyl]-pyrrolidin-1-yl-methanone
[4-(4-Cyclobutylamino-5-trifluoromethyl-pyrimidin-2-ylamino)-2-fluoro-5-methoxy-phenyl]-morpholin-4-yl-methanone
[2-Fluoro-5-methyl-4-(4-methylamino-5-trifluoromethyl-pyrimidin-2-ylamino)-phenyl]-morpholin-4-yl-methanone
[4-(5-Chloro-4-methoxy-pyrimidin-2-ylamino)-5-methoxy-2-methyl-phenyl]-morpholin-4-yl-methanone
[4-(5-Chloro-4-ethoxy-pyrimidin-2-ylamino)-5-methoxy-2-methyl-phenyl]-morpholin-4-yl-methanone
[2-Fluoro-5-fluoromethoxy-4-(4-methylamino-5-trifluoromethyl-pyrimidin-2-ylamino)-phenyl]-pyrrolidin-1-yl-methanone
4-Cyclopropylamino-2-[4-((S)-3-fluoro-pyrrolidine-1-carbonyl)-2-methoxy-phenylamino]-pyrimidine-5-carbonitrile
4-Ethylamino-2-[4-((S)-3-fluoro-pyrrolidine-1-carbonyl)-2-methoxy-5-methyl-phenylamino]-pyrimidine-5-carbonitrile
4-Ethylamino-2-[2-fluoro-4-((S)-3-fluoro-pyrrolidine-1-carbonyl)-phenylamino]-pyrimidine-5-carbonitrile
[5-Isopropoxy-2-methyl-4-(4-methylamino-5-trifluoromethyl-pyrimidin-2-ylamino)-phenyl]-morpholin-4-yl-methanone
[5-Ethoxy-2-methyl-4-(4-methylamino-5-trifluoromethyl-pyrimidin-2-ylamino)-phenyl]-morpholin-4-yl-methanone
[5-Fluoromethoxy-2-methyl-4-(4-methylamino-5-trifluoromethyl-pyrimidin-2-ylamino)-phenyl]-pyrrolidin-1-yl-methanone
[5-Chloro-4-(4-cyclopropylamino-5-trifluoromethyl-pyrimidin-2-ylamino)-2-fluoromethoxy-phenyl]-morpholin-4-yl-methanone
[4-(4-Ethylamino-5-trifluoromethyl-pyrimidin-2-ylamino)-5-fluoro-2-methyl-phenyl]-morpholin-4-yl-methanone
(5-methoxy-2-methyl-4-(4-(methylamino)-5-(trifluoromethyl)pyrimidin-2-ylamino)phenyl)(perdeuteromorpholino)methanone
4-(4-Ethylamino-5-trifluoromethyl-pyrimidin-2-ylamino)-N-(2-hydroxy-2-methyl-propyl)-5-methoxy-2-methyl-benzamide
4-(4-Ethylamino-5-trifluoromethyl-pyrimidin-2-ylamino)-2-fluoro-N-(2-hydroxy-1,1-dimethyl-ethyl)-5-methoxy-benzamide
2-[2-Chloro-4-((S)-3-fluoro-pyrrolidine-1-carbonyl)-5-methyl-phenylamino]-4-ethylamino-pyrimidine-5-carbonitrile
2-[2-Chloro-4-((S)-3-fluoro-pyrrolidine-1-carbonyl)-phenylamino]-4-ethylamino-pyrimidine-5-carbonitrile
[5-Cyclopropylmethoxy-2-methyl-4-(4-methylamino-5-trifluoromethyl-pyrimidin-2-ylamino)-phenyl]-morpholin-4-yl-methanone
[5-Fluoromethoxy-2-methoxy-4-(4-methylamino-5-trifluoromethyl-pyrimidin-2-ylamino)-phenyl]-piperidin-1-yl-methanone
[5-Chloro-4-(4-ethylamino-5-trifluoromethyl-pyrimidin-2-ylamino)-2-fluoromethoxy-phenyl]-pyrrolidin-1-yl-methanone
[4-(4-Cyclobutylamino-5-trifluoromethyl-pyrimidin-2-ylamino)-5-fluoro-2-methoxy-phenyl]-morpholin-4-yl-methanone
[5-Chloro-4-(4-cyclobutylamino-5-trifluoromethyl-pyrimidin-2-ylamino)-2-methoxy-phenyl]-morpholin-4-yl-methanone
4-(4-Ethylamino-5-trifluoromethyl-pyrimidin-2-ylamino)-2-fluoro-N-(1-hydroxymethyl-cyclopropyl)-5-methoxy-benzamide
4-(4-Ethylamino-5-trifluoromethyl-pyrimidin-2-ylamino)-2-fluoro-N-(1-hydroxy-cyclopropylmethyl)-5-methoxy-benzamide
N-(2-Hydroxy-2-methyl-propyl)-5-methoxy-2-methyl-4-(4-methylamino-5-trifluoromethyl-pyrimidin-2-ylamino)-benzamide
2-[2-Chloro-4-((S)-3-fluoro-pyrrolidine-1-carbonyl)-5-methoxy-phenylamino]-4-ethylamino-pyrimidine-5-carbonitrile
[4-(4-Ethylamino-5-trifluoromethyl-pyrimidin-2-ylamino)-2-fluoro-5-methyl-phenyl]-morpholin-4-yl-methanone
[4-(5-Chloro-4-methoxy-pyrimidin-2-ylamino)-5-fluoromethoxy-2-methyl-phenyl]-pyrrolidin-1-yl-methanone
5-Fluoromethoxy-2-methoxy-N,N-dimethyl-4-(4-methylamino-5-trifluoromethyl-pyrimidin-2-ylamino)-benzamide
[4-(4-Ethylamino-5-trifluoromethyl-pyrimidin-2-ylamino)-5-fluoro-2-fluoromethoxy-phenyl]-pyrrolidin-1-yl-methanone
[3-Fluoro-2,6-dimethoxy-4-(4-methylamino-5-trifluoromethyl-pyrimidin-2-ylamino)-phenyl]-morpholin-4-yl-methanone
(4-(4-(2,3-tetradeutero-cyclopropylamino)-5-(trifluoromethyl)pyrimidin-2-ylamino)-2-fluoro-5-methoxyphenyl)(morpholino)methanone
[5-Methoxy-4-(4-methylamino-5-trifluoromethyl-pyrimidin-2-ylamino)-2-trifluoromethoxy-phenyl]-morpholin-4-yl-methanone
[4-(4-Ethylamino-5-trifluoromethyl-pyrimidin-2-ylamino)-2,5-dimethoxy-phenyl]-morpholin-4-yl-methanone
[2-Chloro-5-methyl-4-(4-methylamino-5-trifluoromethyl-pyrimidin-2-ylamino)-phenyl]-morpholin-4-yl-methanone
(4-(4-(ethylamino)-5-(trifluoromethyl)pyrimidin-2-ylamino)-2-fluoro-5-methoxyphenyl)(perdeuteromorpholino)methanone
(5-bromo-2-methoxy-4-(4-(methylamino)-5-(trifluoromethyl)pyrimidin-2-ylamino)phenyl)(morpholino)methanone
[4-(4-Cyclobutylamino-5-trifluoromethyl-pyrimidin-2-ylamino)-5-methoxy-2-methyl-phenyl]-morpholin-4-yl-methanone or
[2,5-Dimethyl-4-(4-methylamino-5-trifluoromethyl-pyrimidin-2-ylamino)-phenyl]-morpholin-4-yl-methanone.

In another embodiment, the LRRK2 modulators are of formula II:

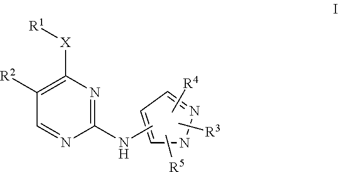

or pharmaceutically acceptable salts thereof,
wherein:

X is: —NR^{a}—; or —O— wherein R^{a} is hydrogen or C₁₋₆alkyl;

R¹ is: C₁₋₆alkyl; C₂₋₆alkenyl; C₂₋₆alkynyl; halo-C₁₋₆alkyl; C₁₋₆alkoxy-C₁₋₆alkyl; hydroxy-C₁₋₆alkyl; amino-C₁₋₆alkyl; C₁₋₆alkylsulfonyl-C₁₋₆alkyl; C₃₋₆cycloalkyl optionally substituted one or more times with C₁₋₆alkyl; C₃₋₆cycloalkyl-C₁₋₆alkyl wherein the C₃₋₆cycloalkyl portion is optionally substituted one or more times with C₁₋₆alkyl; heterocyclyl optionally substituted one or more times with R⁷; or heterocyclyl-C₁₋₆alkyl optionally substituted one or more times with R⁷;

or X and R¹ together form C₁₋₆alkyl; C₁₋₆alkoxy-C₁₋₆alkyl; C₃₋₆cycloalkyl optionally substituted one or more times with R⁶; or C₃₋₆cycloalkyl-C₁₋₆alkyl wherein the C₃₋₆cycloalkyl portion is optionally substituted one or more times with R⁶;

or R¹ and R^{a} together with the atoms to which they are attached may form a three- to six-membered heterocyclic ring optionally substituted one or more times with R⁷;

R² is: C₁₋₆alkyl; halo; C₁₋₆alkoxy; cyano; C₂₋₆alkynyl; C₂₋₆alkenyl; halo-C₁₋₆alkyl; halo-C₁₋₆alkoxy; C₃₋₆cycloalkyl optionally substituted one or more times with R⁶; C₃₋₆cycloalkyl-C₁₋₆alkyl wherein the C₃₋₆cycloalkyl portion is optionally substituted one or more times with R⁶; —OR^{b} wherein R^{b} is C₁₋₆alkyl, C₃₋₆cycloalkyl optionally substituted one or more times with R⁶, or C₃₋₆cycloalkyl-C₁₋₆alkyl wherein the C₃₋₆cycloalkyl portion is optionally substituted one or more times with R⁶; or —C(O)—R^{c} wherein R^{c} is C₁₋₆alkyl, C₁₋₆alkoxy, amino, or heterocyclyl optionally substituted one or more times with R⁷;

R³ is: hydrogen; C₁₋₆alkyl; halo-C₁₋₆alkyl; C₂₋₆alkenyl; C₂₋₆alkynyl; hydroxy-C₁₋₆alkyl; C₁₋₆alkoxy-C₁₋₆alkyl; cyano-C₁₋₆alkyl; C₁₋₆alkylsulfonyl; C₁₋₆alkylsulfonylC₁₋₆alkyl; amino-C₁₋₆alkyl; C₃₋₆cycloalkyl optionally substituted one or more times with R⁶; C₃₋₆cycloalkyl-C₁₋₆alkyl wherein the C₃₋₆cycloalkyl portion is optionally substituted one or more times with R⁶; C₃₋₆cycloalkyl-sulfonyl wherein the C₃₋₆cycloalkyl portion is optionally substituted one or more times with R⁶; heterocyclyl optionally substituted one or more times with R⁷; heterocyclyl-C₁₋₆alkyl wherein the heterocyclyl portion is optionally substituted one or more times with R⁷; aryl optionally substituted one or more times with R⁸; aryl-C₁₋₆alkyl wherein the aryl portion is optionally substituted one or more times with R⁸; heteroaryl optionally substituted one or more times with R⁸; heteroaryl-C₁₋₆alkyl wherein the heteroaryl portion is optionally substituted one or more times with R⁸; or —Y—C(O)—R^{d};

Y is C₂₋₆alkylene or a bond;

R^{d} is C₁₋₆alkyl, C₁₋₆alkoxy, amino, C₁₋₆alkyl-amino, halo-C₁₋₆alkyl-amino, halo-C₁₋₆alkyl, hydroxy-C₁₋₆alkyl, hydroxy, C₁₋₆alkoxy-C₁₋₆alkyl, cyano-C₁₋₆alkyl, C₃₋₆cycloalkyl optionally substituted one or more times with R⁶, C₃₋₆cycloalkyl-C₁₋₆alkyl wherein the C₃₋₆cycloalkyl portion is optionally substituted one or more times with R⁶heterocyclyl optionally substituted one or more times with R⁷, or heterocyclyl-C₁₋₆alkyl wherein the heterocyclyl portion is optionally substituted one or more times with R⁷;

R⁴ is: hydrogen; C₁₋₆alkyl; halo; cyano; halo-C₁₋₆alkyl; C₂₋₆alkenyl; C₂₋₆alkynyl; C₁₋₆alkoxy; C₁₋₆alkoxy-C₁₋₆alkyl; hydroxy-C₁₋₆alkyl; C₃₋₆cycloalkyl optionally substituted one or more times with R⁶; C₃₋₆cycloalkyl-C₁₋₆alkyl wherein the C₃₋₆cycloalkyl portion is optionally substituted one or more times with R⁶; or —Y—C(O)—R^{d};

R⁵ is: hydrogen; or C₁₋₆alkyl;

each R⁶ is independently: C₁₋₆alkyl; halo-C₁₋₆alkyl; C₁₋₆alkoxy; oxo; cyano; halo; or Y—C(O)—R^{d};

each R⁷ is independently: C₁₋₆alkyl; halo-C₁₋₆alkyl; halo; oxo; C₁₋₆alkoxy; C₁₋₆alkylsulfonyl; C₁₋₆alkoxy-C₁₋₆alkyl; cyano; —Y—C(O)—R^{d}; heterocyclyl; heterocyclyl-C₁₋₆alkyl; C₃₋₆cycloalkyl; C₃₋₆cycloalkyl-C₁₋₆alkyl; or C₃₋₆cycloalkylsulfonyl; and

each R⁸ is independently: oxo; C₁₋₆alkyl; halo-C₁₋₆alkyl; halo; C₁₋₆alkyl-sulfonyl; C₁₋₆alkoxy; C₁₋₆alkoxy-C₁₋₆alkyl; cyano; hetoeryclyl; heterocyclyl-C₁₋₆alkyl; —Y—C(O)—R^{d}; C₃₋₆cycloalkyl, C₃₋₆cycloalkyl-C₁₋₆alkyl, or C₃₋₆cycloalkyl-sulfonyl.

In certain embodiments of formula II, X is —NR^{a}— or —O—.

In certain embodiments of formula II, X is —NR^{a}.

In certain embodiments of formula II, X is —O—.

In certain embodiments of formula II, X is —NH— or —O—.

In certain embodiments of formula II, X is —NH—.

In certain embodiments of formula II, X is —O—.

In certain embodiments of formula II, R^{a} is hydrogen.

In certain embodiments of formula II, R^{a} is C₁₋₆alkyl.

In certain embodiments of formula II, R¹ is: C₁₋₆alkyl; C₂₋₆alkenyl; C₂₋₆alkynyl; halo-C₁₋₆alkyl; C₁₋₆alkoxy-C₁₋₆alkyl; hydroxy-C₁₋₆alkyl; amino-C₁₋₆alkyl; C₁₋₆alkylsulfonyl-C₁₋₆alkyl; C₃₋₆cycloalkyl optionally substituted with C₁₋₆alkyl; C₃₋₆cycloalkyl-C₁₋₆alkyl wherein the C₃₋₆cycloalkyl portion is optionally substituted with C₁₋₆alkyl; heterocyclyl; or heterocyclyl-C₁₋₆alkyl.

In certain embodiments of formula II, R¹ is: C₁₋₆alkyl; halo-C₁₋₆alkyl; C₁₋₆alkoxy-C₁₋₆alkyl; amino-C₁₋₆alkyl; C₁₋₆alkylsulfonyl-C₁₋₆alkyl; C₃₋₆cycloalkyl; or C₃₋₆cycloalkyl-C₁₋₆alkyl.

In certain embodiments of formula II, R¹ is: C₁₋₆alkyl; C₃₋₆cycloalkyl optionally substituted with C₁₋₆alkyl; or C₃₋₆cycloalkyl-C₁₋₆alkyl wherein the C₃₋₆cycloalkyl portion is optionally substituted with C₁₋₆alkyl.

In certain embodiments of formula II, R¹ is: C₁₋₆alkyl; halo-C₁₋₆alkyl; C₁₋₆alkoxy-C₁₋₆alkyl; amino-C₁₋₆alkyl; C₁₋₆alkylsulfonyl-C₁₋₆alkyl; tetrahydrofuranyl; tetrahydrofuranyl-C₁₋₆alkyl; oxetanyl; or oxetan-C₁₋₆alkyl.

In certain embodiments of formula II, R¹ is: C₁₋₆alkyl; halo-C₁₋₆alkyl; C₁₋₆alkoxy-C₁₋₆alkyl; amino-C₁₋₆alkyl; or C₁₋₆alkylsulfonyl-C₁₋₆alkyl.

In certain embodiments of formula II, R¹ is C₁₋₆alkyl or halo-C₁₋₆alkyl.

In certain embodiments of formula II, R¹ is C₁₋₆alkyl.

In certain embodiments of formula II, R¹ is halo-C₁₋₆alkyl.

In certain embodiments of formula II, R¹ is C₁₋₆alkoxy-C₁₋₆alkyl.

In certain embodiments of formula II, R¹ is amino-C₁₋₆alkyl.

In certain embodiments of formula II, R¹ is C₁₋₆alkylsulfonyl-C₁₋₆alkyl.

In certain embodiments of formula II, R¹ is C₃₋₆cycloalkyl optionally substituted with C₁₋₆alkyl.

In certain embodiments of formula II, R¹ is C₃₋₆cycloalkyl-C₁₋₆alkyl wherein the C₃₋₆cycloalkyl portion is optionally substituted with C₁₋₆alkyl.

In certain embodiments of formula II, R¹ is heterocyclyl or heterocyclyl-C₁₋₆alkyl.

In embodiments of formula II wherein R¹ is heterocyclyl or heterocyclyl-C₁₋₆alkyl, such heterocyclyl may be piperidinyl, piperazinyl, morpholinyl, tetrahydropyranyl, pyrrolidinyl, tetrahydrofuranyl or oxetanyl, each optionally substituted as defined herein.

In embodiments of formula II wherein R¹ is heterocyclyl or heterocyclyl-C₁₋₆alkyl, such heterocyclyl may be tetrahydropyranyl, piperidinyl, tetrahydrofuranyl or oxetanyl, each optionally substituted as defined herein.

In certain embodiments of formula II, R¹ is tetrahydrofuranyl.

In certain embodiments of formula II, R¹ is tetrahydropyranyl.

In certain embodiments of formula II, R¹ is tetrahydrofuranyl-C₁₋₆alkyl or oxetanyl.

In certain embodiments of formula II, R¹ is tetrahydrofuranyl-C₁₋₆alkyl.

In certain embodiments of formula II, R¹ is oxetanyl.

In certain embodiments of formula II, R¹ is or oxetan-C₁₋₆alkyl.

In certain embodiments of formula II, R¹ is: methyl; ethyl; n-propyl; isopropyl; isobutyl; 3,3-dimethylpropyl; cyclopropyl; cyclobutyl; cyclopentyl; cyclohexyl; cyclopropylmethyl; cyclobutylmethyl; cyclopentylmethyl; cyclopropylethyl; methoxyethyl; oxetanyl; tetrahydropyranyl; 2,2-difluoroethyl; or tetrahydrofuranylmethyl.

In certain embodiments of formula II, R¹ is: methyl; ethyl; n-propyl; isopropyl; isobutyl; 3,3-dimethylpropyl; cyclopentyl; cyclohexyl; cyclopropylmethyl; cyclobutylmethyl; cyclopentylmethyl; cyclopropylethyl; methoxyethyl; oxetanyl; tetrahydropyranyl; 2,2-difluoroethyl; or tetrahydrofuranylmethyl.

In certain embodiments of formula II, R¹ is: methyl; ethyl; n-propyl; isopropyl; isobutyl; 3,3-dimethylpropyl; cyclopentyl; cyclohexyl; cyclopentylmethyl; methoxyethyl; oxetanyl; tetrahydropyranyl; or tetrahydrofuranylmethyl.

In certain embodiments of formula II, R¹ is 2,2-difluoroethyl.

In certain embodiments of formula II, R¹ is: methyl; ethyl; n-propyl; isopropyl; or isobutyl.

In certain embodiments of formula II, R¹ is methyl or ethyl.

In certain embodiments of formula II, R¹ is methyl.

In certain embodiments of formula II, R¹ is ethyl.

In certain embodiments of formula II, R¹ is: cyclopropyl; cyclobutyl; cyclopentyl; cyclohexyl; cyclopropylmethyl; cyclobutylmethyl; cyclopentylmethyl; or cyclopropylethyl.

In certain embodiments of formula II, R¹ is: cyclopentyl; cyclohexyl; or cyclopentylmethyl.

In certain embodiments of formula II, R¹ is: cyclopropyl.

In certain embodiments of formula II, R¹ and R^{a} together with the atoms to which they are attached may form a three- to six-membered heterocyclic ring.

In certain embodiments of formula II, R¹ and R^{a} together with the atoms to which they are attached may form a three-membered heterocyclic ring.

In certain embodiments of formula II, R¹ and R^{a} together with the atoms to which they are attached may form a four-membered heterocyclic ring.

In certain embodiments of formula II, R¹ and R^{a} together with the atoms to which they are attached may form a five-membered heterocyclic ring.

In certain embodiments of formula II, R¹ and R^{a} together with the atoms to which they are attached may form a six-membered heterocyclic ring.

In certain embodiments of formula II, X and R¹ together form C₁₋₆alkyl; C₃₋₆cycloalkyl optionally substituted with C₁₋₆alkyl; or C₃₋₆cycloalkyl-C₁₋₆alkyl wherein the C₃₋₆cycloalkyl portion is optionally substituted with C₁₋₆alkyl.

In certain embodiments of formula II, X and R¹ together form C₁₋₆alkyl.

In certain embodiments of formula II, X and R¹ together form C₃₋₆cycloalkyl optionally substituted with C₁₋₆alkyl.

In certain embodiments of formula II, X and R¹ together form C₃₋₆cycloalkyl-C₁₋₆alkyl wherein the C₃₋₆cycloalkyl portion is optionally substituted with C₁₋₆alkyl.

In certain embodiments of formula II, R² is: C₁₋₆alkyl; halo; C₁₋₆alkoxy; cyano; C₂₋₆alkynyl; C₂₋₆alkenyl; halo-C₁₋₆alkyl; halo-C₁₋₆alkoxy; C₃₋₆cycloalkyl optionally substituted with C₁₋₆alkyl; C₃₋₆cycloalkyl-C₁₋₆alkyl wherein the C₃₋₆cycloalkyl portion is optionally substituted with C₁₋₆alkyl; —OR^{b} wherein R^{b} is C₁₋₆alkyl, C₃₋₆cycloalkyl optionally substituted with C₁₋₆alkyl, or C₃₋₆cycloalkyl-C₁₋₆alkyl wherein the C₃₋₆cycloalkyl portion is optionally substituted with C₁₋₆alkyl; or —C(O)—R^{c}.

In certain embodiments of formula II, R² is: halo; C₁₋₆alkoxy; halo-C₁₋₆alkyl; halo-C₁₋₆alkoxy; C₃₋₆cycloalkyl wherein the C₃₋₆cycloalkyl portion is optionally substituted with C₁₋₆alkyl; C₃₋₆cycloalkyl-C₁₋₆alkyl wherein the C₃₋₆cycloalkyl portion is optionally substituted with C₁₋₆alkyl; tetrahydrofuranyl; tetrahydrofuranyl-C₁₋₆alkyl; oxetanyl; or oxetan-C₁₋₆alkyl.

In certain embodiments of formula II, R² is: halo; C₁₋₆alkoxy; halo-C₁₋₆alkyl; cyano; C₂₋₆alkynyl; C₂₋₆alkenyl; C₃₋₆cycloalkyl; or C₃₋₆cycloalkyl-C₁₋₆alkyl.

In certain embodiments of formula II, R² is: halo; C₁₋₆alkoxy; halo-C₁₋₆alkyl; cyano; C₃₋₆cycloalkyl; or C₃₋₆cycloalkyl-C₁₋₆alkyl.

In certain embodiments of formula II, R² is: halo; C₁₋₆alkoxy; halo-C₁₋₆alkyl; C₃₋₆cycloalkyl; or C₃₋₆cycloalkyl-C₁₋₆alkyl.

In certain embodiments of formula II, R² is: halo; halo-C₁₋₆alkyl or cyano.

In certain embodiments of formula II, R² is: fluoro; bromo; chloro; iodo; trifluoromethyl; or cyano.

In certain embodiments of formula II, R² is: chloro; trifluoromethyl; or cyano.

In certain embodiments of formula II, R² is: halo; or halo-C₁₋₆alkyl.

In certain embodiments of formula II, R² is C₁₋₆alkyl.

In certain embodiments of formula II, R² is halo.

In certain embodiments of formula II, R² is C₁₋₆alkoxy.

In certain embodiments of formula II, R² is halo-C₁₋₆alkoxy.

In certain embodiments of formula II, R² is halo-C₁₋₆alkyl.

In certain embodiments of formula II, R² is C₃₋₆cycloalkyl.

In certain embodiments of formula II, R² is C₃₋₆cycloalkyl-C₁₋₆alkyl.

In certain embodiments of formula II, R² is tetrahydrofuranyl.

In certain embodiments of formula II, R² is tetrahydrofuranyl-C₁₋₆alkyl.

In certain embodiments of formula II, R² is oxetanyl.

In certain embodiments of formula II, R² is oxetan-C₁₋₆alkyl.

In certain embodiments of formula II, R² is fluoro, chloro or bromo.

In certain embodiments of formula II, R² is chloro.

In certain embodiments of formula II, R² is fluoro.

In certain embodiments of formula II, R² is bromo.

In certain embodiments of formula II, R² is bromo.

In certain embodiments of formula II, R² is iodo.

In certain embodiments of formula II, R² is trifluoromethyl.

In certain embodiments of formula II, R² is methoxy.

In certain embodiments of formula II, R² is cyano.

In certain embodiments of formula II, R² is C₂₋₆alkynyl.

In certain embodiments of formula II, R² is C₂₋₆alkenyl.

In certain embodiments of formula II, R² is —OR^{b} wherein R^{b} is C₁₋₆alkyl, C₃₋₆cycloalkyl optionally substituted with C₁₋₆alkyl, or C₃₋₆cycloalkyl-C₁₋₆alkyl wherein the C₃₋₆cycloalkyl portion is optionally substituted with C₁₋₆alkyl.

In certain embodiments of formula I, R² is —C(O)—R^{c} wherein R^{c} is C₁₋₆alkyl, C₁₋₆alkoxy, amino, or heterocyclyl.

In certain embodiments of formula II, R³ is: C₁₋₆alkyl; halo-C₁₋₆alkyl; C₂₋₆alkenyl; C₂₋₆alkynyl; hydroxy-C₁₋₆alkyl; C₁₋₆alkoxy-C₁₋₆alkyl; cyano-C₁₋₆alkyl; C₁₋₆alkylsulfonyl; C₁₋₆alkylsulfonylC₁₋₆alkyl; amino-C₁₋₆alkyl; C₃₋₆cycloalkyl optionally substituted one or more times with R⁶; C₃₋₆cycloalkyl-C₁₋₆alkyl wherein the C₃₋₆cycloalkyl portion is optionally substituted one or more times with R⁶; heterocyclyl optionally substituted one or more times with R⁷; heterocyclyl-C₁₋₆alkyl wherein the heterocyclyl portion is optionally substituted one or more times with R⁷; aryl optionally substituted one or more times with R⁸; heteroaryl optionally substituted one or more times with R⁸; or —Y—C(O)—R^{d}.

In certain embodiments of formula II, R³ is: hydrogen; C₁₋₆alkyl; halo-C₁₋₆alkyl; C₂₋₆alkenyl; C₂₋₆alkynyl; hydroxy-C₁₋₆alkyl; C₁₋₆alkoxy-C₁₋₆alkyl; cyano-C₁₋₆alkyl; C₁₋₆alkylsulfonyl; C₁₋₆alkylsulfonylalkyl; amino-C₁₋₆alkyl; C₃₋₆cycloalkyl optionally substituted with C₁₋₆alkyl; C₃₋₆cycloalkyl-C₁₋₆alkyl wherein the C₃₋₆cycloalkyl portion is optionally substituted with C₁₋₆alkyl; heterocyclyl; heterocyclyl-C₁₋₆alkyl; aryl; heteroaryl; or —C(O)—R^{c}.

In certain embodiments of formula II, R³ is: C₁₋₆alkyl; halo-C₁₋₆alkyl; hydroxy-C₁₋₆alkyl; C₁₋₆alkoxy-C₁₋₆alkyl; C₃₋₆cycloalkyl optionally substituted with C₁₋₆alkyl; C₃₋₆cycloalkyl-C₁₋₆alkyl wherein the C₃₋₆cycloalkyl portion is optionally substituted with C₁₋₆alkyl; heterocyclyl; heterocyclyl-C₁₋₆alkyl; or —C(O)—R^{b} wherein R^{b} is C₁₋₆alkyl, C₁₋₆alkoxy, amino, or heterocyclyl.

In certain embodiments of formula II, R³ is: C₁₋₆alkyl; hydroxy-C₁₋₆alkyl; C₁₋₆alkoxy-C₁₋₆alkyl; C₃₋₆cycloalkyl optionally substituted with C₁₋₆alkyl; C₃₋₆cycloalkyl-C₁₋₆alkyl wherein the C₃₋₆cycloalkyl portion is optionally substituted with C₁₋₆alkyl; heterocyclyl; heterocyclyl-C₁₋₆alkyl; or —C(O)—R^{c} wherein R^{c} is C₁₋₆alkyl, C₁₋₆alkoxy, amino, or heterocyclyl.

In certain embodiments of formula II, R³ is: C₁₋₆alkyl; hydroxy-C₁₋₆alkyl; C₁₋₆alkoxy-C₁₋₆alkyl; C₃₋₆cycloalkyl optionally substituted one or more times with R⁶; C₃₋₆cycloalkyl-C₁₋₆alkyl wherein the C₃₋₆cycloalkyl portion is optionally substituted one or more times with R⁶; heterocyclyl optionally substituted one or more times with R⁷; heterocyclyl-C₁₋₆alkyl wherein the heterocyclyl portion is optionally substituted one or more times with R⁷; or —C(O)—R^{d}.

In certain embodiments of formula II, R³ is: C₁₋₆alkyl; hydroxy-C₁₋₆alkyl; C₁₋₆alkoxy-C₁₋₆alkyl; heterocyclyl; heterocyclyl-C₁₋₆alkyl; or —C(O)—R^{c} wherein R^{c} is C₁₋₆alkyl, C₁₋₆alkoxy, amino, or heterocyclyl.

In certain embodiments of formula II, R³ is: C₁₋₆alkyl; hydroxy-C₁₋₆alkyl; C₁₋₆alkoxy-C₁₋₆alkyl; heterocyclyl; or heterocyclyl-C₁₋₆alkyl.

In certain embodiments of formula II, R³ is: C₁₋₆alkyl; hydroxy-C₁₋₆alkyl; or C₁₋₆alkoxy-C₁₋₆alkyl.

In embodiments of formula II wherein R³ is heterocyclyl or heterocyclyl-C₁₋₆alkyl, such heterocyclyl may be piperidinyl, piperazinyl, morpholinyl, tetrahydropyranyl, pyrrolidinyl, tetrahydrofuranyl or oxetanyl.

In embodiments of formula II wherein R³ is heterocyclyl or heterocyclyl-C₁₋₆alkyl, such heterocyclyl may be piperidinyl, piperazinyl, morpholinyl, tetrahydropyranyl, pyrrolidinyl, azetidinyl, tetrahydrofuranyl or oxetanyl, each optionally substituted one or more times, or one or two times, with R⁷ as defined herein.

In embodiments of formula II wherein R³ is heterocyclyl or heterocyclyl-C₁₋₆alkyl, such heterocyclyl may be piperidinyl, morpholinyl, tetrahydropyranyl, tetrahydrofuranyl or oxetanyl.

In embodiments of formula II wherein R³ is heterocyclyl or heterocyclyl-C₁₋₆alkyl, such heterocyclyl may be piperidinyl, pyrrolidinyl, azetidinyl, morpholinyl, tetrahydropyranyl, tetrahydrofuranyl or oxetanyl, each optionally substituted one or more times, or one or two times, with R⁷ as defined herein.

In certain embodiments of formula II, R³ is: methyl; ethyl; n-propyl; isopropyl; 2-methoxy-ethyl; oxetan-3-yl; 2-(morpholin-4-yl)-ethyl; 2-hydroxy-2-methyl-propan-1-yl; tetrahydropyran-4-yl; or morpholin-4-yl-carbonyl.

In certain embodiments of formula I, R³ is: methyl; ethyl; n-propyl; isopropyl; 2-methoxy-ethyl; oxetan-3-yl; 2-(morpholin-4-yl)-ethyl; 2-hydroxy-2-methyl-propan-1-yl; or tetrahydropyran-4-yl.

In certain embodiments of formula II, R³ is hydrogen.

In certain embodiments of formula II, R³ is C₁₋₆alkyl.

In certain embodiments of formula II, R³ is halo-C₁₋₆alkyl.

In certain embodiments of formula II, R³ is C₂₋₆alkenyl.

In certain embodiments of formula II, R³ is C₂₋₆alkynyl.

In certain embodiments of formula II, R³ is hydroxy-C₁₋₆alkyl.

In certain embodiments of formula II, R³ is C₁₋₆alkoxy-C₁₋₆alkyl.

In certain embodiments of formula II, R³ is C₃₋₆cycloalkyl optionally substituted one or more times with R⁶.

In certain embodiments of formula II, R³ is C₃₋₆cycloalkyl optionally substituted with C₁₋₆alkyl.

In certain embodiments of formula II, R³ is C₃₋₆cycloalkyl-C₁₋₆alkyl wherein the C₃₋₆cycloalkyl portion is optionally substituted one or more times with R⁶.

In certain embodiments of formula II, R³ is C₃₋₆cycloalkyl-C₁₋₆alkyl wherein the C₃₋₆cycloalkyl portion is optionally substituted with C₁₋₆alkyl.

In certain embodiments of formula II, R³ is heterocyclyl optionally substituted one or more times with R⁷.

In certain embodiments of formula II, R³ is heterocyclyl.

In certain embodiments of formula II, R³ is heterocyclyl-C₁₋₆alkyl wherein the heterocyclyl portion is optionally substituted one or more times with R⁷.

In certain embodiments of formula II, R³ is heterocyclyl-C₁₋₆alkyl.

In certain embodiments of formula II, R³ is —C(O)—R^{c}.

In certain embodiments of formula II, R³ is cyano-C₁₋₆alkyl.

In certain embodiments of formula II, R³ is C₁₋₆alkylsulfonyl.

In certain embodiments of formula II, R³ is C₁₋₆alkylsulfonyl-C₁₋₆alkyl.

In certain embodiments of formula II, R³ is amino-C₁₋₆alkyl.

In certain embodiments of formula II, R³ is aryl optionally substituted one or more times with R⁸.

In certain embodiments of formula II, R³ is aryl.

In certain embodiments of formula II, R³ is phenyl optionally substituted one or more times, or one or two times, with R⁸.

In certain embodiments of formula II, R³ is heteroaryl optionally substituted one or more times, or one or two times, with R⁸.

In certain embodiments of formula II, R³ is heteroaryl.

In certain embodiments of formula II, R³ is C₃₋₆cycloalkyl-sulfonyl wherein the C₃₋₆cycloalkyl portion is optionally substituted one or more times with R⁶.

In certain embodiments of formula II, R³ is: hydrogen; methyl; ethyl; propyl; isopropyl; butyl; cyclopropyl; cyclopropylmethyl; cyclobutyl; methanesulfonyl; ethylsulfonyl; cyclopropylsulfonyl; sec-butylsulfonyl; morpholin-4-yl-ethyl; oxetan-3-yl; 2-methoxyethyl; 2-hydroxy-2-methyl-propyl; 3-hydroxy-2-methyl-propan-2-yl; 2-methoxy-propyl; tetrahydro-2H-pyran-4-yl; tetrahydrofuran-3-yl; 2,6-dimethyltetrahydro-2H-pyran-4-yl; tetrahydro-2H-pyran-3-yl); phenyl; 4-(methylsulfonyl)phenyl); 4-cyano-phenyl; 4-fluoro-phenyl; 4-chloro-phenyl; 3,5-difluorophenyl; 4-(dimethylamino-carbonyl)-phenyl); 4-(cyclopropylsulfonyl)phenyl; 2,2,2-trifluoroethyl; 2-fluoroethyl; difluoromethyl; 2-dimethyl-1,3-dioxan-5-yl; 1-methyl-cyclopropyl-carbonyl; 3-methylpyridin-4-yl; 2-methylpyridin-4-yl; pyridin-2-yl; pyrimidin-2-yl; pyrimidin-5-yl; pyridin-2-ylmethyl; 1-(pyridin-2-yl)ethyl; cyclopropylsulfonyl; 1-cyano-1-methyl-ethyl (also called 2-cyano-propan-2-yl); 2-cyano-ethyl; 1-cyano-ethyl; 2-cyano-2-methyl-propyl; 1-(2,2,2-trifluoroethyl)piperidin-4-yl; 1-(methylsulfonyl)azetidin-3-yl; (3-methyloxetan-3-yl)methyl; (1S,5S)-8-oxabicyclo[3.2.1]octan-3-yl; 1-(oxetan-3-yl)piperidin-4-yl; 1-acetyl-piperidin-4-yl; 1-(cyclopropyl-carbonyl)-piperidin-4-yl; 1-methyl-piperidin-4-yl; 1-methyl-2-oxo-piperidin-5-yl; 2-oxo-piperidin-5-yl; 1-(isopropyl-carbonyl)-piperidin-4-yl; 1-(oxetan-3-yl)azetidin-3-yl; 1-(cyclopropyl-carbonyl)-piperidin-4-yl; 2-methoxycyclopentyl; 3-methoxycyclopentyl; 1-methoxy-2-methylpropan-2-yl; tetrahydro-2H-1,1-dioxo-thiopyran-4-yl; 3-fluoro-1-(oxetan-3-yl)piperidin-4-yl; 1-methoxypropan-2-yl; 1-(2,2,2-trifluoroethyl)azetidin-3-yl); 1-(oxetan-3-yl)pyrrolidin-3-yl; 1-isopropyl azetidin-3-yl; 3-fluoro-1-methylpiperidin-4-yl; 1-ethyl-3-fluoropiperidin-4-yl; 1-methylpyrrolidin-3-yl; 2-methoxyethyl)piperidin-4-yl); 1-methyl-1-(methylamino-carbonyl)-ethyl; 2-methyl-2-morpholino-propyl; 4,4-difluorocyclohexyl; morpholin-4-yl-carbonyl; dimethylamino-carbonyl-methyl; methylamino-carbonyl-methyl; 1-methyl-1-(dimethylamino-carbonyl)-ethyl; pyrrolidin-′-yl-carbonyl; 1-cyamo-cyclopropyl; 1-(pyrrolidin-′-yl-carbonyl)-ethyl; 1-(dimethylamino-carbonyl)-ethyl; 1-(methoxy-carbonyl)-ethyl; 1-(tert-butylamino-carbonyl)-1-methyl-ethyl; 1-(2,2,2-trifluoroethyllamino-carbonyl)-1-methyl-ethyl; 1-(ethylamino-carbonyl)-1-methyl-ethyl; 1-(cyclopropylmethylamino-carbonyl)-1-methyl-ethyl; 1-(ethylamino-carbonyl)-cyclobutyl; 1-(isopropylamino-carbonyl)-1-methyl-ethyl; 1-cyano-cyclobutyl; 2-methoxy-1-methyl-ethyl; 1-methyl-1-(methoxy-carbonyl)-ethyl; 2-methoxy-2-methyl-propan-1-yl; 1-(oxetan-3-yl)-pyrrolidin-3-yl; isopropylsulfonyl; butane-2-sulfonyl; 1-(2-fluoroethyl)-piperidin-4-yl; 3-fluoro-1-methyl-piperidin-4-yl; 1-ethyl-3-fluoro-piperidin-4-yl; pyridin-3-ylmethyl; 6-methyl-pyridin-2-ylmethyl; 2-(morpholin-1-yl)-1,1, dimethyl-ethyl; pyrimdin-2-yl-methyl; 3-fluoro-1-(oxetan-3-yl)-piperidin-4-yl; 1-(oxetan-3-yl)-piperidin-3-yl; 1-([1,3]Dioxolan-2-ylmethyl)-piperidin-4-yl; pyridazin-3-ylmethyl; piperidin-3-yl; pyrazin-2-ylmethyl; 2-hydroxy-3-methyl-butan-1-yl; 1-([1,3]Dioxolan-2-ylmethyl)-pyrrolidin-3-yl; pyrimidin-4-ylmethyl; 1-methyl-1H-pyrazol-3-ylmethyl; 1-methyl-1-(4H-[1,2,4]triazol-3-yl)-ethyl; 1-methyl-1-(5-methyl-4H-[1,2,4]triazol-3-yl)-ethyl; 3-fluoro-piperidin-4-yl; 2-hydroxy-cyclopentyl; dimethyl-[1,3]dioxan-5-yl, 2-(5-methyl-1,3,4-oxadiazol-2-yl)propan-2-yl; 2-(4-methyl-4H-1,2,4-triazol-3-yl)propan-2-yl; 2-(1-methyl-1H-1,2,4-triazol-3-yl)propan-2-yl; 2-(1-methyl-1H-pyrazol-4-yl)propan-2-yl; 2-(1-methyl-1H-pyrazol-3-yl)propan-2-yl; 2-(1-methyl-1H-pyrazol-5-yl; 2-(4H-1,2,4-triazol-3-yl)propan-2-yl; or 1-methyl-1H-pyrazole-4-yl.

In certain embodiments of formula II, R³ is: methyl; ethyl; propyl; isopropyl; butyl; cyclopropyl; cyclopropylmethyl; cyclobutyl; methanesulfonyl; ethylsulfonyl; cyclopropylsulfonyl; sec-butylsulfonyl; morpholin-4-yl-ethyl; oxetan-3-yl; 2-methoxyethyl; 2-hydroxy-2-methyl-propyl; 3-hydroxy-2-methyl-propan-2-yl; 2-methoxy-propyl; tetrahydro-2H-pyran-4-yl; tetrahydrofuran-3-yl; 2,6-dimethyltetrahydro-2H-pyran-4-yl; tetrahydro-2H-pyran-3-yl); phenyl; 4-(methylsulfonyl)phenyl); 4-cyano-phenyl; 4-fluoro-phenyl; 4-chloro-phenyl; 3,5-difluorophenyl; 4-(dimethylamino-carbonyl)-phenyl); 4-(cyclopropylsulfonyl)phenyl; 2,2,2-trifluoroethyl; 2-fluoroethyl; difluoromethyl; 2-dimethyl-1,3-dioxan-5-yl; 1-methyl-cyclopropyl-carbonyl; 3-methylpyridin-4-yl; 2-methylpyridin-4-yl; pyridin-2-yl; pyrimidin-2-yl; pyrimidin-5-yl; pyridin-2-ylmethyl; 1-(pyridin-2-yl)ethyl; cyclopropylsulfonyl; 1-cyano-1-methyl-ethyl (also called 2-cyano-propan-2-yl); 2-cyano-ethyl; 1-cyano-ethyl; 2-cyano-2-methyl-propyl; 1-(2,2,2-trifluoroethyl)piperidin-4-yl; 1-(methylsulfonyl)azetidin-3-yl; (3-methyloxetan-3-yl)methyl; (1S,5S)-8-oxabicyclo[3.2.1]octan-3-yl; 1-(oxetan-3-yl)piperidin-4-yl; 1-acetyl-piperidin-4-yl; 1-(cyclopropyl-carbonyl)-piperidin-4-yl; 1-methyl-piperidin-4-yl; 1-methyl-2-oxo-piperidin-5-yl; 2-oxo-piperidin-5-yl; 1-(isopropyl-carbonyl)-piperidin-4-yl; 1-(oxetan-3-yl)azetidin-3-yl; 1-(cyclopropyl-carbonyl)-piperidin-4-yl; 2-methoxycyclopentyl; 3-methoxycyclopentyl; 1-methoxy-2-methylpropan-2-yl; tetrahydro-2H-1,1-dioxo-thiopyran-4-yl; 3-fluoro-1-(oxetan-3-yl)piperidin-4-yl; 1-methoxypropan-2-yl; 1-(2,2,2-trifluoroethyl)azetidin-3-yl); 1-(oxetan-3-yl)pyrrolidin-3-yl; 1-isopropyl azetidin-3-yl; 3-fluoro-1-methylpiperidin-4-yl; 1-ethyl-3-fluoropiperidin-4-yl; 1-methylpyrrolidin-3-yl; 2-methoxyethyl)piperidin-4-yl); 1-methyl-1-(methylamino-carbonyl)-ethyl; 2-methyl-2-morpholino-propyl; 4,4-difluorocyclohexyl; morpholin-4-yl-carbonyl; dimethylamino-carbonyl-methyl; methylamino-carbonyl-methyl; 1-methyl-1-(dimethylamino-carbonyl)-ethyl; pyrrolidin-′-yl-carbonyl; 1-cyamo-cyclopropyl; 1-(pyrrolidin-′-yl-carbonyl)-ethyl; 1-(dimethylamino-carbonyl)-ethyl; 1-(methoxy-carbonyl)-ethyl; 1-(tert-butylamino-carbonyl)-1-methyl-ethyl; 1-(2,2,2-trifluoroethyllamino-carbonyl)-1-methyl-ethyl; 1-(ethylamino-carbonyl)-1-methyl-ethyl; 1-(cyclopropylmethylamino-carbonyl)-1-methyl-ethyl; 1-(ethylamino-carbonyl)-cyclobutyl; 1-(isopropylamino-carbonyl)-1-methyl-ethyl; 1-cyano-cyclobutyl; 2-methoxy-1-methyl-ethyl; 1-methyl-1-(methoxy-carbonyl)-ethyl; 2-methoxy-2-methyl-propan-1-yl; 1-(oxetan-3-yl)-pyrrolidin-3-yl; isopropylsulfonyl; butane-2-sulfonyl; 1-(2-fluoro ethyl)-piperidin-4-yl; 3-fluoro-1-methyl-piperidin-4-yl; 1-ethyl-3-fluoro-piperidin-4-yl; pyridin-3-ylmethyl; 6-methyl-pyridin-2-ylmethyl; 2-(morpholin-1-yl)-1,1, dimethyl-ethyl; pyrimdin-2-yl-methyl; 3-fluoro-1-(oxetan-3-yl)-piperidin-4-yl; 1-(oxetan-3-yl)-piperidin-3-yl; 1-([1,3]Dioxolan-2-ylmethyl)-piperidin-4-yl; pyridazin-3-ylmethyl; piperidin-3-yl; pyrazin-2-ylmethyl; 2-hydroxy-3-methyl-butan-1-yl; 1-([1,3]Dioxolan-2-ylmethyl)-pyrrolidin-3-yl; pyrimidin-4-ylmethyl; 1-methyl-1H-pyrazol-3-ylmethyl; 1-methyl-1-(4H-[1,2,4]triazol-3-yl)-ethyl; 1-methyl-1-(5-methyl-4H-[1,2,4]triazol-3-yl)-ethyl; 3-fluoro-piperidin-4-yl; 2-hydroxy-cyclopentyl; dimethyl-[1,3]dioxan-5-yl; 2-(5-methyl-1,3,4-oxadiazol-2-yl)propan-2-yl; 2-(4-methyl-4H-1,2,4-triazol-3-yl)propan-2-yl; 2-(1-methyl-1H-1,2,4-triazol-3-yl)propan-2-yl; 2-(1-methyl-1H-pyrazol-4-yl)propan-2-yl; 2-(1-methyl-1H-pyrazol-3-yl)propan-2-yl; 2-(1-methyl-1H-pyrazol-5-yl; 2-(4H-1,2,4-triazol-3-yl)propan-2-yl; or 1-methyl-1H-pyrazole-4-yl.

In certain embodiments of formula II, R³ is: hydrogen; methyl; ethyl; n-propyl; isopropyl; 2-methoxy-ethyl; oxetan-3-yl; 2-hydroxy-2-methyl-propan-1-yl; tetrahydropyran-4-yl; or morpholin-4-yl-carbonyl.

In certain embodiments of formula II, R³ is: methyl; ethyl; n-propyl; isopropyl; 2-methoxy-ethyl; oxetan-3-yl; 2-hydroxy-2-methyl-propan-1-yl; or tetrahydropyran-4-yl.

In certain embodiments of formula II, R³ is: methyl; ethyl; isopropyl; 2-methoxy-ethyl; oxetan-3-yl; or 2-hydroxy-2-methyl-propan-1-yl.

In certain embodiments of formula II, R³ is: methyl; ethyl; isopropyl; 2-methoxy-ethyl; oxetan-3-yl; or 2-hydroxy-2-methyl-propan-1-yl.

In certain embodiments of formula II, R³ is: methyl; ethyl; or isopropyl.

In certain embodiments of formula II, R³ is hydrogen.

In certain embodiments of formula II, R³ is methyl.

In certain embodiments of formula II, R³ is ethyl.

In certain embodiments of formula II, R³ is n-propyl.

In certain embodiments of formula II, R³ is isopropyl.

In certain embodiments of formula II, R³ is 2-methoxy-ethyl.

In certain embodiments of formula II, R³ is oxetan-3-yl.

In certain embodiments of formula II, R³ is 2-hydroxy-2-methyl-propan-1-yl.

In certain embodiments of formula II, R³ is tetrahydropyran-4-yl.

In certain embodiments of formula II, R³ is morpholin-4-yl-carbonyl.

In certain embodiments of formula II, R³ is butyl.

In certain embodiments of formula II, R³ is cyclopropyl.

In certain embodiments of formula II, R³ is cyclopropylmethyl.

In certain embodiments of formula II, R³ is cyclobutyl.

In certain embodiments of formula II, R³ is methanesulfonyl.

In certain embodiments of formula II, R³ is ethylsulfonyl.

In certain embodiments of formula II, R³ is cyclopropylsulfonyl.

In certain embodiments of formula II, R³ is sec-butylsulfonyl.

In certain embodiments of formula II, R³ is morpholin-4-yl-ethyl.

In certain embodiments of formula II, R³ is 2-hydroxy-2-methyl-propyl.

In certain embodiments of formula II, R³ is 3-hydroxy-2-methyl-propan-2-yl.

In certain embodiments of formula II, R³ is 2-methoxy-propyl.

In certain embodiments of formula II, R³ is tetrahydro-2H-pyran-4-yl.

In certain embodiments of formula II, R³ is tetrahydrofuran-3-yl.

In certain embodiments of formula II, R³ is 2,6-dimethyltetrahydro-2H-pyran-4-yl.

In certain embodiments of formula II, R³ is tetrahydro-2H-pyran-3-yl).

In certain embodiments of formula II, R³ is phenyl.

In certain embodiments of formula II, R³ is 4-(methylsulfonyl)phenyl).

In certain embodiments of formula II, R³ is 4-cyano-phenyl.

In certain embodiments of formula II, R³ is 4-fluoro-phenyl.

In certain embodiments of formula Iv, R³ is 4-chloro-phenyl.

In certain embodiments of formula II, R³ is 3,5-difluorophenyl.

In certain embodiments of formula II, R³ is 4-(dimethylamino-carbonyl)-phenyl).

In certain embodiments of formula II, R³ is 4-(cyclopropylsulfonyl)phenyl.

In certain embodiments of formula II, R³ is 2,2,2-trifluoroethyl.

In certain embodiments of formula II, R³ is 2-fluoroethyl.

In certain embodiments of formula II, R³ is difluoromethyl.

In certain embodiments of formula II, R³ is 2-dimethyl-1,3-dioxan-5-yl.

In certain embodiments of formula II, R³ is 1-methyl-cyclopropyl-carbonyl.

In certain embodiments of formula II, R³ is 3-methylpyridin-4-yl.

In certain embodiments of formula II, R³ is 2-methylpyridin-4-yl.

In certain embodiments of formula II, R³ is pyridin-2-yl.

In certain embodiments of formula II, R³ is pyrimidin-2-yl.

In certain embodiments of formula II, R³ is pyrimidin-5-yl.

In certain embodiments of formula II, R³ is pyridin-2-ylmethyl.

In certain embodiments of formula II, R³ is 1-(pyridin-2-yl)ethyl.

In certain embodiments of formula II, R³ is cyclopropylsulfonyl.

In certain embodiments of formula II, R³ is 1-cyano-1-methyl-ethyl (also called 2-cyano-propan-2-yl).

In certain embodiments of formula II, R³ is 2-cyano-ethyl.

In certain embodiments of formula II, R³ is 1-cyano-ethyl.

In certain embodiments of formula II, R³ is 2-cyano-2-methyl-propyl.

In certain embodiments of formula II, R³ is 1-(2,2,2-trifluoroethyl)piperidin-4-yl.

In certain embodiments of formula II, R³ is 1-(methylsulfonyl)azetidin-3-yl.

In certain embodiments of formula II, R³ is (3-methyloxetan-3-yl)methyl.

In certain embodiments of formula II, R³ is (1S,5S)-8-oxabicyclo[3.2.1]octan-3-yl.

In certain embodiments of formula II, R³ is 1-(oxetan-3-yl)piperidin-4-yl.

In certain embodiments of formula II, R³ is 1-acetyl-piperidin-4-yl.

In certain embodiments of formula II, R³ is 1-(cyclopropyl-carbonyl)-piperidin-4-yl.

In certain embodiments of formula II, R³ is 1-methyl-piperidin-4-yl.

In certain embodiments of formula II, R³ is 1-methyl-2-oxo-piperidin-5-yl.

In certain embodiments of formula II, R³ is 2-oxo-piperidin-5-yl.

In certain embodiments of formula II, R³ is 1-(isopropyl-carbonyl)-piperidin-4-yl.

In certain embodiments of formula II, R³ is 1-(oxetan-3-yl)azetidin-3-yl.

In certain embodiments of formula II, R³ is 1-(cyclopropyl-carbonyl)-piperidin-4-yl.

In certain embodiments of formula II, R³ is 2-methoxycyclopentyl.

In certain embodiments of formula II, R³ is 3-methoxycyclopentyl.

In certain embodiments of formula II, R³ is 1-methoxy-2-methylpropan-2-yl.

In certain embodiments of formula II, R³ is tetrahydro-2H-1,1-dioxo-thiopyran-4-yl.

In certain embodiments of formula II, R³ is 3-fluoro-1-(oxetan-3-yl)piperidin-4-yl.

In certain embodiments of formula II, R³ is 1-methoxypropan-2-yl.

In certain embodiments of formula II, R³ is 1-(2,2,2-trifluoroethyl)azetidin-3-yl).

In certain embodiments of formula II, R³ is 1-(oxetan-3-yl)pyrrolidin-3-yl.

In certain embodiments of formula II, R³ is 1-isopropylazetidin-3-yl.

In certain embodiments of formula II, R³ is 3-fluoro-1-methylpiperidin-4-yl.

In certain embodiments of formula II, R³ is 1-ethyl-3-fluoropiperidin-4-yl.

In certain embodiments of formula II, R³ is 1-methylpyrrolidin-3-yl.

In certain embodiments of formula II, R³ is 2-methoxyethyl)piperidin-4-yl).

In certain embodiments of formula II, R³ is 1-methyl-1-(methylamino-carbonyl)-ethyl.

In certain embodiments of formula II, R³ is 2-methyl-2-morpholino-propyl.

In certain embodiments of formula II, R³ is 4,4-difluorocyclohexyl.

In certain embodiments of formula II, R³ is dimethylamino-carbonyl-methyl.

In certain embodiments of formula II, R³ is methylamino-carbonyl-methyl.

In certain embodiments of formula II, R³ is 1-methyl-1-(dimethylamino-carbonyl)-ethyl.

In certain embodiments of formula II, R³ is pyrrolidin-′-yl-carbonyl.

In certain embodiments of formula II, R³ is 1-cyano-cyclopropyl.

In certain embodiments of formula II, R³ is 1-(pyrrolidin-′-yl-carbonyl)-ethyl.

In certain embodiments of formula II, R³ is 1-(dimethylamino-carbonyl)-ethyl.

In certain embodiments of formula II, R³ is 1-(methoxy-carbonyl)-ethyl.

In certain embodiments of formula II, R³ is 1-(tert-butylamino-carbonyl)-1-methyl-ethyl.

In certain embodiments of formula II, R³ is 1-(2,2,2-trifluoroethyllamino-carbonyl)-1-methyl-ethyl.

In certain embodiments of formula II, R³ is 1-(ethylamino-carbonyl)-1-methyl-ethyl.

In certain embodiments of formula II, R³ is 1-(cyclopropylmethylamino-carbonyl)-1-methyl-ethyl.

In certain embodiments of formula II, R³ is 1-(ethylamino-carbonyl)-cyclobutyl.

In certain embodiments of formula II, R³ is 1-(isopropylamino-carbonyl)-1-methyl-ethyl.

In certain embodiments of formula II, R³ is 1-cyano-cyclobutyl.

In certain embodiments of formula II, R³ is dimethyl-[1,3]dioxan-5-yl.

In certain embodiments of formula II, R³ is 2-methoxy-2-methyl-propan-1-yl.

In certain embodiments of formula II, R³ is 2-methoxy-1-methyl-ethyl.

In certain embodiments of formula II, R³ is 1-methyl-1-(methoxy-carbonyl)-ethyl.

In certain embodiments of formula II, R³ is 1-oxetan-3-yl-pyrrolidin-3-yl.

In certain embodiments of formula II, R³ is isopropylsulfonyl.

In certain embodiments of formula II, R³ is butane-2-sulfonyl.

In certain embodiments of formula II, R³ is 1-(2-fluoroethyl)-piperidin-4-yl.

In certain embodiments of formula II, R³ is 3-fluoro-1-methyl-piperidin-4-yl.

In certain embodiments of formula II, R³ is 1-ethyl-3-fluoro-piperidin-4-yl. In certain embodiments of formula II, R³ is pyridin-3-ylmethyl.

In certain embodiments of formula II, R³ is 6-methyl-pyridin-2-ylmethyl.

In certain embodiments of formula II, R³ is 2-(morpholin-1-yl)-1,1,dimethyl-ethyl.

In certain embodiments of formula II, R³ is pyrimdin-2-yl-methyl.

In certain embodiments of formula II, R³ is 3-fluoro-1-(oxetan-3-yl)-piperidin-4-yl.

In certain embodiments of formula II, R³ is 1-(oxetan-3-yl)-piperidin-3-yl.

In certain embodiments of formula II, R³ is 1-([1,3]Dioxolan-2-ylmethyl)-piperidin-4-yl.

In certain embodiments of formula II, R³ is pyridazin-3-ylmethyl.

In certain embodiments of formula II, R³ is piperidin-3-yl.

In certain embodiments of formula II, R³ is pyrazin-2-ylmethyl.

In certain embodiments of formula II, R³ is 2-hydroxy-3-methyl-butan-1-yl.

In certain embodiments of formula II, R³ is 1-([1,3]dioxolan-2-ylmethyl)-pyrrolidin-3-yl.

In certain embodiments of formula II, R³ is pyrimidin-4-ylmethyl.

In certain embodiments of formula II, R³ is 1-methyl-1H-pyrazol-3-ylmethyl.

In certain embodiments of formula II, R³ is 1-methyl-1-(5-methyl-4H-[1,2,4]triazol-3-yl)-ethyl.

In certain embodiments of formula II, R³ is 1-methyl-1-(4H-[1,2,4]triazol-3-yl)-ethyl.

In certain embodiments of formula II, R³ is 3-fluoro-piperidin-4-yl; 2-hydroxy-cyclopentyl.

In certain embodiments of formula II, R³ is 2-(5-methyl-1,3,4-oxadiazol-2-yl)propan-2-yl.

In certain embodiments of formula II, R³ is 2-(4-methyl-4H-1,2,4-triazol-3-yl)propan-2-yl.

In certain embodiments of formula II, R³ is 2-(1-methyl-1H-1,2,4-triazol-3-yl)propan-2-yl.

In certain embodiments of formula II, R³ is 2-(1-methyl-1H-pyrazol-4-yl)propan-2-yl; 2-(1-methyl-1H-pyrazol-3-yl)propan-2-yl.

In certain embodiments of formula II, R³ is 2-(1-methyl-1H-pyrazol-5-yl.

In certain embodiments of formula II, R³ is 2-(4H-1,2,4-triazol-3-yl)propan-2-yl.

In certain embodiments of formula II, R³ is 1-methyl-1H-pyrazole-4-yl.

In embodiments of formula II wherein R³ is aryl, such aryl may be unsubstituted phenyl or phenyl substituted one or more times with R⁸, or in certain embodiments, once, twice or three times with a group or groups independently selected from C₁₋₆alkyl, halo, halo-C₁₋₆alkyl, C₁₋₆alkoxy, hydroxy or cyano.

In embodiments of formula II wherein R³ is heteroaryl or heteroaryl-C₁₋₆alkyl, such heteroaryl moiety may be pyrrolyl, pyrazolyl, imidazolyl, thiazolyl, oxazolyl, pyridinyl, pyrimidinyl, pyrazinyl, pyridazinyl, isoxazolyl, isothiazolyl, triazolyl, oxadiaolyl, thiadiazolyl or tetrazolyl, each being unsubstituted or substituted once or twice with R⁸, or in certain embodiments, substituted once or twice with C₁₋₆alkyl.

In embodiments of formula II wherein R³ is heteroaryl or heteroaryl-C₁₋₆alkyl, such heteroaryl moiety may be pyrazolyl, pyridinyl, pyrimidinyl, pyrazinyl, pyridazinyl, triazolyl or oxadiaolyl each being unsubstituted or substituted once or twice with R⁸, or in certain embodiments, substituted once or twice with C₁₋₆alkyl.

In embodiments of formula II wherein R³ is heteroaryl or heteroaryl-C₁₋₆alkyl, such heteroaryl moiety may be pyrrolyl, pyrazolyl, imidazolyl, thiazolyl, oxazolyl, pyridinyl, pyrimidinyl, pyrazinyl or pyridazinyl, each being unsubstituted or substituted one or more times with R⁸.

In embodiments of formula II wherein R³ is heterocyclyl, such heterocyclyl moiety may be piperidinyl, pyrrolidinyl, oxetanyl, tetrahydropyranyl, tetrahydrofuranyl, azetidinyl, [1,3]dioxolanyl or tetrahydrothiopyranyl, each being unsubstituted or substituted one or more times with R⁷.

In embodiments of formula II wherein R³ is heterocyclyl-C₁₋₆alkyl, such heterocyclyl moiety may be piperidinyl, piperazinyl, morpholinyl, thiomorpholinyl, pyrrolidinyl, oxetanyl, tetrahydropyranyl, tetrahydrofuranyl, azetidinyl, [1,3]dioxolanyl or tetrahydrothiopyranyl, each being unsubstituted or substituted one or more times with R⁷.

In certain embodiments of formula II, R³ is —Y—C(O)—R^{d}.

In certain embodiments of formula II, Y is a bond.

In certain embodiments of formula II, Y is C₂₋₆alkylene.

In certain embodiments of formula II, Y is isopropylidine.

In certain embodiments of formula II, Y is methylene.

In certain embodiments of formula II, Y is ethylene.

In certain embodiments of formula II, Y is —C(CH₃)₂—.

In certain embodiments of formula II, Y is —CH₂—.

In certain embodiments of formula II, Y is —CH(CH₃)—.

In certain embodiments of formula II, Y is —CH₂—C(CH₃)₂—.

In certain embodiments of formula II, Y is —C(CH₃)₂—CH₂—.

In certain embodiments of formula II, R^{d} is C₁₋₆alkyl, C₁₋₆alkoxy, amino, or heterocyclyl.

In certain embodiments of formula II, R^{d} is C₁₋₆alkyl.

In certain embodiments of formula II, R^{d} is C₁₋₆alkoxy.

In certain embodiments of formula II, R^{d} is amino.

In certain embodiments of formula II, R^{d} is halo-C₁₋₆alkyl.

In certain embodiments of formula II, R^{d} is hydroxy-C₁₋₆alkyl.

In certain embodiments of formula II, R^{d} is C₁₋₆alkoxy-C₁₋₆alkyl.

In certain embodiments of formula II, R^{d} is cyano-C₁₋₆alkyl.

In certain embodiments of formula II, R^{d} is C₁₋₆alkylsulfonylC₁₋₆alkyl.

In certain embodiments of formula II, R^{d} is amino-C₁₋₆alkyl.

In certain embodiments of formula II, R^{d} is C₃₋₆cycloalkyl optionally substituted one or more times with R⁶.

In certain embodiments of formula II, R^{d} is C₃₋₆cycloalkyl-C₁₋₆alkyl wherein the C₃₋₆cycloalkyl portion is optionally substituted one or more times with R⁶.

In certain embodiments of formula II, R^{d} is heterocyclyl optionally substituted one or more times with R⁷.

In certain embodiments of formula II, R^{d} is heterocyclyl-C₁₋₆alkyl wherein the heterocyclyl portion is optionally substituted one or more times with R⁷.

In certain embodiments of formula II, R^{d} is 1-methyl-cyclopropyl; methylamino; dimethylamino; pyrrolidin-1-yl; methoxy; cyclopropyl-methyl; ethyl; 2,2,2-trifluoro-ethyl; tert-butyl; or isopropyl.

In certain embodiments of formula II, R^{d} is 1-methyl-cyclopropyl.

In certain embodiments of formula II, R^{d} is methylamino

In certain embodiments of formula II, R^{d} is dimethylamino.

In certain embodiments of formula II, R^{d} is pyrrolidin-1-yl.

In certain embodiments of formula II, R^{d} is methoxy.

In certain embodiments of formula II, R^{d} is cyclopropyl-methyl.

In certain embodiments of formula II, R^{d} is ethyl.

In certain embodiments of formula II, R^{d} is 2,2,2-trifluoro-ethyl.

In certain embodiments of formula II, R^{d} is tert-butyl.

In certain embodiments of formula II, R^{d} is isopropyl.

In embodiments of formula II wherein R^{d} is heterocyclyl or heterocyclyl-C₁₋₆alkyl, such heterocyclyl may be piperidinyl, piperazinyl, morpholinyl, tetrahydropyranyl, pyrrolidinyl, azetidinyl, tetrahydrofuranyl or oxetanyl, each optionally substituted one or more times, or one or two times, with R⁷ as defined herein.

In embodiments of formula II wherein R^{d} is heterocyclyl, such heterocyclyl moiety may be piperidinyl, pyrrolidinyl, oxetanyl, tetrahydropyranyl, tetrahydrofuranyl, azetidinyl, [1,3]dioxolanyl or tetrahydrothiopyranyl, each being unsubstituted or substituted one or more times with R⁷.

In embodiments of formula II wherein R^{d} is heterocyclyl-C₁₋₆alkyl, such heterocyclyl moiety may be piperidinyl, piperazinyl, morpholinyl, thiomorpholinyl, pyrrolidinyl, oxetanyl, tetrahydropyranyl, tetrahydrofuranyl, azetidinyl, [1,3]dioxolanyl or tetrahydrothiopyranyl, each being unsubstituted or substituted one or more times with R⁷.

In certain embodiments of formula II, R⁴ is: hydrogen; C₁₋₆alkyl; halo; halo-C₁₋₆alkyl; C₁₋₆alkoxy-C₁₋₆alkyl; hydroxy-C₁₋₆alkyl; C₃₋₆cycloalkyl optionally substituted with C₁₋₆alkyl; C₃₋₆cycloalkyl-C₁₋₆alkyl wherein the C₃₋₆cycloalkyl portion is optionally substituted with C₁₋₆alkyl; or —C(O)—R^{c} wherein R^{c} is C₁₋₆alkyl, C₁₋₆alkoxy, amino, or heterocyclyl.

In certain embodiments of formula II, R⁴ is: C₁₋₆alkyl; halo; halo-C₁₋₆alkyl; C₁₋₆alkoxy-C₁₋₆alkyl; hydroxy-C₁₋₆alkyl; C₃₋₆cycloalkyl optionally substituted with C₁₋₆alkyl; C₃₋₆cycloalkyl-C₁₋₆alkyl wherein the C₃₋₆cycloalkyl portion is optionally substituted with C₁₋₆alkyl; or —C(O)—R^{c} wherein R^{c} is C₁₋₆alkyl, C₁₋₆alkoxy, amino, or heterocyclyl.

In certain embodiments of formula II, R⁴ is: hydrogen; C₁₋₆alkyl; halo; C₃₋₆cycloalkyl optionally substituted with C₁₋₆alkyl; C₃₋₆cycloalkyl-C₁₋₆alkyl wherein the C₃₋₆cycloalkyl portion is optionally substituted with C₁₋₆alkyl; or —C(O)—R^{c} wherein R^{c} is C₁₋₆alkyl, C₁₋₆alkoxy, amino, or heterocyclyl.

In certain embodiments of formula II, R⁴ is: hydrogen; C₁₋₆alkyl; halo; or C₃₋₆cycloalkyl optionally substituted with C₁₋₆alkyl.

In certain embodiments of formula II, R⁴ is hydrogen or C₁₋₆alkyl.

In certain embodiments of formula II, R⁴ is hydrogen.

In certain embodiments of formula II, R⁴ is C₁₋₆alkyl.

In certain embodiments of formula II, R⁴ is halo.

In certain embodiments of formula II, R⁴ is cyano.

In certain embodiments of formula II, R⁴ is halo-C₁₋₆alkyl.

In certain embodiments of formula II, R⁴ is C₁₋₆alkoxy-C₁₋₆alkyl.

In certain embodiments of formula II, R⁴ is hydroxy-C₁₋₆alkyl.

In certain embodiments of formula II, R⁴ is C₃₋₆cycloalkyl optionally substituted with C₁₋₆alkyl.

In certain embodiments of formula II, R⁴ is hydrogen or methyl.

In certain embodiments of formula II, R⁴ is C₃₋₆cycloalkyl-C₁₋₆alkyl wherein the C₃₋₆cycloalkyl portion is optionally substituted with C₁₋₆alkyl.

In certain embodiments of formula II, R⁴ is —C(O)—R^{c} wherein R^{c} is C₁₋₆alkyl, C₁₋₆alkoxy, amino, or heterocyclyl.

In certain embodiments of formula II, R⁴ is —C(O)—R^{c} wherein R^{c} is heterocyclyl.

In embodiments of formula II wherein R^{c} is heterocyclyl, such heterocyclyl may be pyrrolidinyl, piperidinyl, piperazinyl or morpholinyl.

In embodiments of formula II wherein R^{c} is heterocyclyl, such heterocyclyl may be piperidinyl, piperazinyl or morpholinyl.

In certain embodiments of formula II, R⁴ is: hydrogen; methyl; isopropyl; cyclopropyl; chloro; or morpholin-4-yl-carbonyl.

In certain embodiments of formula II, R⁴ is: hydrogen; methyl; isopropyl; cyclopropyl; or chloro.

In certain embodiments of formula II, R⁴ is hydrogen.

In certain embodiments of formula II, R⁴ is methyl.

In certain embodiments of formula II, R⁴ is isopropyl.

In certain embodiments of formula II, R⁴ is cyclopropyl.

In certain embodiments of formula II, R⁴ is chloro.

In certain embodiments of formula II, R⁴ is morpholin-4-yl-carbonyl.

In certain embodiments of formula II, R⁴ is 2-fluoro-ethyl.

In certain embodiments of formula II, R⁴ is C₃₋₆cycloalkyl optionally substituted one or more times, or one or two times, with R⁶.

In certain embodiments of formula II, R⁴ is C₃₋₆cycloalkyl-C₁₋₆alkyl wherein the C₃₋₆cycloalkyl portion is optionally substituted one or more times, or one or two times, with R⁶.

In certain embodiments of formula II, R⁴ is —Y—C(O)—R^{d}.

In certain embodiments of formula II, or R³ and R⁴ together with the atoms to which they are attached may form a 5- or 6-membered ring that optionally includes a heteroatom selected from O, N and S.

In certain embodiments of formula II, R⁵ is hydrogen.

In certain embodiments of formula II, R⁵ is C₁₋₆alkyl.

In certain embodiments of formula II, R⁵ is methyl.

In certain embodiments of formula II, each R⁶ is independently C₁₋₆alkyl; halo-C₁₋₆alkyl; C₁₋₆alkoxy; cyano; or halo.

In certain embodiments of formula II, R⁶ is C₁₋₆alkyl; halo-C₁₋₆alkyl; C₁₋₆alkoxy; or halo.

In certain embodiments of formula II, R⁶ is C₁₋₆alkyl; halo-C₁₋₆alkyl; or halo.

In certain embodiments of formula II, R⁶ is C₁₋₆alkyl.

In certain embodiments of formula II, R⁶ is halo-C₁₋₆alkyl. In certain embodiments of formula II, R⁶ is C₁₋₆alkoxy.

In certain embodiments of formula II, R⁶ is cyano.

In certain embodiments of formula II, R⁶ is halo.

In certain embodiments of formula II, R⁶ is Y—C(O)—R^{d}.

In certain embodiments of formula II, R⁶ is oxo.

In certain embodiments of formula II, each R⁷ is independently C₁₋₆alkyl; halo-C₁₋₆alkyl; halo; C₁₋₆alkylsulfonyl; C₁₋₆alkoxy-C₁₋₆alkyl; cyano; heterocyclyl; or C₃₋₆cycloalkylsulfonyl wherein the C₃₋₆cycloalkyl portion is optionally substituted one or more times with R⁶.

In certain embodiments of formula II, R⁷ is C₁₋₆alkyl.

In certain embodiments of formula II, R⁷ is halo-C₁₋₆alkyl.

In certain embodiments of formula II, R⁷ is halo.

In certain embodiments of formula II, R⁷ is C₁₋₆alkylsulfonyl.

In certain embodiments of formula II, R⁷ is C₁₋₆alkoxy-C₁₋₆alkyl.

In certain embodiments of formula II, R⁷ is cyano.

In certain embodiments of formula II, R⁷ is —Y—C(O)—R^{d}.

In certain embodiments of formula II, R⁷ is heterocyclyl.

In certain embodiments of formula II, R⁷ is C₃₋₆cycloalkylsulfonyl wherein the C₃₋₆cycloalkyl portion is optionally substituted one or more times with R⁶.

In certain embodiments of formula II, R⁷ is oxo.

In certain embodiments of formula II, R⁷ is C₁₋₆alkoxy.

In certain embodiments of formula II, R⁷ is heterocyclyl-C₁₋₆alkyl.

In certain embodiments of formula II, R⁷ is C₃₋₆cycloalkyl.

In certain embodiments of formula II, R⁷ is C₃₋₆cycloalkyl-C₁₋₆alkyl.

In embodiments of formula II wherein R⁷ is heterocyclyl, such heterocyclyl moiety may be piperidinyl, pyrrolidinyl, oxetanyl, tetrahydropyranyl, tetrahydrofuranyl, azetidinyl, [1,3]dioxolanyl or tetrahydrothiopyranyl.

In embodiments of formula II wherein R⁷ is heterocyclyl-C₁₋₆alkyl, such heterocyclyl moiety may be piperidinyl, piperazinyl, morpholinyl, thiomorpholinyl, pyrrolidinyl, oxetanyl, tetrahydropyranyl, tetrahydrofuranyl, azetidinyl, [1,3]dioxolanyl or tetrahydrothiopyranyl.

In certain embodiments of formula II, each R⁸ is independently oxo; C₁₋₆alkyl; halo-C₁₋₆alkyl; halo; C₁₋₆alkoxy; C₁₋₆alkoxy-C₁₋₆alkyl; cyano; C₃₋₆cycloalkyl optionally substituted one or more times with R⁶, C₃₋₆cycloalkyl-C₁₋₆alkyl wherein the C₃₋₆cycloalkyl portion is optionally substituted one or more times with R⁶, or C₃₋₆cycloalkyl-sulfonyl wherein the C₃₋₆cycloalkyl portion is optionally substituted one or more times with R⁶.

In certain embodiments of formula II, R⁸ is oxo. In certain embodiments of formula II, R⁷ is C₁₋₆alkyl.

In certain embodiments of formula II, R⁷ is halo-C₁₋₆alkyl In certain embodiments of formula II, R⁷ is halo. In certain embodiments of formula II, R⁷ is C₁₋₆alkoxy.

In certain embodiments of formula II, R⁷ is C₁₋₆alkoxy-C₁₋₆alkyl.

In certain embodiments of formula II, R⁷ is cyano.

In certain embodiments of formula II, R⁷ is hetoeryclyl.

In certain embodiments of formula II, R⁷ is —Y—C(O)—R^{d}.

In certain embodiments of formula II, R⁷ is C₃₋₆cycloalkyl optionally substituted one or more times with R⁶.

In certain embodiments of formula II, R⁷ is C₃₋₆cycloalkyl-C₁₋₆alkyl wherein the C₃₋₆cycloalkyl portion is optionally substituted one or more times with R⁶.

In certain embodiments of formula II, R⁷ is C₃₋₆cycloalkyl-sulfonyl wherein the C₃₋₆cycloalkyl portion is optionally substituted one or more times with R⁶.

In certain embodiments of formula II, R⁸ is oxo.

In certain embodiments of formula II, R⁸ is C₁₋₆alkyl.

In certain embodiments of formula II, R⁸ is halo-C₁₋₆alkyl.

In certain embodiments of formula II, R⁸ is halo.

In certain embodiments of formula II, R⁸ is C₁₋₆alkyl-sulfonyl.

In certain embodiments of formula II, R⁸ is C₁₋₆alkoxy.

In certain embodiments of formula II, R⁸ is C₁₋₆alkoxy-C₁₋₆alkyl.

In certain embodiments of formula II, R⁸ is cyano; hetoeryclyl.

In certain embodiments of formula II, R⁸ is heterocyclyl-C₁₋₆alkyl.

In certain embodiments of formula II, R⁸ is —Y—C(O)—R^{d}.

In certain embodiments of formula II, R⁸ is C₃₋₆cycloalkyl.

In certain embodiments of formula II, R⁸ is C₃₋₆cycloalkyl-C₁₋₆alkyl-C₃₋₆cycloalkyl-sulfonyl.

In embodiments of formula II wherein R⁸ is heterocyclyl, such heterocyclyl moiety may be piperidinyl, pyrrolidinyl, oxetanyl, tetrahydropyranyl, tetrahydrofuranyl, azetidinyl, [1,3]dioxolanyl or tetrahydrothiopyranyl.

In embodiments of formula II wherein R⁸ is heterocyclyl-C₁₋₆alkyl, such heterocyclyl moiety may be piperidinyl, piperazinyl, morpholinyl, thiomorpholinyl, pyrrolidinyl, oxetanyl, tetrahydropyranyl, tetrahydrofuranyl, azetidinyl, [1,3]dioxolanyl or tetrahydrothiopyranyl.

In certain embodiments of formula II, the compound may be:
N²-(1,5-dimethyl-1H-pyrazol-4-yl)-N⁴-methyl-5-(trifluoromethyl)pyrimidine-2,4-diamine
N⁴-methyl-N²-(1-(2-morpholino ethyl)-1H-pyrazol-4-yl)-5-(trifluoromethyl)pyrimidine-2,4-diamine
N⁴-methyl-N²-(1-methyl-1H-pyrazol-4-yl)-5-(trifluoromethyl)pyrimidine-2,4-diamine
5-chloro-N²-(1-isopropyl-1H-pyrazol-4-yl)-N⁴-methylpyrimidine-2,4-diamine
N⁴-methyl-N²-(1-methyl-1H-pyrazol-5-yl)-5-(trifluoromethyl)pyrimidine-2,4-diamine
N⁴-methyl-N²-(1-methyl-1H-pyrazol-3-yl)-5-(trifluoromethyl)pyrimidine-2,4-diamine
N²-(1,3-dimethyl-1H-pyrazol-4-yl)-N⁴-methyl-5-(trifluoromethyl)pyrimidine-2,4-diamine
5-Chloro-N-(1,5-dimethyl-1H-pyrazol-4-yl)-4-(tetrahydro-2H-pyran-4-yloxy)pyrimidin-2-amine
N⁴-methyl-5-(trifluoromethyl)-N²-(1,3,5-trimethyl-1H-pyrazol-4-yl)pyrimidine-2,4-diamine
5-Chloro-N-(1-isopropyl-1H-pyrazol-4-yl)-4-(tetrahydro-2H-pyran-4-yloxy)pyrimidin-2-amine
5-Chloro-N-(1,5-dimethyl-1H-pyrazol-4-yl)-4-methoxypyrimidin-2-amine
N-(1,5-dimethyl-1H-pyrazol-4-yl)-4-(pyrrolidin-1-yl)-5-(trifluoromethyl)pyrimidin-2-amine
N²-(1-ethyl-5-methyl-1H-pyrazol-4-yl)-N⁴-methyl-5-(trifluoromethyl)pyrimidine-2,4-diamine
5-Chloro-N-(1,3-dimethyl-1H-pyrazol-4-yl)-4-methoxypyrimidin-2-amine
N⁴-methyl-N²-(3-methyl-1-(oxetan-3-yl)-1H-pyrazol-4-yl)-5-(trifluoromethyl)pyrimidine-2,4-diamine
N²-(5-chloro-1-methyl-1H-pyrazol-4-yl)-N⁴-methyl-5-(trifluoromethyl)pyrimidine-2,4-diamine
5-Chloro-4-methoxy-N-(3-methyl-1-(oxetan-3-yl)-1H-pyrazol-4-yl)pyrimidin-2-amine
5-Chloro-4-methoxy-N-(1-(2-methoxyethyl)-3-methyl-1H-pyrazol-4-yl)pyrimidin-2-amine
5-chloro-4-methoxy-N-(1-(2-methoxyethyl)-5-methyl-1H-pyrazol-4-yl)pyrimidin-2-amine
5-Chloro-N-(5-chloro-1-methyl-1H-pyrazol-4-yl)-4-methoxypyrimidin-2-amine
2-Methyl-1-(3-methyl-4-(4-(methylamino)-5-(trifluoromethyl)pyrimidin-2-ylamino)-1H-pyrazol-1-yl)propan-2-ol
2-Methyl-1-(3-methyl-4-(4-(methylamino)-5-chloro-pyrimidin-2-ylamino)-1H-pyrazol-1-yl)propan-2-ol
N²-(1-(2-methoxyethyl)-3-methyl-1H-pyrazol-4-yl)-N⁴-methyl-5-(trifluoromethyl)pyrimidine-2,4-diamine
N²-(1-(2-methoxyethyl)-5-methyl-1H-pyrazol-4-yl)-N⁴-methyl-5-(trifluoromethyl)pyrimidine-2,4-diamine
5-Chloro-N-(1-ethyl-3-methyl-1H-pyrazol-4-yl)-4-methoxypyrimidin-2-amine
5-Chloro-N⁴-methyl-N²-(3-methyl-1-(oxetan-3-yl)-1H-pyrazol-4-yl)pyrimidine-2,4-diamine
N²-(1-isopropyl-3-methyl-1H-pyrazol-4-yl)-N⁴-methyl-5-(trifluoromethyl)pyrimidine-2,4-diamine
5-Chloro-N-(1-isopropyl-3-methyl-1H-pyrazol-4-yl)-4-methoxypyrimidin-2-amine
5-Chloro-N²-(1,3-dimethyl-1H-pyrazol-4-yl)-N⁴-methylpyrimidine-2,4-diamine
5-Chloro-N²-(1-isopropyl-3-methyl-1H-pyrazol-4-yl)-N⁴-methylpyrimidine-2,4-diamine
N⁴-methyl-N²-(3-methyl-1-(tetrahydro-2H-pyran-4-yl)-1H-pyrazol-4-yl)-5-(trifluoromethyl)pyrimidine-2,4-diamine
N⁴-methyl-N²-(5-methyl-1-(tetrahydro-2H-pyran-4-yl)-1H-pyrazol-4-yl)-5-(trifluoromethyl)pyrimidine-2,4-diamine
N²-(2-Ethyl-2H-pyrazol-3-yl)-5-fluoro-N⁴-methyl-pyrimidine-2,4-diamine
5-Fluoro-N⁴-methyl-N²-(2-methyl-2H-pyrazol-3-yl)-pyrimidine-2,4-diamine
5-Fluoro-N⁴-methyl-N⁴-(2-propyl-2H-pyrazol-3-yl)-pyrimidine-2,4-diamine
N²-(2,5-Dimethyl-2H-pyrazol-3-yl)-5-fluoro-N⁴-methyl-pyrimidine-2,4-diamine
N²-(3-isopropyl-1-methyl-1H-pyrazol-5-yl)-N⁴-methyl-5-(trifluoromethyl)pyrimidine-2,4-diamine
5-Chloro-N-(3-cyclopropyl-1-methyl-1H-pyrazol-5-yl)-4-methoxypyrimidin-2-amine
N²-(3-Cyclopropyl-1-methyl-1H-pyrazol-5-yl)-N⁴-methyl-5-(trifluoromethyl)pyrimidine-2,4-diamine
5-Chloro-N-(3-isopropyl-1-methyl-1H-pyrazol-5-yl)-4-methoxypyrimidin-2-amine
5-Chloro-N²-(5-isopropyl-2-methyl-2H-pyrazol-3-yl)-N⁴-methyl-pyrimidine-2,4-diamine
5-Chloro-4-methoxy-N-(1,3,5-trimethyl-1H-pyrazol-4-yl)pyrimidin-2-amine
5-Chloro-N⁴-methyl-N²-(1,3,5-trimethyl-1H-pyrazol-4-yl)-pyrimidine-2,4-diamine
5-Chloro-N²-(5-cyclopropyl-2-methyl-2H-pyrazol-3-yl)-N⁴-methyl-pyrimidine-2,4-diamine
N⁴-Methyl-N²-(5-methyl-1-oxetan-3-yl-1H-pyrazol-4-yl)-5-trifluoromethyl-pyrimidine-2,4-diamine
N²-(1-isopropyl-1H-pyrazol-5-yl)-N⁴-methyl-5-(trifluoromethyl)pyrimidine-2,4-diamine
5-Chloro-N-(1-ethyl-5-methyl-1H-pyrazol-4-yl)-4-methoxypyrimidin-2-amine
5-Chloro-N²-(1-ethyl-5-methyl-1H-pyrazol-4-yl)-N⁴-methylpyrimidine-2,4-diamine
N²-(1-ethyl-3-methyl-1H-pyrazol-4-yl)-N⁴-methyl-5-(trifluoromethyl)pyrimidine-2,4-diamine
5-Chloro-N²-(1-ethyl-3-methyl-1H-pyrazol-4-yl)-N⁴-methylpyrimidine-2,4-diamine
5-chloro-N²-(1-isopropyl-1H-pyrazol-5-yl)-N⁴-methylpyrimidine-2,4-diamine
5-chloro-N-(1-isopropyl-1H-pyrazol-5-yl)-4-methoxypyrimidin-2-amine
5-chloro-4-methoxy-N-(3-methyl-1-(methylsulfonyl)-1H-pyrazol-4-yl)pyrimidin-2-amine
N²-(1-ethyl-1H-pyrazol-3-yl)-N⁴-methyl-5-(trifluoromethyl)pyrimidine-2,4-diamine
5-chloro-4-methoxy-N-(5-methyl-1-phenyl-1H-pyrazol-4-yl)pyrimidin-2-amine
N²-(1-isopropyl-1H-pyrazol-3-yl)-N4-methyl-5-(trifluoromethyl)pyrimidine-2,4-diamine
N⁴⁻methyl-N²-(5-methyl-1-(2,2,2-trifluoro ethyl)-1H-pyrazol-4-yl)-5-(trifluoromethyl)pyrimidine-2,4-diamine
N²-(1-(2,2-dimethyl-1,3-dioxan-5-yl)-3-methyl-1H-pyrazol-4-yl)-N4-methyl-5-(trifluoromethyl)pyrimidine-2,4-diamine
5-chloro-4-methoxy-N-(5-methyl-1-(4-(methylsulfonyl)phenyl)-1H-pyrazol-4-yl)pyrimidin-2-amine
N⁴-ethyl-N²-(1-methyl-1H-pyrazol-3-yl)-5-(trifluoromethyl)pyrimidine-2,4-diamine
5-chloro-N-(1,5-dimethyl-1H-pyrazol-4-yl)-4-(oxetan-3-yloxy)pyrimidin-2-amine
5-chloro-4-(2,2-difluoro ethoxy)-N-(1,5-dimethyl-1H-pyrazol-4-yl)pyrimidin-2-amine
5-chloro-N-(1,5-dimethyl-1H-pyrazol-4-yl)-4-(2,2,2-trifluoro ethoxy)pyrimidin-2-amine
5-chloro-4-methoxy-N-(3-methyl-1-(tetrahydro-2H-pyran-4-yl)-1H-pyrazol-4-yl)pyrimidin-2-amine
(4-(4-(5-chloro-4-methoxypyrimidin-2-ylamino)-3-methyl-1H-pyrazol-1-yl)piperidin-1-yl)(1-methylcyclopropyl)methanone
(4-(4-(5-chloro-4-methoxypyrimidin-2-ylamino)-5-methyl-1H-pyrazol-1-yl)piperidin-1-yl)(1-methylcyclopropyl)methanone
4-(4-(5-chloro-4-methoxypyrimidin-2-ylamino)-3-methyl-1H-pyrazol-1-yl)benzonitrile
5-chloro-4-methoxy-N-(3-methyl-1-(3-methylpyridin-4-yl)-1H-pyrazol-4-yl)pyrimidin-2-amine
5-chloro-N-(1-(cyclopropylsulfonyl)-5-methyl-1H-pyrazol-4-yl)-4-methoxypyrimidin-2-amine
5-chloro-N-(1-(cyclopropylsulfonyl)-3-methyl-1H-pyrazol-4-yl)-4-methoxypyrimidin-2-amine
2-(4-(5-chloro-4-methoxypyrimidin-2-ylamino)-5-methyl-1H-pyrazol-1-yl)-2-methylpropanenitrile
2-(4-(5-chloro-4-methoxypyrimidin-2-ylamino)-3-methyl-1H-pyrazol-1-yl)-2-methylpropanenitrile
5-chloro-4-ethoxy-N-(5-methyl-1-(tetrahydro-2H-pyran-4-yl)-1H-pyrazol-4-yl)pyrimidin-2-amine
(5-Chloro-4-methoxy-pyrimidin-2-yl)-[1-(4-methanesulfonyl-phenyl)-3-methyl-1H-pyrazol-4-yl]-amine
(5-Chloro-4-methoxy-pyrimidin-2-yl)-(3-methyl-1-phenyl-1H-pyrazol-4-yl)-amine
(4-Methoxy-5-trifluoromethyl-pyrimidin-2-yl)-(3-methyl-1-phenyl-1H-pyrazol-4-yl)-amine
(4-Methoxy-5-trifluoromethyl-pyrimidin-2-yl)-(5-methyl-1-phenyl-1H-pyrazol-4-yl)-amine
(5-Chloro-4-methoxy-pyrimidin-2-yl)-(1-methane sulfonyl-3-methyl-1H-pyrazol-4-yl)-amine
(5-Chloro-4-methoxy-pyrimidin-2-yl)-[5-methyl-1-(tetrahydro-pyran-4-yl)-1H-pyrazol-4-yl]-amine
4-[4-(5-Chloro-4-methoxy-pyrimidin-2-ylamino)-3-methyl-pyrazol-1-yl]-N,N-dimethyl-benzamide
4-[4-(5-Chloro-4-methoxy-pyrimidin-2-ylamino)-5-methyl-pyrazol-1-yl]-N,N-dimethyl-benzamide
4-[4-(5-Chloro-4-methoxy-pyrimidin-2-ylamino)-5-methyl-pyrazol-1-yl]-benzonitrile
N²-(5-Methoxy-1-methyl-1H-pyrazol-4-yl)-N⁴-methyl-5-trifluoromethyl-pyrimidine-2,4-diamine
(5-Chloro-4-methoxy-pyrimidin-2-yl)-[5-chloro-1-(tetrahydro-pyran-4-yl)-1H-pyrazol-4-yl]-amine
(5-Chloro-4-methoxy-pyrimidin-2-yl)-{1-[1-(2-fluoro-ethyl)-piperidin-4-yl]-3-methyl-1H-pyrazol-4-yl}-amine
N²-[1-(1-[1,3]Dioxolan-2-ylmethyl-piperidin-4-yl)-5-methyl-1H-pyrazol-4-yl]-N⁴-ethyl-5-trifluoromethyl-pyrimidine-2,4-diamine
N²-[1-(1-[1,3]Dioxolan-2-ylmethyl-piperidin-4-yl)-3-methyl-1H-pyrazol-4-yl]-N⁴-ethyl-5-trifluoromethyl-pyrimidine-2,4-diamine
N²-(1,3-Dimethyl-1H-pyrazol-4-yl)-5-iodo-N⁴-methyl-pyrimidine-2,4-diamine
N4-methyl-N2-(5-methyl-1-(1-(2,2,2-trifluoro ethyl)piperidin-4-yl)-1H-pyrazol-4-yl)-5-(trifluoromethyl)pyrimidine-2,4-diamine
N4-methyl-N2-(3-methyl-1-(1-(2,2,2-trifluoro ethyl)piperidin-4-yl)-1H-pyrazol-4-yl)-5-(trifluoromethyl)pyrimidine-2,4-diamine
5-bromo-N4-methyl-N2-(5-methyl-1-(1-(2,2,2-trifluoro ethyl)piperidin-4-yl)-1H-pyrazol-4-yl)pyrimidine-2,4-diamine
5-bromo-N4-methyl-N2-(3-methyl-1-(1-(2,2,2-trifluoro ethyl)piperidin-4-yl)-1H-pyrazol-4-yl)pyrimidine-2,4-diamine
5-bromo-N4-methyl-N2-(3-methyl-1-(2,2,2-trifluoro ethyl)-1H-pyrazol-4-yl)pyrimidine-2,4-diamine
5-bromo-N4-methyl-N2-(5-methyl-1-(2,2,2-trifluoro ethyl)-1H-pyrazol-4-yl)pyrimidine-2,4-diamine
N4-ethyl-N2-(3-methyl-1-(oxetan-3-yl)-1H-pyrazol-4-yl)-5-(trifluoromethyl)pyrimidine-2,4-diamine
5-chloro-N4-ethyl-N2-(3-methyl-1-(oxetan-3-yl)-1H-pyrazol-4-yl)pyrimidine-2,4-diamine
5-bromo-N4-methyl-N2-(1-methyl-1H-pyrazol-5-yl)pyrimidine-2,4-diamine
2-methyl-1-(5-methyl-4-(4-(methylamino)-5-(trifluoromethyl)pyrimidin-2-ylamino)-1H-pyrazol-1-yl)propan-2-ol
5-chloro-N4-methyl-N2-(3-methyl-1-(methylsulfonyl)-1H-pyrazol-4-yl)pyrimidine-2,4-diamine
N4-methyl-N2-(3-methyl-1-(methylsulfonyl)-1H-pyrazol-4-yl)-5-(trifluoromethyl)pyrimidine-2,4-diamine
N4-methyl-N2-(3-methyl-1H-pyrazol-4-yl)-5-(trifluoromethyl)pyrimidine-2,4-diamine
5-bromo-N4-ethyl-N2-(3-methyl-1-(oxetan-3-yl)-1H-pyrazol-4-yl)pyrimidine-2,4-diamine
N2-(1-(difluoromethyl)-3-methyl-1H-pyrazol-4-yl)-N4-methyl-5-(trifluoromethyl)pyrimidine-2,4-diamine
N2-(1-(difluoromethyl)-5-methyl-1H-pyrazol-4-yl)-N4-methyl-5-(trifluoromethyl)pyrimidine-2,4-diamine
5-bromo-N4-ethyl-N2-(1-ethyl-5-methyl-1H-pyrazol-4-yl)pyrimidine-2,4-diamine
5-bromo-N2-(1-(4-fluorophenyl)-3-methyl-1H-pyrazol-4-yl)-N4-methylpyrimidine-2,4-diamine
5-bromo-N4-methyl-N2-(3-methyl-1-phenyl-1H-pyrazol-4-yl)pyrimidine-2,4-diamine
5-bromo-N4-methyl-N2-(5-methyl-1-phenyl-1H-pyrazol-4-yl)pyrimidine-2,4-diamine
5-bromo-N4-methyl-N2-(1-methyl-1H-pyrazol-4-yl)pyrimidine-2,4-diamine
N4-methyl-N2-(3-methyl-1-(1-(methylsulfonyl)azetidin-3-yl)-1H-pyrazol-4-yl)-5-(trifluoromethyl)pyrimidine-2,4-diamine
5-bromo-N4-methyl-N2-(3-methyl-1-propyl-1H-pyrazol-4-yl)pyrimidine-2,4-diamine
5-chloro-N4-methyl-N2-(3-methyl-1-((3-methyloxetan-3-yl)methyl)-1H-pyrazol-4-yl)pyrimidine-2,4-diamine
5-bromo-N2-(1-(3,5-difluorophenyl)-5-methyl-1H-pyrazol-4-yl)-N4-methylpyrimidine-2,4-diamine
5-bromo-N2-(1-(3,5-difluorophenyl)-3-methyl-1H-pyrazol-4-yl)-N4-methylpyrimidine-2,4-diamine
N4-methyl-N2-(3-methyl-1-(pyridin-2-yl)-1H-pyrazol-4-yl)-5-(trifluoromethyl)pyrimidine-2,4-diamine
N4-methyl-N2-(3-methyl-1-((3-methyloxetan-3-yl)methyl)-1H-pyrazol-4-yl)-5-(trifluoromethyl)pyrimidine-2,4-diamine
N4-methyl-N2-(5-methyl-1-propyl-1H-pyrazol-4-yl)-5-(trifluoromethyl)pyrimidine-2,4-diamine
N4-methyl-N2-(3-methyl-1-propyl-1H-pyrazol-4-yl)-5-(trifluoromethyl)pyrimidine-2,4-diamine
5-bromo-N2-(1-isopropyl-3-methyl-1H-pyrazol-4-yl)-N4-methylpyrimidine-2,4-diamine
5-bromo-N2-(1-(4-chlorophenyl)-5-methyl-1H-pyrazol-4-yl)-N4-methylpyrimidine-2,4-diamine
N2-(1-(4-chlorophenyl)-5-methyl-1H-pyrazol-4-yl)-N4-methyl-5-(trifluoromethyl)pyrimidine-2,4-diamine
N4-methyl-N2-(3-methyl-1-(4-(methyl sulfonyl)phenyl)-1H-pyrazol-4-yl)-5-(trifluoromethyl)pyrimidine-2,4-diamine
N4-methyl-N2-(5-methyl-1-(4-(methyl sulfonyl)phenyl)-1H-pyrazol-4-yl)-5-(trifluoromethyl)pyrimidine-2,4-diamine
N2-(1-((1S,5S)-8-oxabicyclo[3.2.1]octan-3-yl)-3-methyl-1H-pyrazol-4-yl)-N4-methyl-5-(trifluoromethyl)pyrimidine-2,4-diamine
N2-(1-butyl-5-methyl-1H-pyrazol-4-yl)-N4-methyl-5-(trifluoromethyl)pyrimidine-2,4-diamine
N4-methyl-N2-(3-methyl-1-(pyrimidin-2-yl)-1H-pyrazol-4-yl)-5-(trifluoromethyl)pyrimidine-2,4-diamine
N2-(1-(4-chlorophenyl)-3-methyl-1H-pyrazol-4-yl)-N4-methyl-5-(trifluoromethyl)pyrimidine-2,4-diamine
N2-(1-(2-fluoroethyl)-3-methyl-1H-pyrazol-4-yl)-N4-methyl-5-(trifluoromethyl)pyrimidine-2,4-diamine
N4-methyl-N2-(3-methyl-1-(1-(oxetan-3-yl)piperidin-4-yl)-1H-pyrazol-4-yl)-5-(trifluoromethyl)pyrimidine-2,4-diamine
N4-methyl-N2-(5-methyl-1-(1-(oxetan-3-yl)piperidin-4-yl)-1H-pyrazol-4-yl)-5-(trifluoromethyl)pyrimidine-2,4-diamine
N2-(1-(2-fluoroethyl)-5-methyl-1H-pyrazol-4-yl)-N⁴-methyl-5-(trifluoromethyl)pyrimidine-2,4-diamine
1-(4-(4-(4-(ethylamino)-5-(trifluoromethyl)pyrimidin-2-ylamino)-3-methyl-1H-pyrazol-1-yl)piperidin-1-yl)ethanone
cyclopropyl(4-(5-methyl-4-(4-(methylamino)-5-(trifluoromethyl)pyrimidin-2-ylamino)-1H-pyrazol-1-yl)piperidin-1-yl)methanone
cyclopropyl(4-(3-methyl-4-(4-(methylamino)-5-(trifluoromethyl)pyrimidin-2-ylamino)-1H-pyrazol-1-yl)piperidin-1-yl)methanone
1-(4-(4-(4-(ethylamino)-5-(trifluoromethyl)pyrimidin-2-ylamino)-5-methyl-1H-pyrazol-1-yl)piperidin-1-yl)ethanone
N2-(5-chloro-1-isopropyl-1H-pyrazol-4-yl)-N4-methyl-5-(trifluoromethyl)pyrimidine-2,4-diamine
N2-(5-chloro-1-ethyl-1H-pyrazol-4-yl)-N4-methyl-5-(trifluoromethyl)pyrimidine-2,4-diamine
N4-methyl-N2-(3-methyl-1-(pyrimidin-5-yl)-1H-pyrazol-4-yl)-5-(trifluoromethyl)pyrimidine-2,4-diamine
N4-methyl-N2-(4-methyl-1-(1-methylpiperidin-4-yl)-1H-pyrazol-3-yl)-5-(trifluoromethyl)pyrimidine-2,4-diamine
N4-methyl-N2-(5-methyl-1-(2-methylpyridin-4-yl)-1H-pyrazol-4-yl)-5-(trifluoromethyl)pyrimidine-2,4-diamine
N4-methyl-N2-(3-methyl-1-(2-methylpyridin-4-yl)-1H-pyrazol-4-yl)-5-(trifluoromethyl)pyrimidine-2,4-diamine
N4-ethyl-N2-(3-methyl-1-((3-methyloxetan-3-yl)methyl)-1H-pyrazol-4-yl)-5-(trifluoromethyl)pyrimidine-2,4-diamine
N2-(5-chloro-1-cyclopropyl-1H-pyrazol-4-yl)-N4-methyl-5-(trifluoromethyl)pyrimidine-2,4-diamine
N2-(5-chloro-1-(cyclopropylmethyl)-1H-pyrazol-4-yl)-N4-methyl-5-(trifluoromethyl)pyrimidine-2,4-diamine
4-(5-methyl-4-(4-(methylamino)-5-(trifluoromethyl)pyrimidin-2-ylamino)-1H-pyrazol-1-yl)benzonitrile
4-(3-methyl-4-(4-(methylamino)-5-(trifluoromethyl)pyrimidin-2-ylamino)-1H-pyrazol-1-yl)benzonitrile
N4-methyl-N2-(3-methyl-1-(tetrahydrofuran-3-yl)-1H-pyrazol-4-yl)-5-(trifluoromethyl)pyrimidine-2,4-diamine
N4-methyl-N2-(5-methyl-1-(tetrahydrofuran-3-yl)-1H-pyrazol-4-yl)-5-(trifluoromethyl)pyrimidine-2,4-diamine
5-(4-(4-(ethylamino)-5-(trifluoromethyl)pyrimidin-2-ylamino)-5-methyl-1H-pyrazol-1-yl)-1-methylpiperidin-2-one
5-(4-(4-(ethylamino)-5-(trifluoromethyl)pyrimidin-2-ylamino)-3-methyl-1H-pyrazol-1-yl)-1-methylpiperidin-2-one
5-(3-methyl-4-(4-(methylamino)-5-(trifluoromethyl)pyrimidin-2-ylamino)-1H-pyrazol-1-yl)piperidin-2-one
5-(5-methyl-4-(4-(methylamino)-5-(trifluoromethyl)pyrimidin-2-ylamino)-1H-pyrazol-1-yl)piperidin-2-one
N2-(1-isopropyl-5-methyl-1H-pyrazol-4-yl)-N4-methyl-5-(trifluoromethyl)pyrimidine-2,4-diamine
N,N-dimethyl-4-(5-methyl-4-(4-(methylamino)-5-(trifluoromethyl)pyrimidin-2-ylamino)-1H-pyrazol-1-yl)benzamide
4-(4-(4-(ethylamino)-5-(trifluoromethyl)pyrimidin-2-ylamino)-5-methyl-1H-pyrazol-1-yl)-N,N-dimethylbenzamide
N4-ethyl-N2-(5-methyl-1-(tetrahydro-2H-pyran-4-yl)-1H-pyrazol-4-yl)-5-(trifluoromethyl)pyrimidine-2,4-diamine
N4-ethyl-N2-(3-methyl-1-(tetrahydro-2H-pyran-4-yl)-1H-pyrazol-4-yl)-5-(trifluoromethyl)pyrimidine-2,4-diamine
N4-ethyl-N2-(3-methyl-1-(methylsulfonyl)-1H-pyrazol-4-yl)-5-(trifluoromethyl)pyrimidine-2,4-diamine
N2-(1-(4-(cyclopropylsulfonyl)phenyl)-3-methyl-1H-pyrazol-4-yl)-N4-methyl-5-(trifluoromethyl)pyrimidine-2,4-diamine
4-(4-(4-(ethylamino)-5-(trifluoromethyl)pyrimidin-2-ylamino)-5-methyl-1H-pyrazol-1-yl)_{b} enzonitrile
N4-ethyl-N2-(5-methyl-1-(4-(methylsulfonyl)phenyl)-1H-pyrazol-4-yl)-5-(trifluoromethyl)pyrimidine-2,4-diamine
N,N-dimethyl-4-(3-methyl-4-(4-(methylamino)-5-(trifluoromethyl)pyrimidin-2-ylamino)-1H-pyrazol-1-yl)benzamide
N2-(1-(cyclopropylmethyl)-5-methyl-1H-pyrazol-4-yl)-N4-methyl-5-(trifluoromethyl)pyrimidine-2,4-diamine
N2-(1-(cyclopropylmethyl)-3-methyl-1H-pyrazol-4-yl)-N4-methyl-5-(trifluoromethyl)pyrimidine-2,4-diamine
N2-(1-(4-(cyclopropylsulfonyl)phenyl)-5-methyl-1H-pyrazol-4-yl)-N4-methyl-5-(trifluoromethyl)pyrimidine-2,4-diamine
N2-(5-chloro-1-(oxetan-3-yl)-1H-pyrazol-4-yl)-N4-methyl-5-(trifluoromethyl)pyrimidine-2,4-diamine
N4-ethyl-N2-(5-methyl-1 #3-methyloxetan-3-yl)methyl)-1H-pyrazol-4-yl)-5-(trifluoromethyl)pyrimidine-2,4-diamine
N2-(1-(cyclopropylsulfonyl)-3-methyl-1H-pyrazol-4-yl)-N4-methyl-5-(trifluoromethyl)pyrimidine-2,4-diamine
N2-(1-(cyclopropylsulfonyl)-3-methyl-1H-pyrazol-4-yl)-N4-ethyl-5-(trifluoromethyl)pyrimidine-2,4-diamine
5-chloro-N4-(2,2-difluoro ethyl)-N2-(1,5-dimethyl-1H-pyrazol-4-yl)pyrimidine-2,4-diamine
5-chloro-4-methyl-N-(3-methyl-1-(4-(methylsulfonyl)phenyl)-1H-pyrazol-4-yl)pyrimidin-2-amine
N2-(1-(4-(cyclopropylsulfonyl)phenyl)-5-methyl-1H-pyrazol-4-yl)-N4-ethyl-5-(trifluoromethyl)pyrimidine-2,4-diamine
2-methyl-1-(4-(5-methyl-4-(4-(methylamino)-5-(trifluoromethyl)pyrimidin-2-ylamino)-1H-pyrazol-1-yl)piperidin-1-yl)propan-1-one
N4-ethyl-N2-(1-methyl-1H-pyrazol-5-yl)-5-(trifluoromethyl)pyrimidine-2,4-diamine
N2-(3-cyclopropyl-1-methyl-1H-pyrazol-4-yl)-N4-methyl-5-(trifluoromethyl)pyrimidine-2,4-diamine
N2-(5-cyclopropyl-1-methyl-1H-pyrazol-4-yl)-N4-methyl-5-(trifluoromethyl)pyrimidine-2,4-diamine
N4-methyl-N2-(5-methyl-1-((3-methyloxetan-3-yl)methyl)-1H-pyrazol-4-yl)-5-(trifluoromethyl)pyrimidine-2,4-diamine
N2-(5-chloro-1-((3-methyl oxetan-3-yl)methyl)-1H-pyrazol-4-yl)-N4-methyl-5-(trifluoromethyl)pyrimidine-2,4-diamine
1-(4-(4-(4-(ethylamino)-5-(trifluoromethyl)pyrimidin-2-ylamino)-5-methyl-1H-pyrazol-1-yl)piperidin-1-yl)-2-methylpropan-1-one
N4-ethyl-N2-(3-methyl-1-(1-(oxetan-3-yl)azetidin-3-yl)-1H-pyrazol-4-yl)-5-(trifluoromethyl)pyrimidine-2,4-diamine
cyclopropyl(4-(4-(4-(ethylamino)-5-(trifluoromethyl)pyrimidin-2-ylamino)-3-methyl-1H-pyrazol-1-yl)piperidin-1-yl)methanone
cyclopropyl(4-(4-(4-(ethylamino)-5-(trifluoromethyl)pyrimidin-2-ylamino)-5-methyl-1H-pyrazol-1-yl)piperidin-1-yl)methanone
1-(5-chloro-4-(4-(methylamino)-5-(trifluoromethyl)pyrimidin-2-ylamino)-1H-pyrazol-1-yl)-2-methylpropan-2-ol
N4-ethyl-N2-(1-ethyl-1H-pyrazol-3-yl)-5-(trifluoromethyl)pyrimidine-2,4-diamine
(S)—N2-(1-(2-methoxypropyl)-5-methyl-1H-pyrazol-4-yl)-N4-methyl-5-(trifluoromethyl)pyrimidine-2,4-diamine
N2-(1-(2-methoxycyclopentyl)-3-methyl-1H-pyrazol-4-yl)-N4-methyl-5-(trifluoromethyl)pyrimidine-2,4-diamine
(S)—N2-(1-(2-methoxypropyl)-3-methyl-1H-pyrazol-4-yl)-N4-methyl-5-(trifluoromethyl)pyrimidine-2,4-diamine
N2-(1-(1-methoxy-2-methylpropan-2-yl)-3-methyl-1H-pyrazol-4-yl)-N4-methyl-5-(trifluoromethyl)pyrimidine-2,4-diamine
N2-(1-(2,6-dimethyltetrahydro-2H-pyran-4-yl)-3-methyl-1H-pyrazol-4-yl)-N4-methyl-5-(trifluoromethyl)pyrimidine-2,4-diamine
(R)—N2-(1-(2-methoxypropyl)-5-methyl-1H-pyrazol-4-yl)-N4-methyl-5-(trifluoromethyl)pyrimidine-2,4-diamine
N2-(1-(3-methoxycyclopentyl)-3-methyl-1H-pyrazol-4-yl)-N4-methyl-5-(trifluoromethyl)pyrimidine-2,4-diamine
N4-methyl-N2-(1-methyl-5-(methylamino)-1H-pyrazol-4-yl)-5-(trifluoromethyl)pyrimidine-2,4-diamine
N4-methyl-N2-(5-methyl-1-(methylsulfonyl)-1H-pyrazol-4-yl)-5-(trifluoromethyl) pyrimidine-2,4-diamine
N4-methyl-N2-(5-methyl-1-(tetrahydro-2H-1,1-dioxo-thiopyran-4-yl)-1H-pyrazol-4-yl)-5-(trifluoromethyl) pyrimidine-2,4-diamine
2-methyl-1-(4-(4-(methylamino)-5-(trifluoromethyl)pyrimidin-2-ylamino)-5-(trifluoromethyl)-1H-pyrazol-1-yl)propan-2-ol
2-methyl-1-(4-(4-(methylamino)-5-(trifluoromethyl)pyrimidin-2-ylamino)-3-(trifluoromethyl)-1H-pyrazol-1-yl)propan-2-ol
N2-(1-(3-fluoro-1-(oxetan-3-yl)piperidin-4-yl)-3-methyl-1H-pyrazol-4-yl)-N4-methyl-5-(trifluoromethyl)pyrimidine-2,4-diamine
(R)—N2-(1-(1-methoxypropan-2-yl)-3-methyl-1H-pyrazol-4-yl)-N4-methyl-5-(trifluoromethyl)pyrimidine-2,4-diamine
1-(3-tert-butyl-4-(4-(ethylamino)-5-(trifluoromethyl)pyrimidin-2-ylamino)-1H-pyrazol-1-yl)-2-methylpropan-2-ol
N4-methyl-N2-(3-methyl-1-(1-(2,2,2-trifluoroethyl)azetidin-3-yl)-1H-pyrazol-4-yl)-5-(trifluoromethyl)pyrimidine-2,4-diamine
N2-(1-(1-methoxy-2-methylpropan-2-yl)-5-methyl-1H-pyrazol-4-yl)-N4-methyl-5-(trifluoromethyl)pyrimidine-2,4-diamine
(R)—N4-methyl-N2-(3-methyl-1-(1-(oxetan-3-yl)pyrrolidin-3-yl)-1H-pyrazol-4-yl)-5-(trifluoromethyl) pyrimidine-2,4-diamine
(R)—N2-(1-(1-methoxypropan-2-yl)-5-methyl-1H-pyrazol-4-yl)-N4-methyl-5-(trifluoromethyl)pyrimidine-2,4-diamine
N4-methyl-N2-(4-methyl-1H-pyrazol-5-yl)-5-(trifluoromethyl)pyrimidine-2,4-diamine
N4-ethyl-N2-(5-methyl-1-(1-methylpiperidin-4-yl)-1H-pyrazol-4-yl)-5-(trifluoromethyl)pyrimidine-2,4-diamine
N4-ethyl-N2-(3-methyl-1-(1-methylpiperidin-4-yl)-1H-pyrazol-4-yl)-5-(trifluoromethyl)pyrimidine-2,4-diamine
N4-methyl-N2-(3-methyl-1-(1-methylpiperidin-4-yl)-1H-pyrazol-4-yl)-5-(trifluoromethyl)pyrimidine-2,4-diamine
N4-methyl-N2-(5-methyl-1-(1-methylpiperidin-4-yl)-1H-pyrazol-4-yl)-5-(trifluoromethyl)pyrimidine-2,4-diamine
(R)—N4-methyl-N2-(5-methyl-1-(1-(oxetan-3-yl)pyrrolidin-3-yl)-1H-pyrazol-4-yl)-5-(trifluoromethyl)pyrimidine-2,4-diamine
N4-methyl-N2-(5-methyl-1-(pyridin-2-ylmethyl)-1H-pyrazol-4-yl)-5-(trifluoromethyl)pyrimidine-2,4-diamine
N4-methyl-N2-(3-methyl-1-(pyridin-2-ylmethyl)-1H-pyrazol-4-yl)-5-(trifluoromethyl)pyrimidine-2,4-diamine
N2-(1-(1-isopropylazetidin-3-yl)-3-methyl-1H-pyrazol-4-yl)-N4-methyl-5-(trifluoromethyl)pyrimidine-2,4-diamine
1-methyl-4-(4-(methylamino)-5-(trifluoromethyl)pyrimidin-2-ylamino)-1H-pyrazole-5-carbonitrile
N4-ethyl-N2-(1-(isopropylsulfonyl)-3-methyl-1H-pyrazol-4-yl)-5-(trifluoromethyl)pyrimidine-2,4-diamine
N2-(1-(isopropylsulfonyl)-3-methyl-1H-pyrazol-4-yl)-N4-methyl-5-(trifluoromethyl)pyrimidine-2,4-diamine
N2-(1-(isopropylsulfonyl)-5-methyl-1H-pyrazol-4-yl)-N4-methyl-5-(trifluoromethyl)pyrimidine-2,4-diamine
N2-(1-(sec-butylsulfonyl)-5-methyl-1H-pyrazol-4-yl)-N4-methyl-5-(trifluoromethyl)pyrimidine-2,4-diamine
N2-(1-(sec-butylsulfonyl)-3-methyl-1H-pyrazol-4-yl)-N4-methyl-5-(trifluoromethyl)pyrimidine-2,4-diamine
1-(4-(4-(ethylamino)-5-(trifluoromethyl)pyrimidin-2-ylamino)-3-isopropyl-1H-pyrazol-1-yl)-2-methylpropan-2-ol
N2-(1-(3-fluoro-1-methylpiperidin-4-yl)-3-methyl-1H-pyrazol-4-yl)-N4-methyl-5-(trifluoromethyl)pyrimidine-2,4-diamine
N2-(5-isopropyl-1-methyl-1H-pyrazol-4-yl)-N4-methyl-5-(trifluoromethyl)pyrimidine-2,4-diamine
N4-methyl-N2-(3-methyl-1-(1-(pyridin-2-yl)ethyl)-1H-pyrazol-4-yl)-5-(trifluoromethyl)pyrimidine-2,4-diamine
N4-methyl-N2-(5-methyl-1-(1-(pyridin-2-yl)ethyl)-1H-pyrazol-4-yl)-5-(trifluoromethyl)pyrimidine-2,4-diamine
N2-(5-chloro-1-(tetrahydro-2H-pyran-4-yl)-1H-pyrazol-4-yl)-N4-methyl-5-(trifluoromethyl)pyrimidine-2,4-diamine
N2-(3-isopropyl-1-methyl-1H-pyrazol-4-yl)-N4-methyl-5-(trifluoromethyl)pyrimidine-2,4-diamine
N2-(3-cyclobutyl-1-methyl-1H-pyrazol-4-yl)-N4-methyl-5-(trifluoromethyl)pyrimidine-2,4-diamine
N2-(5-cyclobutyl-1-methyl-1H-pyrazol-4-yl)-N4-methyl-5-(trifluoromethyl)pyrimidine-2,4-diamine
N4-methyl-N2-(3-methyl-1-(tetrahydro-2H-pyran-3-yl)-1H-pyrazol-4-yl)-5-(trifluoromethyl)pyrimidine-2,4-diamine
N4-methyl-N2-(5-methyl-1-(tetrahydro-2H-pyran-3-yl)-1H-pyrazol-4-yl)-5-(trifluoromethyl)pyrimidine-2,4-diamine
N2-(1,5-dimethyl-1H-pyrazol-4-yl)-N4-((tetrahydro-2H-pyran-4-yl) methyl)-5-(trifluoromethyl)pyrimidine-2,4-diamine
(R)—N4-methyl-N2-(3-methyl-1-(1-methylpyrrolidin-3-yl)-1H-pyrazol-4-yl)-5-(trifluoromethyl)pyrimidine-2,4-diamine
1-(5-chloro-4-(4-(ethylamino)-5-(trifluoromethyl)pyrimidin-2-ylamino)-1H-pyrazol-1-yl)-2-methylpropan-2-ol
1-(3-cyclopropyl-4-(4-(methylamino)-5-(trifluoromethyl)pyrimidin-2-ylamino)-1H-pyrazol-1-yl)-2-methylpropan-2-ol
1-(3-cyclopropyl-4-(4-(ethylamino)-5-(trifluoromethyl)pyrimidin-2-ylamino)-1H-pyrazol-1-yl)-2-methylpropan-2-ol
2-(5-chloro-4-(4-(methylamino)-5-(trifluoromethyl)pyrimidin-2-ylamino)-1H-pyrazol-1-yl)-N,2-dimethylpropanamide
N2-(1-(1-(2-methoxyethyl)piperidin-4-yl)-3-methyl-1H-pyrazol-4-yl)-N4-methyl-5-(trifluoromethyl)pyrimidine-2,4-diamine
N2-(1-(1-(2-methoxyethyl)piperidin-4-yl)-5-methyl-1H-pyrazol-4-yl)-N4-methyl-5-(trifluoromethyl)pyrimidine-2,4-diamine
(R)—N4-methyl-N2-(5-methyl-1-(1-methylpyrrolidin-3-yl)-1H-pyrazol-4-yl)-5-(trifluoromethyl)pyrimidine-2,4-diamine
N2-(5-chloro-1-(3-fluoro-1-methylpiperidin-4-yl)-1H-pyrazol-4-yl)-N4-methyl-5-(trifluoromethyl)pyrimidine-2,4-diamine
N2-(5-chloro-1-(1-ethyl-3-fluoropiperidin-4-yl)-1H-pyrazol-4-yl)-N4-methyl-5-(trifluoromethyl)pyrimidine-2,4-diamine
N4-ethyl-N2-(1-(ethylsulfonyl)-3-methyl-1H-pyrazol-4-yl)-5-(trifluoromethyl)pyrimidine-2,4-diamine
N4-ethyl-N2-(1-(ethylsulfonyl)-5-methyl-1H-pyrazol-4-yl)-5-(trifluoromethyl)pyrimidine-2,4-diamine
N4-methyl-N2-(3-methyl-1-(2-methyl-2-morpholinopropyl)-1H-pyrazol-4-yl)-5-(trifluoromethyl)pyrimidine-2,4-diamine
N2-(1-(1-ethyl-3-fluoropiperidin-4-yl)-3-methyl-1H-pyrazol-4-yl)-N4-methyl-5-(trifluoromethyl)pyrimidine-2,4-diamine
N2-(5-(dimethylamino)-1-methyl-1H-pyrazol-4-yl)-N4-methyl-5-(trifluoromethyl)pyrimidine-2,4-diamine
2-(5-chloro-4-(4-(methylamino)-5-(trifluoromethyl)pyrimidin-2-ylamino)-1H-pyrazol-1-yl)-2-methylpropan-1-ol
N2-(1-(ethylsulfonyl)-3-methyl-1H-pyrazol-4-yl)-N4-methyl-5-(trifluoromethyl)pyrimidine-2,4-diamine
2-Methyl-1-[3-methyl-4-(4-methylamino-5-trifluoromethyl-pyrimidin-2-ylamino)-pyrazol-1-yl]-propan-2-ol
N2-[1-(2-Methoxy-ethyl)-3-methyl-1H-pyrazol-4-yl]-N4-methyl-5-trifluoromethyl-pyrimidine-2,4-diamine
N2-[1-(2-Methoxy-ethyl)-5-methyl-1H-pyrazol-4-yl]-N4-methyl-5-trifluoromethyl-pyrimidine-2,4-diamine
5-Bromo-N2-(1-ethyl-3-methyl-1H-pyrazol-4-yl)-N4-methyl-pyrimidine-2,4-diamine
N4-Methyl-N2-[3-methyl-1-(2,2,2-trifluoro-ethyl)-1H-pyrazol-4-yl]-5-trifluoromethyl-pyrimidine-2,4-diamine
5-Bromo-N2-(1-difluoromethyl-5-methyl-1H-pyrazol-4-yl)-N4-methyl-pyrimidine-2,4-diamine
5-Bromo-N2-(1-difluoromethyl-3-methyl-1H-pyrazol-4-yl)-N4-methyl-pyrimidine-2,4-diamine
5-Bromo-N2-(1,5-dimethyl-1H-pyrazol-4-yl)-N4-ethyl-pyrimidine-2,4-diamine
5-Bromo-N2-[1-(4-fluoro-phenyl)-5-methyl-1H-pyrazol-4-yl]-N4-methyl-pyrimidine-2,4-diamine
5-Bromo-N4-methyl-N2-(5-methyl-1-propyl-1H-pyrazol-4-yl)-pyrimidine-2,4-diamine
5-Bromo-N2-[1-(4-chloro-phenyl)-3-methyl-1H-pyrazol-4-yl]-N4-methyl-pyrimidine-2,4-diamine
N2-(1,5-Dimethyl-1H-pyrazol-4-yl)-N4-ethyl-5-trifluoromethyl-pyrimidine-2,4-diamine
5-[4-(4-Ethylamino-5-trifluoromethyl-pyrimidin-2-ylamino)-3-methyl-pyrazol-1-yl]-piperidin-2-one
4-[4-(4-Ethylamino-5-trifluoromethyl-pyrimidin-2-ylamino)-3-methyl-pyrazol-1-yl]-N,N-dimethyl-benzamide
N2-[1-(4-Cyclopropanesulfonyl-phenyl)-3-methyl-1H-pyrazol-4-yl]-N4-ethyl-5-trifluoromethyl-pyrimidine-2,4-diamine
4-[4-(4-Ethylamino-5-trifluoromethyl-pyrimidin-2-ylamino)-3-methyl-pyrazol-1-yl]-b enzonitrile
N4-Ethyl-N2-[1-(4-methane sulfonyl-phenyl)-3-methyl-1H-pyrazol-4-yl]-5-trifluoromethyl-pyrimidine-2,4-diamine
1-{4-[4-(4-Ethylamino-5-trifluoromethyl-pyrimidin-2-ylamino)-5-methyl-pyrazol-1-yl]-piperidin-1-yl}-2-methyl-propan-1-one
1-{4-[4-(4-Ethylamino-5-trifluoromethyl-pyrimidin-2-ylamino)-3-methyl-pyrazol-1-yl]-piperidin-1-yl}-2-methyl-propan-1-one
N4-Methyl-N2-[3-methyl-1-(3-methyl-pyridin-4-yl)-1H-pyrazol-4-yl]-5-trifluoromethyl-pyrimidine-2,4-diamine
N2-[1-((R)-2-Methoxy-propyl)-3-methyl-1H-pyrazol-4-yl]-N4-methyl-5-trifluoromethyl-pyrimidine-2,4-diamine
N2-[1-(2,6-Dimethyl-tetrahydro-pyran-4-yl)-5-methyl-1H-pyrazol-4-yl]-N4-methyl-5-trifluoromethyl-pyrimidine-2,4-diamine
N2-[1-(1,1-Dioxo-hexahydro-1$1%6 &-thiopyran-4-yl)-3-methyl-1H-pyrazol-4-yl]-N4-methyl-5-trifluoromethyl-pyrimidine-2,4-diamine
N2-[1-((R)-2-Methoxy-1-methyl-ethyl)-5-methyl-1H-pyrazol-4-yl]-N4-methyl-5-trifluoromethyl-pyrimidine-2,4-diamine
N2-[1-((S)-2-Methoxy-1-methyl-ethyl)-3-methyl-1H-pyrazol-4-yl]-N4-methyl-5-trifluoromethyl-pyrimidine-2,4-diamine
N4-Methyl-N2-[3-methyl-1-((S)-1-oxetan-3-yl-pyrrolidin-3-yl)-1H-pyrazol-4-yl]-5-trifluoromethyl-pyrimidine-2,4-diamine
N4-Methyl-N2-[5-methyl-1-((S)-1-oxetan-3-yl-pyrrolidin-3-yl)-1H-pyrazol-4-yl]-5-trifluoromethyl-pyrimidine-2,4-diamine
N2-[1-(1-Isopropyl-azetidin-3-yl)-5-methyl-1H-pyrazol-4-yl]-N4-methyl-5-trifluoromethyl-pyrimidine-2,4-diamine
N4-Ethyl-N2-[5-methyl-1-(propane-2-sulfonyl)-1H-pyrazol-4-yl]-5-trifluoromethyl-pyrimidine-2,4-diamine
N2-(5-Cyclobutyl-1-methyl-1H-pyrazol-4-yl)-N4-ethyl-5-trifluoromethyl-pyrimidine-2,4-diamine
N2-(3-Cyclobutyl-1-methyl-1H-pyrazol-4-yl)-N4-ethyl-5-trifluoromethyl-pyrimidine-2,4-diamine
N4-Ethyl-N2-{1-[1-(2-methoxy-ethyl)-piperidin-4-yl]-3-methyl-1H-pyrazol-4-yl}-5-trifluoromethyl-pyrimidine-2,4-diamine
N4-Ethyl-N2-{1-[1-(2-methoxy-ethyl)-piperidin-4-yl]-5-methyl-1H-pyrazol-4-yl}-5-trifluoromethyl-pyrimidine-2,4-diamine
N2-{1-[1-(2-Fluoro-ethyl)-piperidin-4-yl]-5-methyl-1H-pyrazol-4-yl}-N4-methyl-5-trifluoromethyl-pyrimidine-2,4-diamine
N2-{1-[1-(2-Fluoro-ethyl)-piperidin-4-yl]-3-methyl-1H-pyrazol-4-yl}-N4-methyl-5-trifluoromethyl-pyrimidine-2,4-diamine
N2-[5-Chloro-1-(3-fluoro-1-methyl-piperidin-4-yl)-1H-pyrazol-4-yl]-N4-methyl-5-trifluoromethyl-pyrimidine-2,4-diamine
N2-(1-Ethanesulfonyl-5-methyl-1H-pyrazol-4-yl)-N4-methyl-5-trifluoromethyl-pyrimidine-2,4-diamine
N4-Methyl-N2-[5-methyl-1-(2-methyl-2-morpholin-4-yl-propyl)-1H-pyrazol-4-yl]-5-trifluoromethyl-pyrimidine-2,4-diamine
N4-Methyl-N2-(3-methyl-1-pyridin-3-ylmethyl-1H-pyrazol-4-yl)-5-trifluoromethyl-pyrimidine-2,4-diamine
N2-(1-Cyclopropanesulfonyl-3-cyclopropyl-1H-pyrazol-4-yl)-N4-methyl-5-trifluoromethyl-pyrimidine-2,4-diamine
N4-Methyl-N2-(5-methyl-1-pyridin-3-ylmethyl-1H-pyrazol-4-yl)-5-trifluoromethyl-pyrimidine-2,4-diamine
(5-Chloro-4-methoxy-pyrimidin-2-yl)-{1-[1-(2-fluoro-ethyl)-piperidin-4-yl]-5-methyl-1H-pyrazol-4-yl}-amine
N4-Methyl-N2-[3-methyl-1-(6-methyl-pyridin-2-ylmethyl)-1H-pyrazol-4-yl]-5-trifluoromethyl-pyrimidine-2,4-diamine
N4-Ethyl-N2-[1-(2-methoxy-ethyl)-3-methyl-1H-pyrazol-4-yl]-5-trifluoromethyl-pyrimidine-2,4-diamine
N4-Ethyl-N2-[1-(2-methoxy-ethyl)-5-methyl-1H-pyrazol-4-yl]-5-trifluoromethyl-pyrimidine-2,4-diamine
1-[4-(4-Ethylamino-5-trifluoromethyl-pyrimidin-2-ylamino)-5-methyl-pyrazol-1-yl]-2-methyl-propan-2-ol
1-[4-(4-Ethylamino-5-trifluoromethyl-pyrimidin-2-ylamino)-3-methyl-pyrazol-1-yl]-2-methyl-propan-2-ol
N2-[1-(1,1-Dimethyl-2-morpholin-4-yl-ethyl)-3-methyl-1H-pyrazol-4-yl]-N4-ethyl-5-trifluoromethyl-pyrimidine-2,4-diamine
N2-[1-(1,1-Dimethyl-2-morpholin-4-yl-ethyl)-3-methyl-1H-pyrazol-4-yl]-N4-methyl-5-trifluoromethyl-pyrimidine-2,4-diamine
N2-[1-(3-Fluoro-1-methyl-piperidin-4-yl)-3-methyl-1H-pyrazol-4-yl]-N4-methyl-5-trifluoromethyl-pyrimidine-2,4-diamine
N4-Cyclopropyl-N2-(1-methanesulfonyl-3-methyl-1H-pyrazol-4-yl)-5-trifluoromethyl-pyrimidine-2,4-diamine
N4-Cyclopropyl-N2-(1-methanesulfonyl-5-methyl-1H-pyrazol-4-yl)-5-trifluoromethyl-pyrimidine-2,4-diamine
1-[3-Chloro-4-(4-methylamino-5-trifluoromethyl-pyrimidin-2-ylamino)-pyrazol-1-yl]-2-methyl-propan-2-ol
2-[4-(4-Ethylamino-5-trifluoromethyl-pyrimidin-2-ylamino)-3-methyl-pyrazol-1-yl]-N-methyl-isobutyramide
N4-Methyl-N2-(3-methyl-1-pyrimidin-2-ylmethyl-1H-pyrazol-4-yl)-5-trifluoromethyl-pyrimidine-2,4-diamine
N2-[5-Chloro-1-(3-fluoro-1-oxetan-3-yl-piperidin-4-yl)-1H-pyrazol-4-yl]-N4-methyl-5-trifluoromethyl-pyrimidine-2,4-diamine
N2-[5-Chloro-1-(3-fluoro-1-oxetan-3-yl-piperidin-4-yl)-1H-pyrazol-4-yl]-N4-ethyl-5-trifluoromethyl-pyrimidine-2,4-diamine
N4-Ethyl-N2-[5-methyl-1-((S)-1-oxetan-3-yl-piperidin-3-yl)-1H-pyrazol-4-yl]-5-trifluoromethyl-pyrimidine-2,4-diamine
N4-Ethyl-N2-[3-methyl-1-((S)-1-oxetan-3-yl-piperidin-3-yl)-1H-pyrazol-4-yl]-5-trifluoromethyl-pyrimidine-2,4-diamine
N4-Methyl-N2-(5-methyl-1-pyridazin-3-ylmethyl-1H-pyrazol-4-yl)-5-trifluoromethyl-pyrimidine-2,4-diamine
N4-Methyl-N2-(3-methyl-1-pyridazin-3-ylmethyl-1H-pyrazol-4-yl)-5-trifluoromethyl-pyrimidine-2,4-diamine
N4-Ethyl-N2-[5-methyl-1-((S)-1-methyl-piperidin-3-yl)-1H-pyrazol-4-yl]-5-trifluoromethyl-pyrimidine-2,4-diamine
N4-Ethyl-N2-[3-methyl-1-((S)-1-methyl-piperidin-3-yl)-1H-pyrazol-4-yl]-5-trifluoromethyl-pyrimidine-2,4-diamine
3-[5-Chloro-4-(4-methylamino-5-trifluoromethyl-pyrimidin-2-ylamino)-pyrazol-1-yl]-2,2-dimethyl-propionitrile
N4-Methyl-N2-[5-methyl-1-(6-methyl-pyridin-2-ylmethyl)-1H-pyrazol-4-yl]-5-trifluoromethyl-pyrimidine-2,4-diamine
N4-Methyl-N2-(5-methyl-1-pyrimidin-2-ylmethyl-1H-pyrazol-4-yl)-5-trifluoromethyl-pyrimidine-2,4-diamine
N4-Methyl-N2-(5-methyl-1-pyrazin-2-ylmethyl-1H-pyrazol-4-yl)-5-trifluoromethyl-pyrimidine-2,4-diamine
N4-Methyl-N2-(3-methyl-1-pyrazin-2-ylmethyl-1H-pyrazol-4-yl)-5-trifluoromethyl-pyrimidine-2,4-diamine
3-[5-Chloro-4-(4-ethylamino-5-trifluoromethyl-pyrimidin-2-ylamino)-pyrazol-1-yl]-2,2-dimethyl-propionitrile
N4-Ethyl-N2-[1-(3-fluoro-1-oxetan-3-yl-piperidin-4-yl)-3-methyl-1H-pyrazol-4-yl]-5-trifluoromethyl-pyrimidine-2,4-diamine
3-Methyl-1-[5-methyl-4-(4-methylamino-5-trifluoromethyl-pyrimidin-2-ylamino)-pyrazol-1-yl]-butan-2-ol
3-Methyl-1-[3-methyl-4-(4-methylamino-5-trifluoromethyl-pyrimidin-2-ylamino)-pyrazol-1-yl]-butan-2-ol
N2-[1-(1-[1,3]Dioxolan-2-ylmethyl-pyrrolidin-3-yl)-3-methyl-1H-pyrazol-4-yl]-N4-ethyl-5-trifluoromethyl-pyrimidine-2,4-diamine
N4-Methyl-N2-(5-methyl-1-pyrimidin-4-ylmethyl-1H-pyrazol-4-yl)-5-trifluoromethyl-pyrimidine-2,4-diamine
N4-Methyl-N2-[5-methyl-1-(1-methyl-1H-pyrazol-3-ylmethyl)-1H-pyrazol-4-yl]-5-trifluoromethyl-pyrimidine-2,4-diamine
N4-Methyl-N2-[3-methyl-1-(1-methyl-1H-pyrazol-3-ylmethyl)-1H-pyrazol-4-yl]-5-trifluoromethyl-pyrimidine-2,4-diamine
3-[3-Chloro-4-(4-methylamino-5-trifluoromethyl-pyrimidin-2-ylamino)-pyrazol-1-yl]-2,2-dimethyl-propionitrile
N4-Ethyl-N2-{3-methyl-1-[1-methyl-1-(4H-[1,2,4]triazol-3-yl)-ethyl]-1H-pyrazol-4-yl}-5-trifluoromethyl-pyrimidine-2,4-diamine
N2-[1-(1-[1,3]Dioxolan-2-ylmethyl-pyrrolidin-3-yl)-5-methyl-1H-pyrazol-4-yl]-N4-ethyl-5-trifluoromethyl-pyrimidine-2,4-diamine
N4-Methyl-N2-(3-methyl-1-pyrimidin-4-ylmethyl-1H-pyrazol-4-yl)-5-trifluoromethyl-pyrimidine-2,4-diamine
N2-(5-Fluoromethyl-1-methyl-1H-pyrazol-4-yl)-N4-methyl-5-trifluoromethyl-pyrimidine-2,4-diamine
N4-Ethyl-N2-{3-methyl-1-[1-methyl-1-(5-methyl-4H-[1,2,4]triazol-3-yl)-ethyl]-1H-pyrazol-4-yl}-5-trifluoromethyl-pyrimidine-2,4-diamine
N4-Methyl-N2-{3-methyl-1-[1-methyl-1-(4H-[1,2,4]triazol-3-yl)-ethyl]-1H-pyrazol-4-yl}-5-trifluoromethyl-pyrimidine-2,4-diamine
N4-Ethyl-N2-[1-(3-fluoro-piperidin-4-yl)-3-methyl-1H-pyrazol-4-yl]-5-trifluoromethyl-pyrimidine-2,4-diamine
2-[5-Methyl-4-(4-methylamino-5-trifluoromethyl-pyrimidin-2-ylamino)-pyrazol-1-yl]-cyclopentanol
2-[3-Methyl-4-(4-methylamino-5-trifluoromethyl-pyrimidin-2-ylamino)-pyrazol-1-yl]-cyclopentanol
N4-ethyl-N2-(3-methyl-1-(2-(5-methyl-1,3,4-oxadiazol-2-yl)propan-2-yl)-1H-pyrazol-4-yl)-5-(trifluoromethyl)pyrimidine-2,4-diamine
N4-ethyl-N2-(3-methyl-1-(2-(4-methyl-4H-1,2,4-triazol-3-yl)propan-2-yl)-1H-pyrazol-4-yl)-5-(trifluoromethyl)pyrimidine-2,4-diamine
N4-ethyl-N2-(3-methyl-1-(2-(1-methyl-1H-1,2,4-triazol-3-yl)propan-2-yl)-1H-pyrazol-4-yl)-5-(trifluoromethyl) pyrimidine-2,4-diamine
N4-methyl-N2-(3-methyl-1-(2-(5-methyl-1,3,4-oxadiazol-2-yl)propan-2-yl)-1H-pyrazol-4-yl)-5-(trifluoromethyl)pyrimidine-2,4-diamine
N4-methyl-N2-(3-methyl-1-(2-(1-methyl-1H-pyrazol-4-yl)propan-2-yl)-1H-pyrazol-4-yl)-5-(trifluoromethyl)pyrimidine-2,4-diamine
N4-methyl-N2-(5-methyl-1-(2-(1-methyl-1H-pyrazol-4-yl)propan-2-yl)-1H-pyrazol-4-yl)-5-(trifluoromethyl) pyrimidine-2,4-diamine
N4-ethyl-N2-(3-methyl-1-(2-(1-methyl-1H-pyrazol-3-yl)propan-2-yl)-1H-pyrazol-4-yl)-5-(trifluoromethyl) pyrimidine-2,4-diamine
N4-ethyl-N2-(3-methyl-1-(2-(1-methyl-1H-pyrazol-5-yl)propan-2-yl)-1H-pyrazol-4-yl)-5-(trifluoromethyl) pyrimidine-2,4-diamine
N4-methyl-N2-(3-methyl-1-(2-(1-methyl-1H-pyrazol-5-yl)propan-2-yl)-1H-pyrazol-4-yl)-5-(trifluoromethyl) pyrimidine-2,4-diamine
N4-methyl-N2-(3-methyl-1-(2-(1-methyl-1H-pyrazol-3-yl)propan-2-yl)-1H-pyrazol-4-yl)-5-(trifluoromethyl) pyrimidine-2,4-diamine
N2-(1′,5-dimethyl-1′H-1,4′-bipyrazol-4-yl)-N4-methyl-5-(trifluoromethyl)pyrimidine-2,4-diamine
N2-(1′,3-dimethyl-1′H-1,4′-bipyrazol-4-yl)-N4-methyl-5-(trifluoromethyl)pyrimidine-2,4-diamine
N2-(1-(2-(4H-1,2,4-triazol-3-yl)propan-2-yl)-3-methyl-1H-pyrazol-4-yl)-N4-methyl-5-(trifluoromethyl)pyrimidine-2,4-diamine
2-(1,3-Dimethyl-1H-pyrazol-4-ylamino)-4-methylamino-pyrimidine-5-carbonitrite
2-(1-ethyl-5-methyl-1H-pyrazol-4-ylamino)-4-(methylamino)pyrimidine-5-carbonitrite
2-(1-isopropyl-3-methyl-1H-pyrazol-4-ylamino)-4-(methylamino)pyrimidine-5-carbonitrite
2-(1-ethyl-3-methyl-1H-pyrazol-4-ylamino)-4-(methylamino)pyrimidine-5-carbonitrite
2-(3-methyl-1-phenyl-1H-pyrazol-4-ylamino)-4-(methylamino)pyrimidine-5-carbonitrile
2-(3-methyl-1-(1-(2,2,2-trifluoroethyl)piperidin-4-yl)-1H-pyrazol-4-ylamino)-4-(methylamino)pyrimidine-5-carbonitrite
2-(5-methyl-1-(1-(2,2,2-trifluoroethyl)piperidin-4-yl)-1H-pyrazol-4-ylamino)-4-(methylamino)pyrimidine-5-carbonitrite
2-(1-methyl-1H-pyrazol-4-ylamino)-4-(methylamino)pyrimidine-5-carbonitrile
2-(5-methyl-1-(2,2,2-trifluoro ethyl)-1H-pyrazol-4-ylamino)-4-(methylamino)pyrimidine-5-carbonitrile
2-(3-methyl-1-(2,2,2-trifluoro ethyl)-1H-pyrazol-4-ylamino)-4-(methylamino)pyrimidine-5-carbonitrile
2-(5-methyl-1-phenyl-1H-pyrazol-4-ylamino)-4-(methylamino)pyrimidine-5-carbonitrile
2-(5-methyl-1-(tetrahydro-2H-pyran-4-yl)-1H-pyrazol-4-ylamino)-4-(methylamino)pyrimidine-5-carbonitrite
2-(3-methyl-1-(tetrahydro-2H-pyran-4-yl)-1H-pyrazol-4-ylamino)-4-(methylamino)pyrimidine-5-carbonitrite
2-(1-ethyl-5-methyl-1H-pyrazol-4-ylamino)-4-(ethylamino)pyrimidine-5-carbonitrile
2-(1-(4-fluorophenyl)-3-methyl-1H-pyrazol-4-ylamino)-4-(methylamino)pyrimidine-5-carbonitrile
2-(1-(difluoromethyl)-3-methyl-1H-pyrazol-4-ylamino)-4-(methylamino)pyrimidine-5-carbonitrile
2-(5-methyl-1-propyl-1H-pyrazol-4-ylamino)-4-(methylamino)pyrimidine-5-carbonitrile
2-(1-(3,5-difluorophenyl)-3-methyl-1H-pyrazol-4-ylamino)-4-(methylamino)pyrimidine-5-carbonitrile
2-(1-(4-chlorophenyl)-3-methyl-1H-pyrazol-4-ylamino)-4-(methylamino)pyrimidine-5-carbonitrile
2-(1-(4-chlorophenyl)-5-methyl-1H-pyrazol-4-ylamino)-4-(methylamino)pyrimidine-5-carbonitrile
2-(3-methyl-1-(pyridin-2-yl)-1H-pyrazol-4-ylamino)-4-(methylamino)pyrimidine-5-carbonitrile
4-(ethylamino)-2-(5-methyl-1-(tetrahydro-2H-pyran-4-yl)-1H-pyrazol-4-ylamino)pyrimidine-5-carbonitrile
4-(ethylamino)-2-(3-methyl-1-(tetrahydro-2H-pyran-4-yl)-1H-pyrazol-4-ylamino)pyrimidine-5-carbonitrile
4-(ethylamino)-2-(3-methyl-1-(oxetan-3-yl)-1H-pyrazol-4-ylamino)pyrimidine-5-carbonitrile
2-(1-isopropyl-5-methyl-1H-pyrazol-4-ylamino)-4-(methylamino)pyrimidine-5-carbonitrile
2-(1,5-dimethyl-1H-pyrazol-4-ylamino)-4-methoxypyrimidine-5-carbonitrile
2-(1,5-dimethyl-1H-pyrazol-4-ylamino)-4-(2,2,2-trifluoroethylamino)pyrimidine-5-carbonitrile
2-(1-ethyl-5-methyl-1H-pyrazol-4-ylamino)-4-methoxypyrimidine-5-carbonitrile
4-(2,2-difluoro ethylamino)-2-(1,5-dimethyl-1H-pyrazol-4-ylamino)pyrimidine-5-carbonitrile
2-(1,5-dimethyl-1H-pyrazol-4-ylamino)-4-(2,2,2-trifluoroethoxy)pyrimidine-5-carbonitrite
2-(1-(cyclopropylmethyl)-3-methyl-1H-pyrazol-4-ylamino)-4-(methylamino) pyrimidine-5-carbonitrile
2-(1-(4,4-difluorocyclohexyl)-5-methyl-1H-pyrazol-4-ylamino)-4-(methylamino) pyrimidine-5-carbonitrile
2-(3-methyl-1-(oxetan-3-yl)-1H-pyrazol-4-ylamino)-4-(2,2,2-trifluoro ethylamino)pyrimidine-5-carbonitrite
2-(5-chloro-1-(tetrahydro-2H-pyran-4-yl)-1H-pyrazol-4-ylamino)-4-(methylamino) pyrimidine-5-carbonitrile
2-(1-Difluoromethyl-5-methyl-1H-pyrazol-4-ylamino)-4-methylamino-pyrimidine-5-carbonitrile
2-(1,5-Dimethyl-1H-pyrazol-4-ylamino)-4-ethylamino-pyrimidine-5-carbonitrile
2-[1-(4-Fluoro-phenyl)-5-methyl-1H-pyrazol-4-ylamino]-4-methylamino-pyrimidine-5-carbonitrile
4-Methylamino-2-(3-methyl-1-propyl-1H-pyrazol-4-ylamino)-pyrimidine-5-carbonitrite
4-Methylamino-2-(5-methyl-1-oxetan-3-yl-1H-pyrazol-4-ylamino)-pyrimidine-5-carbonitrile
4-Methylamino-2-(3-methyl-1-oxetan-3-yl-1H-pyrazol-4-ylamino)-pyrimidine-5-carbonitrile
2-[1-(3,5-Difluoro-phenyl)-5-methyl-1H-pyrazol-4-ylamino]-4-methylamino-pyrimidine-5-carbonitrile
4-(2,2-Difluoro-ethoxy)-2-(1,5-dimethyl-1H-pyrazol-4-ylamino)-pyrimidine-5-carbonitrile
2-[1-(4,4-Difluoro-cyclohexyl)-3-methyl-1H-pyrazol-4-ylamino]-4-methylamino-pyrimidine-5-carbonitrile
N,N-dimethyl-2-(5-methyl-4-(4-(methylamino)-5-(trifluoromethyl)pyrimidin-2-ylamino)-1H-pyrazol-1-yl)acetamide
N,N-dimethyl-2-(3-methyl-4-(4-(methylamino)-5-(trifluoromethyl)pyrimidin-2-ylamino)-1H-pyrazol-1-yl)acetamide
N-methyl-2-(3-methyl-4-(4-(methylamino)-5-(trifluoromethyl)pyrimidin-2-ylamino)-1H-pyrazol-1-yl)acetamide
N-methyl-2-(5-methyl-4-(4-(methylamino)-5-(trifluoromethyl)pyrimidin-2-ylamino)-1H-pyrazol-1-yl)acetamide
N,N,2-trimethyl-2-(5-methyl-4-(4-(methylamino)-5-(trifluoromethyl)pyrimidin-2-ylamino)-1H-pyrazol-1-yl)propanamide
2-methyl-2-(5-methyl-4-(4-(methylamino)-5-(trifluoromethyl)pyrimidin-2-ylamino)-1H-pyrazol-1-yl)-1-(pyrrolidin-1-yl)propan-1-one
2-methyl-2-(5-methyl-4-(4-(methylamino)-5-(trifluoromethyl)pyrimidin-2-ylamino)-1H-pyrazol-1-yl)propanenitrile
1-(3-methyl-4-(4-(methylamino)-5-(trifluoromethyl)pyrimidin-2-ylamino)-1H-pyrazol-1-yl)cyclopropanecarbonitrile
(R)-2-(3-methyl-4-(4-(methylamino)-5-(trifluoromethyl)pyrimidin-2-ylamino)-1H-pyrazol-1-yl)-1-(pyrrolidin-1-yl)propan-1-one
(R)—N,N-dimethyl-2-(3-methyl-4-(4-(methylamino)-5-(trifluoromethyl)pyrimidin-2-ylamino)-1H-pyrazol-1-yl)propanamide
(S)-2-(3-methyl-4-(4-(methylamino)-5-(trifluoromethyl)pyrimidin-2-ylamino)-1H-pyrazol-1-yl)-1-(pyrrolidin-1-yl)propan-1-one
3-(5-methyl-4-(4-(methylamino)-5-(trifluoromethyl)pyrimidin-2-ylamino)-1H-pyrazol-1-yl)propanenitrile
3-(3-methyl-4-(4-(methylamino)-5-(trifluoromethyl)pyrimidin-2-ylamino)-1H-pyrazol-1-yl)propanenitrile
methyl 2-methyl-2-(3-methyl-4-(4-(methylamino)-5-(trifluoromethyl)pyrimidin-2-ylamino)-1H-pyrazol-1-yl)propanoate
methyl 2-methyl-2-(5-methyl-4-(4-(methylamino)-5-(trifluoromethyl)pyrimidin-2-ylamino)-1H-pyrazol-1-yl)propano ate
2-(3-ethyl-4-(4-(methylamino)-5-(trifluoromethyl)pyrimidin-2-ylamino)-1H-pyrazol-1-yl)-2-methylpropanenitrile
(R)-2-(5-methyl-4-(4-(methylamino)-5-(trifluoromethyl)pyrimidin-2-ylamino)-1H-pyrazol-1-yl)-1-(pyrrolidin-1-yl)propan-1-one
(R)—N,N-dimethyl-2-(5-methyl-4-(4-(methylamino)-5-(trifluoromethyl)pyrimidin-2-ylamino)-1H-pyrazol-1-yl)propanamide
(S)-2-(5-methyl-4-(4-(methylamino)-5-(trifluoromethyl)pyrimidin-2-ylamino)-1H-pyrazol-1-yl)-1-(pyrrolidin-1-yl)propan-1-one
(S)—N,N-dimethyl-2-(5-methyl-4-(4-(methylamino)-5-(trifluoromethyl)pyrimidin-2-ylamino)-1H-pyrazol-1-yl)propanamide
(S)-2-(5-methyl-4-(4-(methylamino)-5-(trifluoromethyl)pyrimidin-2-ylamino)-1H-pyrazol-1-yl)propanenitrile
(S)-2-(3-methyl-4-(4-(methylamino)-5-(trifluoromethyl)pyrimidin-2-ylamino)-1H-pyrazol-1-yl)propanenitrile
2-(4-(5-chloro-4-(methylamino)pyrimidin-2-ylamino)-3-methyl-1H-pyrazol-1-yl)-2-methylpropanenitrile
2-(5-chloro-4-(4-(methylamino)-5-(trifluoromethyl)pyrimidin-2-ylamino)-1H-pyrazol-1-yl)-2-methylpropanenitrile
2-(3-cyclopropyl-4-(4-(methylamino)-5-(trifluoromethyl)pyrimidin-2-ylamino)-1H-pyrazol-1-yl)-2-methylpropanenitrile
2,2-dimethyl-3-(3-methyl-4-(4-(methylamino)-5-(trifluoromethyl)pyrimidin-2-ylamino)-1H-pyrazol-1-yl)propanenitrile
2,2-dimethyl-3-(5-methyl-4-(4-(methylamino)-5-(trifluoromethyl)pyrimidin-2-ylamino)-1H-pyrazol-1-yl)propanenitrile
1-(5-chloro-4-(4-(methylamino)-5-(trifluoromethyl)pyrimidin-2-ylamino)-1H-pyrazol-1-yl)cyclopropanecarbonitrile
N-tert-butyl-2-methyl-2-(3-methyl-4-(4-(methylamino)-5-(trifluoromethyl)pyrimidin-2-ylamino)-1H-pyrazol-1-yl)propanamide
2-methyl-2-(3-methyl-4-(4-(methylamino)-5-(trifluoromethyl)pyrimidin-2-ylamino)-1H-pyrazol-1-yl)-N-(2,2,2-trifluoroethyl)propanamide
2-(5-chloro-4-(4-(methylamino)-5-(trifluoromethyl)pyrimidin-2-ylamino)-1H-pyrazol-1-yl)-N-ethyl-2-methylpropanamide
N-(cyclopropylmethyl)-2-methyl-2-(5-methyl-4-(4-(methylamino)-5-(trifluoromethyl)pyrimidin-2-ylamino)-1H-pyrazol-1-yl)propanamide
N-(cyclopropylmethyl)-2-methyl-2-(3-methyl-4-(4-(methylamino)-5-(trifluoromethyl)pyrimidin-2-ylamino)-1H-pyrazol-1-yl)propanamide
N-ethyl-1-(3-methyl-4-(4-(methylamino)-5-(trifluoromethyl)pyrimidin-2-ylamino)-1H-pyrazol-1-yl)cyclobutanecarboxamide
N-isopropyl-2-methyl-2-(5-methyl-4-(4-(methylamino)-5-(trifluoromethyl)pyrimidin-2-ylamino)-1H-pyrazol-1-yl)propanamide
1-(3-methyl-4-(4-(methylamino)-5-(trifluoromethyl)pyrimidin-2-ylamino)-1H-pyrazol-1-yl)cyclobutanecarbonitrile
2-(4-(4-(cyclopropylamino)-5-(trifluoromethyl)pyrimidin-2-ylamino)-3-methyl-1H-pyrazol-1-yl)-2-methylpropanenitrile
N,2-dimethyl-2-(3-methyl-4-(4-(methylamino)-5-(trifluoromethyl)pyrimidin-2-ylamino)-1H-pyrazol-1-yl)propanamide
1-(5-chloro-4-(4-(methylamino)-5-(trifluoromethyl)pyrimidin-2-ylamino)-1H-pyrazol-1-yl)cyclopropanecarbonitrile
2-[4-(5-Chloro-4-methoxy-pyrimidin-2-ylamino)-5-methyl-pyrazol-1-yl]-2-methyl-propionic acid methyl ester
2-[4-(5-Chloro-4-methoxy-pyrimidin-2-ylamino)-3-methyl-pyrazol-1-yl]-2-methyl-propionic acid methyl ester
(S)—N,N-Dimethyl-2-[3-methyl-4-(4-methylamino-5-trifluoromethyl-pyrimidin-2-ylamino)-pyrazol-1-yl]-propionamide
R)-2-[3-Methyl-4-(4-methylamino-5-trifluoromethyl-pyrimidin-2-ylamino)-pyrazol-1-yl]-propionitrile
2-[4-(5-Chloro-4-methoxy-pyrimidin-2-ylamino)-3-cyclopropyl-pyrazol-1-yl]-2-methyl-propionitrile
(R)-2-[5-Methyl-4-(4-methylamino-5-trifluoromethyl-pyrimidin-2-ylamino)-pyrazol-1-yl]-propionitrile
N-Ethyl-2-[3-methyl-4-(4-methylamino-5-trifluoromethyl-pyrimidin-2-ylamino)-pyrazol-1-yl]-isobutyramide
N-Ethyl-2-[5-methyl-4-(4-methylamino-5-trifluoromethyl-pyrimidin-2-ylamino)-pyrazol-1-yl]-isobutyramide
1-[5-Methyl-4-(4-methylamino-5-trifluoromethyl-pyrimidin-2-ylamino)-pyrazol-1-yl]-cyclobutanecarboxylic acid ethylamide
2-[5-Methyl-4-(4-methylamino-5-trifluoromethyl-pyrimidin-2-ylamino)-pyrazol-1-yl]-N-(2,2,2-trifluoro-ethyl)-isobutyramide
N-Isopropyl-2-[3-methyl-4-(4-methylamino-5-trifluoromethyl-pyrimidin-2-ylamino)-pyrazol-1-yl]-isobutyramide
N-Methyl-2-[5-methyl-4-(4-methylamino-5-trifluoromethyl-pyrimidin-2-ylamino)-pyrazol-1-yl]-isobutyramide
1-[5-Methyl-4-(4-methylamino-5-trifluoromethyl-pyrimidin-2-ylamino)-pyrazol-1-yl]-cyclobutanecarbonitrile
N-tert-Butyl-2-[5-methyl-4-(4-methylamino-5-trifluoromethyl-pyrimidin-2-ylamino)-pyrazol-1-yl]-isobutyramide
2-[4-(4-Ethylamino-5-trifluoromethyl-pyrimidin-2-ylamino)-5-methyl-pyrazol-1-yl]-N-methyl-isobutyramide
2-[4-(4-Cyclopropylamino-5-trifluoromethyl-pyrimidin-2-ylamino)-3-methyl-pyrazol-1-yl]-N-methyl-isobutyramide
2-[4-(4-Cyclopropylamino-5-trifluoromethyl-pyrimidin-2-ylamino)-5-methyl-pyrazol-1-yl]-N-methyl-isobutyramide
2-[4-(4-Ethylamino-5-trifluoromethyl-pyrimidin-2-ylamino)-3-methyl-pyrazol-1-yl]-2-methyl-propionitrile or
2-(3-chloro-4-(4-(methylamino)-5-(trifluoromethyl)pyrimidin-2-ylamino)-1H-pyrazol-1-yl)-2-methylpropanenitrile.

### Experimental Examples

### Mass Spectrometry Analysis

Excised gel pieces were reduced, alkylated, and subjected to overnight trypsin digestion. For phosphomapping analyses, digested peptides were extracted, dried, and resuspended in 10% acetonitrile/5% formic acid. For quantitative analysis, a mixture of isotopically labeled internal standard peptides (Cell Signaling Technologies, Danvers, Mass.) was introduced to the digested gel pieces prior to extracting the peptides, and then following extraction, dried peptides were resuspended in 3% acetonitrile/5% formic acid/10% H₂O₂ at least 30 min prior to analysis to allow for complete oxidation of methionine containing peptides. Peptides were separated using a standard gradient comprised of H₂O/acetonitrile/formic acid delivered by a nanoAcquity UPLC (Waters, Milford Mass.) and analyzed on an LTQ-Orbitrap XL (Thermo, San Jose, Calif.) mass spectrometer using conditions previously described (Phu and Kirkpatrick, Mol cell Proteomics 2011) In the present study, the mass spectrometer was operated in data-dependent mode with a 60,000 resolution full MS scan collected in the Orbitrap and MS/MS for the eight most intense ions collected in the ion trap. Spectra data were converted to mzxml and searched using the target-decoy strategy against the human Uniprot database and rough filtered to 5% FDR using a linear discriminant analysis (Huttlin and Gygi, Cell 2011). The localizations of putative phosphorylation sites were optimized and confidence scores assigned using the AScore algorithm (Beausoliel and Gygi, Nat Biotech 2006), with scores of 13 and 19 representing the 95% and 99% confidence levels respectively. Confidently matching phosphopeptides with uncertain localizations (Ascore<13) have not been reported. For the remaining peptide-spectral matches, manual validation of at least one representative spectra for each unique phosphopeptide sequence was required for inclusion herein. For quantitative analysis, full MS scans were integrated using Qualbrowser software (Thermo, San Jose, Calif.) to determine the peak areas for heavy and light m/z ions of the most abundant charge states of each LRRK2 phosphopeptide.

### Western Blotting

Cultured cells and mouse brains were homogenized in RIPA buffer (25 mM Tris-HCl (pH 7.6), 150 mM NaCl, 1% NP-40, 1% sodium deoxycholate, 0.1% SDS) containing Complete protease and phosphotase inhibitor cocktail (Roche). Cell/tissue lysate was cleared via centrifugation at 20,000 g for 30 minutes at 4 C. Protein concentration of the supernatant was measured using BCA assay (Pierce). 20 ug of lysate was loaded onto 3-8% Tris-Acetate gels or 4˜12% Bis-Tris gels (Invitrogen) and immunoblotted with the following antibodies: phospho-LRRK2 (Custom generated), LRRK2 (Epitomics), FLAG, Tubulin (Sigma). The Li-Cor Odyssey system was used for Western blot detection.

### Generation of Stable LRRK2 Cell Line

FLAG-tagged full length human LRRK2 was inserted into pTre2Hygro vector (Clontech). QuickChange Site-directed mutagenesis kit was used to introduce 3 Parkinson's mutations, R1441G, Y1699C and G2019S (Stratagene). The tetracycline regulated Tet-On system was used to generate a LRRK2 stable cell line (Gossen et al). HEK293 Tet-On cells (Clontech) was transfected with the LRRK2 plasmid mentioned above together with GFP. Hygromycin was used for stable cell selection and 48 single clones were selected using clone-disk. LRRK2 expression levels were compared using Western blots to identify the highest expresser line. The concentration of hygromycin used for selection was determined by a killing curve assay. Gossen, M., et al. (1995) Science 268: 1766-1769.

### Inhibitor Treatment of LRRK2 Cells

HEK293 Tet-On LRRK2 G2019S/R144G/Y1699C inducible stable cell line was grown in complete media (High Glucose DMEM, 10% Tet System FBS, 1% Glutamax, 1% NEAA, 100 ug/mL G418, 30 ug/mL Hygromycin B). Cells were induced with 1 ug/mL of doxycycline for 16 hours at 37 degrees Celsius. Cells were treated with inhibitors for one hour, and then lysed with cold MSD Tris Lysis Buffer.

### Immunoassay

Anti-FLAG M2 antibody (Sigma, Saint Louis, Mo., USA) was immobilized onto a standard Binding 96 Well Small Spot Plate (MSD, Gaithersburg, Md., USA). Antibody was diluted in low salt TBS buffer (16.6 mM Tris, 50 mM NaCl, 16.6 mM HEPES, 0.015% Triton X-100) to 333 ug/mL and 5 ul was spotted and allowed to dry overnight. Plates were blocked for 1 hour with Sea Block (Thermo Scientific, Rockford, Ill., USA), then washed with MSD Tris Wash Buffer.

Cell lysate was transferred to the assay plate and incubated for 2 hours at room temperature. After washing with MSD Wash Buffer, Anti-phospho-Ser 1292 pLRRK antibody was diluted to 0.370 ug/mL in Complete Assay Buffer (20 mM Tris, 150 mM NaCl, 0.05% Tween 20,10% Sea Block, 1% normal mouse serum), added and incubated at room temperature for one hour. After washing, SULFOTAG Goat Anti-Rabbit antibody was diluted to 1 ug/mL in Complete Assay Buffer, added and incubated at room temperature for one hour. After washing plates with MSD Wash Buffer, 1×MSD Read Buffer T was added and the plate was measured on the MSD Sector Imager 6000 according to manufactures instructions.

### Hippocampal Culture and Transfection

Dissociated hippocampal neuron cultures were prepared as described (Brewer et al.) Briefly, 200 hippocampal neurons mm⁻² were plated 24-well clear plates coated or S-well culture chamber (BD) with 30 μg/ml poly-D-lysine and 2 μg/ml laminin. For IHC, cells were fixed in 4% formaldehyde, 4% sucrose in PBS for 10 min at room temperature. Antibodies were applied in GDB buffer (0.1% gelatin, 0.3% TX-100, 450 mM NaCl, 32% 0.1M Phosphate buffer [pH 7.4]). Images were acquired using an LSM710 confocal system or GE InCell automated imager. Image analysis was done using InCell Developer.-G. J. Brewer et al., “Optimizing survival of hippocampal neurons in B27-supplemented Neurobasal, a new serum-free medium combination”. J. Neuro. Meth. 170:181-187. Neurons were co-transfected with GFP and LRRK2 (1:20 ratio) at 6 days in vitro using Lipofectamine 2000.

### Immunofluorescence

Cells were fixed with 4% paraformaldehyde/4% sucrose in phosphate-buffered saline (PBS, pH 7.4). Following permeabilization (0.1% Triton-X in PBS), blocking (5% NGS, 0.1% Triton X-100 in PBS) and primary antibody incubation, antibodies were visualized using secondary antibodies conjugated to Alexa 405, Alexa 488 or Alexa 568(Invitrogen). The following antibodies were used for primary immunostaining mouse anti-FLAG (Sigma), rabbit anti-Flag (Cell signaling), chicken anti-GFP (Invitrogen), rabbit anti-MAP2 (Millipore), rabbit beta-tubulin (Abcam), rabbit anti-P62 (Santa Cruz), rabbit anti-LC3 (Cell signaling), rabbit Lys63-specific anti-Ubiquitin (Millipore).

### Image Acquisition and Quantitative Analysis

For neurite outgrowth quantification, neurons were fixed 11 days post-transfection. Cells were stained with GFP and MAP2 for dendrite observation. Confocal images of transfected neurons were obtained using Zeiss LSM 710 20X (NA?) objective. Dendrites from neurons were traced using Neurolucida software (MicroBrightField). Sholl analysis and total dendritic length were analyzed with Neurolucida Explorer (MicroBrightField). Statistical analysis was performed using JMP 9 software. P values were obtained using (non-paired Student's t test, one way ANOVA test ?). All image acquisition and analysis were done in a blind manner.

For LRRK2 cellular localization, neurons were fixed 3-4 days post-transfection. Confocal images of transfected neurons were obtained using Zeiss LSM 710 100X (NA?) or Leica SP5 confocal microscope 63× (NA) with Z-section size of ?. Quantitative analysis of cellular localization of various LRRK2 constructs was performed blind to the experimental conditions.

### Animal Study

G1023 was dissolved in 1% RC591 in water. Mice in treatment groups (n=3 per group) received intraperitoneal injection at 10, 30, or 100 mg/kg. Vehicle group (n=2) received 1% RC591. Dosing volume was calculated based on animal's weight and did not exceed 500 ul. Animals were taken down 6 hours post-dose. The brain, spleen, and lymph nodes were rapidly harvested and dissected. All animals were handled according to IACUC guidelines.

### Cell-Based Assay Using MSD Platform

Based on the foregoing, a cell-based assay was made using anti-FLAG antibody capture of LRRK2 and anti-phospho-Serine 1292-LRRK2 antibody on Meso Scale Discovery (MSD, Gaithersburg Md.) plates, with detection of captured anti-phospho-Serine 1292-LRRK2 antibody with MSD sulfotag goat anti-rabbit antibody using the MSD sector reader according to the procedure in the Example below. FIG. 5A shows the Western blot for a tetracycline-On inducible LRRK2 R1441G/Y1699C/G2019S triple mutant cell line expressing LRRK2 upon treatment with 1 ug/mL of doxycycline, with anti-phospho-Serine 1292-LRRK2 antibody shown in the first (upper) row, and anti-FLAG epitope (control) in the second (lower) row. FIG. 5B is a graphical representation of the Western blot illustration of FIG. 5A, with relative luminescence shown on the vertical axis.

Using the assay with the LRRK2 inhibitor Sutent (SU-11248 or sunitinib, American Custom Chemicals Inc.) LRRK2 activity was measured by detection of anti-phospho-Serine 1292-LRRK2 antibody.

Inhibition of LRRK2 Autophosphorylation In vivo

The in vivo effect of LRRK2 inhibitors on S1292 autophosphorylation, and thus LRRK2 inhibition, can be determined with the invention. Three month old BAC transgenic mice overexpressing LRRK2 G2019S were administered the LRRK2 inhibitor [3-methoxy-4-(4-methylamino-5-trifluoromethyl-pyrimidin-2-ylamino)-phenyl]-morpholin-4-yl-methanone at 10 mg/kg, 30 mg/kg and 100 mg/kg, orally. After six hours, hippocampus, lymph node and spleen tissue were harvested and levels of autophosphorylation at S1292 was determined by detecting anti-phospho-Serine 1292-LRRK2 antibody as described herein.

### HEK293 R1441G/Y1699C/G2019S LRRK2 HEK 293 Tet-On Cell Line

Using standard mutagenesis technique, the R1441G, Y1699C, G2019S mutations and a N-terminal FLAG tag were introduced into the full length human LRRK2 sequence (hererinafter “LRRK2-3M”). LRRK2-3M was cloned into pTre2Hyg vector (Clontech, Cat. No. 631014) using NotI and EcoRV restriction sites. pTre2Hyg-LRRK2-3M in combination with pCaggs_GFP was then introduced into Clontech's Tet-On HEK293 cells following their standard protocol (http://www.clontech.com/images/pt/PT3898-1.pdf).

### LRRK2-3M Cell Line Handling and Induction Procedures

### Materials:

DMEM with high glucose (Site Service)*

### Glutamax 100X (Invitrogen 35050)*

### NEAA 100X (Invitrogen 11140)*

Tet system FBS (Clontech 631105)*
G418 100 mg/ml stock dissolved in H2O (Clontech 631308)*

### Pen/Strep (Invitrogen)*

Doxycycline 1 mg/ml dissolved in H2O (Clontech)*
Hygromycin B 100 mg/ml stock (Invivogen)
* available online

### DMEM Medium for HEK293 Tet_On LRRK2 Triple Mutant GFP Cells:

DMEM with glutamine (1000 ml)

Add 100 ml Tet system FBS, f.c. 10%

Add 10 ml of 100X pen/strep solution

Add 10 ml of 100X Glutamax

Add 10 ml of 100X NEAA

Add 1 ml of 100 mg/ml G418, f.c. 100 ug/ml

Add 300 ul of 100 mg/ml Hygromycin B, f.c. 30 ug/ml

### Day 1:

Plate cells at 20% confluence (Varies based on difference in surface areas of different plates)

### Day 2:

16˜20 hours after plating, directly add Doxycycline (1 mg/ml stock) solution to culture wells yielding 1 ug/ml f.c. of Dox. (if the handling volume is too little, dilute stock Dox to 10X or 100X in culture media first)

### Day 3:

24 hours after Dox induction, cells are ready for compound treatment

pLRRK2 Antibody

Rabbits were immunized with peptide CPNEMGKL-pS-KIWDLPL-amide according to standard procedures (Yenzyme). Serum from immunized rabbits were first purified by passing through an agarose column conjugated with peptide CPNEMGKL-pS-KIWDLPL-amide. Antibodies bound to the column were eluted. The purified material then was passed through an agarose column conjugated with the peptide CPNEMGKLSKIWDLPL-amide to absorb the antibodies cross-reacting to un-phosphorylated peptide.

BAC Mice

BAC mice overexpressing G2019S and/or other Parkinson's disease mutations may be prepared generally by the procedures of Marshall et al. in “Generation of BAC Transgenic Mice”, Methods in Mol. Biol. Vol. 256 (2004) pp. 159-182.

Assay Using Meso Scale Discovery Platform

HEK293 cells were transiently transfected with a plasmid containing FLAG epitope-tagged full-length LRRK2 cDNA under the control of the CMV promotor. Transfections were carried out in 10-cm dishes using Fugene 6 reagent (Roche Applied Science, Mannheim, Germany) according to the manufacturer's instructions. 24 hours post-transfection cells were harvested, counted, seeded into 96-well plates and allowed to attach for 1 hour prior to compound addition.

Cells were treated with compounds for 1 hour, then medium was removed and cells lysed in MSD (Meso Scale Discovery, Gaithersburg, Md.) lysis buffer (Meso Scale Discovery, Gaithersburg, Md.) supplemented with protease and phosphatase inhibitors.

Assay detection plates were prepared by spotting 2 ug of anti-FLAG monoclonal antibody (Sigma Aldrich, St Louis, Mo.) in each well of 96-well MSD plates, followed by blocking with Sea-Block (Thermo Fisher, Rockford, Ill.). Cell Lysates were transferred to MSD plates, incubated for 2 hours, then washed. Captured phospho-LRRK2 was detected by addition of anti-phospho-Ser1292-LRRK2, incubating for 2 hours, followed by washing. Detection of captured antibody with MSD Sulfotag goat anti-rabbit antibody using the MSD Sector reader was carried out according to the manufacturer's instructions.

Other Assay Formats

In a variation of the above assay format, determination of phospho-Ser(1292) in cell lysates is achieved using cells transfected (stably or transiently) or virally transduced with cDNA encoding all or part of LRRK2 protein, with or without fusion to epitope tag protein. Capture of overexpressed LRRK2 or fragment of LRRK2, with or without fused epitope tag or fusion protein partner, from cell lysate is done with immobilized antibody, directed against LRRK2 sequence, other epitope tag or fusion protein. Solid-phase matrix for capture can include ELISA plates, Meso Scale Discovery plates (Meso Scale Discovery, Gaithersberg, Md.), Membranes (e.g. nitrocellulose, PVDF). Detection of captured phospho-Ser1292-LRRK2 with antibody against phospho-Ser1292 by methods including: ELISA, DELFIA, Electrochemiluminescence, fluorescence.

Another variation of the above assay format utilizes capturing phospho-Ser1292-LRRK2 from cell lysates with immobilized anti-phospho-Ser1292-LRRK2 antibody, followed by detection of captured LRRK2 with antibody against LRRK2 sequence, epitope tag or fusion protein partner.

Plate-based detection of endogenous phospho-Ser(1292) from cell lysates in any of the formats described above may use anti-LRRK2 and anti-phospho-Ser(1292) antibodies.

Plate-based detection of phospho-Ser(1292) in intact cells may be done by Immunofluorescent detection of phospho-Ser(1292) in fixed and permeabilized cells by microscopic detection and quantitation of fluorescence intensity, or by bulk fluorescence (e.g. LiCor “In-Cell Western”)

Plate-based detection of phospho-Ser(1292) in intact cells may utilize:

Colorimetric/immunohistochemical detection and quantitation of anti-phospho-Ser(1292) binding to fixed and permeabilized cells; Western Blotting of total cell lysates or immunoprecipitated endogenous or transfected LRRK2 with anti-phospho-Ser(1292); or Western blotting of protein immunoprecipited with anti-phosphoSer(1292) antibody followed by detection with anti-LRRK or antibody against fusion protein/epitope tag.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

1. A method for identifying a patient or subject having Parkinson's disease or at risk of developing Parkinson's disease, the method comprising determining the level of S1292 autophosphorylation in said subject, wherein an elevated level correlates with having Parkinson's disease or risk of developing Parkinson's disease.

2. The method of claim 1, further comprising obtaining or acquiring a LRRK2-containing sample from the subject.

3. The method of claim 2, further comprising measuring, monitoring or otherwise detecting the amount or level of S1292 autophosphorylation in the subject sample.

4. The method of claim 3, further comprising comparing the amount or level of S1292 autophosphorylation in the subject sample with the amount or level of S1292 autophosphorylation in one or more control samples.

5. A method for predicting the sensitivity of a subject to a LRRK2 modulator, or efficacy of a LRRK2 modulator for treating Parkinson's disease, the method comprising determining the level of S1292 autophosphorylation in said subject, whereby elevated levels of S1292 autophosphorylation correlate with efficacy of said inhibitor for treating said disease.

6. The method of claim 5, further comprising obtaining or acquiring a first LRRK2-containing sample from a subject.

7. The method of claim 6, further comprising administering a LRRK2 modulator to the subject.

8. The method of claim 7, further comprising obtaining or acquiring a second LRRK2-containing sample from the subject after administering the LRRK2 modulator to the subject.

9. The method of claim 8, further comprising measuring, monitoring or otherwise detecting the amount or level of S1292 autophosphorylation in the first subject sample.

10. The method of claim 9, further comprising measuring, monitoring or otherwise detecting the amount or level of S1292 autophosphorylation in the second subject sample.

11. The method of claim 10, further comprising comparing the amount or level of S1292 autophosphorylation in the first subject sample with the amount or level of S1292 autophosphorylation in the second subject sample.

12. The method of claim 5, wherein said LRRK2 modulator is a compound of formula I: 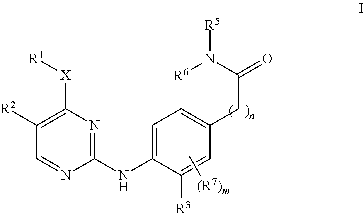
or a pharmaceutically acceptable salt thereof,
wherein:
m is from 0 to 3;
X is: —NR^{a}—; —O—; or —S(O)ᵣ— wherein r is from 0 to 2 and R^{a} is hydrogen or C₁₋₆alkyl;
R¹ is: C₁₋₆alkyl; halo-C₁₋₆alkyl; C₂₋₆alkenyl; C₂₋₆alkynyl; halo-C₁₋₆alkyl; C₁₋₆alkoxy-C₁₋₆alkyl; hydroxy-C₁₋₆alkyl; amino-C₁₋₆alkyl; C₁₋₆alkylsulfonyl-C₁₋₆alkyl; C₃₋₆cycloalkyl optionally substituted with C₁₋₆alkyl or halo; C₃₋₆cycloalkyl-C₁₋₆alkyl wherein the C₃₋₆cycloalkyl portion is optionally substituted with C₁₋₆alkyl; tetrahydropyranyl; tetrahydrofuranyl; tetrahydro furanyl-C₁₋₆alkyl; oxetanyl; or oxetan-C₁₋₆alkyl;
or R¹ and R^{a} together with the atoms to which they are attached may form a three to six membered ring that may optionally include an additional heteroatom selected from O, N and S, and which is substituted with oxo, halo or C₁₋₆alkyl;
R² is: halo; C₁₋₆alkoxy; cyano; C₂₋₆alkynyl; C₂₋₆alkenyl; halo-C₁₋₆alkyl; halo-C₁₋₆alkoxy; C₃₋₆cycloalkyl wherein the C₃₋₆cycloalkyl portion is optionally substituted with C₁₋₆alkyl; C₃₋₆cycloalkyl-C₁₋₆alkyl wherein the C₃₋₆cycloalkyl portion is optionally substituted with C₁₋₆alkyl; tetrahydrofuranyl; tetrahydrofuranyl-C₁₋₆alkyl; acetyl; oxetanyl; or oxetan-C₁₋₆alkyl;
R³ is: —OR⁴; halo; cyano; C₁₋₆alkyl; halo-C₁₋₆alkyl; C₃₋₆cycloalkyl optionally substituted with C₁₋₆alkyl; C₃₋₆cycloalkyl-C₁₋₆alkyl wherein the C₃₋₆cycloalkyl portion is optionally substituted with C₁₋₆alkyl; tetrahydrofuranyl; tetrahydrofuranyl-C₁₋₆alkyl; oxetanyl; or oxetan-C₁₋₆alkyl;
R⁴ is: hydrogen; C₁₋₆alkyl; halo-C₁₋₆alkyl; C₁₋₆alkoxy-C₁₋₆alkyl; C₃₋₆cycloalkyl optionally substituted with C₁₋₆alkyl or halo; C₃₋₆cycloalkyl-C₁₋₆alkyl wherein the C₃₋₆cycloalkyl portion is optionally substituted with C₁₋₆alkyl or halo; tetrahydrofuranyl; tetrahydro furanyl-C₁₋₆alkyl; oxetanyl; or oxetan-C₁₋₆alkyl;
R⁵ is: hydrogen; or C₁₋₆alkyl;
n is 0 or 1;
R⁶ is: hydrogen; C₁₋₆alkyl; C₁₋₆alkoxy-C₁₋₆alkyl; hydroxy-C₁₋₆alkyl; amino-C₁₋₆alkyl; C₃₋₆cycloalkyl; C₃₋₆cycloalkyl-C₁₋₆alkyl; heterocyclyl; or heterocyclyl-C₁₋₆alkyl; wherein the C₃₋₆cycloalkyl, C₃₋₆cycloalkyl-C₁₋₆alkyl, heterocyclyl and heterocyclyl-C₁₋₆alkyl each may be optionally substituted with one, two, three or four groups groups independently selected from: C₁₋₆alkyl; halo-C₁₋₆alkyl; C₁₋₆alkoxy; halo-C₁₋₆alkoxy; hydroxy; hydroxy-C₁₋₆alkyl; halo; nitrile; C₁₋₆alkyl-carbonyl; C₁₋₆alkyl-sulfonyl; C₃₋₆cycloalkyl; C₃₋₆cycloalkyl-C₁₋₆alkyl; C₃₋₆cycloalkyl-carbonyl; amino; or heterocyclyl; or two of the groups together with the atoms to which they are attached may form a five or six-membered ring;
or R⁵ and R⁶ together with the nitrogen atom to which they are attached form a three- to seven-membered ring that optionally includes an additional heteroatom selected from O, N and S(O), and which is optionally substituted with one, two, three or four groups independently selected from: C₁₋₆alkyl; halo-C₁₋₆alkyl; C₁₋₆alkoxy; halo-C₁₋₆alkoxy; hydroxy; C₁₋₆alkoxy-C₁₋₆alkyl; hydroxy-C₁₋₆alkyl; halo, nitrile; C₁₋₆alkyl-carbonyl; C₁₋₆alkyl-sulfonyl; C₃₋₆cycloalkyl; C₃₋₆cycloalkyl-C₁₋₆alkyl; C₃₋₆cycloalkyl-carbonyl; amino; or heterocyclyl; or two of the groups together with the atoms to which they are attached may form a five or six-membered ring; and
R⁷ is: halo; C₁₋₆alkyl; C₁₋₆alkoxy; halo-C₁₋₆alkyl; or halo-C₁₋₆alkoxy.

13. The method of claim 5, wherein said LRRK2 modulator is a compound of formula II: 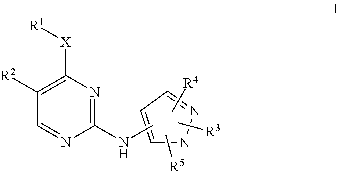
or a pharmaceutically acceptable salt thereof,
wherein:
X is: —NR^{a}—; or —O— wherein R^{a} is hydrogen or C₁₋₆alkyl;
R¹ is: C₁₋₆alkyl; C₂₋₆alkenyl; C₂₋₆alkynyl; halo-C₁₋₆alkyl; C₁₋₆alkoxy-C₁₋₆alkyl; hydroxy-C₁₋₆alkyl; amino-C₁₋₆alkyl; C₁₋₆alkylsulfonyl-C₁₋₆alkyl; C₃₋₆cycloalkyl optionally substituted one or more times with C₁₋₆alkyl; C₃₋₆cycloalkyl-C₁₋₆alkyl wherein the C₃₋₆cycloalkyl portion is optionally substituted one or more times with C₁₋₆alkyl; heterocyclyl optionally substituted one or more times with R⁷; or heterocyclyl-C₁₋₆alkyl optionally substituted one or more times with R⁷;
or X and R¹ together form C₁₋₆alkyl; C₁₋₆alkoxy-C₁₋₆alkyl; C₃₋₆cycloalkyl optionally substituted one or more times with R⁶; or C₃₋₆cycloalkyl-C₁₋₆alkyl wherein the C₃₋₆cycloalkyl portion is optionally substituted one or more times with R⁶;
or R¹ and R^{a} together with the atoms to which they are attached may form a three- to six-membered heterocyclic ring optionally substituted one or more times with R⁷;
R² is: C₁₋₆alkyl; halo; C₁₋₆alkoxy; cyano; C₂₋₆alkynyl; C₂₋₆alkenyl; halo-C₁₋₆alkyl; halo-C₁₋₆alkoxy; C₃₋₆cycloalkyl optionally substituted one or more times with R⁶; C₃₋₆cycloalkyl-C₁₋₆alkyl wherein the C₃₋₆cycloalkyl portion is optionally substituted one or more times with R⁶; —OR^{b} wherein R^{b} is C₁₋₆alkyl, C₃₋₆cycloalkyl optionally substituted one or more times with R⁶, or C₃₋₆cycloalkyl-C₁₋₆alkyl wherein the C₃₋₆cycloalkyl portion is optionally substituted one or more times with R⁶; or —C(O)—R^{c} wherein R^{c} is C₁₋₆alkyl, C₁₋₆alkoxy, amino, or heterocyclyl optionally substituted one or more times with R⁷;
R³ is: hydrogen; C₁₋₆alkyl; halo-C₁₋₆alkyl; C₂₋₆alkenyl; C₂₋₆alkynyl; hydroxy-C₁₋₆alkyl; C₁₋₆alkoxy-C₁₋₆alkyl; cyano-C₁₋₆alkyl; C₁₋₆alkylsulfonyl; C₁₋₆alkylsulfonylC₁₋₆alkyl; amino-C₁₋₆alkyl; C₃₋₆cycloalkyl optionally substituted one or more times with R⁶; C₃₋₆cycloalkyl-C₁₋₆alkyl wherein the C₃₋₆cycloalkyl portion is optionally substituted one or more times with R⁶; C₃₋₆cycloalkyl-sulfonyl wherein the C₃₋₆cycloalkyl portion is optionally substituted one or more times with R⁶; heterocyclyl optionally substituted one or more times with R⁷; heterocyclyl-C₁₋₆alkyl wherein the heterocyclyl portion is optionally substituted one or more times with R⁷; aryl optionally substituted one or more times with R⁸; aryl-C₁₋₆alkyl wherein the aryl portion is optionally substituted one or more times with R⁸; heteroaryl optionally substituted one or more times with R⁸; heteroaryl-C₁₋₆alkyl wherein the heteroaryl portion is optionally substituted one or more times with R⁸; or —Y—C(O)—R^{d};
Y is C₂₋₆alkylene or a bond;
R^{d} is C₁₋₆alkyl, C₁₋₆alkoxy, amino, C₁₋₆alkyl-amino, di-C₁₋₆alkyl-amino, halo-C₁₋₆alkyl-amino, di-halo-C₁₋₆alkyl-amino, halo-C₁₋₆alkyl, hydroxy-C₁₋₆alkyl, hydroxy, C₁₋₆alkoxy-C₁₋₆alkyl, cyano-C₁₋₆alkyl, C₁₋₆alkylsulfonylC₁₋₆alkyl, amino-C₁₋₆alkyl, C₃₋₆cycloalkyl optionally substituted one or more times with R⁶, C₃₋₆cycloalkyl-C₁₋₆alkyl wherein the C₃₋₆cycloalkyl portion is optionally substituted one or more times with R⁶ heterocyclyl optionally substituted one or more times with R⁷, or heterocyclyl-C₁₋₆alkyl wherein the heterocyclyl portion is optionally substituted one or more times with R⁷;
R⁴ is: hydrogen; C₁₋₆alkyl; halo; cyano; halo-C₁₋₆alkyl; C₂₋₆alkenyl; C₂₋₆alkynyl; C₁₋₆alkoxy; C₁₋₆alkoxy-C₁₋₆alkyl; hydroxy-C₁₋₆alkyl; C₃₋₆cycloalkyl optionally substituted one or more times with R⁶; C₃₋₆cycloalkyl-C₁₋₆alkyl wherein the C₃₋₆cycloalkyl portion is optionally substituted one or more times with R⁶; or —Y—C(O)—R^{d};
R⁵ is: hydrogen; or C₁₋₆alkyl;
each R⁶ is independently: C₁₋₆alkyl; halo-C₁₋₆alkyl; C₁₋₆alkoxy; oxo; cyano; halo; or Y—C(O)—R^{d};
each R⁷ is independently: C₁₋₆alkyl; halo-C₁₋₆alkyl; halo; oxo; C₁₋₆alkoxy; C₁₋₆alkylsulfonyl; C₁₋₆alkoxy-C₁₋₆alkyl; cyano; —Y—C(O)—R^{d}; heterocyclyl; heterocyclyl-C₁₋₆alkyl; C₃₋₆cycloalkyl; C₃₋₆cycloalkyl-C₁₋₆alkyl; or C₃₋₆cycloalkylsulfonyl; and
each R⁸ is independently: oxo; C₁₋₆alkyl; halo-C₁₋₆alkyl; halo; C₁₋₆alkyl-sulfonyl; C₁₋₆alkoxy; C₁₋₆alkoxy-C₁₋₆alkyl; cyano; hetoeryclyl; heterocyclyl-C₁₋₆alkyl; —Y—C(O)—R^{d}; C₃₋₆cycloalkyl, C₃₋₆cycloalkyl-C₁₋₆alkyl, or C₃₋₆cycloalkyl-sulfonyl.

14. a method for identifying subjects having Parkinson's disease or that are at risk of developing Parkinson's disease, the method comprising:
obtaining or acquiring a LRRK2-containing sample from a subject;
measuring, monitoring or otherwise detecting the amount or level of S1292 autophosphorylation in the subject sample; and
comparing the amount or level of S1292 autophosphorylation in the subject sample with the amount or level of S1292 autophosphorylation in one or more control samples.

15. A method for determining subject response to therapy involving a LRRK2 modulator, the method comprising:
obtaining or acquiring a first LRRK2-containing sample from a subject;
administering a LRRK2 modulator to the subject;
obtaining or acquiring a second LRRK2-containing sample from the subject after administering the LRRK2 modulator to the subject;
measuring, monitoring or otherwise detecting the amount or level of S1292 autophosphorylation in the first subject sample;
measuring, monitoring or otherwise detecting the amount or level of S1292 autophosphorylation in the second subject sample; and
comparing the amount or level of S1292 autophosphorylation in the first subject sample with the amount or level of S1292 autophosphorylation in the second subject sample.

16. An assay for screening compounds and compositions for inhibition of LRRK2 activity, the method comprising:
exposing LRRK2 or a fragment thereof to a potential inhibitor compound; and
detecting the level or amount of phosphorylation associated with S1292 on the LRRK2.
